(19) United States
(12) Patent Application Publication
(10) Pub. No.: US 2026/0297590 A1
(43) Pub. Date: Oct. 1, 2026

(54) PDE3B RNA INTERFERENCE AGENTS

(71) Applicant: ELI LILLY AND COMPANY, Indianapolis, IN (US)

(72) Inventors: Ricardo J. SAMMS, Carmel, IN (US); Eitaro AIHARA, Carmel, IN (US); Jesline T. ALEXANDER-CHACKO, Carmel, IN (US); Jorge ALSINA-FERNANDEZ, Indianapolis, IN (US); Robert Andrew BROWN, Carmel, IN (US); Christine Chih-Tao CHENG, Carmel, IN (US); Guillermo S. CORTEZ, Indianapolis, IN (US); Supriya DEY, New Palestine, IN (US); Yan DING, Carmel, IN (US); Isabel Cristina GONZALEZ VALCARCEL, Indianapolis, IN (US); William Howard JACKSON, Indianapolis, IN (US); Katarina Lynn KEEL, Hemlock, MI (US); Xiaoyong LU, Carmel, IN (US); Ruben Martin MARTINEZ, Indianapolis, IN (US); Fonny Kartawinata MERCHANT, New Palestine, IN (US); James Warren PERFIELD, II, Indianapolis, IN (US); Francisco Alcides VALENZUELA, Indianapolis, IN (US); Jibo WANG, Carmel, IN (US); Aaron David WROBLESKI, Indianapolis, IN (US); Hugo AVILA, Fishers, IN (US); Seth Andrew BAWEL, Brownsburg, IN (US); Kevin Michael RULEY, Indianapolis, IN (US); Kostas GAVARDINAS, Carmel, IN (US); Regan Elizabeth WEBERDING, Indianapolis, IN (US)

(21) Appl. No.: 19/420,912

(22) Filed: Dec. 16, 2025

Related U.S. Application Data
(60) Provisional application No. 63/735,643, filed on Dec. 18, 2024.
(60) Provisional application No. 63/917,676, filed on Nov. 14, 2025.

(51) Int. Cl. C12N 15/113; A61K 9/00; A61K 31/712; A61K 31/7125; A61K 31/713; A61K 38/00; A61P 3/04; C07K 7/06

(52) U.S. Cl. CPC C12N 15/1137; A61K 9/0019; A61K 31/712; A61K 31/7125; A61K 31/713; A61P 3/04; C07K 7/06; A61K 38/00; C12N 2310/11; C12N 2310/14; C12N 2310/312; C12N 2310/315; C12N 2310/321; C12N 2310/323; C12N 2310/3513; C12N 2310/3515

(57) ABSTRACT

Provided herein are PDE3B RNAi agents, conjugates thereof, and compositions comprising a PDE3B RNAi agent. Also provided herein are methods of using the PDE3B RNAi agents or compositions comprising a PDE3B RNAi agent for reducing PDE3B expression, and/or treating PDE3B associated metabolic disorder in a subject.

### SEQUENCE LISTING

The present application is being filed along with a Sequence Listing in ST.26 XML format. The Sequence Listing is provided as a file titled “31335_US” created Nov. 6, 2025 and is 3,913,216 bytes in size. The Sequence Listing information in the ST.26 XML format is incorporated herein by reference in its entirety.

### FIELD OF INVENTION

The present disclosure relates to the delivery of therapeutics, in some instances oligonucleotides, to adipose tissue, and more particularly, to peptide conjugates of such therapeutics that are targeted to adipose tissue to modulate genes expression.

### BACKGROUND

The human gene PDE3B encodes the enzyme phosphodiesterase 3B, which regulates cellular levels of cyclic AMP (cAMP) and cyclic GMP (cGMP) by breaking them down, including in adipocytes. In adipocytes, PDE3B plays a role in regulation of adipocyte lipolysis, lipid oxidation, and energy homeostasis. Loss-of-function mutations in the PDE3B gene in humans are associated with reduced adiposity and protection from metabolic disease. No direct modulator of PDE3B quantity or activity has been approved as a treatment for any disease or condition.

RNA interference (RNAi) is a highly conserved regulatory mechanism in which RNA molecules are involved in sequence-specific suppression of gene expression by double-stranded RNA molecules (dsRNA) (Fire et al., Nature 391:806-811, 1998).

Adipose tissue is a connective tissue and an endocrine organ, participating in various physiological processes, including energy homeostasis, glucose metabolism, and inflammation. Dysregulation of adipose tissue function, including accumulation of excess adipose, can lead to metabolic disorders, including obesity. Therefore, modulation of adipose tissue gene expression can be a potential strategy for the treatment of these disorders.

There remains a need for therapeutic agents that can inhibit or adjust the expression of PDE3B for treating PDE3B associated metabolic disorders, e.g., by utilizing RNAi, including delivering RNAi agents to adipose tissue.

### SUMMARY OF INVENTION

Provided herein are PDE3B RNAi agents which include a sense strand and an antisense strand, wherein the sense strand and the antisense strand form a duplex, selected from the group consisting of:
the sense strand comprises a first nucleic acid sequence of SEQ ID NO: 80, and the antisense strand comprises a second nucleic acid sequence of SEQ ID NO: 125;
the sense strand comprises a first nucleic acid sequence of SEQ ID NO: 81, and the antisense strand comprises a second nucleic acid sequence of SEQ ID NO: 126;
the sense strand comprises a first nucleic acid sequence of SEQ ID NO: 82, and the antisense strand comprises a second nucleic acid sequence of SEQ ID NO: 127;
the sense strand comprises a first nucleic acid sequence of SEQ ID NO: 83, and the antisense strand comprises a second nucleic acid sequence of SEQ ID NO: 128;
the sense strand comprises a first nucleic acid sequence of SEQ ID NO: 84, and the antisense strand comprises a second nucleic acid sequence of SEQ ID NO: 129;
the sense strand comprises a first nucleic acid sequence of SEQ ID NO: 85, and the antisense strand comprises a second nucleic acid sequence of SEQ ID NO: 130;
the sense strand comprises a first nucleic acid sequence of SEQ ID NO: 86, and the antisense strand comprises a second nucleic acid sequence of SEQ ID NO: 131; and
the sense strand comprises a first nucleic acid sequence of SEQ ID NO: 87, and the antisense strand comprises a second nucleic acid sequence of SEQ ID NO: 132;
the sense strand comprises a first nucleic acid sequence of SEQ ID NO: 88, and the antisense strand comprises a second nucleic acid sequence of SEQ ID NO: 133;
the sense strand comprises a first nucleic acid sequence of SEQ ID NO: 89, and the antisense strand comprises a second nucleic acid sequence of SEQ ID NO: 134;
the sense strand comprises a first nucleic acid sequence of SEQ ID NO: 90, and the antisense strand comprises a second nucleic acid sequence of SEQ ID NO: 135;
the sense strand comprises a first nucleic acid sequence of SEQ ID NO: 91, and the antisense strand comprises a second nucleic acid sequence of SEQ ID NO: 136;
the sense strand comprises a first nucleic acid sequence of SEQ ID NO: 92, and the antisense strand comprises a second nucleic acid sequence of SEQ ID NO: 137;
the sense strand comprises a first nucleic acid sequence of SEQ ID NO: 93, and the antisense strand comprises a second nucleic acid sequence of SEQ ID NO: 138;
the sense strand comprises a first nucleic acid sequence of SEQ ID NO: 94, and the antisense strand comprises a second nucleic acid sequence of SEQ ID NO: 139;
the sense strand comprises a first nucleic acid sequence of SEQ ID NO: 95, and the antisense strand comprises a second nucleic acid sequence of SEQ ID NO: 140;
the sense strand comprises a first nucleic acid sequence of SEQ ID NO: 96, and the antisense strand comprises a second nucleic acid sequence of SEQ ID NO: 141; and
the sense strand comprises a first nucleic acid sequence of SEQ ID NO: 97, and the antisense strand comprises a second nucleic acid sequence of SEQ ID NO: 142;
the sense strand comprises a first nucleic acid sequence of SEQ ID NO: 98, and the antisense strand comprises a second nucleic acid sequence of SEQ ID NO: 143;
the sense strand comprises a first nucleic acid sequence of SEQ ID NO: 99, and the antisense strand comprises a second nucleic acid sequence of SEQ ID NO: 144;
the sense strand comprises a first nucleic acid sequence of SEQ ID NO: 100, and the antisense strand comprises a second nucleic acid sequence of SEQ ID NO: 145;
the sense strand comprises a first nucleic acid sequence of SEQ ID NO: 101, and the antisense strand comprises a second nucleic acid sequence of SEQ ID NO: 146;
the sense strand comprises a first nucleic acid sequence of SEQ ID NO: 102, and the antisense strand comprises a second nucleic acid sequence of SEQ ID NO: 147;
the sense strand comprises a first nucleic acid sequence of SEQ ID NO: 103, and the antisense strand comprises a second nucleic acid sequence of SEQ ID NO: 148;
the sense strand comprises a first nucleic acid sequence of SEQ ID NO: 104, and the antisense strand comprises a second nucleic acid sequence of SEQ ID NO: 149;
the sense strand comprises a first nucleic acid sequence of SEQ ID NO: 105, and the antisense strand comprises a second nucleic acid sequence of SEQ ID NO: 150;
the sense strand comprises a first nucleic acid sequence of SEQ ID NO: 106, and the antisense strand comprises a second nucleic acid sequence of SEQ ID NO: 151; and
the sense strand comprises a first nucleic acid sequence of SEQ ID NO: 107, and the antisense strand comprises a second nucleic acid sequence of SEQ ID NO: 152;
the sense strand comprises a first nucleic acid sequence of SEQ ID NO: 108, and the antisense strand comprises a second nucleic acid sequence of SEQ ID NO: 153;
the sense strand comprises a first nucleic acid sequence of SEQ ID NO: 109, and the antisense strand comprises a second nucleic acid sequence of SEQ ID NO: 154;
the sense strand comprises a first nucleic acid sequence of SEQ ID NO: 110, and the antisense strand comprises a second nucleic acid sequence of SEQ ID NO: 155;
the sense strand comprises a first nucleic acid sequence of SEQ ID NO: 111, and the antisense strand comprises a second nucleic acid sequence of SEQ ID NO: 156;
the sense strand comprises a first nucleic acid sequence of SEQ ID NO: 112, and the antisense strand comprises a second nucleic acid sequence of SEQ ID NO: 157;
the sense strand comprises a first nucleic acid sequence of SEQ ID NO: 113, and the antisense strand comprises a second nucleic acid sequence of SEQ ID NO: 158;
the sense strand comprises a first nucleic acid sequence of SEQ ID NO: 114, and the antisense strand comprises a second nucleic acid sequence of SEQ ID NO: 159;
the sense strand comprises a first nucleic acid sequence of SEQ ID NO: 115, and the antisense strand comprises a second nucleic acid sequence of SEQ ID NO: 160;
the sense strand comprises a first nucleic acid sequence of SEQ ID NO: 116, and the antisense strand comprises a second nucleic acid sequence of SEQ ID NO: 161; and
the sense strand comprises a first nucleic acid sequence of SEQ ID NO: 117, and the antisense strand comprises a second nucleic acid sequence of SEQ ID NO: 162;
the sense strand comprises a first nucleic acid sequence of SEQ ID NO: 118, and the antisense strand comprises a second nucleic acid sequence of SEQ ID NO: 163;
the sense strand comprises a first nucleic acid sequence of SEQ ID NO: 119, and the antisense strand comprises a second nucleic acid sequence of SEQ ID NO: 164;
the sense strand comprises a first nucleic acid sequence of SEQ ID NO: 120, and the antisense strand comprises a second nucleic acid sequence of SEQ ID NO: 165;
the sense strand comprises a first nucleic acid sequence of SEQ ID NO: 121, and the antisense strand comprises a second nucleic acid sequence of SEQ ID NO: 166;
the sense strand comprises a first nucleic acid sequence of SEQ ID NO: 122, and the antisense strand comprises a second nucleic acid sequence of SEQ ID NO: 167;
the sense strand comprises a first nucleic acid sequence of SEQ ID NO: 123, and the antisense strand comprises a second nucleic acid sequence of SEQ ID NO: 168; and
the sense strand comprises a first nucleic acid sequence of SEQ ID NO: 124, and the antisense strand comprises a second nucleic acid sequence of SEQ ID NO: 169,
or a dsRNA 1-45 or 100-158 of Table 4,
wherein optionally one or more nucleotides of the sense strand and the antisense strand are independently modified nucleotides, and
wherein optionally one or more internucleotide linkages of the sense strand and the antisense strand are modified internucleotide linkages.

In certain embodiments, at least one of the sense strand and the antisense strand comprises one or more independently modified nucleotides.

In certain embodiments, at least one of the sense strand and the antisense strand comprises one or more modified internucleotide linkages.

In some embodiments, at least one of the sense strand and the antisense strand comprises one or more independently modified oligonucleotides and one or more modified internucleotide linkages.

In some embodiments, one or more nucleotides of the sense strand are modified nucleotides. In certain embodiments, each nucleotide of the sense strand is a modified nucleotide. In some embodiments, one or more nucleotides of the antisense strand are modified nucleotides. In some embodiments, each nucleotide of the antisense strand is a modified nucleotide.

In some embodiments, the modified nucleotide is a 2′-fluoro modified nucleotide, 2′-O-methyl modified nucleotide, 2′ deoxy nucleotide (DNA), or 2′-O-alkyl modified nucleotide.

In some embodiments, the PDE3B RNAi agent has four 2′-fluoro modified nucleotides at positions 7, 9, 10, and 11 from the 5′ end of the sense strand.

In some embodiments, nucleotides at positions other than positions 7, 9, 10, and 11 of the sense strand are 2′-O-methyl modified nucleotides.

In some embodiments, the antisense strand has four 2′-fluoro modified nucleotides at positions 2, 6, 14, and 16 from the 5′ end of the antisense strand.

In some embodiments, nucleotides at positions other than positions 2, 6, 14 and 16 of the antisense strand are 2′-O-methyl modified nucleotides.

In some embodiments, the sense strand has three 2′-fluoro modified nucleotides at positions 9, 10, and 11 from the 5′ end of the sense strand.

In some embodiments, nucleotides at positions other than positions 9, 10, and 11 of the sense strand are 2′-O-methyl modified nucleotides.

In some embodiments, the antisense strand has five 2′-fluoro modified nucleotides at positions 2, 5, 7, 14, and 16 from the 5′ end of the antisense strand.

In some embodiments, nucleotides at positions other than positions 2, 5, 7, 14, and 16 of the antisense strand are 2′-O-methyl modified nucleotides.

In some embodiments, the antisense strand has five 2′-fluoro modified nucleotides at positions 2, 5, 8, 14, and 16 from the 5′ end of the antisense strand.

In some embodiments, nucleotides at positions other than positions 2, 5, 8, 14, and 16 of the antisense strand are 2′-O-methyl modified nucleotides.

In some embodiments, the sense strand has three 2′-fluoro modified nucleotides at positions 9, 10, and 11 from the 5′ end of the sense strand.

In some embodiments, the antisense strand has five 2′-fluoro modified nucleotides at positions 2, 3, 7, 14, and 16 from the 5′ end of the antisense strand.

In some embodiments, nucleotides at positions other than positions 2, 3, 7, 14, and 16 of the antisense strand are 2′-O-methyl modified nucleotides.

In some embodiments, the antisense strand has five 2′-fluoro modified nucleotides at positions 2, 14, and 16 from the 5′ end of the antisense strand.

In some embodiments, nucleotides at positions other than positions 2, 14, and 16 of the antisense strand are 2′-O-methyl modified nucleotides.

In some embodiments, the sense strand has three 2′-fluoro modified nucleotides at positions 9, 10, and 11 from the 5′ end of the sense strand.

In some embodiments, the sense strand and the antisense strand have one or more modified internucleotide linkages. The modified internucleotide linkage may be a phosphorothioate linkage. In some embodiments, the sense strand has four or five phosphorothioate linkages. In some embodiments, the antisense strand has four or five phosphorothioate linkages.

In some embodiments, the antisense strand has 5′ phosphate or 5′ vinylphosphonate.

In some embodiments, the sense strand comprises an abasic moiety or inverted abasic moiety.

In one embodiment of the PDE3B RNAi agent, the sense strand and the antisense strand comprise a pair of nucleic acid sequences selected from the group consisting of the dsRNAs of Table 4 or of Table 6 herein.

In another embodiment, a PDE3B RNAi agent is provided. The RNAi agent is of the Formula:

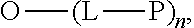

wherein O is a double stranded RNA (dsRNA) comprising a sense stand and an antisense strand, wherein the antisense strand is complementary to PDE3B mRNA; wherein L is a linker or a bond;
wherein P is an NPR-C-binding protein or an NPR-C-binding peptide comprising SEQ ID NO: 1 (GX₁₁IDX₁₄I), wherein X₁₁ is arginine, proline, or hydroxyproline, and X₁₄ is arginine or N-methylarginine; and
wherein n is 1, 2, 3, or 4.

In some embodiments, X₁₁ is R. In some embodiments, X₁₄ is R.

In some embodiments, P comprises SEQ ID NO: 2 (GRIDRI).

In some embodiments, P comprises SEQ ID NO: 3 (SX₇X₈X₉GX₁₁IDX₁₄I), wherein:
X₇ is glycine, alanine, proline, hydroxyproline, serine, or cysteine;
X₈ is phenylalanine, or cyclohexylalanine, and
X₉ is glycine, alanine, or serine.

In some embodiments, X₇ is C. In some embodiments, X₉ is G.

In some embodiments, P comprises SEQ ID NO: 4 (SCFGGRIDRI).

In some embodiments, P comprises SEQ ID NO: 5 (FGGRIDRIGA).

In some embodiments, P comprises SEQ ID NO: 6 (RSSX₇FGGRIDRI), wherein X₇ is serine or cysteine.

In some embodiments, P comprises SEQ ID NO: 7 (RSSX₇FGGRIDRIGA). In some embodiments, X₇ is cysteine. In other embodiments, X₇ is serine.

In some embodiments, P comprises SEQ ID NO: 8 (X₇-Cha-X₉GX₁₁IDX₁₄I), wherein:
X₇ is proline, hydroxyproline, glycine, cysteine, or alanine;
X₉ is alanine, serine, or glycine;
X₁₁ is proline, hydroxyproline, or arginine; and
X₁₄ is arginine or N-methylarginine.

In some embodiments, P comprises SEQ ID NO: 9 (fSp-Cha-aGPIDRI).

In some embodiments, P comprises a sequence selected from any one of SEQ ID NOs: 11-57.

In some embodiments, P is a linear peptide. In other embodiments, P is a cyclic peptide. In such embodiments, P can be cyclized by covalent attachment of a side chain of a first cysteine residue to a side chain of a second cysteine residue. The covalent attachment may include a disulfide bond, or may include a thioacetal moiety.

In some embodiments, P comprises a C-terminal hydroxyl.

In some embodiments, P comprises a C-terminal amide.

In some embodiments, P is an NPR-C-binding antibody, or a fragment thereof.

In some embodiments, L comprises a linker core and one or more spacers. In specific embodiments, L comprises Spacer1-Linker Core-Spacer2. In some embodiments, the Linker Core is selected from Table 7. In some embodiments, Spacer1 and Spacer 2 are selected from Table 8. In some embodiments, L is selected from Table 9.

In some embodiments, n is 1 or 2.

In some embodiments, L is attached to the 5′ end of the sense strand and to the N-terminal end of the peptide. In other embodiments, L is attached to the 5′ end of the sense strand and to the C-terminal end of the peptide. In other embodiments, L is attached to the 3′ end of the sense strand and to the N-terminal end of the peptide. In other embodiments, L is attached to the 3′ end of the sense strand and to the C-terminal end of the peptide.

In some embodiments, the RNAi agent includes a fatty acid (FA). In some embodiments, FA is attached to O. In some embodiments, FA is attached to P.

In some embodiments, the RNAi agent comprises (FA)m-O-L-P or O-L-P-(FA)ₘ, wherein m is an integer of 1 to 4.

In some embodiments, the RNAi agent further comprises a Spacer3. In such embodiments, the RNAi agent may be of the formula (FA-Spacer3)ₘ-O-L-P or O-L-P-(Spacer3-FA)ₘ, wherein m is an integer of 1 to 4. In other embodiments, the RNAi agent may be of the formula O-L-(FA-Spacer3)ₘ-P or O-(Spacer3-FA)ₘ-L-P, wherein m is an integer of 1 to 4. In such embodiments, m may be 1 or 2.

In an RNAi agent comprising formula O-(L-P)ₙ, the sense strand and the antisense strand may form a duplex, selected from the group consisting of: including a sense strand and an antisense strand, wherein the sense strand and the antisense strand form a duplex, comprising a sense strand and an antisense strand, wherein the sense strand and the antisense strand form a duplex, wherein the duplex comprises one of dsRNA 1-45 or 100-158 of Table 4 (such as, for example, a duplex wherein the sense strand is SEQ ID NO: 80 and the antisense strand SEQ ID NO: 125, and so forth), wherein optionally one or more nucleotides of the sense strand and the antisense strand are independently modified nucleotides, and wherein optionally one or more internucleotide linkages of the sense strand and the antisense strand are modified internucleotide linkages.

In specific embodiments, the sense strand and the antisense strand comprise a pair of nucleic acid sequences selected from the group consisting of dsRNA 46-99 or 159-285 of Table 6 (such as, for example, a duplex wherein the sense strand is SEQ ID NO: 170 and the antisense strand SEQ ID NO: 224, and so forth).

In one embodiment, the present disclosure provides a PDE3B RNAi agent of Formula:

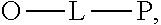

wherein O comprises a comprising a sense stand and an antisense strand, wherein
the antisense strand is complementary to PDE3B mRNA;
wherein L is a linker comprising the formula:

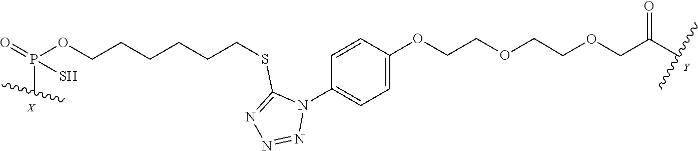

wherein P is an NPR-C-binding peptide comprising SEQ ID NO: 270.

In such an embodiment of the PDE3B RNAi agent the sense strand comprises a first nucleic acid sequence of SEQ ID NO: 112, and the antisense strand comprises a second nucleic acid sequence of SEQ ID NO: 157; or the sense strand comprises a first nucleic acid sequence of SEQ ID NO: 123, and the antisense strand comprises a second nucleic acid sequence of SEQ ID NO: 168; or the sense strand comprises a first nucleic acid sequence of SEQ ID NO: 107, and the antisense strand comprises a second nucleic acid sequence of SEQ ID NO: 321; or the sense strand comprises a first nucleic acid sequence of SEQ ID NO: 111, and the antisense strand comprises a second nucleic acid sequence of SEQ ID NO: 156; or the sense strand comprises a first nucleic acid sequence of SEQ ID NO: 105, and the antisense strand comprises a second nucleic acid sequence of SEQ ID NO: 150; or the sense strand comprises a first nucleic acid sequence of SEQ ID NO: 102, and the antisense strand comprises a second nucleic acid sequence of SEQ ID NO: 147; or the sense strand comprises a first nucleic acid sequence of SEQ ID NO: 104, and the antisense strand comprises a second nucleic acid sequence of SEQ ID NO: 149; or the sense strand comprises a first nucleic acid sequence of SEQ ID NO: 97, and the antisense strand comprises a second nucleic acid sequence of SEQ ID NO: 142.

In one embodiment, the antisense strand is modified with 2′-fluoro at each of positions 2, 5, 7, 14, and 16. In one embodiment, all other positions of the antisense strand are modified with 2′-OMe.

In one embodiment of the PDE3B RNAi agent, the antisense strand comprises phosphorothioate linkages between positions 1 and 2, 2 and 3, 20 and 21, 21 and 22, and 22 and 23. In one embodiment, the antisense comprises a vinylphosphonate moiety conjugated to the 5′ end. In one embodiment, the sense strand is modified with 2′-fluoro at each of positions 9, 10, and 11. In one embodiment, all other positions of the sense strand are modified with 2′-OMe. In one embodiment, the sense strand comprises phosphorothioate linkages between positions 1 and 2, 19 and 20, and 20 and 21. In one embodiment, the sense strand comprises an inverted abasic moiety conjugated at the 5′ end, wherein the inverted abasic moiety is conjugated to the sense strand by a phosphorothioate linkage. In another embodiment, the linker L is attached to a 3′ end of the sense strand.

In some embodiments, the PDE3B RNAi agent is selected from among C1-C37 as disclosed herein.

In some embodiments, provided herein are conjugates comprising Formula (I): 0-(L-P)ₙ, wherein O comprises a PDE3B RNAi agent; wherein L is a linker or a bond; wherein P is an NPR-C-binding antibody or a fragment thereof, and wherein n is an integer of 1 to 4. In some embodiments, P is a heteromab that binds NPR-C. In some embodiments, P is a Fab, VHH or scFv that binds NPR-C.

The present disclosure also provides for a pharmaceutical composition comprising the PDE3B RNAi agent, and a pharmaceutically acceptable carrier.

The present disclosure also provides a method of treating a disease or condition of adipose tissue in a patient in need thereof, comprising administering to the patient an effective amount of the PDE3B RNAi agent or the pharmaceutical composition. The disease or condition may be obesity or obesity-related comorbidity. The RNAi agent or pharmaceutical composition may be administered intravenously or subcutaneously.

The present disclosure also provides for the PDE3B RNAi agent, or the pharmaceutical composition, for use in a therapy.

The present disclosure provides the PDE3B RNAi agent, or the pharmaceutical composition thereof, for use in the treatment of a disease or condition of adipose tissue. The disease or condition may be obesity or an obesity-related comorbidity.

The present disclosure provides for use of PDE3B RNAi agent, or the pharmaceutical composition thereof, in the manufacture of a medicament for treating a disease or condition of adipose tissue. The disease or condition may be obesity or an obesity-related comorbidity.

The present disclosure provides a method of delivering an oligonucleotide to an adipose tissue, comprising administering to a subject PDE3B RNAi agent or a pharmaceutical composition thereof.

### DETAILED DESCRIPTION

Provided herein are PDE3B RNAi agents and compositions comprising a PDE3B RNAi agent. Also provided herein are methods of using the PDE3B RNAi agents or compositions comprising a PDE3B RNAi agent for treating PDE3B associated metabolic disorder, such as obesity and/or an obesity-associated co-morbidity, in a subject.

In one aspect, provided herein are PDE3B RNAi agent comprising a sense strand and an antisense strand, wherein the sense strand and the antisense strand form a duplex, and wherein the antisense strand is complementary to a region of PDE3B mRNA. In some embodiments, provided herein are PDE3B RNAi agent comprising a sense strand and an antisense strand, wherein the sense strand and the antisense strand form a duplex, wherein the sense strand comprises a nucleic acid sequence selected from any one of SEQ ID NOs:80-124, 170-223, 271-316, 369-493, and 577-582; and the antisense strand comprises a nucleic acid sequence selected from any one of SEQ ID NOs: 125-169, 224-268, 317-368, and 464-576; wherein optionally one or more nucleotides of the sense strand and the antisense strand are independently modified nucleotides, and wherein optionally one or more internucleotide linkages of the sense strand and the antisense strand are modified internucleotide linkages.

In another aspect, the PDE3B RNAi agent may be a conjugate including a nucleic acid component. In one instance, the conjugate may include an NPR-C binding peptide and a therapeutic agent, such as an oligonucleotide. The NPR-C binding peptide can deliver the therapeutic agent such as an oligonucleotide to adipose tissue with good selectivity. The conjugates optionally include a linker between the NPR-C binding peptide and the therapeutic agent (e.g., an oligonucleotide). The conjugates can also include one or more fatty acids. The present disclosure also includes methods and uses of treating diseases and conditions associated with adipose tissue (e.g., obesity or obesity-related comorbidity) by the conjugates or pharmaceutical composition described herein.

In one aspect, provided herein are conjugates comprising Formula (I): O-(L-P)ₙ, wherein O comprises an oligonucleotide; wherein L is a linker or a bond; wherein P is an NPR-C-binding peptide, wherein n is an integer of 1 to 4. In some embodiments, O is an antisense oligonucleotide (ASO), a double stranded RNA (dsRNA), such as a dsRNA including a sense strand and an antisense strand, or a guide RNA. In some embodiments, P is any NPR-C-binding peptide described herein, e.g., an NPR-C-binding peptide in Tables 1-3. In some embodiments, P is an NPR-C-binding antibody or a fragment thereof. In some embodiments, n is 1. In some embodiments, n is 2.

In some embodiments, provided herein are conjugates comprising Formula (I): O-(L-P)ₙ, wherein O comprises a PDE3B RNAi agent; wherein L is a linker or a bond; wherein P is an NPR-C-binding antibody or a fragment thereof, and wherein n is an integer of 1 to 4. In some embodiments, P is a heteromab that binds NPR-C. In some embodiments, P is a Fab, VHH or scFv that binds NPR-C.

In some embodiments, L comprises a linker core and one or more spacers. In some embodiments, L comprises Spacer1-Linker Core-Spacer2.

In some embodiments, the conjugate further comprises a fatty acid (FA) or lipid. In some embodiments, FA is attached to O. In some embodiments, FA is attached to P. In some embodiments, FA is attached to L.

In some embodiments, the conjugate comprises (FA)ₘ-O-L-P or O-L-P-(FA)ₘ, wherein m is an integer of 1 to 4. In some embodiments, m is 1 or 2.

In some embodiments, the conjugate further comprises a Spacer3. In some embodiments, the conjugate comprises (FA-Spacer3)ₘ-O-L-P or O-L-P-(Spacer3-FA)ₘ, wherein m is an integer of 1 to 4. In some embodiments, m is 1 or 2. In some embodiments, the conjugate comprises FA-Spacer3-O-L-P or O-L-P-Spacer3-FA. In some embodiments, the conjugate comprises O-L-(FA-Spacer3)ₘ-P or O-(Spacer3-FA)ₘ-L-P, wherein m is an integer of 1 to 4. In some embodiments, m is 1 or 2. In some embodiments, the conjugate comprises O-L-FA-Spacer3-P or O-Spacer3-FA-L-P.

### NPR-C Binding Peptide or Protein (P)

Natriuretic peptides (NPs) are a class of endogenous hormones which confer cardiovascular protection through regulation of body fluid homeostasis. They include several structurally related peptide hormones: Atrial Natriuretic Peptide (ANP) and variants such as Urodilatin and mANP, Brain Natriuretic Peptide (BNP), C-type Natriuretic Peptide (CNP) and Dendroaspis Natriuretic Peptide (DNP). Three subtypes of natriuretic peptide receptors (NPR) have been described and include NPR-A, NPR-B and NPR-C. Of these, NPR-C is enriched in adipose tissue, where it acts in the natriuretic peptide system to bind and clear NPs, removing them from circulating blood (Maack et al., Science 238:675-678 (1987)).

Nucleic acid therapeutics, including RNA interference (RNAi) agents such as small interfering RNA (siRNA) and antisense oligonucleotides (ASO) have the potential to selectively target and modulate individual genes related to a disease state. The effectiveness of such treatment can be related to how much of the therapeutic is delivered to the organ, tissue, or even cell of interest.

The conjugates provided herein include a peptide or protein that binds a human NPR-C receptor (“NPR-C binding peptide” or “NPR-C binding protein”). In some embodiments, the peptide or protein binds NPR-C receptor with good affinity and selectivity.

In some embodiments, P is an NPR-C-binding peptide. In some embodiments, the NPR-C binding peptide is derived from ANP. Wild-type human ANP is a 28 amino acid peptide having a 17 amino acid loop formed by an intramolecular disulfide linkage between two cysteine residues present at positions 7 and 23 (SEQ ID NO: 10). It is a cardiac hormone that generally acts to maintain the cardiovascular system. It is part of the body's natural defense against hypoxia and pathological cardiac wall stress. Wild type ANP exhibits similar binding affinity for the receptors NPR-A and NPR-C, but a synthetic peptide C-ANP₄₋₂₃ has high selectivity for NPR-C, not binding to NPR-A at detectable levels. Both linear and cyclic C-ANP₄₋₂₃ bind NPR-C with high potency.

In some embodiments, the NPR-C binding peptide comprises a consensus sequence GX₁₁IDX₁₄I (SEQ ID NO: 1), wherein X₁₁ is arginine, proline, or hydroxyproline, and X₁₄ is arginine or N-methylarginine. In some embodiments, X₁₁ is R. In some embodiments, X₁₄ is R.

In some embodiments, the NPR-C binding peptide comprises GRIDRI (SEQ ID NO: 2).

In some embodiments, the NPR-C binding peptide comprises SX₇X₈X₉GX₁₁IDX₁₄I (SEQ ID NO: 3), wherein:
X₇ is glycine, alanine, proline, hydroxyproline, serine, or cysteine;
X₈ is phenylalanine, or cyclohexylalanine, and
X₉ is glycine, alanine, or serine.

In some embodiments, X₇ is C. In some embodiments, X₉ is G.

In some embodiments, the NPR-C binding peptide comprises SCFGGRIDRI (SEQ ID NO: 4).

In other embodiments, the NPR-C binding peptide comprises FGGRIDRIGA (SEQ ID NO: 5).

In some embodiments, the NPR-C binding peptide comprises RSSX₇FGGRIDRI (SEQ ID NO: 6), wherein X₇ is serine or cysteine.

In some embodiments, the NPR-C binding peptide comprises SEQ ID NO: 7 (RSSX₇FGGRIDRIGA). In certain embodiments, X₇ is cysteine. In other embodiments, X₇ is serine.

In some embodiments, the NPR-C binding peptide comprises SEQ ID NO: 8 (X₇-Cha-X₉GX₁₁IDX₁₄I), wherein:
X₇ is cysteine, proline, hydroxyproline, glycine, or alanine;
X₉ is alanine, serine, or glycine;
X₁₁ is proline, hydroxyproline, or arginine; and
X₁₄ is arginine or N-methylarginine.

In certain embodiments, the NPR-C binding peptide comprises SEQ ID NO: 9 (fSp-Cha-aGPIDRI).

Exemplary NPR-C binding peptides that can be used in conjugates described herein are provided in Tables 1-3. In some embodiments, the NPR-C binding peptide comprises a sequence selected from any one of SEQ ID NO: 11-57 or 270. In other embodiments, the NPR-C binding peptide of the RNAi agent includes a sequence having at least 9 contiguous amino acids of one of SEQ ID NO: 11-57 or 270. In other embodiments, the NPR-C binding peptide of the RNAi agent includes a sequence having at least 90% sequence identity to one of SEQ ID NO: 11-57. In other embodiments, the NPR-C binding peptide of the RNAi agent includes a sequence having at least 95% sequence identity to one of SEQ ID NO: 11-57 or 270.

TABLE 1
Exemplary NPR-C binding peptides

| SEQ ID NO | Peptide Sequence |
|---|---|
| 11 | RSS[CFGGRIDRIGAQSGLGC]-NH₂ |
| 12 | RSSCFGGRIDRIGAQSGLGC-NH₂ |
| 13 | RSSSFGGRIDRIGAQSGLGS-NH₂ |
| 14 | RSS[CFGGRIDRIGAQSGLGC]-OH |
| 15 | RSSCFGGRIDRIGAQSGLGC-OH |
| 16 | RSSSFGGRIDRIGAQSGLGS-OH |
| 17 | RSSSFGGRIDRIGAQSGLGS-NH₂ |
| 18 | RSS[CFGGRIDRIGAC]-NH₂ |
| 19 | RSS[CFGGRIDRIGAQSGLGC]^{CH2}-OH |
| 20 | RSS[CFGGRIDRIGAQSGLGC]^{CH2}-NH₂ |
| 21 | fSp-Cha-aGPIDRI-NH₂ |
| 22 | fS-(D-Hyp)-Cha-sG-Hyp-ID-Arg(Me)-I- |
| | NHCH3 |
| 23 | fSp-Cha-aGPIDRIGSPSSGAPPPS-NH₂ |
| 24 | fSp-Cha-aGPIDRI-OH |
| 270 | GGGGEGGGGEGGGGEKEKEKGGGGSGGGGS- |
| | fSp-Cha-aGPIDRI-NH₂ |

TABLE 2
Exemplary NPR-C binding peptides

| SEQ ID NO | Peptide Sequence |
|---|---|
| 25 | Orn-fSp-Cha-aGPIDSI-NH₂ |
| 26 | fSp-Cha-aGPIDSI-Dap-NH₂ |
| 27 | fS-(D-Pip)-Cha-aGPIDSI-NH₂ |
| 28 | FSG-Cha-GGRIDRI-NH₂ |
| 29 | FSG-Cha-Aib-GRIDRI-NH₂ |
| 30 | fSp-Cha-aGPIERI-NH₂ |
| 31 | fSp-Cha-aGPID-(NMe-Arg)-I-NH₂ |
| 32 | RSFSGFGGRIDRI-NH₂ |
| 33 | RSFSGFGGRIDRIGAQSGLGS-NH₂ |
| 34 | RSFSGFGGRIDRIG-NH₂ |

TABLE 3
Exemplary NPR-C binding peptides

| SEQ ID NO | Peptide Sequence |
|---|---|
| 35 | C-Cha-GGRIDRIG-NH₂ |
| 36 | Ac-C-Cha-GGRIDRIG-NH₂ |
| 37 | S-Cha-GGRIDRIG-NH₂ |
| 38 | A-Cha-GGRIDRIG-NH₂ |
| 39 | Cha-GGRIDRIG-NH₂ |
| 40 | Ac-Cha-GGRIDRIG-NH₂ |
| 41 | G-Cha-GGRIDRIG-NH₂ |
| 42 | SG-Cha-GGRIDRIG-NH₂ |
| 43 | FSG-Cha-GGRIDRIG-NH₂ |
| 44 | SFSG-Cha-GGRIDRIG-NH₂ |
| 45 | RSFSG-Cha-GGRIDRIG-NH₂ |
| 46 | RSSG-Cha-GGRIDRIG-NH₂ |
| 47 | fSG-Cha-GGRIDRIG-NH₂ |
| 48 | Ac-fSG-Cha-GGRIDRIG-NH₂ |
| 49 | fSp-Cha-GGRIDRIG-NH₂ |
| 50 | fSG-Cha-aGRIDRIG-NH₂ |
| 51 | fSG-Cha-GGPIDRIG-NH₂ |
| 52 | Ac-fSp-Cha-aGPIDRIG-NH₂ |
| 53 | Ac-fSp-Cha-aGPIDRI-NH₂ |
| 54 | Ac-fSp-Cha-aGPIDRI-NHCH₃ |
| 55 | Ac-fSp-Cha-aGPID-Arg(Me)-I-NHCH₃ |
| 56 | Ac-fS-(D-Hyp)-Cha-aG-Hyp-ID-Arg(Me)- |
| | I-NHCH₃ |
| 57 | (HOCH2CO)-fS-(D-Hyp)-Cha-sG-Hyp-ID- |
| | Arg(Me-)I-NHCH₃ |

Tables 1-3 do not represent an exhaustive listing of all NPR-C binding peptides which may be utilized in a conjugate disclosed herein. In some instances, one or more amino acids may be substituted for an equivalent number of amino acids representing conservative mutations to the sequence, as are known in the art. For example, in some instances an isoleucine may be substituted where a leucine is indicated, and vice versa.

Suitable NPR-C binding peptides may be of a variety of lengths. In one aspect, the length of the peptide may be from 6 to 30 amino acids. In another aspect, the length of the peptide may be from 7 to 28 amino acids. In another aspect, the length of the peptide may be from 8 to 26 amino acids. In another aspect, the length of the peptide may be from 9 to 24 amino acids. In another aspect, the length of the peptide may be from 10 to 22 amino acids. The peptide may be 6 amino acids long, or 7 amino acids, or 8 amino acids, or 9 amino acids, or 10 amino acids, or 11 amino acids, or 12 amino acids, or 13 amino acids, or 14 amino acids, or 15 amino acids, or 16 amino acids, or 17 amino acids, or 18 amino acids, or 19 amino acids, or 20 amino acids, or 21 amino acids, or 22 amino acids, or 23 amino acids, or 24 amino acids, or 25 amino acids, or 26 amino acids, or 27 amino acids, or 28 amino acids, or 29 amino acids, or 30 amino acids in length.

As mentioned above, the NPR-C binding peptide may be modified. It may be modified at the N-terminal end, at the C-terminal end, at an internal position, or any combination of these. For example, the C-terminal carboxylic acid moiety of a peptide may be converted to an amide to generate a different peptide having the same amino acid sequence (see for example SEQ ID NO: 16 and SEQ ID NO: 17). In some embodiments, NPR-C binding peptide comprises a C-terminal hydroxyl. In some embodiments, NPR-C binding peptide comprises a C-terminal amide.

In some instances, the NPR-C binding peptide may be a linear peptide; that is, one in which there is no intramolecular bond made between any of the N-terminal end, the C-terminal end, and any of the side chains of the amino acids which constitute the peptide.

In other instances, the NPR-C binding peptide may be a cyclic peptide, in which there is exactly one or at least one intramolecular bond made between any of the N-terminal end, the C-terminal end, and any of the side chains of the amino acids which constitute the peptide. The bond which cyclizes the peptide may in one embodiment include atoms only derived from the parent peptide itself, for example a disulfide bond between cysteine side chains, as shown in Formula II below. As illustrated, Formula II shows the cysteine residues separated by 9 amino acids, with each X representing an amino acid residue. It will be appreciated that the amino acid chain may be of any length:

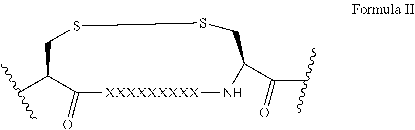

In another embodiment, the peptide may be cyclized using additional atoms in the intramolecular bond, such as an intervening alkyl group between the side chains of two cysteine residues. As illustrated, Formula III shows the cysteine residues separated by 9 amino acids, with each X representing an amino acid residue. It will be appreciated that the amino acid chain may be of any length. The value of z can be any integer from 1 to 20 inclusive.

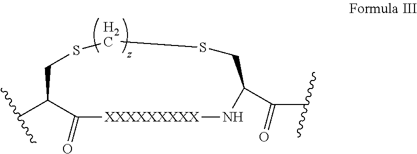

In the formula above, when z=1, the peptide contains a thioacetal moiety, defined by the side chains of the cysteine residues and the methylene group bridging the same.

In some embodiments, the NPR-C binding peptide is cyclized by covalent attachment of a side chain of a first cysteine residue to a side chain of a second cysteine residue. In some embodiments, the covalent attachment comprises a disulfide bond. In some embodiments, the covalent attachment comprises a thioacetal moiety.

In some instances, the NPR-C binding peptide may be a single copy of one of the sequences specified herein. Also envisaged are delivery moieties containing multiple copies of peptides disclosed herein, such as duplications of certain sequences, the use of multiple distinct peptides to generate a single construct, and so forth.

In some embodiments, P is an NPR-C-binding antibody or a fragment thereof. In some embodiments, P is a heteromab that binds NPR-C. In some embodiments, P is a Fab, VHH or scFv that binds NPR-C.

### Oligonucleotide

The conjugates described herein comprise an oligonucleotide. In some embodiments, O is an antisense oligonucleotide, a double stranded RNA (dsRNA), or a guide RNA.

In some embodiments, O is a double stranded RNA (dsRNA) comprising a sense stand and an antisense strand. In some embodiments, at least one nucleotide of the sense strand is a modified nucleotide. In some embodiments, at least one nucleotide of the antisense strand is a modified nucleotide. In some embodiments, at least one internucleotide linkage of the sense strand is a modified internucleotide linkage. In some embodiments, at least one internucleotide linkage of the antisense strand is a modified internucleotide linkage. In some embodiments, the dsRNA comprises a sense strand and an antisense stand, wherein the antisense strand is complementary to a target mRNA, that is, PDE3B mRNA.

I=In some embodiments, oligonucleotides may be provided as salt forms, particularly pharmaceutically acceptable salt forms. The negative charge of the phosphate backbone of the nucleic acid may be balanced by cations, such as sodium, calcium, potassium, or magnesium cations. Salts of the RNAi agents and conjugates provided herein may also be provided.

Exemplary unmodified sense strand and antisense strand sequences of dsRNA targeting PDE3B mRNA are provided in Table 4.

TABLE 4
Unmodified Nucleic Acid Sequences of dsRNA targeting human PDE3B mRNA
(PDE3B siRNA)

| | | | | | Starting |
|---|---|---|---|---|---|
| | | | | | target |
| | | | | | Position on |
| | | | | | gene |
| dsRNA | Unmodified Sense | SEQ ID | Unmodified Antisense | SEQ ID | transcript |
| NO | 5′ to 3′ | NO | 5′ to 3′ | NO | (NM_000922.4) |
| 1 | AACUUGUAGAAAAGAUG | 80 | UUCCCAUCUUUUCUACAA | 125 | 2318 |
| | GGAA | | GUUCA | | |
| 2 | ACCACAAGAUAUGGAAGG | 81 | UUUCCUUCCAUAUCUUGU | 126 | 3473 |
| | AAA | | GGUUU | | |
| 3 | ACUAGCAAAACUCCAAGA | 82 | UAUUCUUGGAGUUUUGCU | 127 | 3219 |
| | AUA | | AGUUG | | |
| 4 | ACUGGCUCUCUAACUAAU | 83 | UCGAUUAGUUAGAGAGCC | 128 | 1846 |
| | CGA | | AGUAG | | |
| 5 | CAAAACUCCAAGAAUCUU | 84 | UAAAAGAUUCUUGGAGUU | 129 | 3224 |
| | UUA | | UUGCU | | |
| 6 | CCCCGGAGGAGGUCCAGC | 85 | UCAGCUGGACCUCCUCCG | 130 | 1141 |
| | UGA | | GGGUC | | |
| 7 | CCUGCAUUAGAAUUGAUG | 86 | UGCCAUCAAUUCUAAUGC | 131 | 2689 |
| | GCA | | AGGAA | | |
| 8 | CUAACUAAUCGAUCACCC | 87 | UAUGGGUGAUCGAUUAGU | 132 | 1855 |
| | AUA | | UAGAG | | |
| 9 | CUCAACUAGCAAAACUCC | 88 | UUUGGAGUUUUGCUAGUU | 133 | 3215 |
| | AAA | | GAGGA | | |
| 10 | CUCUCUAACUAAUCGAUC | 89 | UGUGAUCGAUUAGUUAGA | 134 | 1851 |
| | ACA | | GAGCC | | |
| 11 | CUGCAUUAGAAUUGAUGG | 90 | UAGCCAUCAAUUCUAAUG | 135 | 2690 |
| | CUA | | CAGGA | | |
| 12 | CUGGCUCUCUAACUAAUC | 91 | UUCGAUUAGUUAGAGAGC | 136 | 1847 |
| | GAA | | CAGUA | | |
| 13 | GAAGAAGAAAAAUGUAA | 92 | UGCUUUACAUUUUUCUUC | 137 | 3502 |
| | AGCA | | UUCCU | | |
| 14 | GUUUUAUACAAUGACAGA | 93 | UGAUCUGUCAUUGUAUAA | 138 | 2794 |
| | UCA | | AACUG | | |
| 15 | UAGCAAAACUCCAAGAAU | 94 | UAGAUUCUUGGAGUUUUG | 139 | 3221 |
| | CUA | | CUAGU | | |
| 16 | UCUAACUAAUCGAUCACC | 95 | UUGGGUGAUCGAUUAGUU | 140 | 1854 |
| | CAA | | AGAGA | | |
| 17 | UGGCUCUCUAACUAAUCG | 96 | UAUCGAUUAGUUAGAGAG | 141 | 1848 |
| | AUA | | CCAGU | | |
| 18 | AAGAGUAUGACUCAUUAA | 97 | UUAUUAAUGAGUCAUACU | 142 | 2264 |
| | UAA | | CUUCU | | |
| 19 | AAUUUUCCAAUUUUUGAA | 98 | UAGUUCAAAAAUUGGAAA | 143 | 2302 |
| | CUA | | AUUCC | | |
| 20 | GAAUUUUCCAAUUUUUGA | 99 | UGUUCAAAAAUUGGAAAA | 144 | 2301 |
| | ACA | | UUCCA | | |
| 21 | GUUAUGUAUACCUUAUUU | 100 | UUGAAAUAAGGUAUACAU | 145 | 2365 |
| | CAA | | AACCU | | |
| 22 | AAUUUUUGAACUUGUAG | 101 | UUUUCUACAAGUUCAAAA | 146 | 2310 |
| | AAAA | | AUUGG | | |
| 23 | UAUGUAUACCUUAUUUCA | 102 | UCUUGAAAUAAGGUAUAC | 147 | 2367 |
| | AGA | | AUAAC | | |
| 24 | CAAGUCAAGGAUGCUAUC | 103 | UUAGAUAGCAUCCUUGAC | 148 | 1682 |
| | UAA | | UUGAA | | |
| 25 | AAGAAUCAUUCAAACUUA | 104 | UCAUAAGUUUGAAUGAUU | 149 | 2102 |
| | UGA | | CUUUC | | |
| 26 | AUUGCUUAUAUUUCUUCG | 105 | UUUCGAAGAAAUAUAAGC | 150 | 2620 |
| | AAA | | AAUUC | | |
| 27 | GCUAUCGAGACAUUCCUU | 106 | UAUAAGGAAUGUCUCGAU | 151 | 2459 |
| | AUA | | AGCCA | | |
| 28 | AACUCCAAGAAUCUUUUA | 107 | UGAUAAAAGAUUCUUGGA | 152 | 3227 |
| | UCA | | GUUUU | | |
| 29 | UGGAAUUUUCCAAUUUUU | 108 | UUCAAAAAUUGGAAAAUU | 153 | 2299 |
| | GAA | | CCAGU | | |
| 30 | GAAAGAAUCAUUCAAACU | 109 | UUAAGUUUGAAUGAUUCU | 154 | 2100 |
| | UAA | | UUCGA | | |
| 31 | UUUAUUGGAAAUAUUUA | 110 | UUUUUAAAUAUUUCCAAU | 155 | 2394 |
| | AAAA | | AAACC | | |
| 32 | AAACUCCAAGAAUCUUUU | 111 | UAUAAAAGAUUCUUGGAG | 156 | 3226 |
| | AUA | | UUUUG | | |
| 33 | GAAGAAGAAAACAUUUUC | 112 | UGAGAAAAUGUUUUCUUC | 157 | 2080 |
| | UCA | | UUCAC | | |
| 34 | ACGGUGAAGAAUUAGAU | 113 | UUGUAUCUAAUUCUUCAC | 158 | 3356 |
| | ACAA | | CGUCU | | |
| 35 | AGGGACUAAAUAGGAAU | 114 | UACUAUUCCUAUUUAGUC | 159 | 1547 |
| | AGUA | | CCUUG | | |
| 36 | AGAAAUGGAAAACAAUCU | 115 | UUUAGAUUGUUUUCCAUU | 160 | 3384 |
| | AAA | | UCUUC | | |
| 37 | AAGGUCAUCUUCUGUAUC | 116 | UGUGAUACAGAAGAUGAC | 161 | 1743 |
| | ACA | | CUUUG | | |
| 38 | CAGUCCAUUCAUGGAUCG | 117 | UAACGAUCCAUGAAUGGA | 162 | 3189 |
| | UUA | | CUGAU | | |
| 39 | UUUUCCAAUUUUUGAACU | 118 | UCAAGUUCAAAAAUUGGA | 163 | 2304 |
| | UGA | | AAAUU | | |
| 40 | CUCAACAAUUUAUGAACU | 119 | UAUAGUUCAUAAAUUGUU | 164 | 2420 |
| | AUA | | GAGUG | | |
| 41 | CGAAUUGCUUAUAUUUCU | 120 | UGAAGAAAUAUAAGCAAU | 165 | 2617 |
| | UCA | | UCGCC | | |
| 42 | GAAUUGCUUAUAUUUCUU | 121 | UCGAAGAAAUAUAAGCAA | 166 | 2618 |
| | CGA | | UUCGC | | |
| 43 | CAAUUUUUGAACUUGUAG | 122 | UUUCUACAAGUUCAAAAA | 167 | 2309 |
| | AAA | | UUGGA | | |
| 44 | AGGAAGAAGAAAAAUGU | 123 | UUUUACAUUUUUCUUCUU | 168 | 3500 |
| | AAAA | | CCUCU | | |
| 45 | UGGUAUAAGCCUCAUUAU | 124 | UUGAUAAUGAGGCUUAUA | 169 | 1279 |
| | CAA | | CCAUU | | |
| 100 | UGGUAUAAGCCUCAUUAU | 124 | UUGAUAAUGAGGCUUAUA | 317 | 1279 |
| | CAA | | CCAUU | | |
| 101 | AAGAGUAUGACUCAUUAA | 97 | UUAUUAAUGAGUCAUACU | 318 | 2264 |
| | UAA | | CUUGG | | |
| 102 | AAUUUUCCAAUUUUUGAA | 98 | UAGUUCAAAAAUUGGAAA | 319 | 2302 |
| | CUA | | AUUGG | | |
| 103 | AAGAAUCAUUCAAACUUA | 104 | UCAUAAGUUUGAAUGAUU | 320 | 2102 |
| | UGA | | CUUGG | | |
| 104 | AACUCCAAGAAUCUUUUA | 107 | UGAUAAAAGAUUCUUGGA | 321 | 3227 |
| | UCA | | GUUGG | | |
| 105 | AGAAAUGGAAAACAAUCU | 115 | UUUAGAUUGUUUUCCAUU | 322 | 3384 |
| | AAA | | UCUGG | | |
| 106 | AGGAAGAAGAAAAAUGU | 123 | UUUUACAUUUUUCUUCUU | 323 | 3500 |
| | AAAA | | CCUGG | | |
| 107 | CUAUGGCAGUUGCAAAAU | 271 | UAUAUUUUGCAACUGCCA | 324 | 1191 |
| | AUA | | UAGUA | | |
| 108 | UAUGGCAGUUGCAAAAUA | 272 | UAAUAUUUUGCAACUGCC | 325 | 1192 |
| | UUA | | AUAGU | | |
| 109 | AUGGCAGUUGCAAAAUAU | 273 | UGAAUAUUUUGCAACUGC | 326 | 1193 |
| | UCA | | CAUAG | | |
| 110 | GGCAGUUGCAAAAUAUUC | 274 | UCUGAAUAUUUUGCAACU | 327 | 1195 |
| | AGA | | GCCAU | | |
| 111 | GCAGUUGCAAAAUAUUCA | 275 | UCCUGAAUAUUUUGCAAC | 328 | 1196 |
| | GGA | | UGCCA | | |
| 112 | CAAUGGUAUAAGCCUCAU | 276 | UUAAUGAGGCUUAUACCA | 329 | 1276 |
| | UAA | | UUGUU | | |
| 113 | AUGGUAUAAGCCUCAUUA | 277 | UGAUAAUGAGGCUUAUAC | 330 | 1278 |
| | UCA | | CAUUG | | |
| 114 | GUAUAAGCCUCAUUAUCA | 278 | UUUUGAUAAUGAGGCUUA | 331 | 1281 |
| | AAA | | UACCA | | |
| 115 | UAUAAGCCUCAUUAUCAA | 279 | UUUUUGAUAAUGAGGCUU | 332 | 1282 |
| | AAA | | AUACC | | |
| 116 | AAGGGACUAAAUAGGAA | 280 | UCUAUUCCUAUUUAGUCC | 333 | 1546 |
| | UAGA | | CUUGU | | |
| 117 | GGGACUAAAUAGGAAUA | 281 | UAACUAUUCCUAUUUAGU | 334 | 1548 |
| | GUUA | | CCCUU | | |
| 118 | AAAGGUCAUCUUCUGUAU | 282 | UUGAUACAGAAGAUGACC | 335 | 1742 |
| | CAA | | UUUGC | | |
| 119 | UUCCUGAUACUGCUGAUU | 283 | UAAAAUCAGCAGUAUCAG | 336 | 1880 |
| | UUA | | GAAAU | | |
| 120 | UGAAGAAGAAAACAUUU | 284 | UAGAAAAUGUUUUCUUCU | 337 | 2079 |
| | UCUA | | UCACC | | |
| 121 | UCUCGAAAGAAUCAUUCA | 285 | UUUUGAAUGAUUCUUUCG | 338 | 2096 |
| | AAA | | AGAAA | | |
| 122 | CUCGAAAGAAUCAUUCAA | 286 | UGUUUGAAUGAUUCUUUC | 339 | 2097 |
| | ACA | | GAGAA | | |
| 123 | UCGAAAGAAUCAUUCAAA | 287 | UAGUUUGAAUGAUUCUUU | 340 | 2098 |
| | CUA | | CGAGA | | |
| 124 | UAGAAGAGUAUGACUCAU | 288 | UUAAUGAGUCAUACUCUU | 341 | 2261 |
| | UAA | | CUACU | | |
| 125 | AGAGUAUGACUCAUUAAU | 289 | UCUAUUAAUGAGUCAUAC | 342 | 2265 |
| | AGA | | UCUUC | | |
| 126 | UUGCUUAUAUUUCUUCGA | 290 | UCUUCGAAGAAAUAUAAG | 343 | 2621 |
| | AGA | | CAAUU | | |
| 127 | UCCUUCUUCAUCUUGAUC | 291 | UAUGAUCAAGAUGAAGAA | 344 | 2879 |
| | AUA | | GGAAG | | |
| 128 | AAAAGUUCGAGACUUGCA | 292 | UAAUGCAAGUCUCGAACU | 345 | 3096 |
| | UUA | | UUUGC | | |
| 129 | AAAGUUCGAGACUUGCAU | 293 | UAAAUGCAAGUCUCGAAC | 346 | 3097 |
| | UUA | | UUUUG | | |
| 130 | UUCGAGACUUGCAUUUGA | 294 | UUUUCAAAUGCAAGUCUC | 347 | 3101 |
| | AAA | | GAACU | | |
| 131 | ACUCCAAGAAUCUUUUAU | 295 | UUGAUAAAAGAUUCUUGG | 348 | 3228 |
| | CAA | | AGUUU | | |
| 132 | AAGACGGUGAAGAAUUA | 296 | UAUCUAAUUCUUCACCGU | 349 | 3353 |
| | GAUA | | CUUCA | | |
| 133 | GACGGUGAAGAAUUAGA | 297 | UGUAUCUAAUUCUUCACC | 350 | 3355 |
| | UACA | | GUCUU | | |
| 134 | AAUGGAAAACAAUCUAAA | 298 | UGAUUUAGAUUGUUUUCC | 351 | 3387 |
| | UCA | | AUUUC | | |
| 135 | GGAAGAAGAAAAAUGUA | 299 | UCUUUACAUUUUUCUUCU | 352 | 3501 |
| | AAGA | | UCCUC | | |
| 136 | AAGAGUAUGACUCAUUAA | 97 | UUAUUAAUGAGUCAUACU | 353 | 2264 |
| | UAA | | CUUUU | | |
| 137 | GGAAGAGUAUGACUCAUU | 300 | UUAUUAAUGAGUCAUACU | 354 | 2264 |
| | AAUAA | | CUUCC | | |
| 138 | AAGAGUAUGACUCAUUAA | 97 | UUAUUAAUGAGUCAUACU | 355 | 2264 |
| | UAA | | CUU | | |
| 139 | CAGAGUAUGACUCAUUAA | 301 | UUAUUAAUGAGUCAUACU | 356 | 2264 |
| | UAA | | CUGCU | | |
| 140 | AGAGUAUGACUCAUUAAU | 302 | UUAUUAAUGAGUCAUACU | 357 | 2264 |
| | AA | | CUUU | | |
| 141 | AAACUCCAAGAATCUUUU | 303 | UAUAAAAGAUUCUUGGAG | 156 | 3226 |
| | AUA | | UUUUG | | |
| 142 | GGAAACUCCAAGAAUCUU | 304 | UAUAAAAGAUUCUUGGAG | 358 | 3226 |
| | UUAUA | | UUUCC | | |
| 143 | AAACUCCAAGAAUCUUUU | 111 | UAUAAAAGAUUCUUGGAG | 359 | 3226 |
| | AUA | | UUU | | |
| 144 | AAACUCCAAGAAUCUUUU | 111 | UAUAAAAGAUUCUUGGAG | 360 | 3226 |
| | AUA | | UUUGG | | |
| 145 | CAACUCCAAGAAUCUUUU | 305 | UAUAAAAGAUUCUUGGAG | 361 | 3226 |
| | AUA | | UUGUG | | |
| 146 | AAACUCCAAGAAUCUUUU | 111 | UAUAAAAGAUUCUUGGAG | 362 | 3226 |
| | AUA | | UUUU | | |
| 147 | AACUCCAAGAAUCUUUUA | 306 | UAUAAAAGAUUCUUGGAG | 362 | 3226 |
| | UA | | UUUU | | |
| 148 | AAACUCCAAGAAUCUUUU | 307 | UAUAAAAGAUUCUUGGAG | 156 | 3226 |
| | AUG | | UUUUG | | |
| 149 | CACUCCAAGAAUCUUUUA | 308 | UGAUAAAAGAUUCUUGGA | 363 | 3227 |
| | UCA | | GUUCC | | |
| 150 | AACUCCAAGAAUCUUUUA | 107 | UGAUAAAAGAUUCUUGGA | 364 | 3227 |
| | UCA | | GUU | | |
| 151 | GGAACUCCAAGAAUCUUU | 309 | UGAUAAUAGAUUCUUGGA | 365 | 3227 |
| | UAUCA | | GUUUU | | |
| 152 | AACUCCAAGAAUCUAUUA | 310 | UGAUAAAAGAUUCUUGGA | 152 | 3227 |
| | UCA | | GUUUU | | |
| 153 | AACUCCAAGAAUCUUUUA | 311 | UGAUAAAAGAUUCUUGGA | 152 | 3227 |
| | UCATT | | GUUUU | | |
| 154 | AACUCCAAGAAUCUUUUA | 312 | UGAUAAAAGAUUCUUGGA | 366 | 3227 |
| | UCG | | GUGUU | | |
| 155 | CGGAAGAAGAAAAAUGU | 313 | UUUUACAUUUUUCUUCUU | 367 | 3500 |
| | AAAA | | CCGCU | | |
| 156 | GGAAGAAGAAAAAUGUA | 314 | UUUUACAUUUUUCUUCUU | 368 | 3500 |
| | AAA | | CCUU | | |
| 157 | AGGAAGAAGAAAAAUGU | 315 | UUUUACAUUUUUCUUCUU | 168 | 3500 |
| | AAAG | | CCUCU | | |
| 158 | AGGAAGAAGGAAAAUGU | 316 | UUUUACAUUUUUCUUCUU | 168 | 3500 |
| | AAAA | | CCUCU | | |

The dsRNA can include modifications. The modifications can be made to one or more nucleotides of the sense and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), or 2′-O-alkyl (e.g., 2′-O—C₁₆ alkyl).

In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl (e.g., 2′-O—C₁₆ alkyl) modified nucleotide. In some embodiments, each nucleotide of the sense strand and the antisense strand is independently a modified nucleotide, e.g., a 2′-fluoro modified nucleotide, 2′-O-methyl modified nucleotide, or 2′-O-alkyl (e.g., 2′-O—C₁₆ alkyl) modified nucleotide.

In some embodiments, the sense strand has four 2′-fluoro modified nucleotides, e.g., at positions 7, 9, 10, 11 from the 5′ end of the sense strand. In some embodiments, the other nucleotides of the sense strand are 2′-O-methyl modified nucleotides. In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, the other nucleotides of the sense strand are 2′-O-methyl modified nucleotides. In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the antisense strand has three 2′-fluoro modified nucleotides, e.g., at positions 2, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety, e.g., a moiety shown in Table 5.

TABLE 5
Abasic or inverted abasic (iAb) moieties

| | Structure |
|---|---|
| 1 (abasic) | 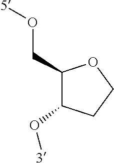 |
| 2 (iAb) | 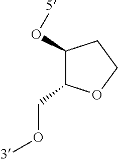 |

“5′” and “3′” indicate the 5′ to 3′ direction of the sequences.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

In some embodiments, the abasic moiety or inverted abasic moiety may be attached to the sense strand or the antisense strand via a phosphorothioate moiety. In one example, an inverted abasic moiety attached via a phosphorothioate internucleotide linkage has a formula as follows, where 3′ indicates the direction of the sequence:

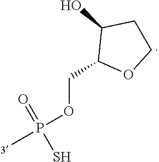

In some embodiments, the sense strand or antisense strand comprises a modified nucleotide with a lipid moiety at the 2′ position of a ribose (e.g., 2′-O—C₁₆ alkyl). In some embodiments, the modified nucleic acid is at position 6 from the 5′ end of the sense strand.

Exemplary modified sense strand and antisense strand sequences of dsRNA targeting PDE3B mRNA are provided in Table 6.

TABLE 6
Modified Nucleic Acid Sequences of dsRNA targeting human PDE3B mRNA
(PDE3B siRNA)

| dsRNA | SEQ ID | | |
|---|---|---|---|
| NO | NO | Strand | Modified sequence (5′ to 3′) |
| 46 | 170 | s | mA*mA*mCmUmUmGmUmAfGfAfAmAmAmGmAmUmGmGmG*mA* |
| | | | mA |
| | 224 | as | mU*fU*mCmCfCmAfUmCmUmUmUmUmCfUmAfCmAmAmGmUmU* |
| | | | mC*mA |
| 47 | 171 | s | mA*mC*mCmAmCmAmAmGfAfUfAmUmGmGmAmAmGmGmA*mA* |
| | | | mA |
| | 225 | as | mU*fU*mUmCfCmUfUmCmCmAmUmAmUfCmUfUmGmUmGmGmU* |
| | | | mU*mU |
| 48 | 172 | s | mA*mC*mUmAmGmCmAmAfAfAfCmUmCmCmAmAmGmAmA*mU* |
| | | | mA |
| | 226 | as | mU*fA*mUmUfCmUfUmGmGmAmGmUmUfUmUfGmCmUmAmGmU* |
| | | | mU*mG |
| 49 | 173 | s | mA*mC*mUmGmGmCmUmCfUfCfUmAmAmCmUmAmAmUmC*mG* |
| | | | mA |
| | 227 | as | mU*fC*mGmAfUmUfAmGmUmUmAmGmAfGmAfGmCmCmAmGmU* |
| | | | mA*mG |
| 50 | 174 | s | mC*mA*mAmAmAmCmUmCfCfAfAmGmAmAmUmCmUmUmU*mU* |
| | | | mA |
| | 228 | as | mU*fA*mAmAfAmGfAmUmUmCmUmUmGfGmAfGmUmUmUmUmG* |
| | | | mC*mU |
| 51 | 175 | s | mC*mC*mCmCmGmGmAmGfGfAfGmGmUmCmCmAmGmCmU*mG* |
| | | | mA |
| | 229 | as | mU*fC*mAmGfCmUfGmGmAmCmCmUmCfCmUfCmCmGmGmGmG* |
| | | | mU*mC |
| 52 | 176 | s | mC*mC*mUmGmCmAmUmUfAfGfAmAmUmUmGmAmUmGmG*mC* |
| | | | mA |
| | 230 | as | mU*fG*mCmCfAmUfCmAmAmUmUmCmUfAmAfUmGmCmAmGmG* |
| | | | mA*mA |
| 53 | 177 | s | mC*mU*mAmAmCmUmAmAfUfCfGmAmUmCmAmCmCmCmA*mU* |
| | | | mA |
| | 231 | as | mU*fA*mUmGfGmGfUmGmAmUmCmGmAfUmUfAmGmUmUmAmG* |
| | | | mA*mG |
| 54 | 178 | s | mC*mU*mCmAmAmCmUmAfGfCfAmAmAmAmCmUmCmCmA*mA* |
| | | | mA |
| | 232 | as | mU*fU*mUmGfGmAfGmUmUmUmUmGmCfUmAfGmUmUmGmAmG* |
| | | | mG*mA |
| 55 | 179 | s | mC*mU*mCmUmCmUmAmAfCfUfAmAmUmCmGmAmUmCmA*mC* |
| | | | mA |
| | 233 | as | mU*fG*mUmGfAmUfCmGmAmUmUmAmGfUmUfAmGmAmGmAmG* |
| | | | mC*mC |
| 56 | 180 | s | mC*mU*mGmCmAmUmUmAfGfAfAmUmUmGmAmUmGmGmC*mU* |
| | | | mA |
| | 234 | as | mU*fA*mGmCfCmAfUmCmAmAmUmUmCfUmAfAmUmGmCmAmG* |
| | | | mG*mA |
| 57 | 181 | s | mC*mU*mGmGmCmUmCmUfCfUfAmAmCmUmAmAmUmCmG*mA* |
| | | | mA |
| | 235 | as | mU*fU*mCmGfAmUfUmAmGmUmUmAmGfAmGfAmGmCmCmAmG* |
| | | | mU*mA |
| 58 | 182 | s | mG*mA*mAmGmAmAmGmAfAfAfAmAmUmGmUmAmAmAmG*mC* |
| | | | mA |
| | 236 | as | mU*fG*mCmUfUmUfAmCmAmUmUmUmUfUmCfUmUmCmUmUmC* |
| | | | mC*mU |
| 59 | 183 | s | mG*mU*mUmUmUmAmUmAfCfAfAmUmGmAmCmAmGmAmU*mC* |
| | | | mA |
| | 237 | as | mU*fG*mAmUfCmUfGmUmCmAmUmUmGfUmAfUmAmAmAmAmC* |
| | | | mU*mG |
| 60 | 184 | s | mU*mA*mGmCmAmAmAmAfCfUfCmCmAmAmGmAmAmUmC*mU* |
| | | | mA |
| | 238 | as | mU*fA*mGmAfUmUfCmUmUmGmGmAmGfUmUfUmUmGmCmUmA* |
| | | | mG*mU |
| 61 | 185 | s | mU*mC*mUmAmAmCmUmAfAfUfCmGmAmUmCmAmCmCmC*mA* |
| | | | mA |
| | 239 | as | mU*fU*mGmGfGmUfGmAmUmCmGmAmUfUmAfGmUmUmAmGmA* |
| | | | mG*mA |
| 62 | 186 | s | mU*mG*mGmCmUmCmUmCfUfAfAmCmUmAmAmUmCmGmA*mU* |
| | | | mA |
| | 240 | as | mU*fA*mUmCfGmAfUmUmAmGmUmUmAfGmAfGmAmGmCmCmA* |
| | | | mG*mU |
| 63 | 187 | s | mA*mA*mGmAmGmUmAmUfGfAfCmUmCmAmUmUmAmAmU*mA* |
| | | | mA |
| | 241 | as | mU*fU*mAmUfUmAfAmUmGmAmGmUmCfAmUfAmCmUmCmUmU* |
| | | | mC*mU |
| 64 | 188 | s | mA*mA*mUmUmUmUmCmCfAfAfUmUmUmUmUmGmAmAmC*mU* |
| | | | mA |
| | 242 | as | mU*fA*mGmUfUmCfAmAmAmAmAmUmUfGmGfAmAmAmAmUmU* |
| | | | mC*mC |
| 65 | 189 | s | mG*mA*mAmUmUmUmUmCfCfAfAmUmUmUmUmUmGmAmA*mC* |
| | | | mA |
| | 243 | as | mU*fG*mUmUfCmAfAmAmAmAmUmUmGfGmAfAmAmAmUmUmC* |
| | | | mC*mA |
| 66 | 190 | s | mG*mU*mUmAmUmGmUmAfUfAfCmCmUmUmAmUmUmUmC*mA* |
| | | | mA |
| | 244 | as | mU*fU*mGmAfAmAfUmAmAmGmGmUmAfUmAfCmAmUmAmAmC* |
| | | | mC*mU |
| 67 | 191 | s | mA*mA*mUmUmUmUmUmGfAfAfCmUmUmGmUmAmGmAmA*mA* |
| | | | mA |
| | 245 | as | mU*fU*mUmUfCmUfAmCmAmAmGmUmUfCmAfAmAmAmAmUmU* |
| | | | mG*mG |
| 68 | 192 | s | mU*mA*mUmGmUmAmUmAfCfCfUmUmAmUmUmUmCmAmA*mG* |
| | | | mA |
| | 246 | as | mU*fC*mUmUfGmAfAmAmUmAmAmGmGfUmAfUmAmCmAmUmA* |
| | | | mA*mC |
| 69 | 193 | s | mC*mA*mAmGmUmCmAmAfGfGfAmUmGmCmUmAmUmCmU*mA* |
| | | | mA |
| | 247 | as | mU*fU*mAmGfAmUfAmGmCmAmUmCmCfUmUfGmAmCmUmUmG* |
| | | | mA*mA |
| 70 | 194 | s | mA*mA*mGmAmAmUmCmAfUfUfCmAmAmAmCmUmUmAmU*mG* |
| | | | mA |
| | 248 | as | mU*fC*mAmUfAmAfGmUmUmUmGmAmAfUmGfAmUmUmCmUmU* |
| | | | mU*mC |
| 71 | 195 | s | mA*mU*mUmGmCmUmUmAfUfAfUmUmUmCmUmUmCmGmA*mA* |
| | | | mA |
| | 249 | as | mU*fU*mUmCfGmAfAmGmAmAmAmUmAfUmAfAmGmCmAmAmU* |
| | | | mU*mC |
| 72 | 196 | s | mG*mC*mUmAmUmCmGmAfGfAfCmAmUmUmCmCmUmUmA*mU* |
| | | | mA |
| | 250 | as | mU*fA*mUmAfAmGfGmAmAmUmGmUmCfUmCfGmAmUmAmGmC* |
| | | | mC*mA |
| 73 | 197 | s | mA*mA*mCmUmCmCmAmAfGfAfAmUmCmUmUmUmUmAmU*mC* |
| | | | mA |
| | 251 | as | mU*fG*mAmUfAmAfAmAmGmAmUmUmCfUmUfGmGmAmGmUmU* |
| | | | mU*mU |
| 74 | 198 | s | mU*mG*mGmAmAmUmUmUfUfCfCmAmAmUmUmUmUmUmG*mA* |
| | | | mA |
| | 252 | as | mU*fU*mCmAfAmAfAmAmUmUmGmGmAfAmAfAmUmUmCmCmA* |
| | | | mG*mU |
| 75 | 199 | s | mG*mA*mAmAmGmAmAmUfCfAfUmUmCmAmAmAmCmUmU*mA* |
| | | | mA |
| | 253 | as | mU*fU*mAmAfGmUfUmUmGmAmAmUmGfAmUfUmCmUmUmUmC* |
| | | | mG*mA |
| 76 | 200 | s | mU*mU*mUmAmUmUmGmGfAfAfAmUmAmUmUmUmAmAmA*mA* |
| | | | mA |
| | 254 | as | mU*fU*mUmUfUmAfAmAmUmAmUmUmUfCmCfAmAmUmAmAmA* |
| | | | mC*mC |
| 77 | 201 | s | mA*mA*mAmCmUmCmCmAfAfGfAmAmUmCmUmUmUmUmA*mU* |
| | | | mA |
| | 255 | as | mU*fA*mUmAfAmAfAmGmAmUmUmCmUfUmGfGmAmGmUmUmU* |
| | | | mU*mG |
| 78 | 202 | s | mG*mA*mAmGmAmAmGmAfAfAfAmCmAmUmUmUmUmCmU*mC* |
| | | | mA |
| | 256 | as | mU*fG*mAmGfAmAfAmAmUmGmUmUmUfUmCfUmUmCmUmUmC* |
| | | | mA*mC |
| 79 | 203 | s | mA*mC*mGmGmUmGmAmAfGfAfAmUmUmAmGmAmUmAmC*mA* |
| | | | mA |
| | 257 | as | mU*fU*mGmUfAmUfCmUmAmAmUmUmCfUmUfCmAmCmCmGmU* |
| | | | mC*mU |
| 80 | 204 | s | mA*mG*mGmGmAmCmUmAfAfAfUmAmGmGmAmAmUmAmG*mU* |
| | | | mA |
| | 258 | as | mU*fA*mCmUfAmUfUmCmCmUmAmUmUfUmAfGmUmCmCmCmU* |
| | | | mU*mG |
| 81 | 205 | s | mA*mG*mAmAmAmUmGmGfAfAfAmAmCmAmAmUmCmUmA*mA* |
| | | | mA |
| | 259 | as | mU*fU*mUmAfGmAfUmUmGmUmUmUmUfCmCfAmUmUmUmCmU* |
| | | | mU*mC |
| 82 | 206 | s | mA*mA*mGmGmUmCmAmUfCfUfUmCmUmGmUmAmUmCmA*mC* |
| | | | mA |
| | 260 | as | mU*fG*mUmGfAmUfAmCmAmGmAmAmGfAmUfGmAmCmCmUmU* |
| | | | mU*mG |
| 83 | 207 | s | mC*mA*mGmUmCmCmAmUfUfCfAmUmGmGmAmUmCmGmU*mU* |
| | | | mA |
| | 261 | as | mU*fA*mAmCfGmAfUmCmCmAmUmGmAfAmUfGmGmAmCmUmG* |
| | | | mA*mU |
| 84 | 208 | s | mU*mU*mUmUmCmCmAmAfUfUfUmUmUmGmAmAmCmUmU*mG* |
| | | | mA |
| | 262 | as | mU*fC*mAmAfGmUfUmCmAmAmAmAmAfUmUfGmGmAmAmAmA* |
| | | | mU*mU |
| 85 | 209 | s | mC*mU*mCmAmAmCmAmAfUfUfUmAmUmGmAmAmCmUmA*mU* |
| | | | mA |
| | 263 | as | mU*fA*mUmAfGmUfUmCmAmUmAmAmAfUmUfGmUmUmGmAmG* |
| | | | mU*mG |
| 86 | 210 | s | mC*mG*mAmAmUmUmGmCfUfUfAmUmAmUmUmUmCmUmU*mC* |
| | | | mA |
| | 264 | as | mU*fG*mAmAfGmAfAmAmUmAmUmAmAfGmCfAmAmUmUmCmG* |
| | | | mC*mC |
| 87 | 211 | s | mG*mA*mAmUmUmGmCmUfUfAfUmAmUmUmUmCmUmUmC*mG* |
| | | | mA |
| | 265 | as | mU*fC*mGmAfAmGfAmAmAmUmAmUmAfAmGfCmAmAmUmUmC* |
| | | | mG*mC |
| 88 | 212 | s | mC*mA*mAmUmUmUmUmUfGfAfAmCmUmUmGmUmAmGmA*mA* |
| | | | mA |
| | 266 | as | mU*fU*mUmCfUmAfCmAmAmGmUmUmCfAmAfAmAmAmUmUmG* |
| | | | mG*mA |
| 89 | 213 | s | mA*mG*mGmAmAmGmAmAfGfAfAmAmAmAmUmGmUmAmA*mA* |
| | | | mA |
| | 267 | as | mU*fU*mUmUfAmCfAmUmUmUmUmUmCfUmUfCmUmUmCmCmU* |
| | | | mC*mU |
| 90 | 214 | s | mU*mG*mGmUmAmUmAmAfGfCfCmUmCmAmUmUmAmUmC*mA* |
| | | | mA |
| | 268 | as | mU*fU*mGmAfUmAfAmUmGmAmGmGmCfUmUfAmUmAmCmCmA* |
| | | | mU*mU |
| 91 | 215 | s | mU*mG*mGmCmU[Chd]mUmCfUfAfAmCmUmAmAmUmCmGmA*mU |
| | | | *mA |
| | 240 | as | mU*fA*mUmCfGmAfUmUmAmGmUmUmAfGmAfGmAmGmCmCmA* |
| | | | mG*mU |
| 92 | 216 | s | mG*mA*mAmGmA[Ahd]mGmAfAfAfAmAmUmGmUmAmAmAmG*m |
| | | | C*mA |
| | 236 | as | mU*fG*mCmUfUmUfAmCmAmUmUmUmUfUmCfUmUmCmUmUmC* |
| | | | mC*mU |
| 93 | 217 | s | mA*mA*mCmUmU[Ghd]mUmAfGfAfAmAmAmGmAmUmGmGmG*m |
| | | | A*mA |
| | 224 | as | mU*fU*mCmCfCmAfUmCmUmUmUmUmCfUmAfCmAmAmGmUmU* |
| | | | mC*mA |
| 94 | 218 | s | mC*mU*mGmCmA[Uhd]mUmAfGfAfAmUmUmGmAmUmGmGmC*m |
| | | | U*mA |
| | 234 | as | mU*fA*mGmCfCmAfUmCmAmAmUmUmCfUmAfAmUmGmCmAmG* |
| | | | mG*mA |
| 95 | 219 | s | mC*mU*mCmUmC[Uhd]mAmAfCfUfAmAmUmCmGmAmUmCmA*mC |
| | | | *mA |
| | 233 | as | mU*fG*mUmGfAmUfCmGmAmUmUmAmGfUmUfAmGmAmGmAmG* |
| | | | mC*mC |
| 96 | 220 | s | mA*mC*mUmGmG[Chd]mUmCfUfCfUmAmAmCmUmAmAmUmC*mG |
| | | | *mA |
| | 227 | as | mU*fC*mGmAfUmUfAmGmUmUmAmGmAfGmAfGmCmCmAmGmU* |
| | | | mA*mG |
| 97 | 221 | s | mU*mC*mUmAmA[Chd]mUmAfAfUfCmGmAmUmCmAmCmCmC*mA |
| | | | *mA |
| | 239 | as | mU*fU*mGmGfGmUfGmAmUmCmGmAmUfUmAfGmUmUmAmGmA* |
| | | | mG*mA |
| 98 | 222 | s | mC*mU*mAmAmC[Uhd]mAmAfUfCfGmAmUmCmAmCmCmCmA*mU |
| | | | *mA |
| | 231 | as | mU*fA*mUmGfGmGfUmGmAmUmCmGmAfUmUfAmGmUmUmAmG* |
| | | | mA*mG |
| 99 | 223 | s | mC*mC*mUmGmC[Ahd]mUmUfAfGfAmAmUmUmGmAmUmGmG*mC |
| | | | *mA |
| | 230 | as | mU*fG*mCmCfAmUfCmAmAmUmUmCmUfAmAfUmGmCmAmGmG* |
| | | | mA*mA |
| 159 | 187 | s | mA*mA*mGmAmGmUmAmUfGfAfCmUmCmAmUmUmAmAmU*mA* |
| | | | mA |
| | 464 | as | mU*fU*mAmUfUmAfAmUmGmAmGmUmCfAmUfAmCmUmCmUmU* |
| | | | mG*mG |
| 160 | 188 | s | mA*mA*mUmUmUmUmCmCfAfAfUmUmUmUmUmGmAmAmC*mU* |
| | | | mA |
| | 465 | as | mU*fA*mGmUfUmCfAmAmAmAmAmUmUfGmGfAmAmAmAmUmU* |
| | | | mG*mG |
| 161 | 194 | s | mA*mA*mGmAmAmUmCmAfUfUfCmAmAmAmCmUmUmAmU*mG* |
| | | | mA |
| | 466 | as | mU*fC*mAmUfAmAfGmUmUmUmGmAmAfUmGfAmUmUmCmUmU* |
| | | | mG*mG |
| 162 | 197 | s | mA*mA*mCmUmCmCmAmAfGfAfAmUmCmUmUmUmUmAmU*mC* |
| | | | mA |
| | 467 | as | mU*fG*mAmUfAmAfAmAmGmAmUmUmCfUmUfGmGmAmGmUmU* |
| | | | mG*mG |
| 163 | 205 | s | mA*mG*mAmAmAmUmGmGfAfAfAmAmCmAmAmUmCmUmA*mA* |
| | | | mA |
| | 468 | as | mU*fU*mUmAfGmAfUmUmGmUmUmUmUfCmCfAmUmUmUmCmU* |
| | | | mG*mG |
| 164 | 213 | s | mA*mG*mGmAmAmGmAmAfGfAfAmAmAmAmUmGmUmAmA*mA* |
| | | | mA |
| | 469 | as | mU*fU*mUmUfAmCfAmUmUmUmUmUmCfUmUfCmUmUmCmCmU* |
| | | | mG*mG |
| 165 | 369 | s | iAb*mA*mAmGmAmGmUmAmUfGfAfCmUmCmAmUmUmAmAmU*m |
| | | | A*mA |
| | 470 | as | mU*fU*mAmUfUmAfAmUmGmAmGmUmCfAmUfAmCmUmCmU*mU |
| | | | *mC*mU |
| 166 | 370 | s | iAb*mC*mAmAmGmUmCmAmAfGfGfAmUmGmCmUmAmUmCmU*m |
| | | | A*mA |
| | 471 | as | mU*fU*mAmGfAmUfAmGmCmAmUmCmCfUmUfGmAmCmUmU*mG |
| | | | *mA*mA |
| 167 | 371 | s | iAb*mG*mAmAmAmGmAmAmUfCfAfUmUmCmAmAmAmCmUmU*m |
| | | | A*mA |
| | 472 | as | mU*fU*mAmAfGmUfUmUmGmAmAmUmGfAmUfUmCmUmUmU*mC |
| | | | *mG*mA |
| 168 | 372 | s | iAb*mA*mAmGmAmAmUmCmAfUfUfCmAmAmAmCmUmUmAmU*m |
| | | | G*mA |
| | 473 | as | mU*fC*mAmUfAmAfGmUmUmUmGmAmAfUmGfAmUmUmCmU*mU |
| | | | *mU*mC |
| 169 | 373 | s | iAb*mU*mAmUmGmUmAmUmAfCfCfUmUmAmUmUmUmCmAmA*m |
| | | | G*mA |
| | 474 | as | mU*fC*mUmUfGmAfAmAmUmAmAmGmGfUmAfUmAmCmAmU*mA |
| | | | *mA*mC |
| 170 | 374 | s | iAb*mA*mUmUmGmCmUmUmAfUfAfUmUmUmCmUmUmCmGmA*m |
| | | | A*mA |
| | 475 | as | mU*fU*mUmCfGmAfAmGmAmAmAmUmAfUmAfAmGmCmAmA*mU |
| | | | *mU*mC |
| 171 | 375 | s | iAb*mA*mAmAmCmUmCmCmAfAfGfAmAmUmCmUmUmUmUmA*m |
| | | | U*mA |
| | 476 | as | mU*fA*mUmAfAmAfAmGmAmUmUmCmUfUmGfGmAmGmUmU*mU |
| | | | *mU*mG |
| 172 | 376 | s | iAb*mG*mAmAmGmAmAmGmAfAfAfAmCmAmUmUmUmUmCmU*m |
| | | | C*mA |
| | 477 | as | mU*fG*mAmGfAmAfAmAmUmGmUmUmUfUmCfUmUmCmUmU*mC |
| | | | *mA*mC |
| 173 | 377 | s | iAb*mA*mGmGmAmAmGmAmAfGfAfAmAmAmAmUmGmUmAmA* |
| | | | mA*mA |
| | 478 | as | mU*fU*mUmUfAmCfAmUmUmUmUmUmCfUmUfCmUmUmCmC*mU |
| | | | *mC*mU |
| 174 | 378 | s | iAb*mA*mAmCmUmCmCmAmAfGfAfAmUmCmUmUmUmUmAmU*m |
| | | | C*mA |
| | 479 | as | mU*fG*mAmUfAmAfAmAmGmAmUmUmCfUmUfGmGmAmGmU*mU |
| | | | *mG*mG |
| 175 | 378 | s | iAb*mA*mAmCmUmCmCmAmAfGfAfAmUmCmUmUmUmUmAmU*m |
| | | | C*mA |
| | 480 | as | mU*fG*mAmUfAmAfAmAmGmAmUmUmCfUmUfGmGmAmGmU*mU |
| | | | *mU*mU |
| 176 | 379 | s | iAb*mA*mAmUmUmUmUmUmGfAfAfCmUmUmGmUmAmGmAmA* |
| | | | mA*mA |
| | 481 | as | mU*fU*mUmUfCmUfAmCmAmAmGmUmUfCmAfAmAmAmAmU*mU |
| | | | *mG*mG |
| 177 | 380 | s | iAb*mG*mCmUmAmUmCmGmAfGfAfCmAmUmUmCmCmUmUmA*m |
| | | | U*mA |
| | 482 | as | mU*fA*mUmAfAmGfGmAmAmUmGmUmCfUmCfGmAmUmAmG*mC |
| | | | *mC*mA |
| 178 | 381 | s | iAb*mC*mUmAmAmCmUmAmAfUfCfGmAmUmCmAmCmCmCmA*m |
| | | | U*mA |
| | 483 | as | mU*fA*mUmGfGmGfUmGmAmUmCmGmAfUmUfAmGmUmUmA*mG |
| | | | *mA*mG |
| 179 | 382 | s | mC*mU*mAmUmGmGmCmAfGfUfUmGmCmAmAmAmAmUmA*mU* |
| | | | mA |
| | 484 | as | mU*fA*mUmAfUmUfUmUmGmCmAmAmCfUmGfCmCmAmUmAmG* |
| | | | mU*mA |
| 180 | 383 | s | mU*mA*mUmGmGmCmAmGfUfUfGmCmAmAmAmAmUmAmU*mU* |
| | | | mA |
| | 48.5 | as | mU*fA*mAmUfAmUfUmUmUmGmCmAmAfCmUfGmCmCmAmUmA* |
| | | | mG*mU |
| 181 | 384 | s | mA*mU*mGmGmCmAmGmUfUfGfCmAmAmAmAmUmAmUmU*mC* |
| | | | mA |
| | 486 | as | mU*fG*mAmAfUmAfUmUmUmUmGmCmAfAmCfUmGmCmCmAmU* |
| | | | mA*mG |
| 182 | 385 | s | mG*mG*mCmAmGmUmUmGfCfAfAmAmAmUmAmUmUmCmA*mG* |
| | | | mA |
| | 487 | as | mU*fC*mUmGfAmAfUmAmUmUmUmUmGfCmAfAmCmUmGmCmC* |
| | | | mA*mU |
| 183 | 386 | s | mG*mC*mAmGmUmUmGmCfAfAfAmAmUmAmUmUmCmAmG*mG* |
| | | | mA |
| | 488 | as | mU*fC*mCmUfGmAfAmUmAmUmUmUmUfGmCfAmAmCmUmGmC* |
| | | | mC*mA |
| 184 | 387 | s | mC*mA*mAmUmGmGmUmAfUfAfAmGmCmCmUmCmAmUmU*mA* |
| | | | mA |
| | 489 | as | mU*fU*mAmAfUmGfAmGmGmCmUmUmAfUmAfCmCmAmUmUmG* |
| | | | mU*mU |
| 185 | 388 | s | mA*mU*mGmGmUmAmUmAfAfGfCmCmUmCmAmUmUmAmU*mC* |
| | | | mA |
| | 490 | as | mU*fG*mAmUfAmAfUmGmAmGmGmCmUfUmAfUmAmCmCmAmU* |
| | | | mU*mG |
| 186 | 389 | s | mG*mU*mAmUmAmAmGmCfCfUfCmAmUmUmAmUmCmAmA*mA* |
| | | | mA |
| | 491 | as | mU*fU*mUmUfGmAfUmAmAmUmGmAmGfGmCfUmUmAmUmAmC* |
| | | | mC*mA |
| 187 | 390 | s | mU*mA*mUmAmAmGmCmCfUfCfAmUmUmAmUmCmAmAmA*mA* |
| | | | mA |
| | 492 | as | mU*fU*mUmUfUmGfAmUmAmAmUmGmAfGmGfCmUmUmAmUmA* |
| | | | mC*mC |
| 188 | 391 | s | mA*mA*mGmGmGmAmCmUfAfAfAmUmAmGmGmAmAmUmA*mG* |
| | | | mA |
| | 493 | as | mU*fC*mUmAfUmUfCmCmUmAmUmUmUfAmGfUmCmCmCmUmU* |
| | | | mG*mU |
| 189 | 392 | s | mG*mG*mGmAmCmUmAmAfAfUfAmGmGmAmAmUmAmGmU*mU* |
| | | | mA |
| | 494 | as | mU*fA*mAmCfUmAfUmUmCmCmUmAmUfUmUfAmGmUmCmCmC* |
| | | | mU*mU |
| 190 | 393 | s | mA*mA*mAmGmGmUmCmAfUfCfUmUmCmUmGmUmAmUmC*mA* |
| | | | mA |
| | 495 | as | mU*fU*mGmAfUmAfCmAmGmAmAmGmAfUmGfAmCmCmUmUmU* |
| | | | mG*mC |
| 191 | 394 | s | mU*mU*mCmCmUmGmAmUfAfCfUmGmCmUmGmAmUmUmU*mU* |
| | | | mA |
| | 496 | as | mU*fA*mAmAfAmUfCmAmGmCmAmGmUfAmUfCmAmGmGmAmA* |
| | | | mA*mU |
| 192 | 395 | s | mU*mG*mAmAmGmAmAmGfAfAfAmAmCmAmUmUmUmUmC*mU* |
| | | | mA |
| | 497 | as | mU*fA*mGmAfAmAfAmUmGmUmUmUmUfCmUfUmCmUmUmCmA* |
| | | | mC*mC |
| 193 | 396 | s | mU*mC*mUmCmGmAmAmAfGfAfAmUmCmAmUmUmCmAmA*mA* |
| | | | mA |
| | 498 | as | mU*fU*mUmUfGmAfAmUmGmAmUmUmCfUmUfUmCmGmAmGmA* |
| | | | mA*mA |
| 194 | 397 | s | mC*mU*mCmGmAmAmAmGfAfAfUmCmAmUmUmCmAmAmA*mC* |
| | | | mA |
| | 499 | as | mU*fG*mUmUfUmGfAmAmUmGmAmUmUfCmUfUmUmCmGmAmG* |
| | | | mA*mA |
| 195 | 398 | s | mU*mC*mGmAmAmAmGmAfAfUfCmAmUmUmCmAmAmAmC*mU* |
| | | | mA |
| | 500 | as | mU*fA*mGmUfUmUfGmAmAmUmGmAmUfUmCfUmUmUmCmGmA* |
| | | | mG*mA |
| 196 | 399 | s | mU*mA*mGmAmAmGmAmGfUfAfUmGmAmCmUmCmAmUmU*mA* |
| | | | mA |
| | 501 | as | mU*fU*mAmAfUmGfAmGmUmCmAmUmAfCmUfCmUmUmCmUmA* |
| | | | mC*mU |
| 197 | 400 | s | mA*mG*mAmGmUmAmUmGfAfCfUmCmAmUmUmAmAmUmA*mG* |
| | | | mA |
| | 502 | as | mU*fC*mUmAfUmUfAmAmUmGmAmGmUfCmAfUmAmCmUmCmU* |
| | | | mU*mC |
| 198 | 401 | s | mU*mU*mGmCmUmUmAmUfAfUfUmUmCmUmUmCmGmAmA*mG* |
| | | | mA |
| | 503 | as | mU*fC*mUmUfCmGfAmAmGmAmAmAmUfAmUfAmAmGmCmAmA* |
| | | | mU*mU |
| 199 | 402 | s | mU*mC*mCmUmUmCmUmUfCfAfUmCmUmUmGmAmUmCmA*mU* |
| | | | mA |
| | 504 | as | mU*fA*mUmGfAmUfCmAmAmGmAmUmGfAmAfGmAmAmGmGmA* |
| | | | mA*mG |
| 200 | 403 | s | mA*mA*mAmAmGmUmUmCfGfAfGmAmCmUmUmGmCmAmU*mU* |
| | | | mA |
| | 505 | as | mU*fA*mAmUfGmCfAmAmGmUmCmUmCfGmAfAmCmUmUmUmU* |
| | | | mG*mC |
| 201 | 404 | s | mA*mA*mAmGmUmUmCmGfAfGfAmCmUmUmGmCmAmUmU*mU* |
| | | | mA |
| | 506 | as | mU*fA*mAmAfUmGfCmAmAmGmUmCmUfCmGfAmAmCmUmUmU* |
| | | | mU*mG |
| 202 | 405 | s | mU*mU*mCmGmAmGmAmCfUfUfGmCmAmUmUmUmGmAmA*mA* |
| | | | mA |
| | 507 | as | mU*fU*mUmUfCmAfAmAmUmGmCmAmAfGmUfCmUmCmGmAmA* |
| | | | mC*mU |
| 203 | 406 | s | mA*mC*mUmCmCmAmAmGfAfAfUmCmUmUmUmUmAmUmC*mA* |
| | | | mA |
| | 508 | as | mU*fU*mGmAfUmAfAmAmAmGmAmUmUfCmUfUmGmGmAmGmU* |
| | | | mU*mU |
| 204 | 407 | s | mA*mA*mGmAmCmGmGmUfGfAfAmGmAmAmUmUmAmGmA*mU* |
| | | | mA |
| | 509 | as | mU*fA*mUmCfUmAfAmUmUmCmUmUmCfAmCfCmGmUmCmUmU* |
| | | | mC*mA |
| 205 | 408 | s | mG*mA*mCmGmGmUmGmAfAfGfAmAmUmUmAmGmAmUmA*mC* |
| | | | mA |
| | 510 | as | mU*fG*mUmAfUmCfUmAmAmUmUmCmUfUmCfAmCmCmGmUmC* |
| | | | mU*mU |
| 206 | 409 | s | mA*mA*mUmGmGmAmAmAfAfCfAmAmUmCmUmAmAmAmU*mC* |
| | | | mA |
| | 511 | as | mU*fG*mAmUfUmUfAmGmAmUmUmGmUfUmUfUmCmCmAmUmU* |
| | | | mU*mC |
| 207 | 410 | s | mG*mG*mAmAmGmAmAmGfAfAfAmAmAmUmGmUmAmAmA*mG* |
| | | | mA |
| | 512 | as | mU*fC*mUmUfUmAfCmAmUmUmUmUmUfCmUfUmCmUmUmCmC* |
| | | | mU*mC |
| 208 | 411 | s | iAb*mC*mUmAmUmGmGmCmAfGfUfUmGmCmAmAmAmAmUmA*m |
| | | | U*mA |
| | 513 | as | mU*fA*mUmAfUmUfUmUmGmCmAmAmCfUmGfCmCmAmUmA*mG |
| | | | *mU*mA |
| 209 | 412 | s | iAb*mU*mAmUmGmGmCmAmGfUfUfGmCmAmAmAmAmUmAmU*m |
| | | | U*mA |
| | 541 | as | mU*fA*mAmUfAmUfUmUmUmGmCmAmAfCmUfGmCmCmAmU*mA |
| | | | *mG*mU |
| 210 | 413 | s | iAb*mA*mUmGmGmCmAmGmUfUfGfCmAmAmAmAmUmAmUmU*m |
| | | | C*mA |
| | 515 | as | mU*fG*mAmAfUmAfUmUmUmUmGmCmAfAmCfUmGmCmCmA*mU |
| | | | *mA*mG |
| 211 | 414 | s | iAb*mG*mGmCmAmGmUmUmGfCfAfAmAmAmUmAmUmUmCmA*m |
| | | | G*mA |
| | 516 | as | mU*fC*mUmGfAmAfUmAmUmUmUmUmGfCmAfAmCmUmGmC*mC |
| | | | *mA*mU |
| 212 | 415 | s | iAb*mG*mCmAmGmUmUmGmCfAfAfAmAmUmAmUmUmCmAmG*m |
| | | | G*mA |
| | 517 | as | mU*fC*mCmUfGmAfAmUmAmUmUmUmUfGmCfAmAmCmUmG*mC |
| | | | *mC*mA |
| 213 | 416 | s | iAb*mC*mAmAmUmGmGmUmAfUfAfAmGmCmCmUmCmAmUmU*m |
| | | | A*mA |
| | 518 | as | mU*fU*mAmAfUmGfAmGmGmCmUmUmAfUmAfCmCmAmUmU*mG |
| | | | *mU*mU |
| 214 | 417 | s | iAb*mA*mUmGmGmUmAmUmAfAfGfCmCmUmCmAmUmUmAmU*m |
| | | | C*mA |
| | 519 | as | mU*fG*mAmUfAmAfUmGmAmGmGmCmUfUmAfUmAmCmCmA*mU |
| | | | *mU*mG |
| 215 | 418 | s | iAb*mG*mUmAmUmAmAmGmCfCfUfCmAmUmUmAmUmCmAmA*m |
| | | | A*mA |
| | 520 | as | mU*fU*mUmUfGmAfUmAmAmUmGmAmGfGmCfUmUmAmUmA*mC |
| | | | *mC*mA |
| 216 | 419 | s | iAb*mU*mAmUmAmAmGmCmCfUfCfAmUmUmAmUmCmAmAmA*m |
| | | | A*mA |
| | 521 | as | mU*fU*mUmUfUmGfAmUmAmAmUmGmAfGmGfCmUmUmAmU*mA |
| | | | *mC*mC |
| 217 | 420 | s | iAb*mA*mAmGmGmGmAmCmUfAfAfAmUmAmGmGmAmAmUmA* |
| | | | mG*mA |
| | 522 | as | mU*fC*mUmAfUmUfCmCmUmAmUmUmUfAmGfUmCmCmCmU*mU |
| | | | *mG*mU |
| 218 | 421 | s | iAb*mG*mGmGmAmCmUmAmAfAfUfAmGmGmAmAmUmAmGmU* |
| | | | mU*mA |
| | 523 | as | mU*fA*mAmCfUmAfUmUmCmCmUmAmUfUmUfAmGmUmCmC*mC |
| | | | *mU*mU |
| 219 | 422 | s | iAb*mA*mAmAmGmGmUmCmAfUfCfUmUmCmUmGmUmAmUmC*m |
| | | | A*mA |
| | 524 | as | mU*fU*mGmAfUmAfCmAmGmAmAmGmAfUmGfAmCmCmUmU*mU |
| | | | *mG*mC |
| 220 | 423 | s | iAb*mU*mUmCmCmUmGmAmUfAfCfUmGmCmUmGmAmUmUmU*m |
| | | | U*mA |
| | 525 | as | mU*fA*mAmAfAmUfCmAmGmCmAmGmUfAmUfCmAmGmGmA*mA |
| | | | *mA*mU |
| 221 | 424 | s | iAb*mU*mGmAmAmGmAmAmGfAfAfAmAmCmAmUmUmUmUmC*m |
| | | | U*mA |
| | 526 | as | mU*fA*mGmAfAmAfAmUmGmUmUmUmUfCmUfUmCmUmUmC*mA |
| | | | *mC*mC |
| 222 | 425 | s | iAb*mU*mCmUmCmGmAmAmAfGfAfAmUmCmAmUmUmCmAmA*m |
| | | | A*mA |
| | 527 | as | mU*fU*mUmUfGmAfAmUmGmAmUmUmCfUmUfUmCmGmAmG*mA |
| | | | *mA*mA |
| 223 | 426 | s | iAb*mC*mUmCmGmAmAmAmGfAfAfUmCmAmUmUmCmAmAmA*m |
| | | | C*mA |
| | 528 | as | mU*fG*mUmUfUmGfAmAmUmGmAmUmUfCmUfUmUmCmGmA*mG |
| | | | *mA*mA |
| 224 | 427 | s | iAb*mU*mCmGmAmAmAmGmAfAfUfCmAmUmUmCmAmAmAmC*m |
| | | | U*mA |
| | 529 | as | mU*fA*mGmUfUmUfGmAmAmUmGmAmUfUmCfUmUmUmCmG*mA |
| | | | *mG*mA |
| 225 | 428 | s | iAb*mU*mAmGmAmAmGmAmGfUfAfUmGmAmCmUmCmAmUmU*m |
| | | | A*mA |
| | 530 | as | mU*fU*mAmAfUmGfAmGmUmCmAmUmAfCmUfCmUmUmCmU*mA |
| | | | *mC*mU |
| 226 | 429 | s | iAb*mA*mGmAmGmUmAmUmGfAfCfUmCmAmUmUmAmAmUmA*m |
| | | | G*mA |
| | 531 | as | mU*fC*mUmAfUmUfAmAmUmGmAmGmUfCmAfUmAmCmUmC*mU |
| | | | *mU*mC |
| 227 | 430 | s | iAb*mU*mUmGmCmUmUmAmUfAfUfUmUmCmUmUmCmGmAmA*m |
| | | | G*mA |
| | 532 | as | mU*fC*mUmUfCmGfAmAmGmAmAmAmUfAmUfAmAmGmCmA*mA |
| | | | *mU*mU |
| 228 | 431 | s | iAb*mU*mCmCmUmUmCmUmUfCfAfUmCmUmUmGmAmUmCmA*m |
| | | | U*mA |
| | 533 | as | mU*fA*mUmGfAmUfCmAmAmGmAmUmGfAmAfGmAmAmGmG*mA |
| | | | *mA*mG |
| 229 | 432 | s | iAb*mA*mAmAmAmGmUmUmCfGfAfGmAmCmUmUmGmCmAmU*m |
| | | | U*mA |
| | 534 | as | mU*fA*mAmUfGmCfAmAmGmUmCmUmCfGmAfAmCmUmUmU*mU |
| | | | *mG*mC |
| | | | mU*fA*mAmUfGmCfAmAmGmUmCmUmCfGmAfAmCmUmUmU*mU |
| 230 | 433 | s | iAb*mA*mAmAmGmUmUmCmGfAfGfAmCmUmUmGmCmAmUmU*m |
| | | | U*mA |
| | 535 | as | mU*fA*mAmAfUmGfCmAmAmGmUmCmUfCmGfAmAmCmUmU*mU |
| | | | *mU*mG |
| 231 | 434 | s | iAb*mU*mUmCmGmAmGmAmCfUfUfGmCmAmUmUmUmGmAmA*m |
| | | | A*mA |
| | 536 | as | mU*fU*mUmUfCmAfAmAmUmGmCmAmAfGmUfCmUmCmGmA*mA |
| | | | *mC*mU |
| 232 | 435 | s | iAb*mA*mCmUmCmCmAmAmGfAfAfUmCmUmUmUmUmAmUmC*m |
| | | | A*mA |
| | 537 | as | mU*fU*mGmAfUmAfAmAmAmGmAmUmUfCmUfUmGmGmAmG*mU |
| | | | *mU*mU |
| 233 | 436 | s | iAb*mA*mAmGmAmCmGmGmUfGfAfAmGmAmAmUmUmAmGmA* |
| | | | mU*mA |
| | 538 | as | mU*fA*mUmCfUmAfAmUmUmCmUmUmCfAmCfCmGmUmCmU*mU |
| | | | *mC*mA |
| 234 | 437 | s | iAb*mG*mAmCmGmGmUmGmAfAfGfAmAmUmUmAmGmAmUmA* |
| | | | mC*mA |
| | 539 | as | mU*fG*mUmAfUmCfUmAmAmUmUmCmUfUmCfAmCmCmGmU*mC |
| | | | *mU*mU |
| 235 | 438 | s | iAb*mA*mAmUmGmGmAmAmAfAfCfAmAmUmCmUmAmAmAmU*m |
| | | | C*mA |
| | 540 | as | mU*fG*mAmUfUmUfAmGmAmUmUmGmUfUmUfUmCmCmAmU*mU |
| | | | *mU*mC |
| 236 | 439 | s | iAb*mG*mGmAmAmGmAmAmGfAfAfAmAmAmUmGmUmAmAmA* |
| | | | mG*mA |
| | 541 | as | mU*fC*mUmUfUmAfCmAmUmUmUmUmUfCmUfUmCmUmUmC*mC |
| | | | *mU*mC |
| 237 | 369 | s | iAb*mA*mAmGmAmGmUmAmUfGfAfCmUmCmAmUmUmAmAmU*m |
| | | | A*mA |
| | 542 | as | mU*fU*mAmUfUmAfAmUmGmAmGmUmCfAmUfAmCmUmCmU*mU |
| | | | *PrON*PrON |
| 238 | 440 | s | iAb*[GL]*[GL]mAmAmGmAmGmUmAmUfGfAfCmUmCmAmUmUmA |
| | | | mAmU*mA*mA |
| | 543 | as | mU*fU*mAmUfUmAfAmUmGmAmGmUmCfAmUfAmCmUmCmU*mU |
| | | | *mC*mC |
| 239 | 369 | s | iAb*mA*mAmGmAmGmUmAmUfGfAfCmUmCmAmUmUmAmAmU*m |
| | | | A*mA |
| | 544 | as | mU*fU*mAmUfUmAfAmUmGmAmGmUmCfAmUfAmCmUmCmU*mU |
| | | | *mG*mG |
| 240 | 369 | s | iAb*mA*mAmGmAmGmUmAmUfGfAfCmUmCmAmUmUmAmAmU*m |
| | | | A*mA |
| | 545 | as | mU*fU*mAmUfUmAfAmUmGmAmGmUmCfAmUfAmCmU*mC*PrON |
| | | | *PrON |
| 241 | 441 | s | iAb*mC*mAmGmAmGmUmAmUfGfAfCmUmCmAmUmUmAmAmU*m |
| | | | A*mA |
| | 546 | as | mU*fU*mAmUfUmAfAmUmGmAmGmUmCfAmUfAmCmUmCmU*mG |
| | | | *mC*mU |
| 242 | 442 | s | iAb*mA*mGmAmGmUmAmUfGfAfCmUmCmAmUmUmAmAmU*mA* |
| | | | mA |
| | 547 | as | mU*fU*mAmUfUmAfAmUmGmAmGmUmCfAmUfAmCmUmC*mU*Pr |
| | | | ON*PrON |
| 243 | 443 | s | iAb*mA*[DAP]mGmAmGmUmAmUfGfAfCmUmCmAmUmUmA[DAP] |
| | | | mU*mA*mA |
| | 470 | as | mU*fU*mAmUfUmAfAmUmGmAmGmUmCfAmUfAmCmUmCmU*mU |
| | | | *mC*mU |
| 244 | 444 | s | iAb*[AL]*[AL]mGmAmGmUmAmUfGfAfCmU[CL]mAmUmUmAmAm |
| | | | U*mA*mA |
| | 470 | as | mU*fU*mAmUfUmAfAmUmGmAmGmUmCfAmUfAmCmUmCmU*mU |
| | | | *mC*mU |
| 245 | 445 | s | iAb*[DAP]*[DAP]mGmAmGmUmAmUfGfAfCmU[C5P-C] |
| | | | mAmUmUmAmAmU*mA*mA |
| | 470 | as | mU*fU*mAmUfUmAfAmUmGmAmGmUmCfAmUfAmCmUmCmU*mU |
| | | | *mC*mU |
| 246 | 446 | s | iAb*[AL]*[AL]mAmCmUmCmCmAfAfGfAmA[TL]mCmUmUmUmUmA |
| | | | *mU*mA |
| | 548 | as | mU*fA*mUmAfAmAfAmGmAmUmUmCmUfUmGfGmA[UNA-G] |
| | | | mUmU*mU*mU*mG |
| 247 | 446 | s | iAb*[AL]*[AL]mAmCmUmCmCmAfAfGfAmA[TL]mCmUmUmUmUmA |
| | | | *mU*mA |
| | 549 | as | mU*fA*mUmAfAmAfAmGmAmUmU[UNA-C] |
| | | | mUfUmGfGmAmGmUmU*mU*mU*mG |
| 248 | 446 | s | iAb*[AL]*[AL]mAmCmUmCmCmAfAfGfAmA[TL]mCmUmUmUmUmA |
| | | | *mU*mA |
| | 476 | as | mU*fA*mUmAfAmAfAmGmAmUmUmCmUfUmGfGmAmGmUmU*mU |
| | | | *mU*mG |
| 249 | 447 | s | iAb*[GL]*[GL]mAmAmAmCmUmCmCmAfAfGfAmAmUmCmUmUmU |
| | | | mUmA*mU*mA |
| | 550 | as | mU*fA*mUmAfAmAfAmGmAmUmUmCmUfUmGfGmAmGmUmU*mU |
| | | | *mC*mC |
| 250 | 375 | s | iAb*mA*mAmAmCmUmCmCmAfAfGfAmAmUmCmUmUmUmUmA*m |
| | | | U*mA |
| | 551 | as | mU*fA*mUmAfAmAfAmGmAmUmUmCmUfUmGfGmAmG*mU*PrON |
| | | | *PrON |
| 251 | 375 | s | iAb*mA*mAmAmCmUmCmCmAfAfGfAmAmUmCmUmUmUmUmA*m |
| | | | U*mA |
| | 552 | as | mU*fA*mUmAfAmAfAmGmAmUmUmCmUfUmGfGmAmGmUmU*mU |
| | | | *mG*mG |
| 252 | 375 | s | iAb*mA*mAmAmCmUmCmCmAfAfGfAmAmUmCmUmUmUmUmA*m |
| | | | U*mA |
| | 553 | as | mU*fA*mUmAfAmAfAmGmAmUmUmCmUfUmGfGmAmGmUmU*mU |
| | | | *PRON*mG |
| 253 | 448 | s | iAb*mC*mAmAmCmUmCmCmAfAfGfAmAmUmCmUmUmUmUmA*m |
| | | | U*mA |
| | 554 | as | mU*fA*mUmAfAmAfAmGmAmUmUmCmUfUmGfGmAmGmUmU*mG |
| | | | *mU*mG |
| 254 | 449 | s | iAb*eA*eAmAmCmUmCmCmAfAfGfAmAmUmCmUmUmUmUmA*mU |
| | | | *mA |
| | 476 | as | mU*fA*mUmAfAmAfAmGmAmUmUmCmUfUmGfGmAmGmUmU*mU |
| | | | *mU*mG |
| 255 | 375 | s | iAb*mA*mAmAmCmUmCmCmAfAfGfAmAmUmCmUmUmUmUmA*m |
| | | | U*mA |
| | 555 | as | mU*fA*mUmAfAmAfAmGmAmUmUmCmUfUmGfGmAmGmU*mU*Pr |
| | | | ON*PrON |
| 256 | 450 | s | iAb*mA*mAmCmUmCmCmAfAfGfAmAmUmCmUmUmUmUmA*mU* |
| | | | mA |
| | 556 | as | mU*fA*mUmAfAmAfAmGmAmUmUmCmUfUmGfGmAmGmU*mU*Pr |
| | | | ON*PrON |
| 257 | 45 | s | iAb*mA*mAmAmCmUmCmCmAfAfGfAmAmUmCmUmUmUmUmA*m |
| | | | U*mG |
| | 476 | as | mU*fA*mUmAfAmAfAmGmAmUmUmCmUfUmGfGmAmGmUmU*mU |
| | | | *mU*mG |
| 258 | 375 | s | iAb*mA*mAmAmCmUmCmCmAfAfGfAmAmUmCmUmUmUmUmA*m |
| | | | U*mA |
| | 557 | as | mU*fA*mUdAmAmAdAmGmAmUmUmCmUfUmGfGmAmGmUmU*m |
| | | | U*mU*mG |
| 259 | 446 | s | iAb*[AL]*[AL]mAmCmUmCmCmAfAfGfAmA[TL]mCmUmUmUmUmA |
| | | | *mU*mA |
| | 557 | as | mU*fA*mUdAmAmAdAmGmAmUmUmCmUfUmGfGmAmGmUmU*m |
| | | | U*mU*mG |
| 260 | 375 | s | iAb*mA*mAmAmCmUmCmCmAfAfGfAmAmUmCmUmUmUmUmA*m |
| | | | U*mA |
| | 558 | as | mU*fA*mUmAfAmAmAfGmAmUmUmCmUfUmGfGmAmGmUmU*mU |
| | | | *mU*mG |
| 261 | 378 | s | iAb*mA*mAmCmUmCmCmAmAfGfAfAmUmCmUmUmUmUmAmU*m |
| | | | C*mA |
| | 559 | as | mU*fG*mAmUfAmAfAmAmGmAmUmUmCfUmUfGmGmAmGmU*mU |
| | | | *PrON*PrON |
| 262 | 452 | s | iAb*[GL]*[GL]mAmAmCmUmCmCmAmAfGfAfAmUmCmUmUmUmU |
| | | | mAmU*mC*mA |
| | 560 | as | mU*fG*mAmUfAmAfAmAmGmAmUmUmCfUmUfGmGmAmGmU*mU |
| | | | *mC*mC |
| 263 | 378 | s | iAb*mA*mAmCmUmCmCmAmAfGfAfAmUmCmUmUmUmUmAmU*m |
| | | | C*mA |
| | 561 | as | mU*fG*mAmUfAmAfAmAmGmAmUmUmCfUmUfGmGmA*mG*PrON |
| | | | *PrON |
| 264 | 453 | s | iAb*mA*mAmCmUmCmCmAmAfGfAfAmUmCmUmAmUmUmAmU*m |
| | | | C*mA |
| | 562 | as | mU*fG*mAmUfAmAfUmAmGmAmUmUmCfUmUfGmGmAmGmU*mU |
| | | | *mU*mU |
| 265 | 378 | s | iAb*mA*mAmCmUmCmCmAmAfGfAfAmUmCmUmUmUmUmAmU*m |
| | | | C*mA |
| | 563 | as | mU*fG*mAmUfAmAfAmAmGmAmUmUmCfUYfGmGmAmGY*Y*mU |
| | | | *mU |
| 266 | 454 | s | iAb*mA*mAmCYmCmCmAmAfGfAfAmCmUmUmUmUmAmU*mC* |
| | | | mA |
| | 480 | as | mU*fG*mAmUfAmAfAmAmGmAmUmUmCfUmUfGmGmAmGmU*mU |
| | | | *mU*mU |
| 267 | 378 | s | iAb*mA*mAmCmUmCmCmAmAfGfAfAmUmCmUmUmUmUmAmU*m |
| | | | C*mA |
| | 564 | as | mU*fG*mAYfAmAfAmAmGmAYYmCfUmUfGmGmAmGmU*mU*mU |
| | | | *mU |
| 268 | 378 | s | iAb*mA*mAmCmUmCmCmAmAfGfAfAmUmCmUmUmUmUmAmU*m |
| | | | C*mA |
| | 565 | as | mU*fG*mAmUfAmAfAmAmGmAYmCfUmUfGmGmAmGmU*mU*m |
| | | | U*mU |
| 269 | 455 | s | iAb*mA*mAmCmUmCmCmAmAfGfAfAmUmCmUmUmUmUmAmU*m |
| | | | C*mG |
| | 480 | as | mU*fG*mAmUfAmAfAmAmGmAmUmUmCfUmUfGmGmAmGmU*mU |
| | | | *mU*mU |
| 270 | 456 | s | iAb*mA*mAmCmUmCmCmAmAfGfAfAmUmCmUmUmUmUmAmU*m |
| | | | C*mAdTdT |
| | 480 | as | mU*fG*mAmUfAmAfAmAmGmAmUmUmCfUmUfGmGmAmGmU*mU |
| | | | *mU*mU |
| 271 | 457 | s | iAb*mC*mAmCmUmCmCmAmAfGfAfAmUmCmUmUmUmUmAmU*m |
| | | | C*mA |
| | 566 | as | mU*fG*mAmUfAmAfAmAmGmAmUmUmCfUmUfGmGmAmGmU*mG |
| | | | *mU*mU |
| 272 | 458 | s | iAb*[AL]*[GL]mGmAmAmGmAmAfGfAfAmA[AL]mAmUmGmUmAm |
| | | | A*mA*mA |
| | 478 | as | mU*fU*mUmUfAmCfAmUmUmUmUmUmCfUmUfCmUmUmCmC*mU |
| | | | *mC*mU |
| 273 | 459 | s | iAb*mC*mGmGmAmAmGmAmAfGfAfAmAmAmAmUmGmUmAmA* |
| | | | mA*mA |
| | 567 | as | mU*fU*mUmUfAmCfAmUmUmUmUmUmCfUmUfCmUmUmCmC*mG |
| | | | *mC*mU |
| 274 | 460 | s | iAb*eA*eGmGmAmAmGmAmAfGfAfAmAmAmAmUmGmUmAmA*m |
| | | | A*mA |
| | 478 | as | mU*fU*mUmUfAmCfAmUmUmUmUmUmCfUmUfCmUmUmCmC*mU |
| | | | *mC*mU |
| 275 | 46 | s | iAb*mG*mGmAmAmGmAmAfGfAfAmAmAmAmUmGmUmAmA*mA* |
| | | | mA |
| | 568 | as | mU*fU*mUmUfAmCfAmUmUmUmUmUmCfUmUfCmUmUmC*mC*Pr |
| | | | ON*PrON |
| 276 | 462 | s | iAb*mA*mGmGmAmAmGmAmAfGfAfAmAmAmAmUmGmUmAmA* |
| | | | mA*mG |
| | 478 | as | mU*fU*mUmUfAmCfAmUmUmUmUmUmCfUmUfCmUmUmCmC*mU |
| | | | *mC*mU |
| 277 | 463 | s | iAb*mA*mGmGmAmAmGmAmAfGfGfAmAmAmAmUmGmUmAmA* |
| | | | mA*mA |
| | 478 | as | mU*fU*mUmUfAmCfAmUmUmUmUmUmCfUmUfCmUmUmCmC*mU |
| | | | *mC*mU |
| 278 | 376 | s | iAb*mG*mAmAmGmAmAmGmAfAfAfAmCmAmUmUmUmUmCmU*m |
| | | | C*mA |
| | 569 | as | VPmU*fG*mAmGfAmAfAmAmUmGmUmUmUfUmCfUmUmCmUmU* |
| | | | mC*mA*mC |
| 279 | 377 | s | iAb*mA*mGmGmAmAmGmAmAfGfAfAmAmAmAmUmGmUmAmA* |
| | | | mA*mA |
| | 570 | as | VPmU*fU*mUmUfAmCfAmUmUmUmUmUmCfUmUfCmUmUmCmC* |
| | | | mU*mC*mU |
| 280 | 378 | s | iAb*mA*mAmCmUmCmCmAmAfGfAfAmUmCmUmUmUmUmAmU*m |
| | | | C*mA |
| | 571 | as | VPmU*fG*mAmUfAmAfAmAmGmAmUmUmCfUmUfGmGmAmGmU* |
| | | | mU*mG*mG |
| 281 | 375 | s | iAb*mA*mAmAmCmUmCmCmAfAfGfAmAmUmCmUmUmUmUmA*m |
| | | | U*mA |
| | 572 | as | VPmU*fA*mUmAfAmAfAmGmAmUmUmCmUfUmGfGmAmGmUmU* |
| | | | mU*mU*mG |
| 282 | 374 | s | iAb*mA*mUmUmGmCmUmUmAfUfAfUmUmUmCmUmUmCmGmA*m |
| | | | A*mA |
| | 573 | as | VPmU*fU*mUmCfGmAfAmGmAmAmAmUmAfUmAfAmGmCmAmA* |
| | | | mU*mU*mC |
| 283 | 373 | s | iAb*mU*mAmUmGmUmAmUmAfCfCfUmUmAmUmUmUmCmAmA*m |
| | | | G*mA |
| | 574 | as | VPmU*fC*mUmUfGmAfAmAmUmAmAmGmGfUmAfUmAmCmAmU* |
| | | | mA*mA*mC |
| 284 | 372 | s | iAb*mA*mAmGmAmAmUmCmAfUfUfCmAmAmAmCmUmUmAmU*m |
| | | | G*mA |
| | 575 | as | VPmU*fC*mAmUfAmAfGmUmUmUmGmAmAfUmGfAmUmUmCmU* |
| | | | mU*mU*mC |
| 285 | 369 | s | iAb*mA*mAmGmAmGmUmAmUfGfAfCmUmCmAmUmUmAmAmU*m |
| | | | A*mA |
| | 576 | as | VPmU*fU*mAmUfUmAfAmUmGmAmGmUmCfAmUfAmCmUmCmU* |
| | | | mU*mC*mU |
| 286 | 577 | s | iAb*mA*mGmGmAmAmGmAmAfGfAfAmAmAmAmU[Ghd]mUmAmA |
| | | | *mA*mA |
| | 478 | as | mU*fU*mUmUfAmCfAmUmUmUmUmUmCfUmUfCmUmUmCmC*mU |
| | | | *mC*mU |
| 287 | 578 | s | iAb*mG*mAmAmGmAmAmGmAfAfAfAmCmAmUmU[Uhd]mUmCmU |
| | | | *mC*mA |
| | 569 | as | VPmU*fG*mAmGfAmAfAmAmUmGmUmUmUfUmCfUmUmCmUmU* |
| | | | mC*mA*mC |
| 288 | 579 | s | iAb*mA*mAmCmUmCmCmAmAfGfAfAmUmCmUmU[Uhd]mUmAmU* |
| | | | mC*mA |
| | 571 | as | VPmU*fG*mAmUfAmAfAmAmGmAmUmUmCfUmUfGmGmAmGmU* |
| | | | mU*mG*mG |
| 289 | 580 | s | iAb*mA*mAmAmCmUmCmCmAfAfGfAmAmUmCmU[Uhd]mUmUmA* |
| | | | mU*mA |
| | 572 | as | VPmU*fA*mUmAfAmAfAmGmAmUmUmCmUfUmGfGmAmGmUmU* |
| | | | mU*mU*mG |
| 290 | 581 | s | iAb*mA*mUmUmGmCmUmUmAfUfAfUmUmUmCmU[Uhd]mCmGmA |
| | | | *mA*mA |
| | 573 | as | VPmU*fU*mUmCfGmAfAmGmAmAmAmUmAfUmAfAmGmCmAmA* |
| | | | mU*mU*mC |
| 291 | 582 | s | iAb*mU*mAmUmGmUmAmUmAfCfCfUmUmAmUmU[Uhd]mCmAmA |
| | | | *mG*mA |
| | 574 | as | VPmU*fC*mUmUfGmAfAmAmUmAmAmGmGfUmAfUmAmCmAmU* |
| | | | mA*mA*mC |
| 292 | 583 | s | iAb*mA*mAmGmAmAmUmCmAfUfUfCmAmAmAmC[Uhd]mUmAmU |
| | | | *mG*mA |
| | 575 | as | VPmU*fC*mAmUfAmAfGmUmUmUmGmAmAfUmGfAmUmUmCmU* |
| | | | mU*mU*mC |
| 293 | 584 | s | iAb*mA*mAmGmAmGmUmAmUfGfAfCmUmCmAmU[Uhd]mAmAmU |
| | | | *mA*mA |
| | 576 | as | VPmU*fU*mAmUfUmAfAmUmGmAmGmUmCfAmUfAmCmUmCmU* |
| | | | mU*mC*mU |

Abbreviations-“m” indicates 2′-OMe; “f” indicated 2′-fluoro; “*” indicates phosphorothioate linkage; “Uhd” indicates 2′-O-hexadecyl uridine; “Ahd” indicates 2′-O-hexadecyl adenosine; “Chd” indicates 2′O-hexadecyl cytidine; “Ghd” indicates 2′-O-hexadecyl guanosine; “PrON” indicates racemic 2-propyl-o-uridine; “L” indicates locked nucleic acid; “e” indicates 2′-methoxyethyl; “[iAb]” indicates inverted abasic; “Y” indicates pseudouridine,; “d” indicates 2′-deoxy; “UNA” indicates unlocked nucleic acid; “[C5P-C]” indicates 5-propynylcytosine; “[DAP]” indicates 2,6-diaminopurine; “s” means the sense strand; “as” means the antisense strand, the 5′ of the antisense may have either a P or VP.

The sense strand and antisense strand of dsRNA can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphotriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a dsRNA.

### Linker

The conjugates described herein can include a linker (L).

In some embodiments, L comprises a linker core (LC) and one or more spacers. Exemplary LC structures are provided in Table 7. The spacers may be small organic groups or polymer chains or the like. In some embodiments, L comprises Spacer1-Linker Core-Spacer2. In some embodiments, Spacer1 and Spacer 2 are selected from Table 8. Exemplary linkers are provided in Table 9.

TABLE 7
Exemplary Linker Core (LC) Structures

| Linker Core | Structure |
|---|---|
| LC1 | —CO—C2—CO—DBCO/N3—CH2—CO—- |
| | 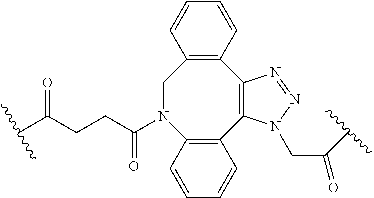 [SMILES: *C(=O)CCC(=O)N1Cc2ccccc2-c2nnn(CC(*)=O)c2-c2ccccc21] |
| LC2 | —CO—SMCC—Cys—CO— |
| | 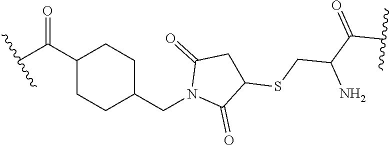 [SMILES: *C(=O)C(N)CSC1CC(=O)N(CC2CCC(C(*)=O)CC2)C1=O] |
| LC3 | —CO—SMCC/Mpa—CO— |
| | 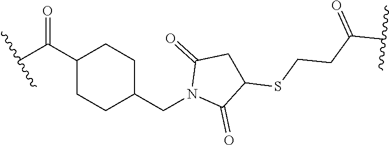 [SMILES: *C(=O)CCSC1CC(=O)N(CC2CCC(C(*)=O)CC2)C1=O] |
| LC4 | —CO—PEG—POD/Mpa—CO— |
| | 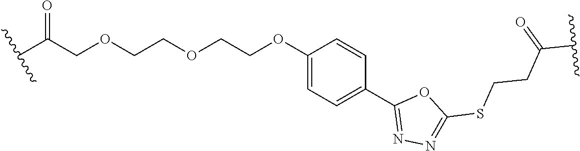 [SMILES: *C(=O)CCSc1nnc(-c2ccc(OCCOCCOCC(*)=O)cc2)o1] |
| LC5 | —CO—PEG—PT/Mpa—CO— |
| | 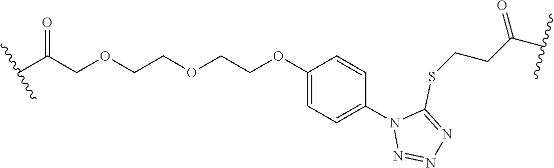 [SMILES: *C(=O)CCSc1nnnn1-c1ccc(OCCOCCOCC(*)=O)cc1] |
| LC6 | —CO—CH2—Mpa—CO— |
| | 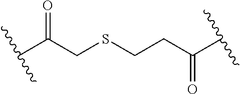 [SMILES: *C(=O)CCSCC(*)=O] |
| LC7 | —CO—Mpa/Mal(RO)-Dap- |
| | Regioisomer 1: |
| | 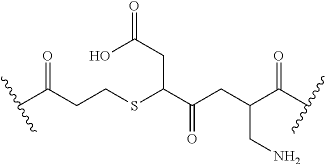 [SMILES: *C(=O)CCSC(CC(=O)O)C(=O)CC(CN)C(*)=O] |
| | Regioisomer 2: |
| | 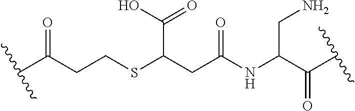 [SMILES: *C(=O)CCSC(CC(=O)NC(CN)C(*)=O)C(=O)O] |
| LC8 | —CO—SMCC(RO)/Mpa—CO— |
| | Regioisomer 1: |
| | 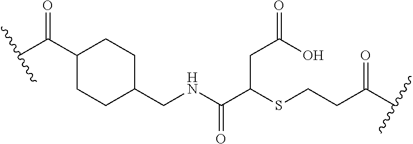 [SMILES: *C(=O)CCSC(CC(=O)O)C(=O)NCC1CCC(C(*)=O)CC1] |
| | Regioisomer 2: |
| | 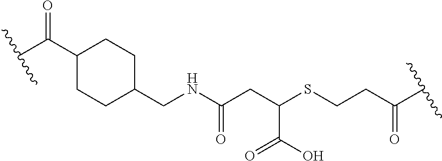 [SMILES: *C(=O)CCSC(CC(=O)NCC1CCC(C(*)=O)CC1)C(=O)O] |
| LC9 | —/Mal(RO)-Dap—CO— |
| | Regioisomer 1: |
| | 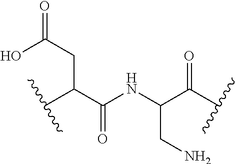 [SMILES: *C(=O)C(CN)NC(=O)C(*)CC(=O)O] |
| | Regioisomer 2: |
| | 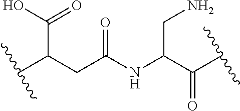 [SMILES: *C(=O)C(CN)NC(=O)CC(*)C(=O)O] |
| LC10 | —CO-Mpa/PT—PEG—CO— |
| | 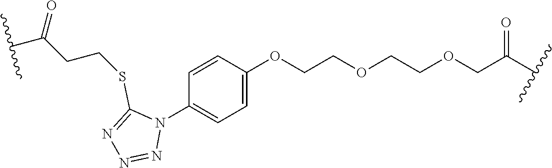 [SMILES: *C(=O)CCSc1nnnn1-c1ccc(OCCOCCOCC(*)=O)cc1] |
| LC11 | —/PT—PEG—CO |
| | 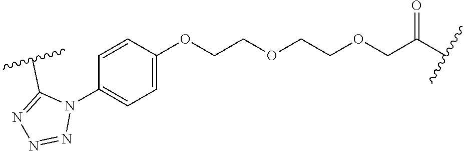 [SMILES: *C(=O)COCCOCCOc1ccc(-n2nnnc2*)cc1] |

It will be appreciated that in some instances, the liner structures above represent final structural configurations of the linkers where precursor molecules on different portions of the conjugate have been reacted to yield such structures. That is, a first precursor to the linker may be covalently attached to the oligonucleotide, and a second precursor to the linker may be covalently attached to the peptide. Then the two precursors are brought into proximity with one another under proper conditions and form a covalent linkage yielding one of the structures shown herein.

TABLE 8
Exemplary Linker Spacers (SL, SP)

| Spacer | |
|---|---|
| name | Spacer structure |
| SL1 | —P(O)SH—O—C6—NH— |
| | 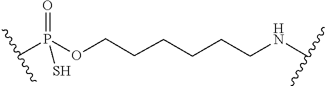 [SMILES: *NCCCCCCOP(*)(=O)S] |
| SL2 | (2′'—OH)—C3—NH— |
| | 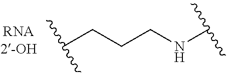 [SMILES: *CCCN*] |
| SL3 | —P(O)SH—O—C6—S— |
| | 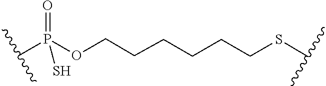 [SMILES: *SCCCCCCOP(*)(=O)S] |
| SL4 | P(O)OH—O—C6—NH— |
| | 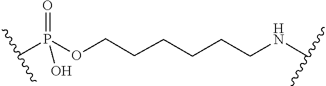 [SMILES: *NCCCCCCOP(*)(=O)O] |
| SL5 | P(O)OH—O—C6—S— |
| | 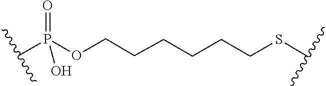 [SMILES: *SCCCCCCOP(*)(=O)O] |
| SP1 | —NH—AEEA3—CO— |
| | 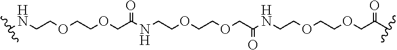 [SMILES: *NCCOCCOCC(=O)NCCOCCOCC(=O)NCCOCCOCC(*)=O] |
| SP2 | AEEA3-fSp-Cha-aGPIDRIGGGGSGGGGS |
| (SEQ | |
| ID: 58) | |
| SP3 | GGGGSGGGGSfSp-Cha-aGPIDRIGGGGSGGGGS |
| (SEQ | |
| ID: 59) | |
| SP4 | GGGGSGGGGSGGGGSGGGGS |
| (SEQ | |
| ID: 60) | |
| SP5 | AEEA3-GGGGSGGGGSGGGGSGGGGS |
| (SEQ | |
| ID: 61) | |
| SP6 | —NH—AEEA6—CO— |
| | 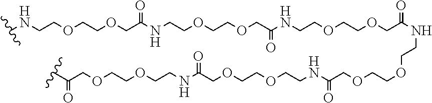 [SMILES: *NCCOCCOCC(=O)NCCOCCOCC(=O)NCCOCCOCC(=O)NCCOCCOCC(=O)NCCOCCOCC(=O)NCCOCCOCC(*)=O] |
| SP7 | —NH—AEEA8—CO— |
| | 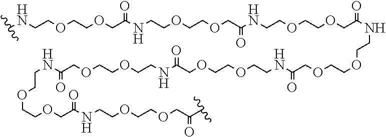 [SMILES: *NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(*)=O] |
| SP8 | GGGGHGGGGHGGGGHGGGGH |
| (SEQ | |
| ID: 62) | |
| SP9 | GGGG-4Pal-GGGG-4Pal-GGGG-4Pal-GGGG-4Pal |
| (SEQ | |
| ID: 63) | |
| SP10 | GGGGSHHHGGGGSHHHGGGGS |
| (SEQ | |
| ID: 64) | |
| SP11 | AEEA3-GGGGSGGGGS |
| (SEQ | |
| ID: 65) | |
| SP12 | GGGGSGGGGSGGGGSGGGGSGGGGSGGGGS |
| (SEQ | |
| ID: 66) | |
| SP13 | —NH—PEG24—CO— |
| | 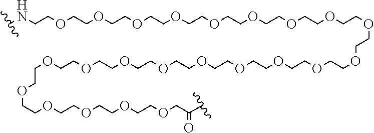 [SMILES: *NCCOCCOCCOCCOCCOCCOCCOCCOCCOCCOCCOCCOCCOCCOCCOCCOCCOCCOCCOCCOCCOCCOCCOCC(*)=O] |
| SP14 | GGGGSGGGGSGGGGSGGGGSP |
| (SEQ | |
| ID: 67) | |
| SP15 | GGGG—4Pal—GGGG—4Pal—GGGG—4Pal—GGGG—4Pal—P |
| (SEQ | |
| ID: 68) | |
| SP16 | GGGG—4Pal—GGGG—4Pal—GGGG—4Pal—GGGG—4Pal—GGGG—4Pal—GGGG—4Pal |
| (SEQ | |
| ID: 69) | |
| SP17 | GGGGEGGGGEGGGGSGGGGSGGGGSGGGGS |
| (SEQ | |
| ID: 70) | |
| SP18 | GGGGEGGGGSGGGGEGGGGSGGGGEGGGGS |
| (SEQ | |
| ID: 71) | |
| SP19 | GGGGEGGGGEGGGGEKEKEKGGGGSGGGGS |
| (SEQ | |
| ID: 72) | |

TABLE 9
Exemplary Linkers (L)

| Linker Name | Spacer1(SL)-Linker Core(LC)-Spacer2(SP) |
|---|---|
| L1 | SL1-LC1-SP1 |
| L2 | SL1-LC2-SP1 |
| L3 | SL1-LC3-SP1 |
| L4 | SL1-LC4-SP1 |
| L5 | SL1-LC5-SP1 |
| L6 | SL1-LC6-SP1 |
| L7 | SL1-LC7-SP1 |
| L8 | SL1-LC8-SP1 |
| L9 | SL-LC10-SP1 |
| L10 | SL3-LC9-SP1 |
| L11 | SL2-LC3-SP1 |
| L12 | SL1-LC3-SP2 |
| L13 | SL1-LC3-SP3 |
| L14 | SL1-LC3-SP7 |
| L15 | SL1-LC3-SP8 |
| L16 | SL1-LC3-SP9 |
| L17 | SL1-LC3-SP10 |
| L18 | SL1-LC3-SP12 |
| L19 | SL1-LC5-SP12 |
| L20 | SL1-LC10-SP12 |
| L21 | SL1-LC7-SP12 |
| L22 | SL3-LC9-SP12 |
| L23 | SL3-LC11-SP12 |
| L24 | SL3-LC11-SP1 |
| L25 | SL3-LC11-SP17 |
| L26 | SL3-LC11-SP18 |
| L27 | SL3-LC11-SP19 |

### Fatty Acids

In some embodiments, the conjugates further comprise a fatty acid (FA) or lipid. The FA can be saturated or unsaturated. In some embodiments, the FA is a C₁₂-C₂₂ fatty acid. Exemplary fatty acids are provided in Table 10.

In some embodiments, FA is attached to O. In some embodiments, FA is attached to P. In some embodiments, FA is attached to L.

In some embodiments, the conjugate comprises (FA)ₘ-O-L-P or O-L-P-(FA)ₘ, wherein m is an integer of 1 to 4. In some embodiments, m is 1 or 2.

In some embodiments, the conjugate further comprises a Spacer3. Exemplary fatty acid spacers (Spacer3) are provided in Table 11.

Exemplary FA-Spacer3 pairs are provided in Table 12.

In some embodiments, the conjugate comprises (FA-Spacer3)ₘ-O-L-P or O-L-P-(Spacer3-FA)ₘ, wherein m is an integer of 1 to 4. In some embodiments, m is 1 or 2. In some embodiments, the conjugate comprises FA-Spacer3-O-L-P or O-L-P-Spacer3-FA. In some embodiments, the conjugate comprises FA-Spacer3-O-Spacer1-LinkerCore-Spacer2-P or O-Spacer1-LinkerCore-Spacer2-P-Spacer3-FA.

In some embodiments, the conjugate comprises O-L-(FA-Spacer3)ₘ-P or 0-(Spacer3-FA)ₘ-L-P, wherein m is an integer of 1 to 4. In some embodiments, m is 1 or 2. In some embodiments, the conjugate comprises O-L-FA-Spacer3-P or O-Spacer3-FA-L-P. In some embodiments, the conjugate comprises O-Spacer1-LinkerCore-Spacer2-FA-Spacer3-P or O-Spacer3-FA-Spacer1-LinkerCore-Spacer2-P.

TABLE 10
Exemplary Fatty Acids (FA)

| Fatty | |
|---|---|
| Acid | Structure |
| FA1 | Ac—L—Cpa(/L—Cys—CO—)—K(AEEA2—yE—C20 diacid)—NH2 |
| | 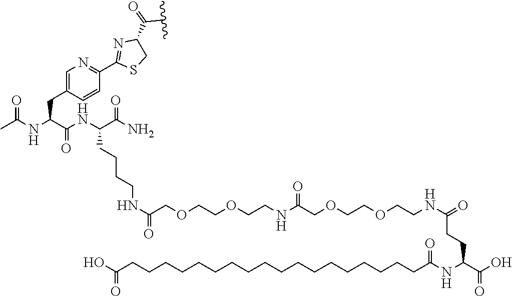 [SMILES: *C(=O)[C@@H]1CSC(c2ccc(C[C@H](NC(C)=O)C(=O)N[C@@H](CCCCNC(=O)COCCOCCNC(=O)COCCOCCNC(=O)CC[C@H](NC(=O)CCCCCCCCCCCCCCCCCCC(=O)O)C(=O)O)C(N)=O)cn2)=N1] |
| FA2 | —CO—AEEA2—yE—C20 diacid |
| | 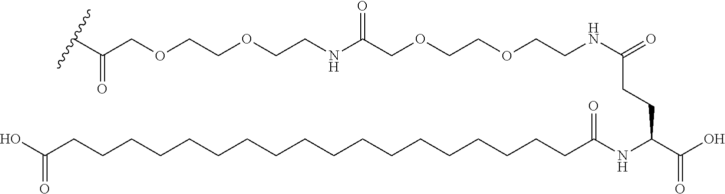 [SMILES: *C(=O)COCCOCCNC(=O)COCCOCCNC(=O)CC[C@H](NC(=O)CCCCCCCCCCCCCCCCCCC(=O)O)C(=O)O] |
| FA3 | —K(AEEA2—yE—C20 diacid)—NH2 |
| | 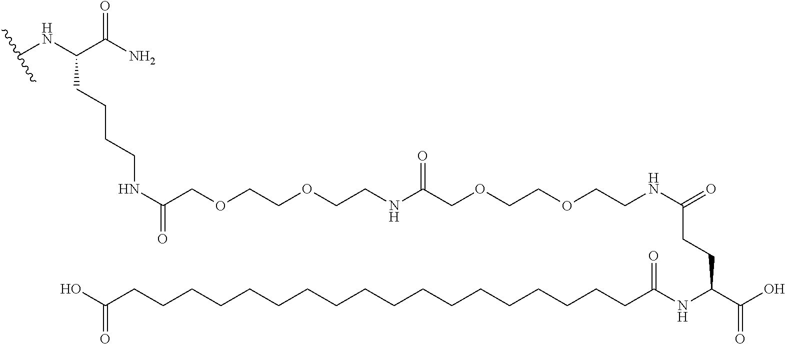 [SMILES: *N[C@@H](CCCCNC(=O)COCCOCCNC(=O)COCCOCCNC(=O)CC[C@H](NC(=O)CCCCCCCCCCCCCCCCCCC(=O)O)C(=O)O)C(N)=O] |
| FA4 | —K(AEEA2—yE—C20 diacid)— |
| | 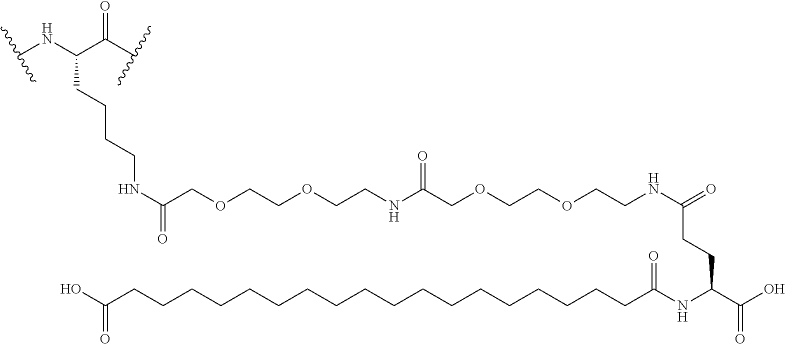 [SMILES: *N[C@@H](CCCCNC(=O)COCCOCCNC(=O)COCCOCCNC(=O)CC[C@H](NC(=O)CCCCCCCCCCCCCCCCCCC(=O)O)C(=O)O)C(*)=O] |
| FA5 | —P(O)SH—O—C16 |
| | 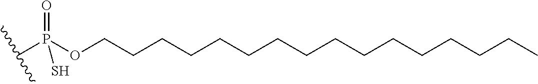 [SMILES: *P(=O)(S)OCCCCCCCCCCCCCCCC] |
| FA6 | —P(O)SH—O—C22 |
| | 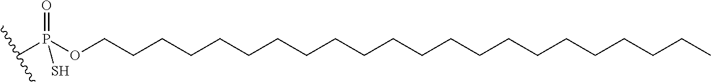 [SMILES: *P(=O)(S)OCCCCCCCCCCCCCCCCCCCCCC] |
| FA7 | —P(O)SH—O—C18 unsaturated (-oleyl) |
| | 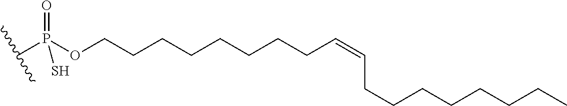 [SMILES: *P(=O)(S)OCCCCCCCC/C=C\CCCCCCCC] |
| FA8 | —P(O)SH—O—C20 branched |
| | 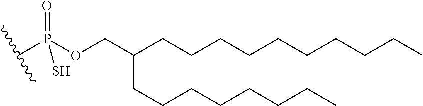 [SMILES: *P(=O)(S)OCC(CCCCCCCC)CCCCCCCCCC] |
| FA9 | —P(O)SH—O—C12 caged (-ethyl adamantyl) |
| | 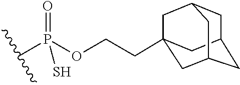 [SMILES: *P(=O)(S)OCCC12CC3CC(CC(C3)C1)C2] |
| FA10 | —C20 diacid |
| | 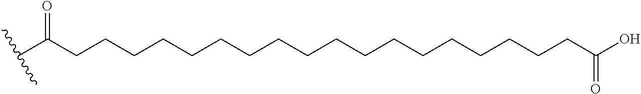 [SMILES: *C(=O)CCCCCCCCCCCCCCCCCCC(=O)O] |
| FA11 | —P(O)OH—O—C16 |
| | 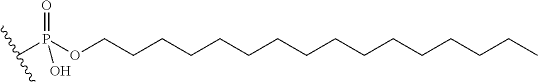 [SMILES: *P(=O)(O)OCCCCCCCCCCCCCCCC] |
| FA12 | —P(O)OH—O—C22 |
| | 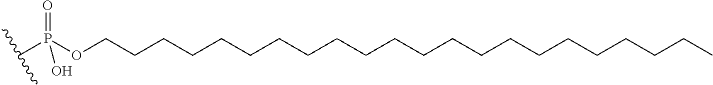 [SMILES: *P(=O)(O)OCCCCCCCCCCCCCCCCCCCCCC] |
| FA13 | —P(O)OH—O—C18 unsaturated (-oleyl) |
| | 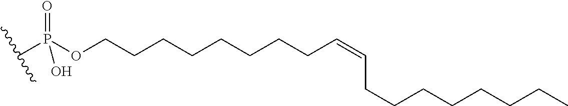 [SMILES: *P(=O)(O)OCCCCCCCC/C=C\CCCCCCCC] |
| FA14 | —P(O)OH—O—C20 branched |
| | 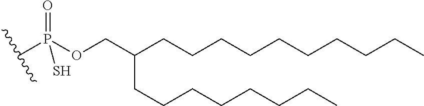 [SMILES: *P(=O)(S)OCC(CCCCCCCC)CCCCCCCCCC] |
| FA15 | —P(O)OH—O—C12 caged (-ethyl adamantyl) |
| | 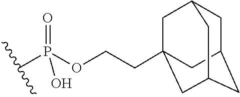 [SMILES: *P(=O)(O)OCCC12CC3CC(CC(C3)C1)C2] |
| FA16 | —P(O)SH—O—glycol—(P(O-C16)SH)2 doubler |
| | 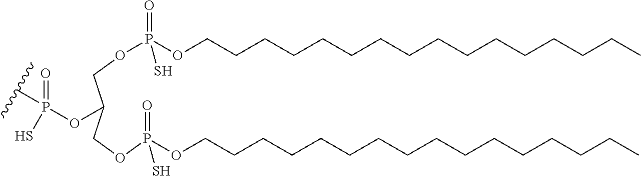 [SMILES: *P(=O)(S)OC(COP(=O)(S)OCCCCCCCCCCCCCCCC)COP(=O)(S)OCCCCCCCCCCCCCCCC] |
| FA17 | —P(O)SH—O—glycol—(P(O-C16)OH)2 doubler |
| | 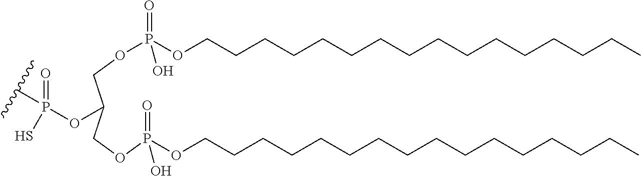 [SMILES: *P(=O)(S)OC(COP(=O)(O)OCCCCCCCCCCCCCCCC)COP(=O)(O)OCCCCCCCCCCCCCCCC] |
| FA18 | —P(O)OH—O—glycol—(P(O-C16)OH)2 doubler |
| | 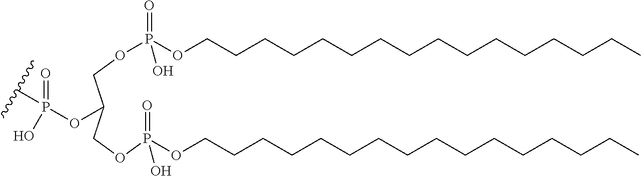 [SMILES: *P(=O)(O)OC(COP(=O)(O)OCCCCCCCCCCCCCCCC)COP(=O)(O)OCCCCCCCCCCCCCCCC] |
| FA19 | —P(O)SH—O—C16-Acid |
| | 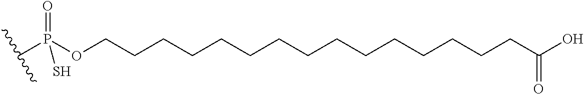 [SMILES: *P(=O)(S)OCCCCCCCCCCCCCCCC(=O)O] |
| FA20 | —P(O)SH—O—C18-Acid |
| | 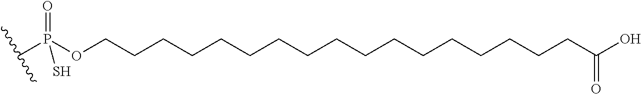 [SMILES: *P(=O)(S)OCCCCCCCCCCCCCCCCCC(=O)O] |
| FA21 | —P(O)SH—O—C20-Acid |
| | 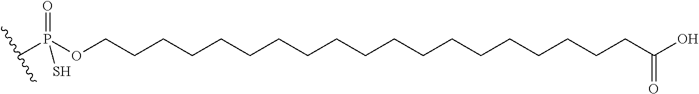 [SMILES: *P(=O)(S)OCCCCCCCCCCCCCCCCCCCC(=O)O] |
| FA22 | —P(O)OH—O—C16-Acid |
| | 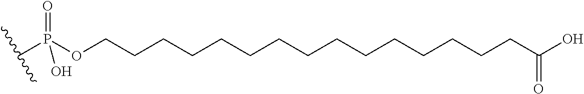 [SMILES: *P(=O)(O)OCCCCCCCCCCCCCCCC(=O)O] |
| FA23 | —P(O)OH—O—C18-Acid |
| | 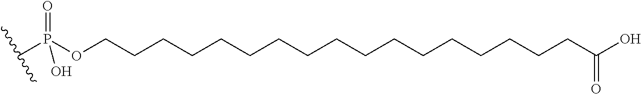 [SMILES: *P(=O)(O)OCCCCCCCCCCCCCCCCCC(=O)O] |
| FA24 | —P(O)OH—O—C20-Acid |
| | 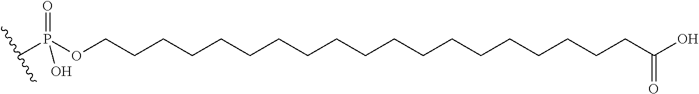 [SMILES: *P(=O)(O)OCCCCCCCCCCCCCCCCCCCC(=O)O] |
| FA25 | —yE—C16 diacid |
| | 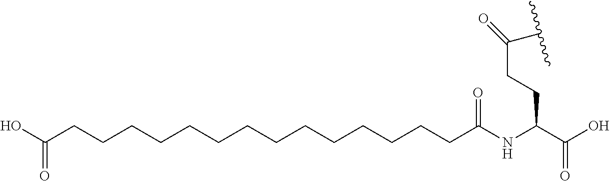 [SMILES: *C(=O)CC[C@H](NC(=O)CCCCCCCCCCCCCCC(=O)O)C(=O)O] |
| FA26 | —C16 diacid |
| | 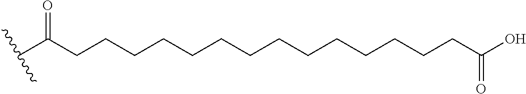 [SMILES: *C(=O)CCCCCCCCCCCCCCC(=O)O] |
| FA27 | —C20 |
| | 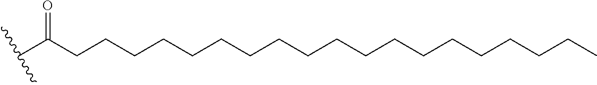 [SMILES: *C(=O)CCCCCCCCCCCCCCCCCCC] |

TABLE 11
Exemplary Spacers for Fatty Acids (Spacer3)

| | Spacers For Fatty Acids (Spacer3) |
|---|---|
| SF1* | (2′—OH)—C3—NH— |
| | 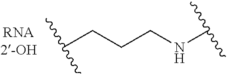 [SMILES: *CCCN*] |
| SF2 | —P(O)OH—O—cHex—pNH— |
| | 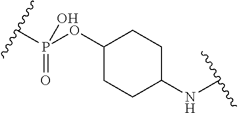 [SMILES: *NC1CCC(OP(*)(=O)O)CC1] |
| SF3 | —P(O)SH—O—C6—NH— |
| | 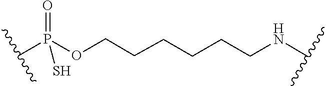 [SMILES: *NCCCCCCOP(*)(=O)S] |
| SF4 | GSPSSGAPPPS |
| (SEQ ID: | |
| 73) | |
| SF5 | —NH—AEEA3—CO— |
| | 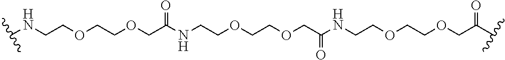 [SMILES: *NCCOCCOCC(=O)NCCOCCOCC(=O)NCCOCCOCC(*)=O] |
| SF6 | —P(O)SH—O—cHex—pNH— |
| | 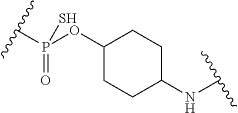 [SMILES: *NC1CCC(OP(*)(=O)S)CC1] |
| SF7 | —P(O)OH—O—C6—NH— |
| | 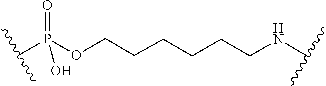 [SMILES: *NCCCCCCOP(*)(=O)O] |
| SF8 | —P(O)SH—O—C2—NH— |
| | 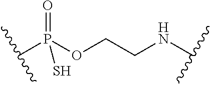 [SMILES: *NCCOP(*)(=O)S] |
| SF9 | —P(O)OH—O—C2—NH— |
| | 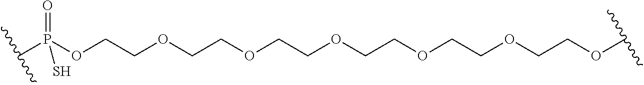 [SMILES: *OCCOCCOCCOCCOCCOCCOP(*)(=O)S] |
| SF10 | —P(O)SH—O—PEG6—O— |
| | 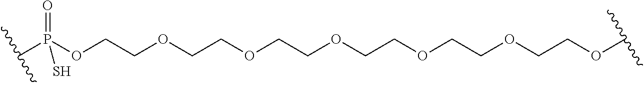 [SMILES: *OCCOCCOCCOCCOCCOCCOP(*)(=O)S] |
| SF11 | —P(O)OH—O—PEG6—O— |
| | 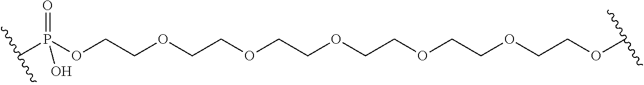 [SMILES: *OCCOCCOCCOCCOCCOCCOP(*)(=O)O] |
| SF12 | —P(O)SH—O—PEG6—O—P(O)SH—O—C2—NH— |
| | 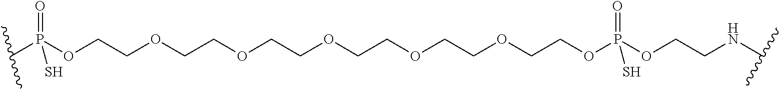 [SMILES: *NCCOP(=O)(S)OCCOCCOCCOCCOCCOCCOP(*)(=O)S] |
| SF13 | —P(O)SH—O—PEG6—O—P(O)OH—O—C2—NH— |
| | 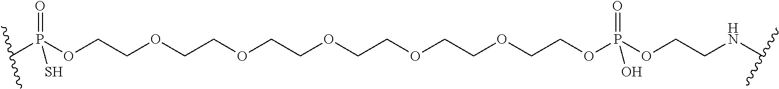 [SMILES: *NCCOP(=O)(O)OCCOCCOCCOCCOCCOCCOP(*)(=O)S] |
| SF14 | —P(O)OH—O—PEG6—O—P(O)SH—O—C2—NH— |
| | 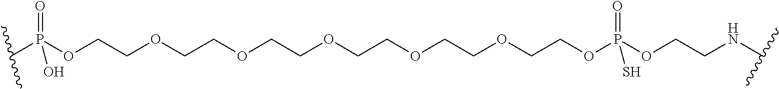 [SMILES: *NCCOP(=O)(S)OCCOCCOCCOCCOCCOCCOP(*)(=O)O] |
| SF15 | —P(O)OH—O—PEG6—O—P(O)OH—O—C2—NH— |
| | 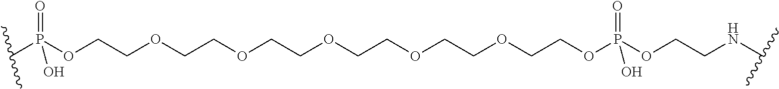 [SMILES: *NCCOP(=O)(O)OCCOCCOCCOCCOCCOCCOP(*)(=O)O] |

*For SF1, “(2′-OH)—C3—NH—” the aminopropyl moiety extends from the position where the 2′-hydroxyl hydrogen of an RNA would otherwise exist, with reference to the Upa 2′-O-propylamino uridine modified base.

TABLE 12
Exemplary FA-Spacer3 Pairs

| Pair No. | Fatty Acid-Spacer3 Pairs |
|---|---|
| 1 | FA1-SF2 |
| 2 | FA2-SF3 |
| 3 | FA3-SF4 |
| 4 | FA4-SF5 |
| 5 | FA4-SF4 |
| 6 | FA10-SF8 |
| 7 | FA5-SF10 |
| 8 | FA6-SF10 |
| 9 | FA19-SF10 |
| 10 | FA20-SF10 |
| 11 | FA21-SF10 |
| 12 | FA10-SF12 |
| 13 | FA25-SF10 |
| 14 | FA26-SF8 |

### Conjugates

Certain exemplary conjugates of the present disclosure are provided in Table 13:

TABLE 13
Exemplary conjugates comprising NPR-C binding peptide and dsRNA

| | | | dsRNA (R) | | Linker | | Peptide (P) | Configuration |
|---|---|---|---|---|---|---|---|---|
| Conjugate | FA | SF | SEQ ID | SL | Core (LC) | SP | SEQ | (5′ end to 3′ |
| NO | NO | NO | NO | NO | NO | NO | ID NO | end) |
| C1 | — | — | 53 | 1 | 3 | 1 | 20 | R-SL-LC-SP- |
| | | | | | | | | P |
| C2 | — | — | 52 | 1 | 3 | 1 | 20 | R-SL-LC-SP- |
| | | | | | | | | P |
| C3 | — | — | 49 | 1 | 3 | 1 | 20 | R-SL-LC-SP- |
| | | | | | | | | P |
| C4 | — | — | 50 | 1 | 10 | 1 | 21 | R-SL-LC-SP- |
| | | | | | | | | P |
| C5 | — | — | 73 | 1 | 10 | 1 | 21 | R-SL-LC-SP- |
| | | | | | | | | P |
| C6 | — | — | 77 | 1 | 10 | 1 | 21 | R-SL-LC-SP- |
| | | | | | | | | P |
| C7 | — | — | 54 | 1 | 10 | 1 | 21 | R-SL-LC-SP- |
| | | | | | | | | P |
| C8 | — | — | 89 | 1 | 10 | 1 | 21 | R-SL-LC-SP- |
| | | | | | | | | P |
| C9 | — | — | 53 | 1 | 10 | 1 | 21 | R-SL-LC-SP- |
| | | | | | | | | P |
| C10 | — | — | 49 | 1 | 10 | 1 | 21 | R-SL-LC-SP- |
| | | | | | | | | P |
| C11 | 10 | 8 | 173 | 3 | 11 | 1 | 21 | FA-SF-R-SL- |
| | | | | | | | | LC-SP-P |
| C12 | — | — | 173 | 3 | 11 | 1 | 21 | R-SL-LC-SP- |
| | | | | | | | | P |
| C13 | 10 | 8 | 173 | 3 | 11 | 19 | 21 | FA-SF-R-SL- |
| | | | | | | | | LC-SP-P |
| C14 | — | — | 173 | 3 | 11 | 19 | 21 | R-SL-LC-SP- |
| | | | | | | | | P |
| C15 | — | — | 278 | 3 | 11 | 19 | 21 | R-SL-LC-SP- |
| | | | | | | | | P |
| C16 | — | — | 280 | 3 | 11 | 19 | 21 | R-SL-LC-SP- |
| | | | | | | | | P |
| C17 | — | — | 281 | 3 | 11 | 19 | 21 | R-SL-LC-SP- |
| | | | | | | | | P |
| C18 | — | — | 282 | 3 | 11 | 19 | 21 | R-SL-LC-SP- |
| | | | | | | | | P |
| C19 | — | — | 283 | 3 | 11 | 19 | 21 | R-SL-LC-SP- |
| | | | | | | | | P |
| C20 | — | — | 284 | 3 | 11 | 19 | 21 | R-SL-LC-SP- |
| | | | | | | | | P |
| C21 | — | — | 285 | 3 | 11 | 19 | 21 | R-SL-LC-SP- |
| | | | | | | | | P |
| C22 | 26 | 8 | 173 | 3 | 11 | 19 | 21 | FA-SF-R- |
| | | | | | | | | SL-LC-SP-P |
| C23 | 26 | 8 | 278 | 3 | 11 | 19 | 21 | FA-SF-R- |
| | | | | | | | | SL-LC-SP-P |
| C24 | 26 | 8 | 280 | 3 | 11 | 19 | 21 | FA-SF-R- |
| | | | | | | | | SL-LC-SP-P |
| C25 | 26 | 8 | 281 | 3 | 11 | 19 | 21 | FA-SF-R- |
| | | | | | | | | SL-LC-SP-P |
| C26 | 26 | 8 | 282 | 3 | 11 | 19 | 21 | FA-SF-R- |
| | | | | | | | | SL-LC-SP-P |
| C27 | 26 | 8 | 283 | 3 | 11 | 19 | 21 | FA-SF-R- |
| | | | | | | | | SL-LC-SP-P |
| C28 | 26 | 8 | 284 | 3 | 11 | 19 | 21 | FA-SF-R- |
| | | | | | | | | SL-LC-SP-P |
| C29 | 26 | 8 | 285 | 3 | 11 | 19 | 21 | FA-SF-R- |
| | | | | | | | | SL-LC-SP-P |
| C30 | — | — | 286 | 3 | 11 | 19 | 21 | R-SL-LC-SP- |
| | | | | | | | | P |
| C31 | — | — | 287 | 3 | 11 | 19 | 21 | R-SL-LC-SP- |
| | | | | | | | | P |
| C32 | — | — | 288 | 3 | 11 | 19 | 21 | R-SL-LC-SP- |
| | | | | | | | | P |
| C33 | — | — | 289 | 3 | 11 | 19 | 21 | R-SL-LC-SP- |
| | | | | | | | | P |
| C34 | — | — | 290 | 3 | 11 | 19 | 21 | R-SL-LC-SP- |
| | | | | | | | | P |
| C35 | — | — | 291 | 3 | 11 | 19 | 21 | R-SL-LC-SP- |
| | | | | | | | | P |
| C36 | — | — | 292 | 3 | 11 | 19 | 21 | R-SL-LC-SP- |
| | | | | | | | | P |
| C37 | — | — | 293 | 3 | 11 | 19 | 21 | R-SL-LC-SP- |
| | | | | | | | | P |

*dsRNAs listed in this conjugate table were, or may be, made with a 5′-vinylphosphonate modification on the antisense strand.

The conjugates described herein can be made by a variety of procedures known to one of ordinary skill in the art, some of which are illustrated in the preparations and examples below. One of ordinary skill in the art recognizes that the specific synthetic steps for each of the routes described may be combined in different ways, or in conjunction with steps from different schemes, to prepare conjugates. The product of each step can be recovered by conventional methods well known in the art, including extraction, evaporation, precipitation, chromatography, filtration, trituration, and crystallization. The reagents and starting materials are readily available to one of ordinary skill in the art.

### Pharmaceutical Composition

In another aspect, provided herein are pharmaceutical compositions comprising any of the oligonucleotides, RNAi agents, or conjugates described herein and a pharmaceutically acceptable carrier. Such pharmaceutical compositions target PDE3B in a patient in need of treatment. The pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

### Method of Treatment and Therapeutic Use

In another aspect, provided herein are methods of treating a disease or condition of adipose tissue, e.g., obesity or obesity-related comorbidity, in a patient in need thereof, and such the method comprises administering to the patient an effective amount of an oligonucleotide, RNAi agent, conjugate, or pharmaceutical composition described herein. The conjugate or pharmaceutical composition can be administered to the patient intravenously or subcutaneously.

Also provided herein are methods of delivering an oligonucleotide to an adipose tissue, comprising administering to a subject a conjugate or pharmaceutical composition described herein.

Dosage regimens for the conjugates described herein may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are conjugates or pharmaceutical compositions described herein for use in a therapy. In some embodiments, provided herein are conjugates or pharmaceutical compositions described herein for use in the treatment of a disease or condition involving PDE3B, e.g., obesity or obesity-related comorbidity.

Also provided herein are uses of conjugates or pharmaceutical compositions described herein in the manufacture of a medicament for treating a disease or condition involving PDE3B, including in or of adipose tissue, e.g., obesity or obesity-related comorbidity.

Also provided herein are methods of delivering an oligonucleotide to an adipose tissue, comprising administering to a subject a conjugate or pharmaceutical composition described herein.

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a protein or conjugate may vary according to factors such as the disease state, age, sex, and weight of the individual, and the ability of the protein or conjugate to elicit a desired response in the individual. An effective amount is also one in which any toxic or detrimental effects of the protein or conjugate are outweighed by the therapeutically beneficial effects.

The term “knockdown” or “expression knockdown” refers to reduced mRNA or protein expression of a gene after treatment of a reagent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, or 2′-O-alkyl (e.g., 2′-O—C₁₆ alkyl) modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 6.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, “oligonucleotide” means a polymer of linked nucleotides, each of which can be modified or unmodified. An oligonucleotide is typically less than about 100 nucleotides in length.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

As used herein, “polypeptide” or “peptide” means a polymer of amino acid residues comprising two (2) or more amino acids and/or amino acid derivatives which, in general, are linked via peptide bonds. The term applies to polymers comprising naturally occurring amino acids and polymers comprising one or more non-naturally occurring amino acids. Embodiments may include modifications or amino acid derivatives, including synthetic modifications, some of which may resemble post-translational modifications such as, phosphorylation, hydroxylation, sulfonation, acylation, glycosylation and disulfide formation.

As used herein, “iRNA,” “iRNA agent,” “RNAi,” “RNAi agent” and “RNA interference agent” means an agent that contains RNA and mediates the targeted cleavage of a RNA transcript via RNA interference, e.g., through a RNA-induced silencing complex (RISC) pathway. In some embodiments, the RNAi agent has a sense strand and an antisense strand, and the sense strand and the antisense strand form a duplex. In some embodiments, the sense and antisense strands of RNAi agent are 21-23 nucleotides in length. In other embodiments, the sense and antisense strands can be longer, for example 25-30 nucleotides in length, in which case the longer RNAi sequences are first processed by the Dicer enzyme. The iRNA attenuates, inhibits, modulates, or reduces PDE3B expression in a cell. The RNAi agent as disclosed herein may include portions or moieties other than nucleic acids, including but not limited to a peptide portion (such as an NPR-C binding peptide); a fatty acid; and linkers and spaces, among others.

In some embodiments, the peptides described herein include a fatty acid conjugated, for example, by way of a direct bond or linker to a natural or non-natural amino acid with a functional group available for conjugation. Such a conjugation is sometimes referred to as acylation. In certain instances, the amino acid with a functional group available for conjugation can be K, C, E, and D. In particular instances, the amino acid with a functional group available for conjugation is K, where the conjugation is to an F-amino group of a K side-chain. In some embodiments, the peptides described herein are amidated. In some embodiments, the peptides described herein have a modification of the C-terminal group, wherein the modification is NH₂. In some embodiments, the peptides described herein have a modification of the C-terminal group where the modification is absent.

Amino acids which may be incorporated into the peptides of the present disclosure include the twenty standard or canonical amino acids, and a number of other nonstandard amino acids. Some of the nonstandard amino acids are shown below in Table 16.

TABLE 14
Selected nonstandard amino acids

| NAME | ABBREVIATION(S) | STRUCTURE |
|---|---|---|
| D-Alanine | a, D-Ala | 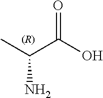 |
| D-Phenylalanine | f, D-Phe | 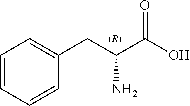 |
| D-Proline | p, D-Pro | 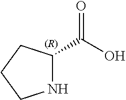 |
| D-Serine | s, D-Ser | 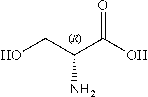 |
| D-Tyrosine | y, D-Tyr | 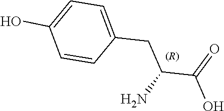 |
| 2-Aminoisobutryic Acid | Aib | 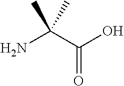 |
| β-Cyclohexyl-L-Alanine | Cha | 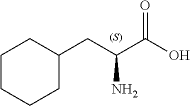 |
| L-2,3-Diaminopropionic Acid | Dap | 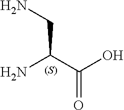 |
| L-Hydroxyproline | Hyp | 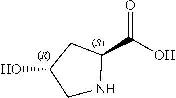 |
| D-Hydroxyproline | D-Hyp | 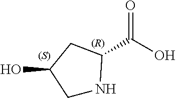 |
| L-Ornithine | Orn | 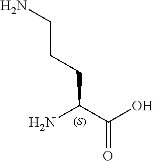 |
| Alpha-Methyl-2-Fluoro-L- Phenylalanie | αMePhe(2F) | 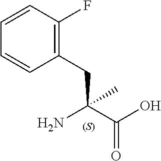 |
| 3-Mercaptopropanoic Acid | Mpa | 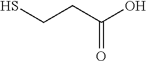 |
| N^{W}-Methyl-L-Arginine | Arg(Me) | 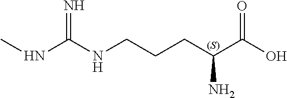 |
| 4-Pyridyl-L-Alanine | 4-Pal | 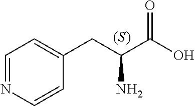 |
| 3-(2-Naphthyl)-L-Alanine | 2Nal | 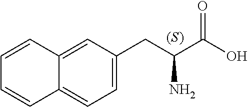 |
| L-Homophenylalanine | hPhe | 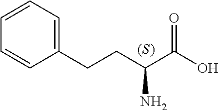 |
| D-Homoproline | D-Pip | 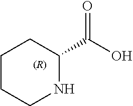 |
| L-Homoglutamic Acid | Aad | 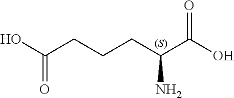 |
| N-Methyl-L- Arginine | NMe-Arg | 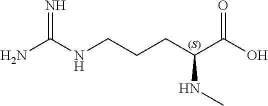 |
| Gamma-L-Glutamic Acid | γE | 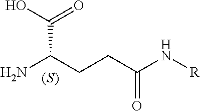 |
| 2-[2-(2-amino-ethoxy)-ethoxy]- acetic acid | AEEA | 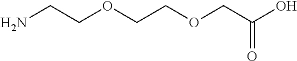 [SMILES: NCCOCCOCC(=O)O] |

As used herein, where the full name of an amino acid is spelled out (“arginine,” etc.), all stereoisomers of that amino acid is encompassed (e.g., L-arginine and D-arginine.) Where a single-letter abbreviation is used, an uppercase letter denotes the L isomer (that is, the isomer incorporated into polypeptides in nature), and a lowercase letter denotes the D isomer. Noncanonical amino acids are generally denoted by a three-letter abbreviation rather than a single letter, or as specified in the table above.

The peptides described herein may react with any number of inorganic and organic acids/bases to form pharmaceutically acceptable acid/base addition salts. Pharmaceutically acceptable salts and common techniques for preparing them are well known in the art (see, e.g, Stahl et. al, Handbook of Pharmaceutical Salts: Properties, Selection, and Use, 2^{nd} Revised Edition (Wiley-VCH, 2011)). Pharmaceutically acceptable salts for use herein include sodium, potassium, trifluoroacetate, hydrochloride and/or acetate salts. The disclosure also provides and therefore encompasses novel intermediates and methods of synthesizing the polypeptides described herein, or pharmaceutically acceptable salts thereof. The intermediates and polypeptides described herein can be prepared by a variety of techniques known in the art. For example, a method using chemical synthesis is illustrated in the Examples below.

The specific synthetic steps for each of the routes described may be combined in different ways to prepare the polypeptides described herein. The reagents and starting materials are readily available to one of skill in the art.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

In one aspect, provided herein are PDE3B RNAi agents comprising a sense strand and an antisense strand, wherein the sense strand and the antisense strand form a duplex, and wherein the antisense strand is complementary to a region of PDE3B mRNA. In some embodiments, the sense strand and the antisense strand are each 15-30 nucleotides in length, e.g., 20-25 nucleotides in length. In some embodiments, provided herein are PDE3B RNAi agents comprising a sense strand of 21 nucleotides and an antisense strand of 23 nucleotides. In some embodiments, the sense strand and antisense strand of the PDE3B RNAi agent may have overhangs at either the 5′ end or the 3′ end (i.e., 5′ overhang or 3′ overhang). For example, the sense strand and the antisense strand may have 5′ or 3′ overhangs of 1 to 5 nucleotides.

In some embodiments, provided herein are PDE3B RNAi agent comprising a sense strand and an antisense strand, wherein the sense strand and the antisense strand form a duplex, wherein the sense strand comprises a nucleic acid sequence selected from any one of SEQ ID NOs: 78-153 and 396-456, and the antisense strand comprises a nucleic acid sequence selected from any one of SEQ ID NOs: 154-229 and 457-518, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand is linked to a GalNAc (N-acetylgalactosamine) delivery moiety. The delivery moiety can facilitate the entry of RNAi agent into the cells. In some embodiments, the delivery moiety is linked to the 3′ end of the sense strand, optionally via a linker. In some embodiments, the delivery moiety comprises the following formula (Ac means acetyl):

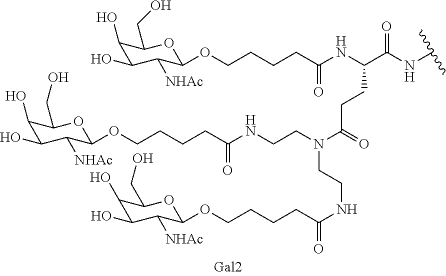 [SMILES: *NC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O]

In another aspect, provided herein are PDE3B RNAi agents comprising Formula R-L_{G}-D, wherein R is a double stranded RNA (dsRNA) comprising a sense stand and an antisense strand, wherein the antisense strand is complementary to PDE3B mRNA, wherein L_{G} is a linker, or optionally absent, and wherein D is a delivery moiety comprising Formula:

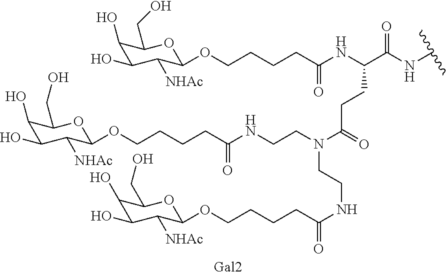 [SMILES: *NC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O]

In some embodiments, the PDE3B delivery moiety is conjugated to the 3′ end of the sense stand via a linker (L_{G}). Suitable linkers are known in the art. In some embodiments, linker (L_{G}) comprises Formula (III) or Formula (IV):

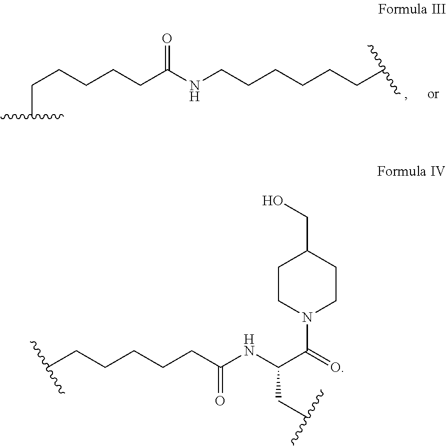 [SMILES: *CCCCCCNC(=O)CCCCC* ; *CCCCCC(=O)N[C@@H](C*)C(=O)N1CCC(CO)CC1]

In some embodiments, the sense strand comprises a first nucleic acid sequence having at least 90% sequence identity to SEQ ID NO: 78-153 or 396-456, and the antisense strand comprises a second nucleic acid sequence having at least 90% sequence identity to SEQ ID NO: 154-229 or 457-518, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand comprises a first nucleic acid sequence having at least 95% sequence identity to SEQ ID NO: 78-153 or 396-456, and the antisense strand comprises a second nucleic acid sequence having at least 95% sequence identity to SEQ ID NO: 154-229 or 457-518, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand comprises a first nucleic acid sequence having at least 18 contiguous nucleotides of one of SEQ ID NO: 78-153 or 396-456, and the antisense strand comprises a second nucleic acid sequence having at least 18 contiguous nucleotides of one of SEQ ID NO: 154-229 or 457-518, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the first nucleic acid sequence (of the sense strand) has at least 19 contiguous nucleotides of one of SEQ ID NO: 78-153 or 396-456, or at least 20 contiguous nucleotides of one of SEQ ID NO: 78-153 or 396-456.

In some embodiments, the second nucleic acid sequence (of the antisense strand) has at least 19 contiguous nucleotides of one of SEQ ID NO: 154-229 or 457-518, or at least 20 contiguous nucleotides of one of SEQ ID NO: 154-229 or 457-518, or at least 21 contiguous nucleotides of one of SEQ ID NO: 154-229 or 457-518, or at least 22 contiguous nucleotides of one of SEQ ID NO: 154-229 or 457-518.

In some embodiments, the sense strand and the antisense strand comprise a pair of nucleic acid sequences selected from the group consisting of: the sense strand comprises a first nucleic acid sequence of SEQ ID NO: 115, and the antisense strand comprises a second nucleic acid sequence of SEQ ID NO: 191; or the sense strand comprises a first nucleic acid sequence of SEQ ID NO: 117, and the antisense strand comprises a second nucleic acid sequence of SEQ ID NO: 193; or the sense strand comprises a first nucleic acid sequence of SEQ ID NO: 133, and the antisense strand comprises a second nucleic acid sequence of SEQ ID NO: 209; or the sense strand comprises a first nucleic acid sequence of SEQ ID NO: 134, and the antisense strand comprises a second nucleic acid sequence of SEQ ID NO: 210; or the sense strand comprises a first nucleic acid sequence of SEQ ID NO: 135, and the antisense strand comprises a second nucleic acid sequence of SEQ ID NO: 211, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand comprise a pair of nucleic acid sequences wherein the sense strand comprises a first nucleic acid sequence including 18 consecutive nucleotides of SEQ ID NO: 78-153 or SEQ ID NO: 396-456, and the antisense strand comprises a second nucleic acid sequence including 18 consecutive nucleotides of SEQ ID NO: 154-229 or SEQ ID NO: 457-518; wherein optionally one or more nucleotides of the sense strand and the antisense strand are independently modified nucleotides, and wherein optionally one or more internucleotide linkages of the sense strand and the antisense strand are modified internucleotide linkages.

As used herein, “NPR-C” refers to natriuretic receptor C protein or polypeptide. NPR-C is also known as NPR3 (natriuretic peptide receptor 3). NPR-C and NPR3 are used interchangeably throughout this disclosure. Several human NPR-C isoforms exist.

The amino acid sequence of the longest human NPR-C isoform (isoform 1) can be found at NP_001191304.1:

| (SEQ ID NO: 74) | |
|---|---|
| 1 | MPSLLVLTES PCVLLGWALL AGGTGGGGVG GGGGGAGIGG GRQEREALPP QKIEVLVLLP |
| 61 | QDDSYLFSLT RVRPAIEYAL RSVEGNGTGR RLLPPGTRFQ VAYEDSDCGN RALFSLVDRV |
| 121 | AAARGAKPDL ILGPVCEYAA APVARLASHW DLPMLSAGAL AAGFQHKDSE YSHLTRVAPA |
| 181 | YAKMGEMMLA LFRHHHWSRA ALVYSDDKLE RNCYFTLEGV HEVFQEEGLH TSIYSFDETK |
| 241 | DLDLEDIVRN IQASERVVIM CASSDTIRSI MLVAHRHGMT SGDYAFFNIE LENSSSYGDG |
| 301 | SWKRGDKHDF EAKQAYSSLQ TVILLRTVKP EFEKFSMEVK SSVEKQGLNM EDYVNMFVEG |
| 361 | FHDAILLYVL ALHEVLRAGY SKKDGGKIIQ QTWNRIFEGI AGQVSIDANG DRYGDESVIA |
| 421 | MTDVEAGTQE VIGDYFGKEG RFEMRPNVKY PWGPLKLRID ENRIVEHTNS SPCKSSGGLE |
| 481 | ESAVTGIVVG ALLGAGLLMA FYFFRKKYRI TIERRTQQEE SNLGKHRELR EDSIRSHFSV |
| 541 | A. |

The amino acid sequence of NPR-C isoform 2 can be found at NP_000899.1:

| (SEQ ID NO: 75) | |
|---|---|
| 1 | MPSLLVLTES PCVLLGWALL AGGTGGGGVG GGGGGAGIGG GRQEREALPP QKIEVLVLLP |
| 61 | QDDSYLFSLT RVRPAIEYAL RSVEGNGTGR RLLPPGTRFQ VAYEDSDCGN RALFSLVDRV |
| 121 | AAARGAKPDL ILGPVCEYAA APVARLASHW DLPMLSAGAL AAGFQHKDSE YSHLTRVAPA |
| 181 | YAKMGEMMLA LFRHHHWSRA ALVYSDDKLE RNCYFTLEGV HEVFQEEGLH TSIYSEDETK |
| 241 | DLDLEDIVRN IQASERVVIM CASSDTIRSI MLVAHRHGMT SGDYAFFNIE LENSSSYGDG |
| 301 | SWKRGDKHDF EAKQAYSSLQ TVILLRTVKP EFEKFSMEVK SSVEKQGLNM EDYVNMFVEG |
| 361 | FHDAILLYVL ALHEVLRAGY SKKDGGKIIQ QTWNRIFEGI AGQVSIDANG DRYGDESVIA |
| 421 | MTDVEAGTQE VIGDYFGKEG RFEMRPNVKY PWGPLKLRID ENRIVEHINS SPCKSCGLEE |
| 481 | SAVTGIVVGA LLGAGLLMAF YFFRKKYRIT IERRTQQEES NLGKHRELRE DSIRSHFSVA |

Other NPR-C isoforms include isoform 3 (NP_001191305.1), isoform 4 (NP_001350581.1), isoform 5 (NP_001351387.1), isoform 6 (NP_001351389.1).

The nucleic acid sequence of human PDE3B mRNA can be found at NM_000922.4 (SEQ ID NO: 76). The amino acid sequence of human PDE3B protein can be found at NP_000913.2 (SEQ ID NO: 77). The nucleic acid sequence of mouse PDE3B mRNA can be found at NM_011055.2 (SEQ ID NO: 78). The amino acid sequence of mouse PDE3B protein can be found at NP_035185.2 (SEQ ID NO: 79).

As used herein, “PDE3B associated metabolic disorder” means a metabolic disorder associated with abnormal PDE3B expression, activity, or function. Such a metabolic disorder can for example be obesity or a co-morbidity associated with obesity.

As used herein, “subject” means a mammal, including cat, dog, mouse, rat, chimpanzee, ape, monkey, and human. Preferably the subject is human.

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

In one aspect, provided herein are PDE3B RNAi agent comprising a sense strand and an antisense strand, wherein the sense strand and the antisense strand form a duplex, and wherein the antisense strand is complementary to a region of PDE3B mRNA. In some embodiments, the sense strand and the antisense strand are each 15-30 nucleotides in length, e.g., 20-25 nucleotides in length. In some embodiments, provided herein are PDE3B RNAi agents comprising a sense strand of 21 nucleotides and an antisense strand of 23 nucleotides. In some embodiments, the sense strand and antisense strand of the PDE3B RNAi agent may have overhangs at either the 5′ end or the 3′ end (i.e., 5′ overhang or 3′ overhang). For example, the sense strand and the antisense strand may have 5′ or 3′ overhangs of 1 to 5 nucleotides.

In some embodiments, provided herein are PDE3B RNAi agent comprising a sense strand and an antisense strand, wherein the sense strand and the antisense strand form a duplex, wherein the sense strand comprises a nucleic acid sequence selected from any one of SEQ ID NOs: SEQ ID NOs: 80-124, 170-223, 271-316, 369-493, and 577-582; and the antisense strand comprises a nucleic acid sequence selected from any one of SEQ ID NOs: 125-169, 224-268, 317-368, and 464-576, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand comprises a first nucleic acid sequence having at least 90% sequence identity to SEQ ID NO: 80-124 and 271-316, and the antisense strand comprises a second nucleic acid sequence having at least 90% sequence identity to SEQ ID NO: 125-169 and 317-368, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand comprises a first nucleic acid sequence having at least 95% sequence identity to SEQ ID NO: 80-124 and 271-316, and the antisense strand comprises a second nucleic acid sequence having at least 95% sequence identity to SEQ ID NO: 125-169 and 317-368, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand comprises a first nucleic acid sequence having at least 18 contiguous nucleotides of one of SEQ ID NO: 80-124 and 271-316, and the antisense strand comprises a second nucleic acid sequence having at least 18 contiguous nucleotides of one of SEQ ID NO: 125-169 and 317-368, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the first nucleic acid sequence (of the sense strand) has at least 19 contiguous nucleotides of one of SEQ ID NO: 80-124 and 271-316, or at least 20 contiguous nucleotides of one of SEQ ID NO: 80-124 and 271-316.

In some embodiments, the second nucleic acid sequence (of the antisense strand) has at least 19 contiguous nucleotides of one of SEQ ID NO: 125-169 and 317-368, or at least 20 contiguous nucleotides of one of SEQ ID NO: 125-169 and 317-368, or at least 21 contiguous nucleotides of one of SEQ ID NO: 125-169 and 317-368, or at least 22 contiguous nucleotides of one of SEQ ID NO: 125-169 and 317-368.

In some embodiments, the sense strand and the antisense strand comprise a pair of nucleic acid sequences selected from the group consisting of:
the sense strand comprises a first nucleic acid sequence of SEQ ID NO: 80-124, and the antisense strand comprises a second nucleic acid sequence of SEQ ID NO: 125-169;
the sense strand comprises a first nucleic acid sequence of SEQ ID NO: 170-223, and the antisense strand comprises a second nucleic acid sequence of SEQ ID NO: 224-268;
the sense strand comprises a first nucleic acid sequence of SEQ ID NO: 271-316, and the antisense strand comprises a second nucleic acid sequence of SEQ ID NO: 317-368; and
the sense strand comprises a first nucleic acid sequence of SEQ ID NO: 369-463 or 577-582, and the antisense strand comprises a second nucleic acid sequence of SEQ ID NO: 464-576.
wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand comprise a pair of nucleic acid sequences wherein the sense strand comprises a first nucleic acid sequence including 18 consecutive nucleotides of SEQ ID NO: 80-124 and 271-316, and the antisense strand comprises a second nucleic acid sequence including 18 consecutive nucleotides of SEQ ID NO: 125-169 and 317-368; wherein optionally one or more nucleotides of the sense strand and the antisense strand are independently modified nucleotides, and wherein optionally one or more internucleotide linkages of the sense strand and the antisense strand are modified internucleotide linkages.

PDE3B associated metabolic disorder, such as obesity or an obesity-related comorbidity, in a patient in need thereof, and such method comprises administering to the patient an effective amount of a PDE3B RNAi agent or a pharmaceutical composition described herein. In some embodiments, the PDE3B RNAi agent or the pharmaceutical composition is administered to the patient intravenously or subcutaneously.

In some embodiments, such methods further comprise administering an incretin to the patient. Examples of incretin include glucagon like peptide-1 (GLP-1) or GLP-1 analogs, glucose-dependent insulinotropic polypeptide (GIP) or GIP analogs, oxyntomodulin or oxyntomodulin analogs; dual GIP and GLP-1 receptor agonists; GCG, and GIP receptor agonist and GLP-1 receptor tri-agonists.

RNAi dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are PDE3B RNAi agents or the pharmaceutical composition comprising a PDE3B RNAi agent for use in a therapy. Also provided are PDE3B RNAi agents or pharmaceutical compositions comprising a PDE3B RNAi agent for use in the treatment of a PDE3B associated metabolic disorder, such as obesity or an obesity-related comorbidity. Also provided herein are uses of PDE3B RNAi agents in the manufacture of a medicament for the treatment of a PDE3B associated metabolic disorder, such as obesity or an obesity-related comorbidity.

The following examples are offered to illustrate, but not to limit, the claimed inventions.

### EXAMPLES

### Example 1: General Strategy for Synthesis of dsRNA-Peptide Conjugates

As depicted in Route 1, NPR-C binding peptides (Route 1A), RNA sense strands (Route 1B) and RNA antisense strands (Route 1C) were all synthesized using standard solid phase peptide or oligonucleotide synthesis techniques. Further functionalization steps to incorporate optional spacers (S_{L}, S_{P}, S_{F}), fatty acids (FA), and linker fragments activated for subsequent conjugation (L_{F}), were performed either directly on solid support, or in solution following cleavage from the solid support, depending on the chemistry involved.

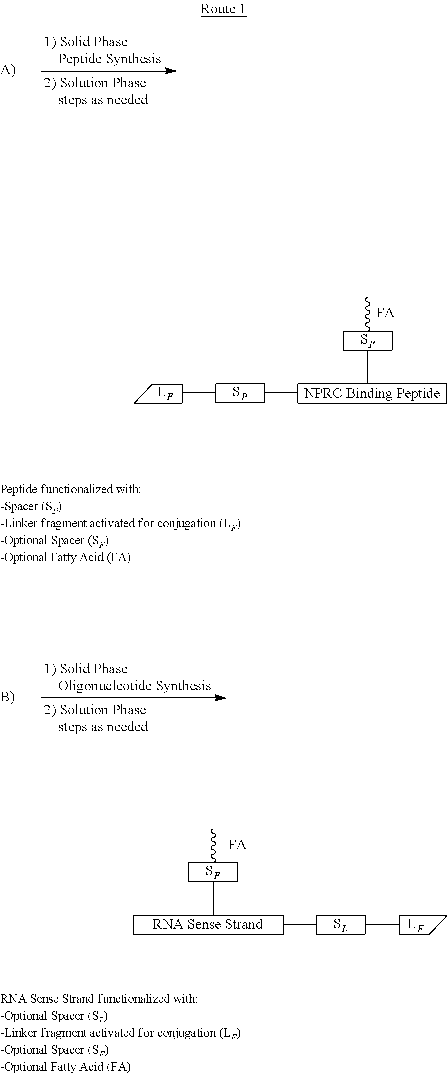

As depicted in Route 2, functionalized RNA sense strand intermediates were conjugated in solution to functionalized NPR-C binding peptides using appropriate conditions. The resulting RNA sense strand-peptide conjugate intermediates were then annealed to the corresponding RNA antisense strand, to provide the dsRNA-peptide conjugate, with appropriate purification steps.

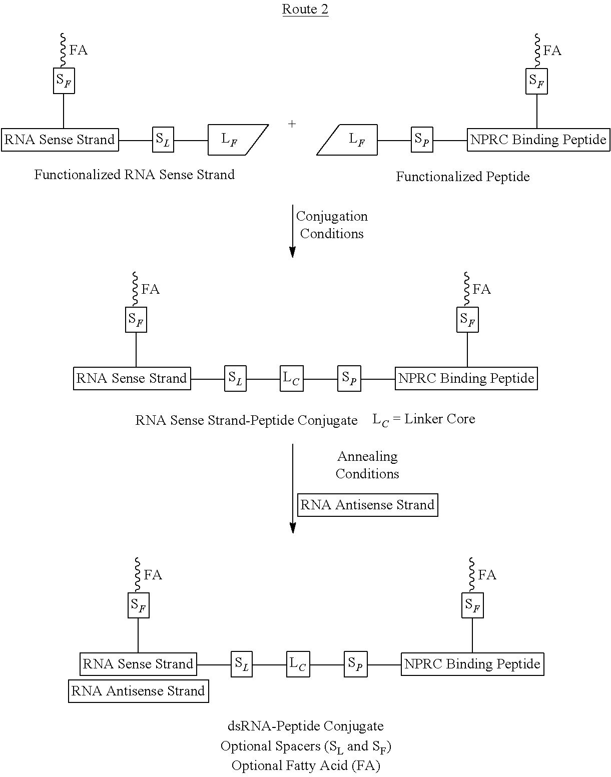

As depicted in Route 3, an alternative synthetic approach entails annealing the functionalized RNA sense strand to the corresponding RNA antisense strand, prior to conjugation. Using appropriate conditions and purification steps, the functionalized NPR-C binding peptide can then be conjugated to the functionalized dsRNA in solution, to provide the dsRNA-peptide conjugate.

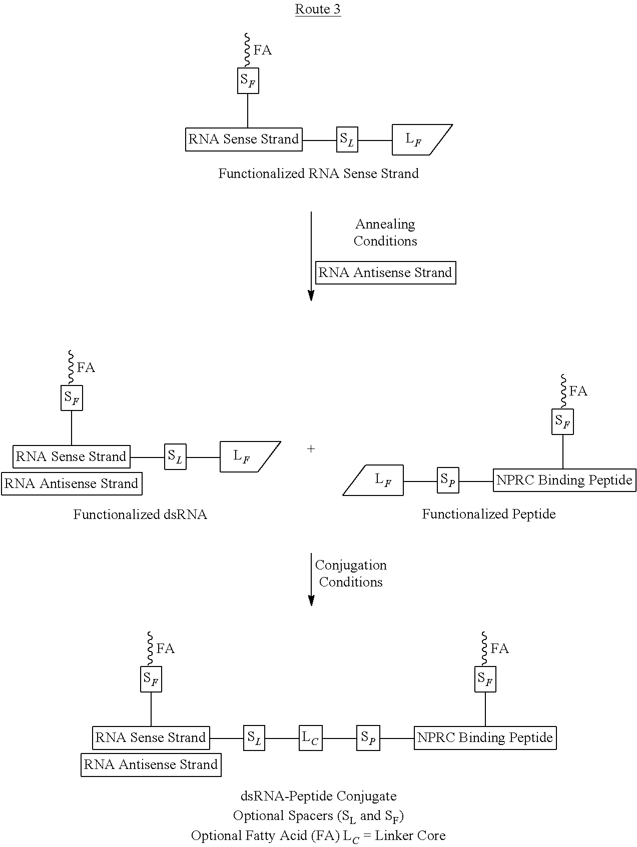

### Example 2: Preparation of Functionalized Peptide Intermediates for Use in Synthesis of dsRNA-NPR-C-Binding Peptide Conjugates

### Examples 2A-2E

### Example 2A: General Procedure for Solid Phase Synthesis of NPR-C Binding Peptides

Peptide synthesis was carried out using standard 9-fluorenyl-methyloxycarbonyl (Fmoc) tert-Butyl (t-Bu) solid phase peptide chemistry protocols on either a Symphony 12-channel multiplex peptide synthesizer or a Symphony-X 24-channel multiplex peptide synthesizer (Gyros Protein Technologies, Inc.), at a 0.1 mmol scale.

The solid support used was pre-loaded H-Cys(Trt)-2-CTC resin (S-trityl-L-cysteine-2-Chlorotrityl Resin, Peptides International), (100-200 mesh) with a 1% DVB cross-linked polystyrene core and a substitution of ~0.50 meq/g for the generation of peptide acids, or Low Loading 4-(2′,4′-Dimethoxyphenyl-Fmoc-aminomethyl)-phenoxyacetamido-norleucyl-4-Methylbenzhydrylamine resin (Fmoc-Rink-MBHA Low Loading resin, EMD Millipore), (100-200 mesh) with a 1% DVB cross-linked polystyrene core and a substitution range of 0.3-0.4 meq/g for peptide amides. Standard sidechain protecting groups were used for all Fmoc-L-Amino Acids utilized. Non-standard amino acids used in the syntheses herein can be found in Table 15 or 16.

Fmoc deprotection prior to each coupling step was accomplished by treatments with 20% piperidine (PIP; Sigma Aldrich) in DMF (Fisher Chemicals), 2×7 minutes with nitrogen mixing, followed by 8×DMF wash cycles. For the synthesis of peptide acids on 2-chlorotrityl resin, typical unhindered couplings were performed for 1 hour using the Fmoc amino acid (0.3 M in DMF), N,N,N′,N′-Tetramethyl-O-(1H-benzotriazol-1-yl)uronium hexafluorophosphate (HBTU, Ambeed Inc.; 0.9 M in DMF) and N,N-diisopropylethylamine (DIPEA, Sigma Aldrich; 1.2 M in DMF), at a 9-fold molar excess of AA/HBTU and a 12-fold molar excess of DIPEA over the reported resin loading level.

For the coupling of hindered building blocks such as Fmoc-Arg(Pbf)-OH, Fmoc-N-Methyl-AAs or Fmoc-αMeAAs, a double coupling protocol was used, and the coupling time was extended to 2 hours each. For the synthesis of peptide amides on Fmoc-Rink-MBHA resin, typical unhindered couplings were performed for 1 hour using the Fmoc amino acid (0.3 M, Advanced ChemTech, in DMF), N,N′-diisopropylcarbodiimide (DIC, Chemlmpex, 1.2 M in DCM) and ethyl cyanohydroxyiminoacetate (Oxyma Pure, Chemlmpex; 0.9 M in DMF), at a 9-fold molar excess of AA/Oxyma and a 12-fold molar excess of DIC over the reported resin loading level. For hindered couplings such as Fmoc-Arg(Pbf)-OH, Fmoc-N-Methyl-AAs or Fmoc-αMeAA, the coupling time was extended to 6 hours.

For the solid phase coupling of active esters, such as MSPT-PEG2-NHS, a manual addition of a 3× excess of the ester along with a 10× excess of DIPEA was used after the final Fmoc was removed from the peptidyl resin. After the primary sequence of the desired peptide was completed, the peptidyl resin was transferred as a DCM slurry to disposable fritted plastic syringe fitted with Teflon stopcock. To prepare the resin for cleavage, further washes with DCM were done, and the resin was thoroughly dried in vacuo.

TFA cleavage: The dry peptidyl resins were treated with 10 mL of cleavage cocktail consisting of trifluoroacetic acid (TFA, Acros Organics), Water, Thioanisole (Sigma-Aldrich) and Triisopropylsilane (TIPS; Acros Organics), (TFA:Water:Thioanisole:TIPS; 90.5:2.5:2.5 v/v) or TFA, water, 3,6-dioxa-1,8-octanedithiol (DODT; Sigma Aldrich), triisopropylsilane, (TFA:Water:DODT:TIPS; 90:5:2.5:2.5 v/v) for 2 hours at room temperature. After the 2 hour cleavage incubation, the resin was filtered off, washed twice with 2 mL of neat TFA, and the combined filtrates/washes were collected in a 50 ml conical disposable tube. The cleavage solution was then treated with 35 mL of cold diethyl ether (Fisher Chemicals) (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo.

TABLE 15
Nonstandard amino acids and other peptide components

| | | AA/Residue |
|---|---|---|
| Building Block | AA/Residue | Abbreviation |
| 2-Azidoacetic Acid | 2-Azidoacetic Acid | |
| Fmoc-L-Hyp(tBu)-OH | L-Hydroxyproline | Hyp |
| Fmoc-D-Hyp(tBu)-OH | D-Hydroxyproline | D-Hyp |
| Fmoc-2-Aminoisobutyric Acid | 2-Aminoisobutyric Acid | Aib |
| Fmoc-L-Orn(Boc)-OH | L-Ornithine | Orn |
| Fmoc-L-Dap(Boc)-OH | L-2,3-Diaminopropionic Acid | Dap |
| Fmoc-αMe-L-Phe(2F)-OH | alpha-methyl-2-fluoro-L-phenylalanine | αMePhe(2F) |
| Trtityl-3-Mercaptopropionic Acid | 3-Mercaptopropionic Acid | Mpa |
| Fmoc-L-Cha-OH | β-cyclohexyl-L-alanine | Cha |
| Fmoc-AEEA-OH | 2-[2-(2-amino-ethoxy)-ethoxy]-acetyl | AEEA |
| Fmoc-L-Arg(Me, Pbf)-OH | N^{ω}-methyl-L-arginine | Arg(Me) |
| Fmoc-L-4-Pal-OH | 4-Pyridyl-L-Alanine | 4-Pal |
| Fmoc-L-2-Nal-OH | 3-(2-naphthyl)-L-alanine | 2Nal |
| Fmoc-L-hPhe-OH | L-Homophenylalanine | hPhe |
| Fmoc-D-Pip-OH | D-Pipecolic Acid (D-Homoproline) | D-Pip |
| Fmoc-L-Glu-OtBu | gamma-L-Glutamic acid | γE |
| Fmoc-Aad(OtBu)-OH | L-Homoglutamic Acid (Aminoadipic Acid) | Aad |
| Fmoc-N-Methyl-L-Arg(Pbf)-OH | N-Methyl-L-arginine | NMe-Arg |
| Mal-Dap(Boc)-OH | Maleimido-L-2,3-Diaminopropionic Acid | Mal-Dap |
| MSPT-PEG2-NHS Ester | 2-(2-(2-(4-(5-(methylsulfonyl)-1H-tetrazol-1- | PT-PEG2 |
| | yl)phenoxy)ethoxy)ethoxy)acetic acid | |
| MSPOD-PEG2-NHS Ester | 2-(2-(2-(4-(5-(methylsulfonyl)-1,3,4-oxadiazol-2- | POD-PEG2 |
| | yl)phenoxy)ethoxy)ethoxy)acetic acid | |
| Fmoc-Cpa-OH | Fmoc-3-(2-cyano-4-pyridyl)alanine | Cpa |
| HO-C20-OtBu | Eicosanedioic acid | HO-C20- |
| C15-COOH | Palmitic Acid | C16- |

### Example 2B: General Procedure for Disulfide Bridge Formation in NPR-C Binding Peptides

Crude peptides were solubilized and diluted, in a suitable glass vessel, with 25% aqueous acetic acid to relatively low concentration (0.2-0.5 mg/ml crude peptide). The solution was then placed on magnetic stirrer with a spin vane, mixed vigorously and titrated with a few drops a saturated Iodine in methanol solution until a faint yellow endpoint was achieved. After reaching the yellow endpoint, the reaction was incubated at RT for 15 min, at which point the excess Iodine was quenched by the addition of a few drops of 0.1 M aqueous ascorbic acid (Sigma Aldrich).

### Example 2C: General Procedure for Thioacetal Bridge Formation in NPR-C Binding Peptides

Peptides were solubilized in 3 mL of water; the solution was then diluted with water and AcCN to achieve about a 30/70 mixture of Water/AcCN (~400 mL total volume) with a low concentration of peptide (~0.2-0.5 mg/ml). The solution was then adjusted to pH 8 with triethylamine (TEA, Acros Organics) ~10 equivalents, reducing conditions to prevent disulfide bridge formation were achieved by the addition of 2-4 equivalents of Tris(2-carboxyethyl) phosphine hydrochloride (TCEP-HCl, Sigma Aldrich) reducing agent. The thioacetal bridge was formed by the addition of 7-10 equivalents of Diiodomethane (CH₂I₂, Alfa Aesar). The thioacetal formation reaction was carried out by incubating the solution for 18 hours at RT with magnetic stirring. Progress of the reaction was monitored using analytical LC-MS and by observing the change in mass of +12 Daltons from the starting reduced peptide molecular weight and an accompanying shift in retention of the starting material HPLC peak.

### Example 2D: General Procedure for Solution-Phase Acylation of NPR-C Peptide N-Terminus with Linker Precursors: Mal-Dap(Boc)-OPfp, MSPT-PEG2-NHS, Mpa-NHS, and Fmoc-Cys(Trt)-Opfp

For cyclic peptides containing a Thioacetal bridge, a solution phase acylation step was necessary to introduce the requisite reactive handle for conjugation. In a 15 mL falcon tube equipped with a spin vane, the purified, lyophilized precursor peptide was dissolved in 1000 μL of DMSO (Acros Organics) to it were added 75-100 μL of DIPEA (TCI America) to adjust the pH to ~8-9. The reaction was magnetically stirred at RT. The active ester of the desired residue/moiety was dissolved in DMSO (~200-300 uL), 50 μL aliquots were added one at time while monitoring the progression of the reaction by LC-MS. Once the starting material was consumed, the reaction was stopped by the addition of ~10 mL cold diethyl ether (Fisher Chemicals) (−20° C.). The tube was mixed vigorously and centrifuged to force the peptide into a pellet or oil phase. Another fresh volume of cold diethyl ether was added, and the process was repeated, this time a white, tacky precipitate was formed as the final crude product.

### Additional Steps Following Solution Phase Coupling of Fmoc-Cys(Trt)-OPfp:

After the acylation and ether precipitation step, the pellet was further treated with 1 ml of 20% PIP in DMF (for ~15 min) to remove the Fmoc protecting group. Another round of precipitation was carried out using cold diethyl ether, washed and additional time with the cold ether. The pellet was finally treated with TFA (with 2% TIPS) to remove the Trityl protecting group from the Cys residue. Cold diethyl ether precipitation was once again used to isolate the final crude product.

### Additional Steps Following Solution Phase Coupling of Mal-Dap(Boc)-OPfp:

After the acylation and ether precipitation step, the pellet was further treated with neat TFA to remove the Boc protecting group from the Dap residue. Cold diethyl ether precipitation was once again used to isolate the final crude product.

### Example 2E: General Procedure for Reverse-Phase HPLC Purification of Peptides

Preparative HPLC was carried out using either a Waters 2545 Binary HPLC Systems or a Shimadzu LC-8A Binary HPLC Systems, both equipped with a column heater; using a Luna Phenyl-Hexyl RP-HPLC column (Phenomenex Inc.; 5 μm, 100 Å; 250×21.2 mm). The running buffers used were either Formic acid; A: 0.1% Formic Acid/H2O and B: 0.1% Formic/Acetonitrile (AcCN, Fisher Chemicals) or TFA; A: 0.1% TFA/H2O and B: 0.1% TFA/Acetonitrile (AcCN, Fisher Chemicals). The initial loadings of the peptides were typically done either at 0%, 5% B, or 10% depending on the hydrophobicity of the peptide, with a 10 min isocratic step after loading at the respective starting conditions for column equilibration. Linear gradients of 0-60% B, or 5-65% B or 10-70% B over 60 min, at a flow of 15, 20 or 25 mL/min, with column heating set at 60° C. were used. UV monitoring was done at either 214 or 220 nm. Fractions that were determined to contain the desired product, as confirmed by LC-MS analysis (Agilent LC/MSD XT), were pooled together. Typically, the solutions were then frozen and lyophilized to give a white amorphous solid product, as the TFA salts of the peptides (TFA was added to the pooled fraction isolated with Formic acid buffers). The purity was assessed by RP-HPLC (Agilent Infinity II), and MW was confirmed by LC-MS analysis (Agilent LC/MSD XT).

### Example 3: Representative Examples of NPRC-Binding Peptide Synthesis and Functionalization

### Examples 3A-3G

### Example 3A: Synthesis of Azidoacetyl-(SP1)-(SEQ ID: 11)

Below is a depiction of the structure of the title compound using the standard single letter code for L-Amino Acids except for the 2-(2-(2-Aminoethoxy)-ethoxy) acetic acid spacer (AEEA), L-Cysteine (Cys) residues at positions 7 and 23, and Azidoacetic Acid at the N-Terminus, where the structures of the residues have been expanded.

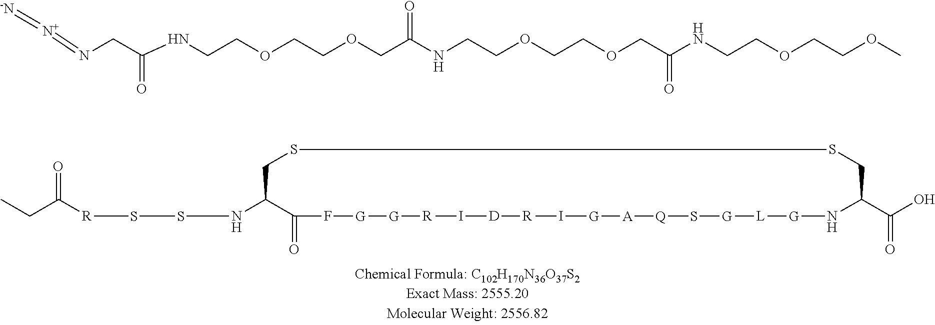

The primary peptide sequence of the title compound was synthesized using standard 9-Fluorenyl-methyloxycarbonyl (Fmoc) tert-Butyl (t-Bu) solid phase peptide chemistry protocols on a Symphony, 12-channel multiplex peptide synthesizer (Gyros Protein Technologies, Inc.), at a 0.1 mmol scale. The solid support used consisted of pre-loaded H-Cys(Trt)-2-CTC resin (S-trityl-L-cysteine-2-Chlorotrityl Resin, Peptides International), (100-200 mesh) with a 1% DVB cross-linked polystyrene core and a substitution of ~0.50 meq/g. Standard sidechain protecting groups were used for all Fmoc-L-Amino Acids utilized. The non-standard amino acid used in the synthesis of the title compound was 2-[2-[2-(Fmoc-amino) ethoxy]ethoxy]acetic acid (Fmoc-AEEA-OH, AappTec Peptides). Azidoacetic Acid was used to cap the N-terminus of the sequence; the residue provides a reactive handle for conjugation chemistries. Fmoc deprotection prior to each coupling step was accomplished by treatments with 20% Piperidine (PIP; Sigma Aldrich) in Dimethylformamide (DMF; Fisher Chemicals), 2×7 minutes with nitrogen mixing, followed by 8×DMF wash cycles. All amino acid couplings were performed for 1 hour using the Fmoc Amino Acid (0.3 M in DMF), N, N, N′,N′-Tetramethyl-O-(1H-benzotriazol-1-yl)uronium hexafluorophosphate (HBTU, Ambeed Inc.; 0.9 M in DMF) and N,N-Diisopropylethylamine (DIPEA, Sigma Aldrich; 1.2 M in DMF), at a 9-fold molar excess of AA/HBTU and a 12-fold molar excess of DIPEA over the reported resin loading level. After the primary sequence of the peptide was synthesized up the third Fmoc-AEEA-OH residue, the final N-Terminal Fmoc was removed with PIP, and the DMF washes were carried out, the peptidyl-resin was capped with a 9-fold excess of Azidoacetic Acid. Coupling was done using DIC/Oxyma (1.2M/0.9M); the coupling time was extended to 5 hrs. After coupling, 3×DMF washes were done, then the peptidyl resin was transferred as a DCM slurry to disposable fritted plastic syringe fitted with Teflon stopcock. Further washes with DCM were done, and finally, the resin was thoroughly dried in vacuo. The dry resin was then treated with 10 mL of cleavage cocktail consisting of trifluoroacetic acid (TFA, Acros Organics), water, and Triisopropylsilane (TIPS; Acros Organics), (TFA:Water:TIPS; 92.5:5:2.5 v/v) for 2 hours at room temperature. After the 2 hr cleavage incubation, the resin was filtered off, washed twice with 2 mL of neat TFA, and the combined filtrates/washes were collected in a 50 ml conical disposable tube. The cleavage solution was then treated with 35 mL of cold diethyl ether (Fisher Chemicals) (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo.

### Disulfide Bridge Formation

The crude peptide was solubilized, in a suitable glass vessel, with 25% aqueous Acetic Acid to a relatively low concentration (0.2-0.5 mg/ml crude peptide). The solution was then placed on magnetic stirrer with the necessary spin vane, mixed vigorously and titrated with a few drops a saturated Iodine in methanol solution until a faint yellow endpoint was achieved. After reaching the yellow endpoint, the reaction was incubated at RT for 15 min, at which point the excess Iodine was quenched by the addition of a few drops of 0.1 M aqueous ascorbic acid (Sigma Aldrich).

### HPLC Purification

The reaction solution containing the crude oxidized peptide was then loaded, via injection valve, onto a preparative HPLC system (Shimadzu LC-8A Binary Preparative HPLC Systems) using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/Acetonitrile (ACCN, Fisher Chemicals). The initial loading was done at 0% B, with 10 min isocratic equilibration after loading. The sample was eluted using a linear 0-60% B gradient over 60 min, at a flow of 25 mL/min, with column heating set at 60° C. Fractions containing the desired product (analysis by Agilent LC-MS) were pooled, frozen and lyophilized to give a white amorphous solid product, as the TFA salt of the title compound. The purity assessed by RP-HPLC 1 (Agilent HPLC System) was found to be >95%, with the observed LC-MS molecular weight of 2556.20 Dalton; matching the theoretical calculated molecular weight of 2556.82 Dalton.

### Example 3B: Synthesis of Cys-(SP1)-(SEQ ID: 20)

Below is a depiction of the structure of the title compound using the standard single letter code for L-Amino Acids except for the 2-(2-(2-Aminoethoxy)-ethoxy) acetic acid spacer (AEEA) and L-Cysteine (Cys) residues at the N-Terminus and at positions 7 and 23 where the structures of the residues have been expanded.

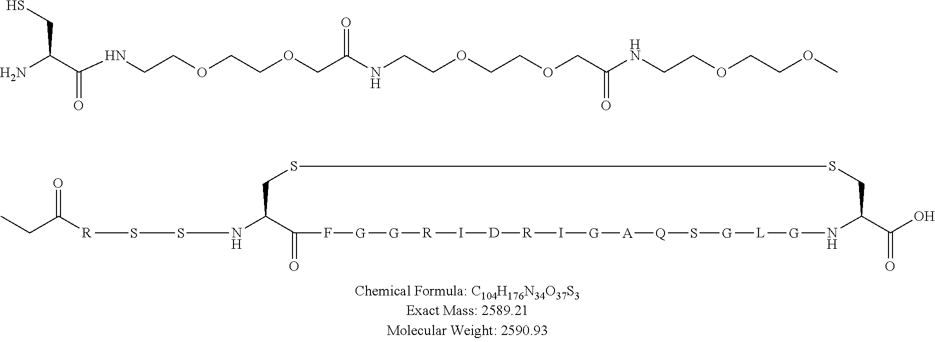

The primary peptide sequence of the title compound was synthesized using standard 9-Fluorenyl-methyloxycarbonyl (Fmoc) tert-Butyl (t-Bu) solid phase peptide chemistry protocols on a Symphony-X, 24-channel multiplex peptide synthesizer (Gyros Protein Technologies, Inc.), at a 0.1 mmol scale. The N-terminal Cysteine residue is omitted from the solid phase peptide synthesis protocol and was added in solution after the isolation of the thioacetal bridged pre-cursor peptide: H-AEEA-AEEA-AEEA-RSS[CFGGRIDRIGAQSGLGC]-OH (Thioacetal Bridge Cys7-Cys23). The solid support used consisted of pre-loaded Fmoc-Cys(Trt)-2-CTC resin (Fmoc-S-trityl-L-cysteine-2-Chlorotrityl Resin, Chemlmpex), (200-400 mesh) with a 1% DVB cross-linked polystyrene core and a substitution range of 0.3-0.8 meq/g. Standard sidechain protecting groups were used for all Fmoc-L-Amino Acids used. The non-standard amino acid used in the synthesis of the title compound was 2-[2-[2-(Fmoc-amino) ethoxy]ethoxy]acetic acid (Fmoc-AEEA-OH, AappTec Peptides). Fmoc deprotection prior to each coupling step was accomplished by treatments with 20% Piperidine (PIP; Sigma Aldrich) in Dimethylformamide (DMF; Fisher Chemicals), 2×7 minutes with nitrogen mixing, followed by 8×DMF washing cycles. All amino acid couplings were performed for 1 hour using the Fmoc Amino Acid (0.3 M in DMF), N, N, N′,N′-Tetramethyl-O-(1H-benzotriazol-1-yl)uronium hexafluorophosphate (HBTU, Ambeed Inc.; 0.9 M in DMF) and N,N-Diisopropylethylamine (DIPEA, Sigma Aldrich; 1.2 M in DMF), at a 9-fold molar excess of AA/HBTU and a 12-fold molar excess of DIPEA over the reported resin loading level. After the primary sequence of the peptide was synthesized up the third Fmoc-AEEA-OH residue, the final N-Terminal Fmoc was removed, the required DMF washes were completed, the peptidyl resin was transferred as a DCM slurry to disposable fritted plastic syringe fitted with Teflon stopcock. Further washes with DCM were done, and finally, the resin was thoroughly dried in vacuo. The dry resin was then treated with 10 mL of cleavage cocktail consisting of trifluoroacetic acid (TFA, Acros Organics), water, 3,6-dioxa-1,8-octanedithiol (DODT; Sigma Aldrich), triisopropylsilane (TIPS; Acros Organics), (TFA:Water:DODT:TIPS; 90:5:2.5:2.5 v/v) for 2 hours at room temperature. After the 2 hr cleavage incubation, the resin was filtered off, washed twice with 2 mL of neat TFA, and the combined filtrates/washes were collected in a 50 ml conical disposable tube, the solution was then treated with 35 mL of cold diethyl ether (Fisher Chemicals) (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo.

### Disulfide Bridge Formation

The crude peptide was solubilized, in a suitable glass vessel, with 25% aqueous Acetic Acid to relatively low concentration (0.2-0.5 mg/ml crude peptide). The solution was then placed on magnetic stirrer with the requisite spin vane, mixed vigorously and titrated with a few drops a saturated Iodine in methanol solution until a faint yellow endpoint was achieved. After reaching the yellow endpoint, the reaction was incubated at RT for 15 min, at which point the excess iodine was quenched by the addition of a few drops of 0.1 M aqueous ascorbic acid (Sigma Aldrich).

### HPLC Purification

The oxidation solution containing the crude peptide was loaded directly onto a preparative HPLC system (Waters 2545 Binary Systems) equipped with a column heater and using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/Acetonitrile (AcCN, Fisher Chemicals). The initial loading was done at 5% B, with 10 min isocratic wash after loading for column equilibration. The sample was eluted using a linear 5-65% B gradient over 60 min, at a flow of 20 mL/min, with column heating set at 60° C. Fractions that were determined to contain the desired product (analysis by LC-MS) were pooled, frozen and lyophilized to give a white amorphous solid product, as the TFA salt of the title compound. The purity assessed by RP-HPLC was found to be >95%, with the observed LC-MS molecular weight of 2473.73 Dalton, matching the theoretical calculated molecular weight of 2473.77 Dalton.

### Thioacetal Bridge Formation

After the purification of the disulfide bridged peptide, the lyophilized disulfide material was solubilized in 3 mL of water, the solution was then diluted with water and AcCN to achieve about a 30/70 mixture of Water/AcCN (~200-400 mL total volume) with a low concentration of peptide (~0.2-0.5 mg/ml). The solution was then adjusted to pH 8 with Triethylamine (TEA, Acros Organics) ~10 equivalents, and the peptide's disulfide bridge was reduced with the addition of 2-4 equivalents of Tris(2-carboxyethyl) phosphine hydrochloride (TCEP-HCl, Sigma Aldrich) reducing agent. After the disulfide bridge reduction, the thioacetal bridge was formed by the addition of 7-10 equivalents of Diiodomethane (CH₂12, Alfa Aesar). The Thioacetal formation reaction was carried out by incubating the solution for 18 hours at RT with magnetic stirring. Progress of the reaction was monitored using analytical LC-MS and by observing the change in mass of +12 Daltons from the starting reduced peptide molecular weight and an accompanying shift in retention of the starting material HPLC peak.

### HPLC Purification

The thioacetal reaction solution was loaded directly onto a preparative HPLC system (Waters 2545 Binary Systems) equipped with a column heater and using a Luna Phenyl-Hexyl RP-HPLC column (Phenomenex Inc.; 5 μm, 100 Å; 250×21.2 mm). The running buffers used were A: 0.1% Formic Acid/H₂O and B: 0.1% Formic/Acetonitrile (AcCN, Fisher Chemicals). The initial loading was done at 5% B, with 10 min isocratic wash after loading equilibration. The sample was eluted using a linear 5-65% B gradient over 60 min, at a flow of 15 mL/min, with column heating set at 60° C. Fractions that were determined to contain the desired product (analysis by LC-MS) were pooled, 0.05 mL of TFA were added to convert the final product to a TFA salt, the solution was then frozen and lyophilized to give a white amorphous solid product, as the TFA salt of the title compound. The purity assessed by RP-HPLC was found to be >95%, with the observed LC-MS molecular weight of 2487.77 Dalton, matching the theoretical calculated molecular weight of 2487.79 Dalton.

Below is a depiction of the structure of intermediate product H-AEEA-AEEA-AEEA-RSS[CFGGRIDRIGAQSGLGC]-OH (Thioacetal Bridge Cys7-Cys23), using the standard single letter code for L-Amino Acids except for the 2-(2-(2-Aminoethoxy)-ethoxy) acetic acid spacer (AEEA) where the structures of the residues have been expanded.

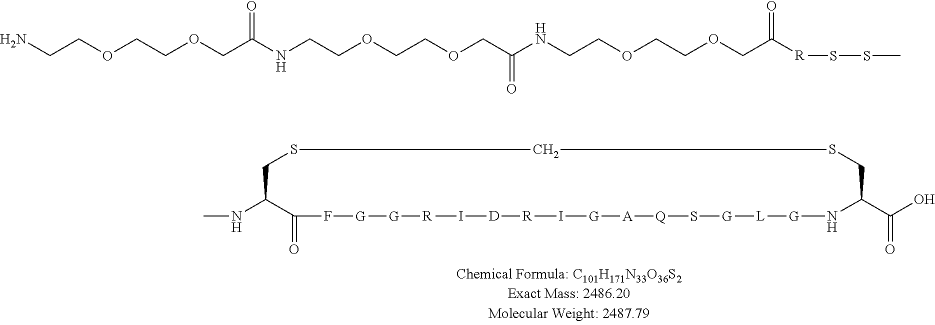

Acylation of Intermediate Thioacetal Peptide with Fmoc-Cys(Trt)-Opfp

In a 15 mL falcon tube equipped with a spin vane, the purified, lyophilized precursor peptide was dissolved in 1000 μL of DMSO (Acros Organics) to it were added 75 μL of DIPEA (TCI America) to adjust the pH to ~8-9, the reaction was magnetically stirred. Fmoc-Cys(Trt)-Opfp (~200 mg, Chemlmpex) was dissolved in DMSO, 50 μL aliquots were added one at time while monitoring the progression of the reaction by LC-MS. Once the starting material was consumed, the reaction was stopped by the addition of ~10 mL cold diethyl ether (Fisher Chemicals) (−20° C.). The tube was mixed vigorously and centrifuged to force the peptide into a pellet (oil phase). Another fresh volume of cold diethyl ether was added, and the process was repeated, this time a white tacky precipitate was formed. The pellet was treated with 1 ml of 20% PIP in DMF (for ~15 min). Another round of precipitation was carried out using cold diethyl ether, washed and additional time with the cold ether. The pellet was finally treated with TFA (2% TIPS) to remove the Trityl protecting group from the Cys residue. The cold diethyl ether precipitation was once again used to isolate the final crude product.

### HPLC Purification

The crude peptide was dissolved in 15 mL of water and then loaded, via injection valve onto a preparative HPLC system (Shimadzu LC-8A Binary Systems) using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/Acetonitrile (AcCN, Fisher Chemicals). The loading was done at 0% B, with 5 min isocratic wash at 0% B for column equilibration. The sample was eluted using a linear 0-60% B gradient over 60 min, at a flow of 25 mL/min, with column heating set at 60° C. Fractions that were determined to contain the desired product (analysis by LC-MS) were pooled, frozen and lyophilized to give a white amorphous solid product, as the TFA salt of the title compound. The purity assessed by RP-HPLC 1 was found to be >95%, with the observed molecular weight of 2590.40 Dalton; matching the theoretical calculated molecular weight of 2590.93 Dalton.

### Example 3C: Synthesis of Cys-(SP1)-(SEQ ID: 17)

Below is a depiction of the structure of the title compound using the standard single letter code for L-Amino Acids except for the 2-(2-(2-Aminoethoxy)-ethoxy) acetic acid spacer (AEEA) and L-Cysteine (Cys) at the N-Terminus where the structures of the residues have been expanded.

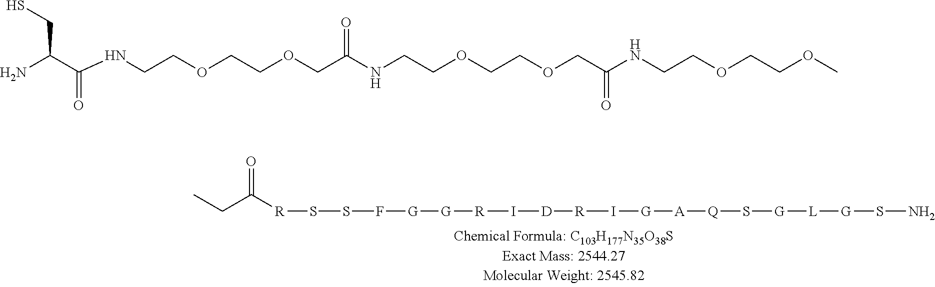

The primary peptide sequence of the title compound is essentially similar to Example 3B. For this iteration, the disulfide bridge was removed by replacing Cys7 and Cys23 with Ser7 and Ser23 residues resulting in a linear analog without a connecting disulfide or thioacetal bridge. The synthesis of the title compound was carried out in a similar manner as Example 3B, with the noted Cys->Ser replacements, and was made as a C-Terminal Amide instead of a C-Terminal acid. The solid support resin used consists of low loading 4-(2′,4′-Dimethoxyphenyl-Fmoc-aminomethyl)-phenoxyacetamido-norleucyl-4-Methylbenzhydrylamine resin (Fmoc-Rink-MBHA Low Loading resin, EMD Millipore), (100-200 mesh) with a 1% DVB cross-linked polystyrene core and a substitution range of 0.3-0.4 meq/g. Amide couplings were performed for 1 hour using the Fmoc Amino Acid (0.3 M, Advanced ChemTech, in DMF), N,N′-Diisopropylcarbodiimide (DIC, ChemImpex, 1.2 M in DCM) and Ethyl Cyanohydroxyiminoacetate (Oxyma Pure, Chemlmpex; 0.9 M in DMF), at a 9-fold molar excess of AA/Oxyma and a 12-fold molar excess of DIC over the reported resin loading level. After the SPPS was complete, the peptidyl resin was transferred as a DCM slurry to disposable fritted plastic syringe fitted with Teflon stopcock. Further washes with DCM were done, and finally, the resin was thoroughly dried in vacuo. The dry resin was then treated with 10 mL of cleavage cocktail consisting of trifluoroacetic acid (TFA, Acros Organics), water, 3,6-dioxa-1,8-octanedithiol (DODT; Sigma Aldrich), triisopropylsilane (TIPS; Acros Organics), (TFA:Water:DODT:TIPS; 90:5:2.5:2.5 v/v) for 2 hours at room temperature. After the 2 hr cleavage incubation, the resin was filtered off, washed twice with 2 mL of neat T′FA, and the combined filtrates/washes were collected in a 50 ml conical disposable tube, the solution was then treated with 35 mL of cold diethyl ether (Fisher Chemicals) (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo.

### HPLC Purification

The crude peptide was dissolved in 15 mL of water and then loaded, via injection valve onto a preparative HPLC system (Shimadzu LC-8A Binary Systems) using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/Acetonitrile (AcCN, Fisher Chemicals). The loading was done at 0% B, with 5 min isocratic wash at 0% B for equilibration. The sample was eluted using a linear 0-60% B gradient over 60 min, at a flow of 25 mL/min, with column heating set at 60° C. Fractions that were determined to contain the desired product (analysis by LC-MS) were pooled, frozen and lyophilized to give a white amorphous solid product, as the TFA salt of the title compound. The purity assessed by RP-HPLC 1 was found to be >95%, with the observed molecular weight of 2545.60 Dalton; matching the theoretical calculated molecular weight of 2545.82 Dalton.

### Example 3D: Synthesis of Mpa-(SP1)-(SEQ ID: 22)

Below is a depiction of the structure of the title compound with all residues expanded.

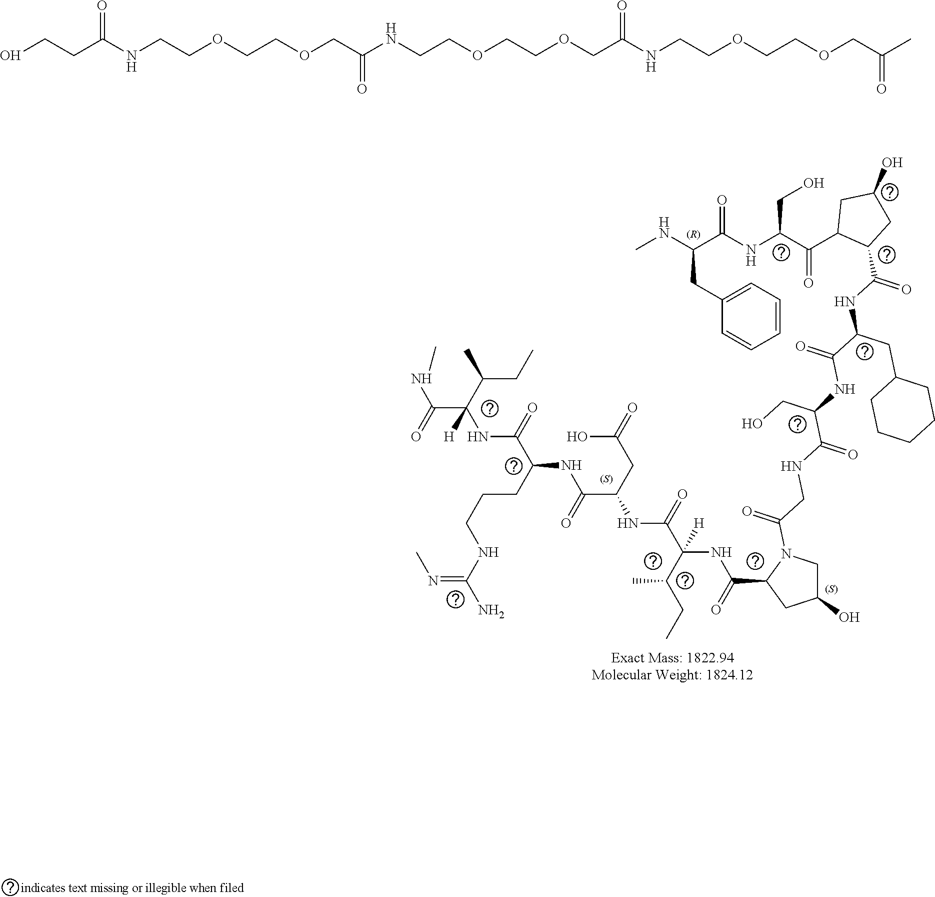

The protocol used for the synthesis of the title compound is essentially similar as that used in Example 3C. In this iteration, the starting resin used was 2 [3-((Methyl-Fmoc-amino)-methyl)indol-1-yl]acetyl AM resin (Methyl Indole AM Resin), (100-200 mesh) with a 1% DVB cross-linked polystyrene core with a substitution range of 0.3-0.4 meq/g. This solid support generates an N-methyl substituted carboxamide containing peptide (—NH—CH₃). The previously outlined cleavage and purification protocols were used for the workup of the material.

### Example 3E: Synthesis of MalDap-(SP12)-(SEQ ID: 21)

Below is a depiction of the structure of the title compound using the standard single letter code for L-Amino Acids, except for the 2-(2-(2-Aminoethoxy)-ethoxy) acetic acid spacer (AEEA), D-Phe, D-Pro, D-Ala, O-cyclohexyl-L-alanine (Cha), and Mal-Dap-at the N-Terminus where the structures of the residues have been expanded.

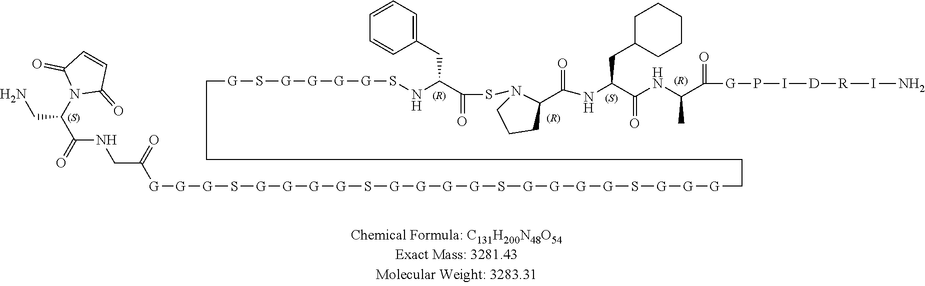

The synthesis of the title compound was carried out in a similar manner as previous preparations. The solid support resin used Fmoc-Rink-MBHA Low Loading resin(EMD Millipore), (100-200 mesh) with a 1% DVB cross-linked polystyrene core and a substitution range of 0.3-0.4 meq/g. Standard 1 hour couplings were performed using Fmoc Amino Acid (0.3 M, Advanced ChemTech, in DMF), N,N′-Diisopropylcarbodiimide (DIC, Chemlmpex, 1.2 M in DCM) and Ethyl Cyanohydroxyiminoacetate (Oxyma Pure, ChemImpex; 0.9 M in DMF), at a 9-fold molar excess of AA/Oxyma and a 12-fold molar excess of DIC over the reported resin loading level. Of note, Fmoc-Gly-Gly-OH dipeptide (Chemlmpex) was used to build the (GGGGS)6 (SEQ ID: 79) repeat, coupling time was extended to 2 hrs for the dipeptide. Mal-Dap(Boc)-OH (Chemlmpex) was used to cap the N-terminus of the peptide using standard coupling conditions. The previously outlined cleavage and purification 97 rotocolls were used for the workup of the material.

### Example 3F: Synthesis of Mpa-(SP1)-(SEQ ID: 21)-(SF4)-(FA4)

Below is a depiction of the structure of the title compound using the standard single letter code for L-Amino Acids, except for the 2-(2-(2-Aminoethoxy)-ethoxy) acetic acid spacer (AEEA), γ-Glutamic Acid, D-Phe, D-Pro, D-Ala, O-cyclohexyl-L-alanine (Cha), C-Terminal Lys, and 3-Mercaptopropionic Acid at the N-Terminus where the structures of the residues have been expanded.

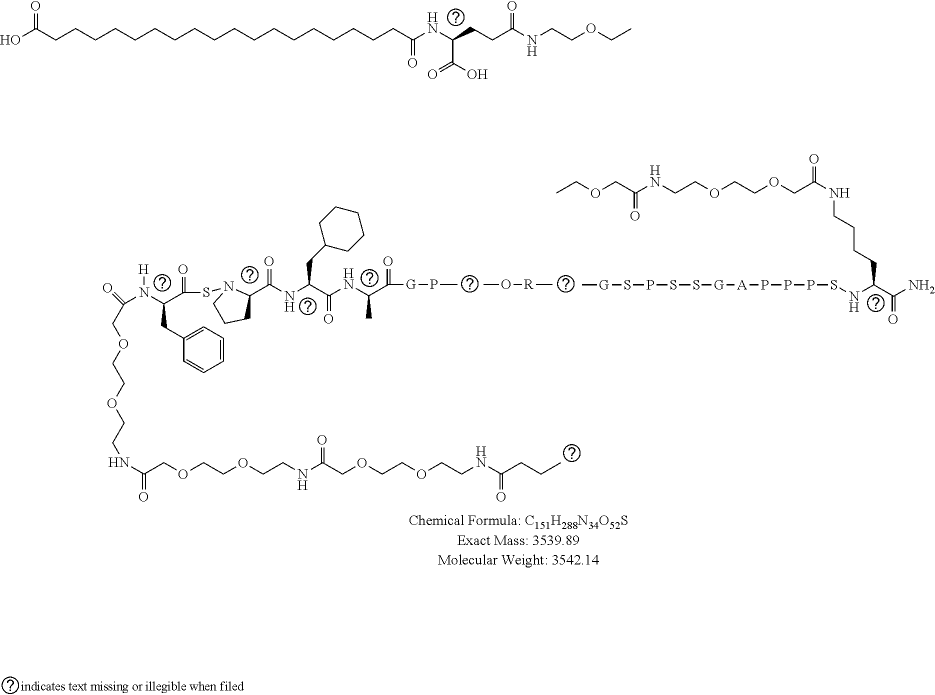

The synthesis of the title compound was carried out in a similar manner as described previously. The solid support resin used Fmoc-Rink-MBHA Low Loading resin (EMD Millipore), (100-200 mesh) with a 1% DVB cross-linked polystyrene core and a substitution range of 0.3-0.4 meq/g. Standard 1 hour couplings were performed using Fmoc Amino Acid (0.3 M, Advanced ChemTech, in DMF), N,N′-Diisopropylcarbodiimide (DIC, ChemImpex, 1.2 M in DCM) and Ethyl Cyanohydroxyiminoacetate (Oxyma Pure, ChemImpex; 0.9 M in DMF), at a 9-fold molar excess of AA/Oxyma and a 12-fold molar excess of DIC over the reported resin loading level. The first amino acid coupled to the resin was Fmoc-Lys(Mtt)-OH (Advanced ChemTech) and was done using standard coupling conditions. The use of orthogonal Mtt protection on Lysine residues allows for selective on-resin side chain deprotection (s-amino group) and directed lipidation of the peptides. For the Mtt deprotection, the resin is thoroughly washed with DCM after the last coupling step of Trt-3-Mpa-OH and then treated 4 times with a sufficient volume of 30% 1,1,1,3,3,3-Hexafluoroisopropanol (HFIP, ChemIpex) in DCM. Each HFIP treatment is done for 30 minutes with nitrogen mixing. After Mtt removal the resin was thoroughly washed with DCM (8×), and subsequently DMF (8×). Acylation with OtBu-C20-(γGlu-OtBu)-AEEA-AEEA-OH afforded the side chain linker fatty diacid, coupling was done using DIC/Oxyma at 3× excess for ~18 hrs. The previously outlined cleavage and purification protocols were used for the workup of the material.

### Example 3G: Synthesis of FA1

Below is a depiction of the structure of the title compound:

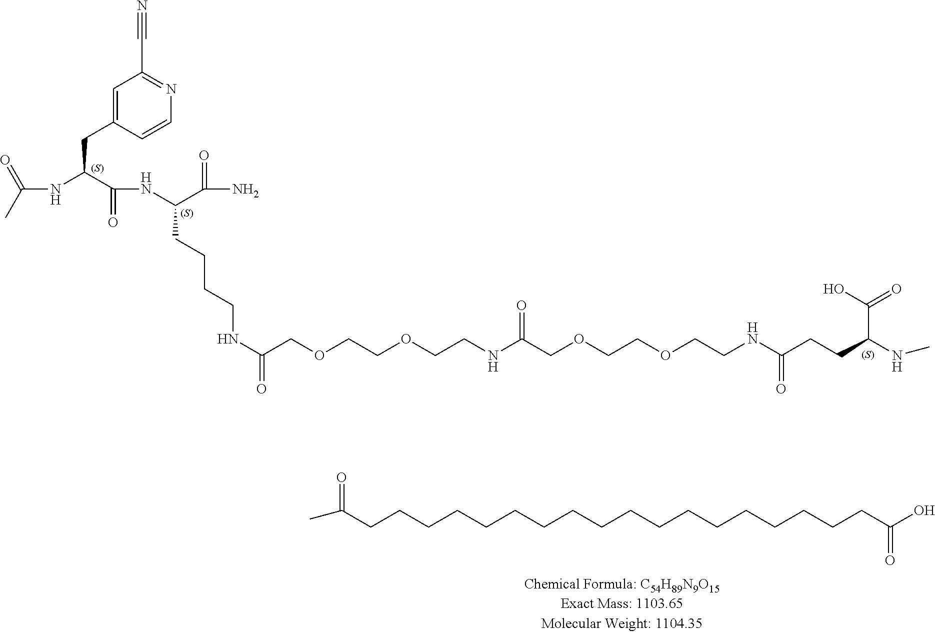

The title compound was synthesized manually in a fritted glass reaction vessel (50 mL) using standard 9-Fluorenyl-methyloxycarbonyl (Fmoc) tert-Butyl (t-Bu) solid phase peptide chemistry protocols at a 1.0 mmol scale. The solid support used consisted of 4-(2′,4′-Dimethoxyphenyl-Fmoc-aminomethyl)-phenoxy resin (Rink-Resin LS, CreoSalus), (100-200 mesh) with a 1% DVB cross-linked polystyrene core and a substitution of ~0.29 meq/g. The building blocks used for the synthesis were: Fmoc-L-Lys(Mtt)-OH (AappTec Peptides), Fmoc-3-(2-cyano-4-pyridyl)alanine (Fmoc-Cpa-OH, WuXi), Fmoc-Glu-OtBu (Advanced ChemTech), 1-Acetylimidazole (Acros Organics) Fmoc-AEEA-OH, and 20-(tert-Butoxy)-20-oxoicosanoic acid (HO-C20-OtBu, WuXi). Fmoc deprotection prior to each coupling step was accomplished by treatments with 20% Piperidine (PIP; Sigma Aldrich) in Dimethylformamide (DMF; Fisher Chemicals), 2×10 minutes with nitrogen mixing, followed by 8×DMF wash cycles. Fmoc-Lys(Mtt)-OH and Fmoc-Cpa-OH were performed for 3 hours using a 5× excess of the Fmoc Amino Acids, DIC and Oxyma. Capping or acetylation of the N-terminus was done using Acetyl-imidazole in DCM (6× excess) reaction time 2 hrs. After the capping step, the resin was washed with DCM 8×. The Mtt side chain deprotection of Lysine was done with using 20% HFIP in DCM (3×20 min), the resin washed 3×DCM, 4×DMF. Coupling of Fmoc-AEEA-OH residues and Fmoc-Glu-OtBu were performed for 5 hours using a 5× excess of the Fmoc Amino Acids, DIC and Oxyma. Coupling of OtBu-C20-OH was done using PyBop/DIPEA 3× excess (overnight). After the coupling of the fatty diacid, the resin washed 3×DMF, 3×DCM, 2 Ether and dried in vacuo. The dry resin was then treated with 100 mL of cleavage cocktail consisting of trifluoroacetic acid (TFA, Acros Organics), water, triisopropylsilane (TIPS; Acros Organics), (TFA:Water:TIPS; 90:5:5 v/v) for 2 hours at room temperature. After the 2 hr cleavage incubation, the resin was filtered off, washed 00 mL of DCM TFA, and the combined filtrates/washes were collected in six 50 ml conical disposable tubes, the solutions were then treated with 35 mL of cold diethyl ether (Fisher Chemicals) (−20° C.) each to precipitate the crude product. The ether suspensions were then centrifuged at 4000 rpm for 2 min to form solid pellets, the supernatant was decanted, and the solid pellets were triturated with fresh ether and the process was repeated two additional times, finally drying the pellets in vacuo. The crude material was used without further purification.

### Example 4: Synthesis of Oligonucleotides for Targeting PDE3B (e.g., siRNA) and Reagents for Oligonucleotide Functionalization

### Examples 4A-4WW

Single strands (sense and antisense) were synthesized on solid support via a MerMade 12 (LGC Biosearch Technologies), K&A H-8 SE (K&A Labs GmbH) oligonucleotide synthesizer. The sequences of the sense and antisense strands were shown in Tables 4 and 6. In addition to the strand sequence, appropriate amidites and CPGs were utilized to append appropriate linkers to enable the conjugates in Table 13.

Strands that were conjugated to peptides were synthesized using an appropriate conjugatable CPG such as “3′-Amino Modifier C-3 lcaa CPG 500 Å” (part number N-8271-05, Chemgenes) to provide a reactive amino on the 3′-end, or “3′ Thiol Modifier C6 SS” (part number ON-526-UC, Hongene—C6SSC6-CPG) to provide a reactive thiol on the 3′-end. Antisense strands used standard 2′-O-Methyl supports (LGC Biosearch Technologies). Strands that were conjugated to GalNAc moieties were synthesized using an appropriate pre-functionalized CPG such as commercially available GalNAc (TEG)-CPG (MRS1279038APT, Amerigo Scientific) for the dsRNAs herein bearing the GalNAc referred to as Gal-1, or custom CPG bearing the GalNAc moiety referred to below as Gal-2, which was synthesized according to procedures found in WO2022271806A1.

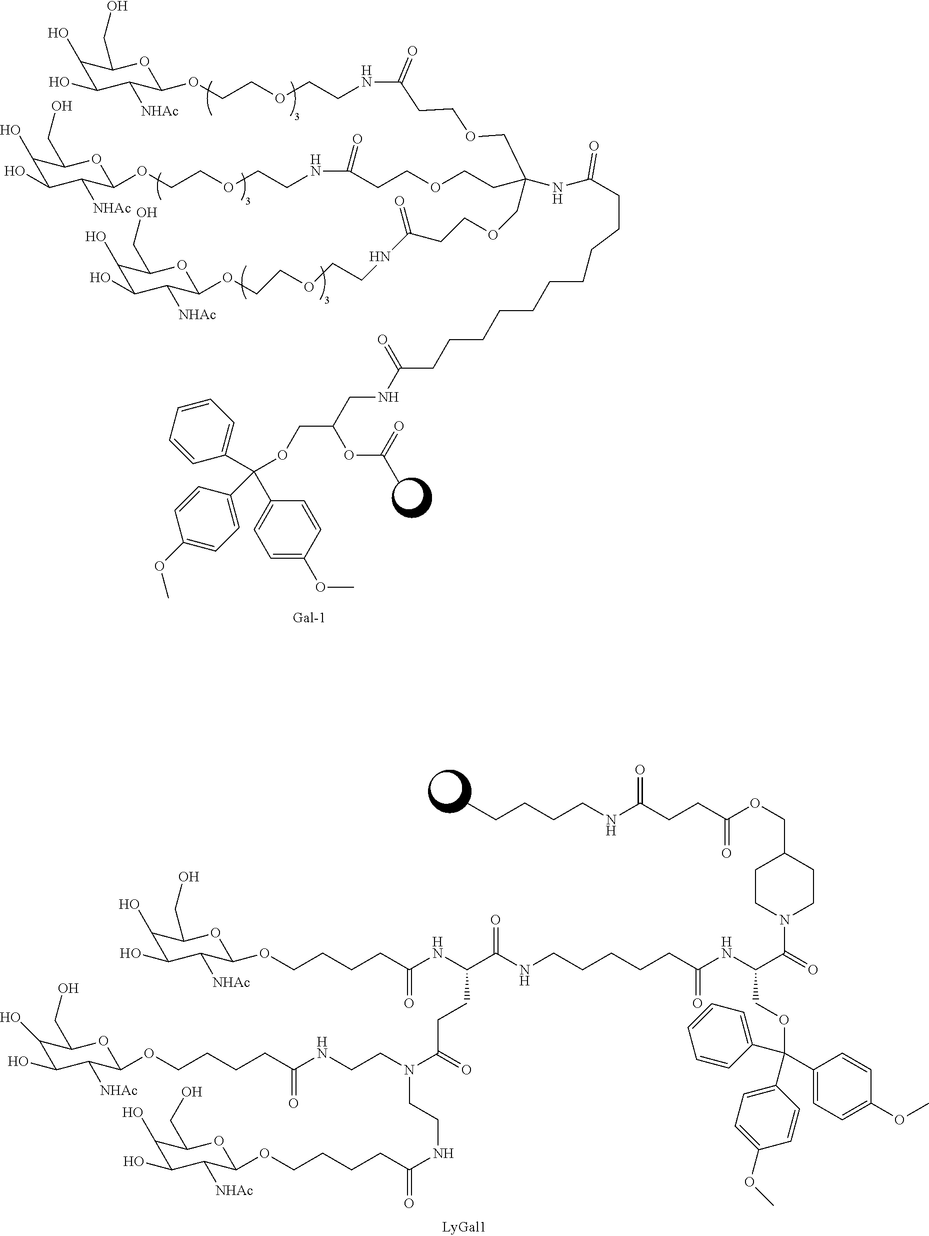

The oligonucleotides were synthesized via phosphoramidite chemistry at an appropriate scale for in-vitro or in-vivo experimentation. Standard reagents were used in the oligo synthesis (Table 16), where 0.1M xanthane hydride in pyridine was used as the sulfurization reagent and 20% DEA in ACN was used as an auxiliary wash post synthesis. For antisense strands, the 20% DEA wash was typically omitted and ACN wash was used instead. All monomers (Table 17, phosphoramidites) were made at 0.1M in ACN and contained a molecular sieves trap bag. When necessary, inclusion of 10% volume equivalents of DMF or 50% volume equivalents of DCM were utilized to maintain amidite solubility (i.e. mU amidite).

The sense strands made with 3′-phthalimido C6 amino CPG were typically cleaved and deprotected from the CPG using 50% (methylamine/ammonia hydroxide 28-30%) at ambient temperature (about 25° C.) for 2-3 hrs. The sense strands containing a 5′-fatty acid moiety made with C20-Diacid-CE phosphoramidite were typically cleaved and deprotected using 0.4 M NaOH in 4:1 MeOH/H2O at ambient temperature (about 25° C.) for 24 hours and GalNAc sense strands were typically cleaved and deprotected (C/D) at 38 to 45° C. for 17 hours using a solution of 3% DEA in ammonia hydroxide (28-30%, cold).C/D was determined complete by IP-RP LCMS when the resulting mass data confirmed the identity of sequence. Dependent on scale, the CPG was filtered via 0.45 um PVDF syringeless filter, 0.22 μm PVDF Steriflip® vacuum filtration or 0.22 μm PVDF Stericup® Quick release.

The CPG was typically back washed/rinsed with RNAse free water or 30% EtOH/RNAse free water, then filtered through the same filtering device and combined with the first filtrate. This was repeated twice. The material was then divided evenly into conical centrifuge tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

Sense strands containing C6SSC6 protected thiol moiety installed on solid phase either originating from a 3′-modification via C6SSC6-CPG or 5′-modification via C6SSC6-PA were typically treated with an excess (tris(2-carboxyethyl)phosphine)-HCl to reduce the dithio bond after removal of volatiles and prior to purification, unless functionalization of an amino moiety is necessary prior to functionalization of the liberated thiol moiety. In this case, purification as below may take place and disulfide cleavage may be similarly employed following amino functionalization.

The crude oligonucleotides were purified via AKTA™ Pure purification system using anion-exchange (AEX) or reverse-phase (RP) chromatography utilizing a gradient of Mobile Phase A (MPA) to Mobile Phase B as appropriate for the product. For AEX, an ES Industry Source™ 15Q column with MPA: 20 mM NaH₂PO₄, 15% ACN, pH 7.4 and MPB: 20 mM NaH₂PO₄, 1M NaBr, 15% ACN, pH 7.4. For RP, an ES Industry Source™ 15RPC with MPA: 10 mM NaOAc 2% Acetonitrile and MPB: 80% Acetonitrile in water. Fractions were analyzed by IP-RP LCMS and those which contained a mass purity greater than 85% without impurities >5% were combined.

The purified oligonucleotides were typically desalted using 15 mL 3K MWCO centrifugal spin tubes at 3500×g for ~30 min. The oligonucleotides were rinsed with RNAse free water until the eluent conductivity reached <100 usemi/cm. After desalting was complete, 2-3 mL of RNAse free water was added then aspirated 10×, the retainment was transferred to an appropriately sized conical tube, this was repeated until complete transfer of oligo by measuring concentration of compound on filter via nanodrop. The final oligonucleotide was then typically nano filtered via 15 mL 100K MWCO centrifugal spin tubes at 3500×g for 2 min. The final desalted oligonucleotides were analyzed for concentration (nanodrop at A260). The molecular weight of the product was confirmed by LC-MS analysis using a Linear Ion Trap Mass Spectrometer (LTQ XL, Thermo Fisher), and purity was confirmed by UPLC analysis.

Oligonucleotide UPLC analysis was typically conducted under Ion-Pairing Reversed-Phase Ultra Performance Liquid Chromatography (IP-RP UPLC) conditions using a Waters™ ACQUITY™ UPLC Oligonucleotide BEH C18 Column 2.1×50 mm, 1.7 μm column. The gradient used Mobile Phase A (MPA) of 7 mM triethylamine with 100 mM hexafluoroisopropanol, and a Mobile Phase B (MPB) of 7:3 Methanol to Acetonitrile. Oligonucleotides with low lipophilicity were typically analyzed over a gradient of 0 to 25% MPB over 10 minutes while oligonucleotides with high lipophilicity were analyzed over a gradient of 5 to 90% MPB over 10 minutes.

TABLE 16
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 17
Phosphoramidites

| | | | Catalog | |
|---|---|---|---|---|
| Phosphoramidite | Abbreviation | Supplier | # | CAS |
| DMT-2′-F-A(Bz)-CE | fA | Hongene | PD1-001 | 136834-22-5 |
| Phosphoroamidite | | | | |
| DMT-2′-F-C(Ac)-CE | fC | Hongene | PD3-001 | 159414-99-0 |
| Phosphoroamidite | | | | |
| DMT-2′-F-G(iBu)-CE | fG | Hongene | PD2-002 | 144089-97-4 |
| Phosphoroamidite | | | | |
| DMT-2′-F-U-CE | fU | Hongene | PD5-001 | 146954-75-8 |
| Phosphoroamidite | | | | |
| DMT-2′-O-Me-A(Bz)-CE | mA | Hongene | PR1-001 | 110782-31-5 |
| Phosphoroamidite | | | | |
| DMT-2′-O-Me-C(Ac)-CE | mC | Hongene | PR3-001 | 199593-09-4 |
| Phosphoroamidite | | | | |
| DMT-2′-O-Me-G(iBu)-CE | mG | Hongene | PR2-002 | 150780-67-9 |
| Phosphoroamidite | | | | |
| DMT-2′-O-Me-U-CE | mU | Hongene | PR5-001 | 110764-79-9 |
| Phosphoroamidite | | | | |
| 5′bis(POM) vinyl phosphate- | POM-VPmU | Hongene | PR5-032 | BVPMUP23B2A1 |
| 2′-Ome-U3′CE | | | | |
| phosphoroamidite | | | | |
| Reverse Abasic | iAb | Chemgenes | ANP- | 401813-16-9 |
| phosphoroamidite | | | 1422 | |
| Abasic phosphoroamidite | Aba | Chemgenes | ANP- | 129821-76-7 |
| | | | 7058 | |
| 2′-O-Trifluoroacetamido propyl | Upa | Chemgenes | ANP- | 165381-49-7 |
| Uridine CED phosphoramidite | | | 7115 | |
| 5′-Thio-modifier C6 S-S | C6SSC6-PA | Hongene | OP-043 | 148254-21-1 |
| phosphoramidite | | | | |
| 5′-Amino-Modifier C6-TFA | 5′-C6Am-PA | Hongene | OP-007 | 133975-85-6 |
| phosphoramidite | | | | |
| Hexadecyl A | Ahd | Lilly | N/A | 2382942-35-8 |
| phosphoroamidite | | | | |
| Hexadecyl G | Ghd | Lilly | N/A | 2382942-32-5 |
| phosphoroamidite | | | | |
| Hexadecyl C | Chd | Lilly | N/A | 2382942-38-1 |
| phosphoroamidite | | | | |
| Hexadecyl U | Uhd | Lilly | N/A | 2382942-83-6 |
| phosphoroamidite | | | | |
| S-mercaptotertbutyl-L-cystine- | tBuCys | Lilly | N/A | 294172-35-3 |
| transcyclohexylamido | | | | |
| phosphoramidite | | | | |
| C20-Diacid-CE phosphoramidite | C20DA | Lilly | N/A | N/A |
| C20-Acid-Ethanolamide | C20AEA | Lilly | N/A | N/A |
| phosphoramidite | | | | |
| Oleyl phosphoramidite | Ole | Lilly | N/A | 847595-74-8 |
| Cetyl phosphoramidite | Cet | Lilly | N/A | 141186-19-8 |
| C22-Docosyl phosphoramidite | Doco | Lilly | N/A | 2093462-10-1 |
| Adamantaneethanol | Adam | Lilly | N/A | 143723-72-2 |
| phosphoramidite | | | | |
| racemic 2-propyl-o-uridine | PrON-U | Lilly | N/A | N/A |
| LNA-T-CE-Phosphoramidite | TL | LGC | LK2065 | 206055-75-6 |
| LNA-5-Me-C (Bz) CE- | CL | LGC | LK2062 | N/A |
| Phosphoramidite | | | | |
| LNA-A (Bz) CE- | AL | LGC | LK2061 | N/A |
| Phosporamidite | | | | |
| LNA-G (dmf) CE- | GL | LGC | LK2063 | N/A |
| Phosphoramidite | | | | |
| DMT-2′-O-MOE-A(Bz)-CE- | eA | Hongene | PR1-004 | 251647-53-7 |
| Phosphoramidite | | | | |
| DMT-2′-O-MOE-G(iBu)-CE- | eG | Hongene | PR2-006 | 251647-55-9 |
| Phosphoramidite | | | | |
| DMT-dT-CE- | dT | Hongene | PD4-002 | 98796-51-1 |
| Phosphoramidite | | | | |
| DMT-dA(Bz)-CE | dA | Hongene | PD1-004 | 98796-53-3 |
| Phosphoramidite | | | | |
| UNA-G(iBu)-CE | UNA-G | Hongene | PR2-014 | N/A |
| Phosphoramidite | | | | |
| UNA-C(Ac)-CE | UNA-C | Hongene | PR3-018 | 1120329-56-7 |
| Phosphoramidite | | | | |
| N4- | C5P-C | Hongene | PR3-165 | N/A |
| diiosbutylaminomethylidene- | | | | |
| 5′-ODMT-2′OMe-5-(1- | | | | |
| Propynyl)-C-3′-CE- | | | | |
| phosphoramidite | | | | |
| 2,6-diamino(N2,N6-diiBu)- | DAP-A | Hongene | N/A | N/A |
| DMT-2′-O-MOE-purine | | | | |
| riboside-CE-Phosphoramidite | | | | |
| Pseudouridine | psU | A2BChem | AX53402 | 199737-09-2 |
| 2′-OMe-I-CE- | I | Glen | 10-3140 | 128219-85-2 |
| Phosphoramidite | | Research | | |

N/A denotes not applicable.

### Example 4A: Hexadecyl A Phosphoroamidite

### N-[9-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]purin-6-yl]benzamide

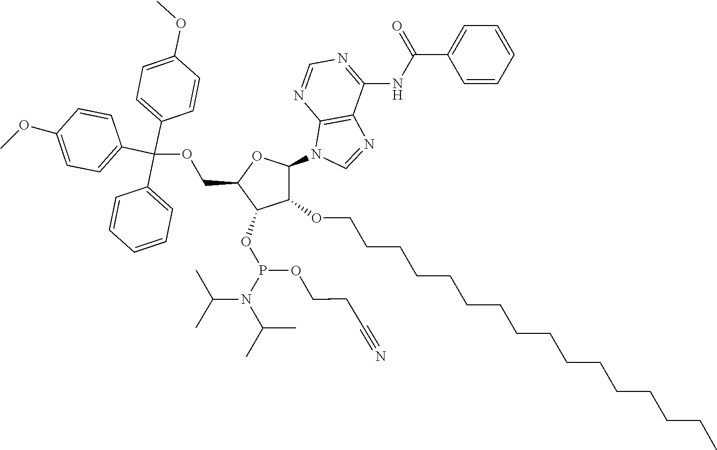 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1cnc2c(NC(=O)c3ccccc3)ncnc21]

Prepared the title compound, referred to herein as Hexadecyl A phosphoroamidite, according to the protocols described in WO2019217459. ¹H-NMR (CD₃CN) δ 9.37 (s, 1H), 8.57 (d, J=9.4 Hz, 1H), 8.27 (d, J=10.3 Hz, 1H), 7.99 (d, J=7.6 Hz, 2H), 7.61 (d, J=7.4 Hz, 1H), 7.52 (t, J=7.6 Hz, 2H), 7.42 (t, J=7.3 Hz, 2H), 7.34-7.16 (m, 7H), 6.85-6.77 (m, 4H), 6.11 (dd, J=5.0, 2.5 Hz, 1H), 4.80 (m, 1H), 4.69 (m, 1H), 4.32 (m, 1H), 3.97-3.78 (m, 1H), 3.74 (d, J=3.1 Hz, 7H), 3.64 (m, 4H), 3.56-3.40 (m, 2H), 3.33 (m, 1H), 2.73-2.59 (m, 1H), 2.50 (t, J=6.0 Hz, 1H), 1.52-1.45 (m, 2H), 1.33-1.12 (m, 37H), 1.09 (d, J=6.8 Hz, 3H), 0.87 (t, J=6.8 Hz, 3H). ³¹P NMR (CD₃CN) δ 151.19, 150.78.

### Example 4B: Hexadecyl U Phosphoroamidite

### 3-[[(2R,3R,4R,5R)-2-[[Bis(4-methoxyphenyl)-phenyl-methoxy]methyl]-4-hexadecoxy-5-(2-hydroxy-4-oxo-pyrimidin-1-yl)THF-3-yl]oxy-(diisopropylamino)phosphanyl]oxypropanenitrile

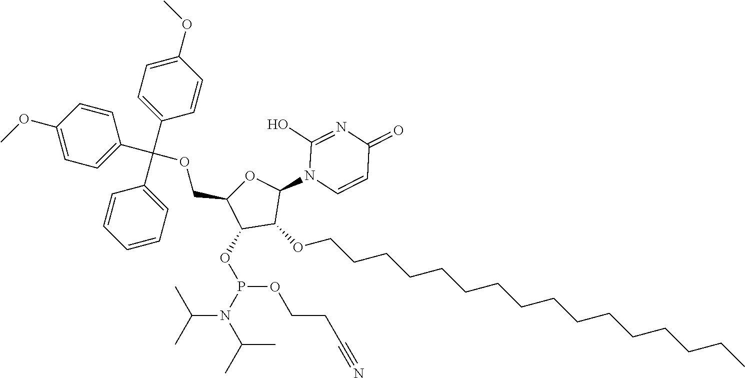 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1ccc(=O)nc1O]

Prepared the title compound, referred to herein as Hexadecyl U phosphoramidite, according to the protocols described in WO2019217459. 1H NMR (CD3CN): 7.86-7.73 (m, 1H), 7.51-7.43 (m, 2H), 7.40-7.23 (m, 7H), 6.95-6.87 (m, 4H), 5.90-5.84 (m, 1H), 5.29-5.21 (m, 1H), 4.54-4.40 (m, 1H), 4.21-4.13 (m, 1H), 4.10-3.56 (m, 13H), 3.50-3.34 (m, 2H), 2.75-2.62 (m, 1H), 2.55 (t, J=6.0 Hz, 1H), 1.66-1.51 (m, 2H), 1.40-1.14 (m, 35H), 1.08 (d, J=6.8 Hz, 3H), 0.91 (t, J=6.8 Hz, 3H). 31P NMR (CD3CN): 149.6, 149.2.

### Example 4C: Hexadecyl C Phosphoroamidite

### N-[1-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]-2-oxo-pyrimidin-4-yl]acetamide

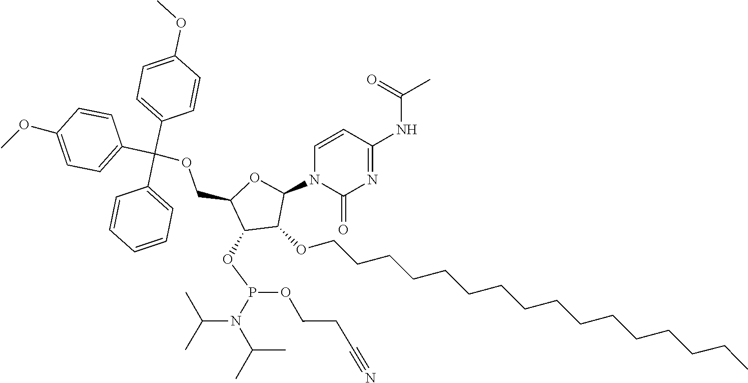 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1ccc(NC(C)=O)nc1=O]

The title compound, referred to herein as Hexadecyl C phosphoramidite, was prepared according to the protocols described in WO2019217459. 1H-NMR (CD3CN) 9.15 (s, 1H), 8.46 (dd, J=7.5 Hz, 1H), 7.95 (d, J=7.6 Hz, 2H), 7.63 (t, J=7.5 Hz, 1H), 7.57-7.41 (m, 5H), 7.41-7.31 (m, 6H), 7.28 (m, 1H), 7.04 (d, J=15.8 Hz, 1H), 6.90 (t, J=7.9 Hz, 4H), 5.90 (d, J=7.8 Hz, 1H), 4.51 (m, 1H), 4.20 (dd, J=10.6, 8.1 Hz, 1H), 4.04 (dd, J=31.3, 4.6 Hz, 1H), 3.91-3.81 (m, 2H), 3.79 (d, J=3.1 Hz, 6H), 3.74 (m, 2H), 3.69-3.41 (m, 6H), 2.67-2.59 (m, 1H), 2.54-2.48 (m, 1H), 1.58 (m, 2H), 1.36 (m, 2H), 1.25 (d, J=4.7 Hz, 26H), 1.21-1.09 (m, 10H), 1.04 (d, J=6.8 Hz, 3H), 0.87 (t, J=6.8 Hz, 3H). 31P NMR (CD3CN) δ 151.10, 150.19.

### Example 4D: Hexadecyl G Phosphoroamidite

### N-[9-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]-6-oxo-1H-purin-2-yl]-2-methyl-propanamide

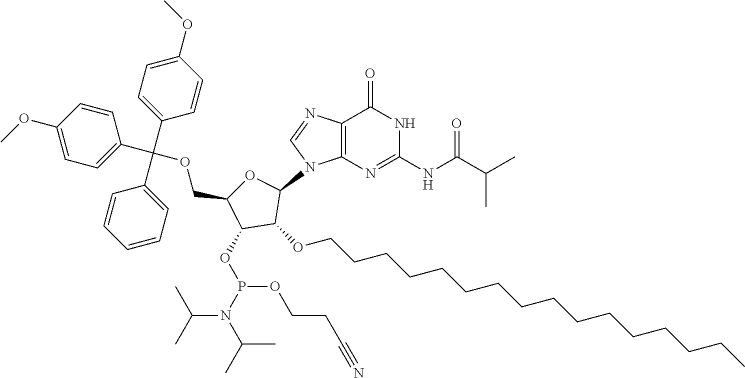 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1cnc2c(=O)[nH]c(NC(=O)C(C)C)nc21]

Prepared the title compound according to the protocols described in WO2019217459. 1H-NMR (CDCl3) δ 12.01-11.96 (m, 1H), 7.82-7.78 (m, 1H), 7.59-7.53 (m, 1H), 7.47-7.42 (m, 1H), 7.41-7.37 (m, 2H), 7.34-7.29 (m, 2H), 7.27-7.22 (m, 3H), 6.85-6.80 (m, 4H), 5.99-5.82 (m, 1H), 4.40-4.36 (m, 1H), 4.17-4.11 (m, 1H), 3.80-3.77 (m, 6H), 3.76-3.68 (m, 6H), 3.22-3.17 (m, 1H), 2.84-2.79 (m, 1H), 1.60-1.54 (m, 4H), 1.35-1.30 (m, 6H), 1.27 (s, 19H), 1.24-1.15 (m, 13H), 1.06-1.03 (m, 5H), 0.93-0.88 (m, 6H), 0.74-0.70 (m, 1H). 31P NMR (CDCl3) δ 150.20, 149.92.

### Example 4E: S-mercaptotertbutyl-L-cystine-transcyclohexylamideo phosphoramidite

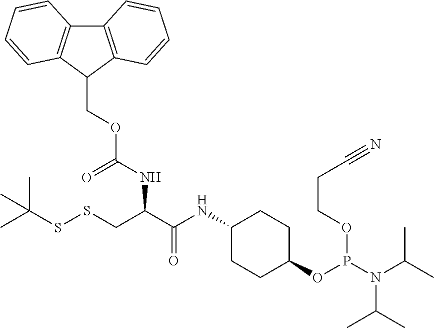 [SMILES: CC(C)N(C(C)C)P(OCCC#N)O[C@H]1CC[C@H](NC(=O)[C@@H](CSSC(C)(C)C)NC(=O)OCC2c3ccccc3-c3ccccc32)CC1]

(9H-fluoren-9-yl)methyl ((2S)-3-(tert-butyldisulfaneyl)-1-(((trans)-4-(((2-cyanoethoxy)(diisopropylamino)phosphaneyl)oxy)cyclohexyl)amino)-1-oxopropan-2-yl)carbamate, referred to herein as S-mercaptotertbutyl-L-cystine-transcyclohexylamido phosphoramidite, was prepared according to the protocol described in Nucleosides, Nucleotides & Nucleic Acids (2000), 19(10-12), 1751-1764.

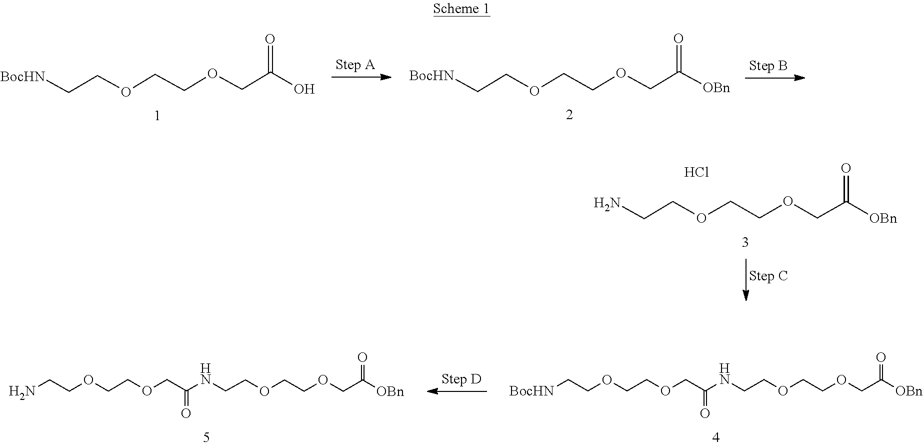 [SMILES: CC(C)(C)OC(=O)NCCOCCOCC(=O)O ; CC(C)(C)OC(=O)NCCOCCOCC(=O)OCc1ccccc1 ; NCCOCCOCC(=O)OCc1ccccc1 ; Cl ; NCCOCCOCC(=O)NCCOCCOCC(=O)OCc1ccccc1 ; CC(C)(C)OC(=O)NCCOCCOCC(=O)NCCOCCOCC(=O)OCc1ccccc1]

Scheme 1, step A shows the alkylative esterification of commercially available 2,2-dimethyl-4-oxo-3,8,11-trioxa-5-azatridecan-13-oic acid (1, CAS number 108466-89-3) which took place utilizing benzyl bromide in acetone in presence of the base potassium carbonate to give compound (2). Step B shows the acidic deprotection of compound (2) which took place by treating with hydrochloric acid in the solvent ethyl acetate to give compound (3). Step C shows the amide coupling of (3) with (1) which took place using the amide coupling reagent EDCI in presence of HOBt and the base DIEA in the solvent DCM to give compound (4). Step D shows the acidic deprotection of compound (4) which took place with hydrochloric acid in the solvent ethyl acetate to give compound (5). One skilled in the art will recognize that a variety of coupling reagents, bases, and solvents can be used to perform an amide coupling, and a variety of acids can be used to perform a BOC deprotection.

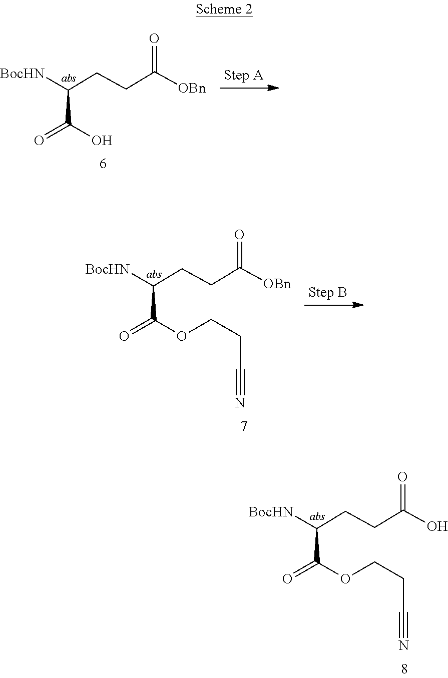 [SMILES: CC(C)(C)OC(=O)N[C@@H](CCC(=O)OCc1ccccc1)C(=O)O ; CC(C)(C)OC(=O)N[C@@H](CCC(=O)OCc1ccccc1)C(=O)OCCC#N ; CC(C)(C)OC(=O)N[C@@H](CCC(=O)O)C(=O)OCCC#N]

Scheme 2, step A shows the coupling of commercially available N-Boc-L-glutamic acid 5-benzyl ester (6, CAS number 13574-13-5) with 3-hydroxypropionitrile which took place under coupling conditions utilizing DCC in presence of the base DMAP in the solvent DCM to give compound (7). Step B shows the hydrogenative debenzylation of compound (7) which took place in presence of hydrogen gas and the catalyst palladium on carbon in the solvent THF to give compound (8). One skilled in the art will recognize that a variety coupling reagents, bases and solvents can be used to perform an ester coupling, and a variety of catalysts and hydrogen sources can be used to perform a benzyl ester removal.

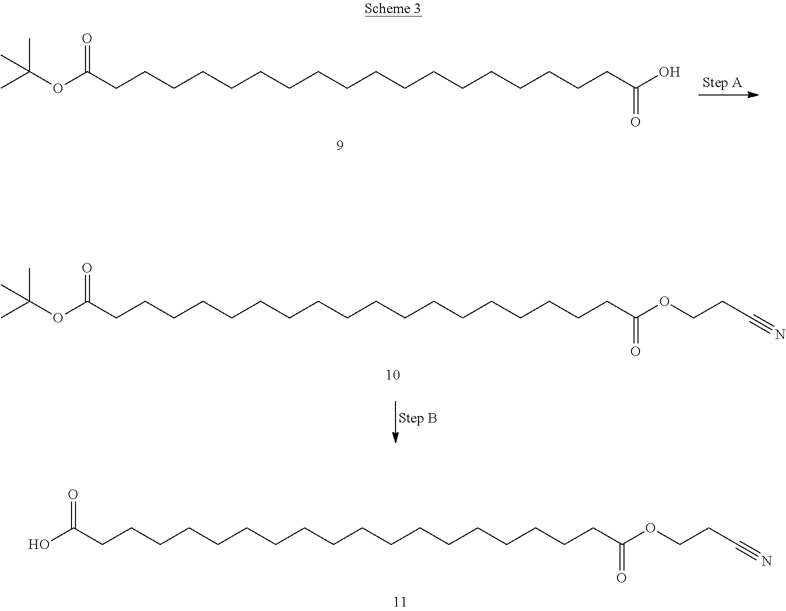 [SMILES: CC(C)(C)OC(=O)CCCCCCCCCCCCCCCCCCC(=O)O ; CC(C)(C)OC(=O)CCCCCCCCCCCCCCCCCCC(=O)OCCC#N ; N#CCCOC(=O)CCCCCCCCCCCCCCCCCCC(=O)O]

Scheme 3, step A shows the coupling of commercially available 20-(tert-Butoxy)-20-oxoicosanoic acid (9, CAS number 683239-16-9) with 3-hydroxypropionitrile which took place under coupling conditions utilizing DCC in presence of the base DMAP in the solvent DCM to give compound (10). Step B shows the acidic deprotection of compound (10) which took place with the acid TFA in the solvent DCM to give compound (11). One skilled in the art will recognize that a variety coupling reagents, bases and solvents can be used to perform an ester coupling, and a variety of acids can be used to perform a BOC deprotection.

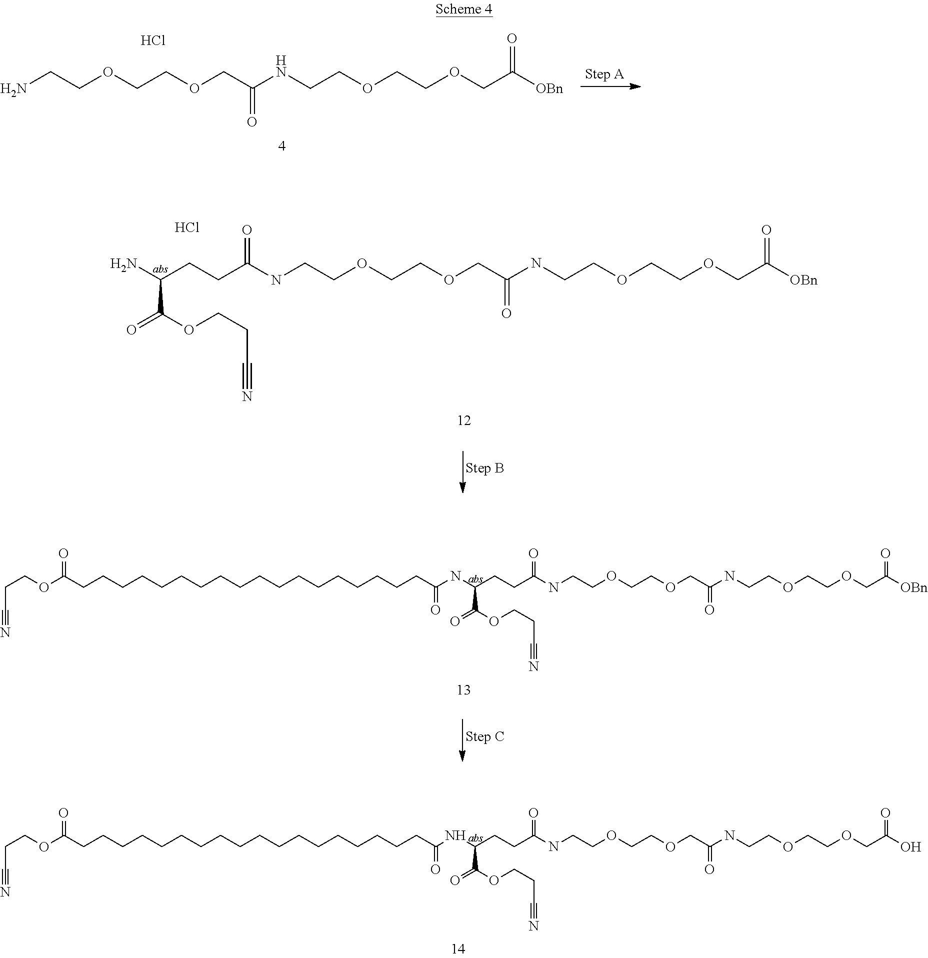

Scheme 4, step A shows the amide coupling of (4) with compound (8) which took place using the amide coupling reagent EDCI in presence of HOBt and the base DIEA in the solvent DCM followed by acidic deprotection with hydrochloric acid in the solvent ethyl acetate to give compound (12). Step B shows the amide coupling of (12) with compound (11) using the amide coupling reagent EDCI in presence of HOBt and the base DIEA in the solvent DCM to give compound (13). Step C shows the hydrogenative debenzylation of compound (13) which took place in presence of hydrogen gas and the catalyst palladium on carbon in the solvent ethyl acetate to give compound (14). One skilled in the art will recognize that a variety coupling reagents, bases and solvents can be used to perform an ester coupling, and a variety of acids can be used to perform a BOC deprotection. One skilled in the art will also recognize a variety of catalysts and hydrogen sources can be used to perform a benzyl ester removal.

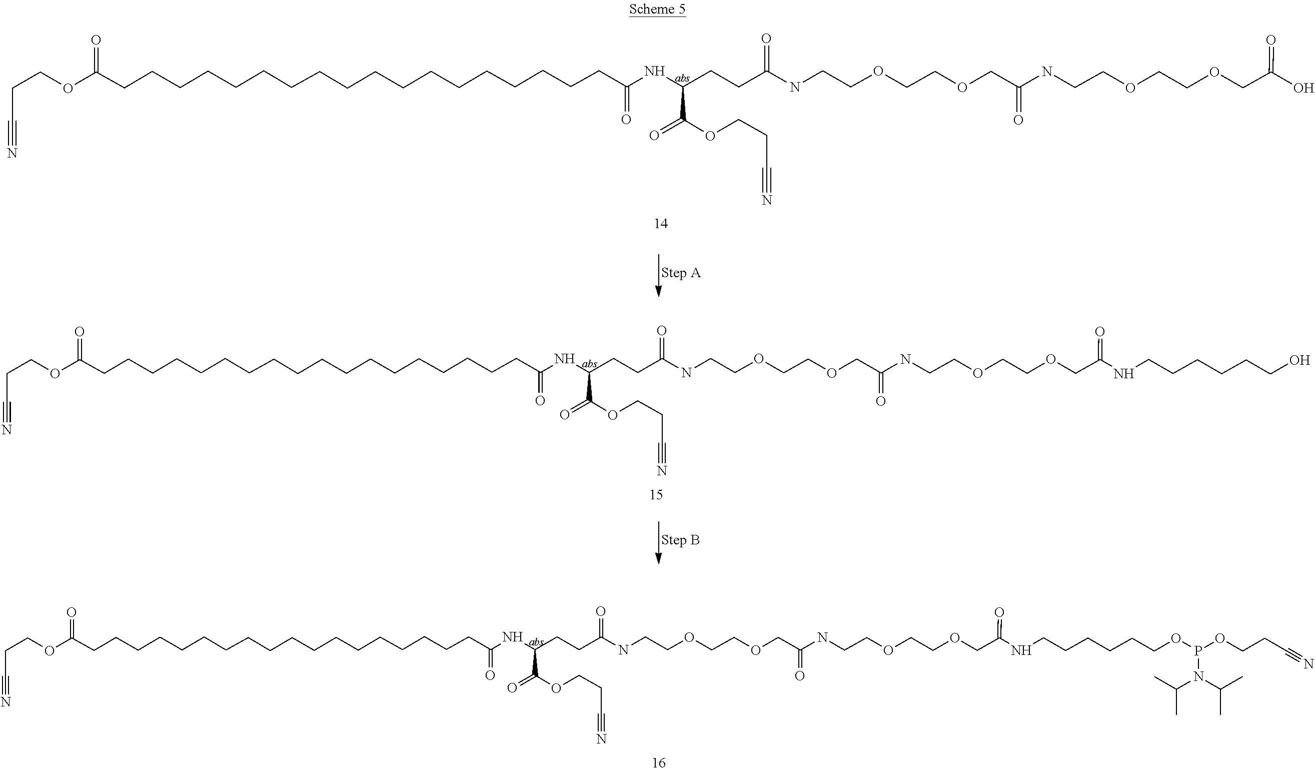

Scheme 5, step A shows the amide coupling of (14) with 6-amino-1-hexanol which took place in presence of the amide coupling reagent EDC in the solvent DCM to give compound (15). Step B shows the conversion of alcohol (15) to a phosphoramidite which took place by treatment with the phosphoramidite precursor 3-((bis(diisopropylamino)phosphaneyl)oxy)propanenitrile in presence of the activator 5-(ethylthio)-1H-tetrazole in the solvent DCM to give compound (16) “C20-Diacid-CE phosphoramidite”. One skilled in the art will recognize a variety of amide coupling reagents and solvents can be used to perform an amide coupling, and a variety of phosphoramidite precursor reagents and activators can be used to form a phosphoramidite.

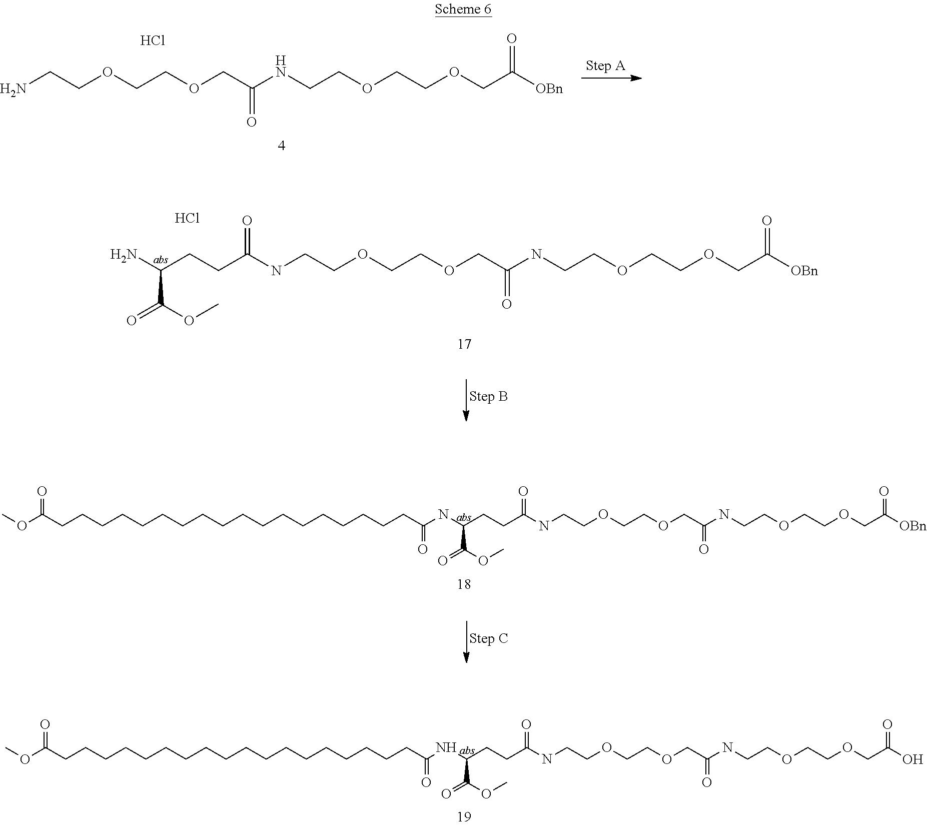

Scheme 6, step A shows the amide coupling of (4) with commercially available N-Boc-L-glutamic acid 5-methyl ester (CAS number 45214-91-3) which took place using the amide coupling reagent EDCI in presence of HOBt and the base DIEA in the solvent DCM followed by acidic deprotection with hydrochloric acid in the solvent ethyl acetate to give compound (17). Step B shows the amide coupling of (17) with commercially available 20-Methoxy-20-oxoicosanoic acid (CAS number 1767-98-2) which took place using the amide coupling reagent EDCI in presence of HOBt and the base DIEA in the solvent DCM to give compound (13). Step C shows the hydrogenative debenzylation of compound (13) which took place in presence of hydrogen gas and the catalyst palladium on carbon in the solvent ethyl acetate to give compound (19). One skilled in the art will recognize that a variety coupling reagents, bases and solvents can be used to perform an ester coupling, and a variety of acids can be used to perform a BOC deprotection. One skilled in the art will also recognize a variety of catalysts and hydrogen sources can be used to perform a benzyl ester removal.

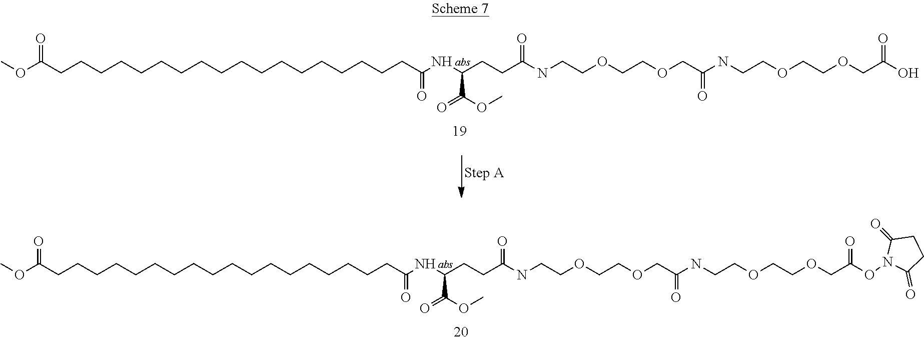

Scheme 7, step A shows the formation of an activated N-hydroxysuccinimide ester by treatment of compound (19) with 1-hydroxypyrrolidine-2,5-dione in presence of a coupling reagent such as EDC in a solvent such as DCM to give compound (20) referred herein as “C20-Diacid-ME NHS Ester.” One skilled in the art will recognize that a variety coupling reagents and solvents can be used to perform an activated ester formation coupling.

### Example 4F: Benzyl 2,2-dimethyl-4-oxo-3,8,11-trioxa-5-azatridecan-13-oate

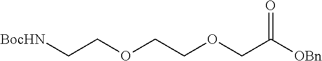 [SMILES: CC(C)(C)OC(=O)NCCOCCOCC(=O)OCc1ccccc1]

To a mixture of 2,2-dimethyl-4-oxo-3,8,11-trioxa-5-azatridecan-13-oic acid (50.0 g, 99% Wt, 1 Eq, 188 mmol) in Acetone (500 mL) were added Benzyl bromide (36.1 g, 25.1 mL, 98% Wt, 1.1 Eq, 207 mmol) and Potassium carbonate (65.6 g, 99% Wt, 2.5 Eq, 470 mmol) at 0° C., then the reaction mixture was stirred at 65° C. for 90 min under N2. This procedure was repeated twice and the reaction mixtures of were combined to work-up and purify. The reaction mixture was filtered and the filter cake was washed with EtOAc (100 mL*3). The filtrate was concentrated under reduced pressure to give a residue. To the residue was added H2O (500 mL) and extracted with EtOAc (500 mL*2). The combined organic layers were washed with brine (1000 mL*2), dried over anhydrous Na2SO4, filtered and concentrated under reduced pressure to give benzyl 2,2-dimethyl-4-oxo-3,8,11-trioxa-5-azatridecan-13-oate (128 g, 0.35 mol, 95%, 97% Purity) as a yellow oil. LCMS m/z=354.1 (M+1). ¹H NMR (400 MHz, DMSO-d₆) δ ppm 7.20-7.47 (m, 5H) 6.73 (br t, J=5.20 Hz, 1H) 5.14 (s, 2H) 4.18 (s, 2H) 3.56-3.64 (m, 2H) 3.47-3.54 (m, 2H) 3.34-3.37 (m, 2H) 3.05 (q, J=6.00 Hz, 2H) 1.36 (s, 9H).

### Example 4G: Benzyl 2-(2-(2-aminoethoxy)ethoxy)acetate hydrochloride

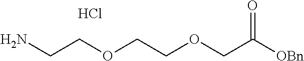 [SMILES: NCCOCCOCC(=O)OCc1ccccc1 ; Cl]

A mixture of benzyl 2,2-dimethyl-4-oxo-3,8,11-trioxa-5-azatridecan-13-oate (60 g, 93% Wt, 1 Eq, 0.16 mol) in 2.0 M HCl in EtOAc (43.8 g, 600 mL, 2 molar, 7.6 Eq, 1.20 mol) was stirred at 26° C. for 60 min. The reaction mixture was concentrated under reduced pressure to give benzyl 2-(2-(2-aminoethoxy)ethoxy)acetate hydrochloride (50 g, 0.16 mol, 99%, 91% Purity) as a pink oil. LCMS m/z=254.1 (M+1).

### Example 4H: benzyl 2,2-dimethyl-4,13-dioxo-3,8,11,17,20-pentaoxa-5,14-diazadocosan-22-oate

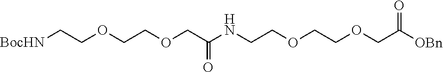 [SMILES: CC(C)(C)OC(=O)NCCOCCOCC(=O)NCCOCCOCC(=O)OCc1ccccc1]

To a mixture of benzyl 2-(2-(2-aminoethoxy)ethoxy)acetate hydrochloride (54 g, 90% Wt, 1 Eq, 0.17 mol) in DCM (500 mL) were added boc-8-amino-3,6-dioxaoctanoic acid (46 g, 40 mL, 95% Wt, 1 Eq, 0.17 mol),1-(3-Dimethylaminopropyl)-3-ethylcarbodiimideHydrochloride (EDCI) (49 g, 99% Wt, 1.5 Eq, 0.25 mol),1-Hydroxy-1H-benzotriazole (34 g, 99% Wt, 1.5 Eq, 0.25 mol) and Diisopropylethylamine (0.11 kg, 0.15 L, 99% Wt, 5 Eq, 0.84 mol) at 0° C., then the reaction mixture was stirred at 27° C. for 16 hour. The reaction mixture was concentrated under reduced pressure to give the crude product. The crude product was purified by flash silica gel chromatography (Biotage®; Agela®Flash Column Silica-CS (330×2 g), Eluent of 0-50% EtOAc/petroleum ether gradient @100 mL/min) to give benzyl 2,2-dimethyl-4,13-dioxo-3,8,11,17,20-pentaoxa-5,14-diazadocosan-22-oate (85 g, 0.16 mol, 97%, 95% Purity) as a colorless gel. LCMS m/z=499.3 (M+1).

### Example 41: benzyl 17-amino-10-oxo-3,6,12,15-tetraoxa-9-azaheptadecanoate hydrochloride

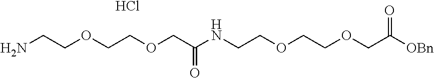 [SMILES: Cl ; NCCOCCOCC(=O)NCCOCCOCC(=O)OCc1ccccc1]

A mixture of benzyl 2,2-dimethyl-4,13-dioxo-3,8,11,17,20-pentaoxa-5,14-diazadocosan-22-oate (60 g, 80% Wt, 1 Eq, 96 mmol) in 2.0 M HCl in EtOAc (43.8 g, 600 mL, 2 molar, 12 Eq, 1.20 mol) was stirred at 26° C. for 60 min. The reaction mixture was concentrated under reduced pressure to give benzyl 17-amino-10-oxo-3,6,12,15-tetraoxa-9-azaheptadecanoate hydrochloride (50 g, 92 mmol, 96%, 80% Purity) as pink gel. LCMS m/z=399.2. ¹H NMR (400 MHz, DMSO-d₆) δ ppm 8.02-8.29 (m, 3H) 7.75 (br t, J=5.20 Hz, 1H) 7.23-7.47 (m, 5H) 5.14 (s, 2H) 4.19 (s, 2H) 3.89 (s, 2H) 3.58-3.65 (m, 8H) 3.50-3.54 (m, 2H) 3.43 (t, J=6.00 Hz, 2H) 3.26 (q, J=6.00 Hz, 2H) 2.90-2.99 (m, 2H).

### Example 4J: 5-benzyl 1-(2-cyanoethyl) (tert-butoxycarbonyl)-L-glutamate

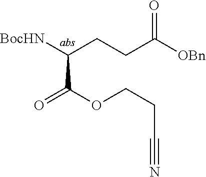 [SMILES: CC(C)(C)OC(=O)N[C@@H](CCC(=O)OCc1ccccc1)C(=O)OCCC#N]

To a solution of (S)-5-(benzyloxy)-2-((tert-butoxycarbonyl)amino)-5-oxopentanoic acid (50.0 g, 1 Eq, 148 mmol) in DCM (500 mL) were added 4-Dimethylaminopyridine (3.62 g, 0.2 Eq, 29.6 mmol) and Dicyclohexylcarbodiimide (61.2 g, 2 Eq, 296 mmol) and Ethylene cyanohydrin (12.6 g, 12.1 mL, 1.2 Eq, 178 mmol) at 0° C. The solution was stirred at 26° C. for 16 hour. The reaction mixture was filtered and the filter cake was washed with DCM (100 mL*3). The filtrate was concentrated under reduced pressure to give the crude product. The crude product was purified by flash silica gel chromatography (Biotage®; Agela®Flash Column Silica-CS (330 g), Eluent of 0-30% EtOAc/hexanes gradient @100 mL/min) to give 5-benzyl 1-(2-cyanoethyl) (tert-butoxycarbonyl)-L-glutamate (50 g, 0.10 mol, 69%, 80% Purity) as a white solid. LCMS m/z=291.1 (M+1-100). ¹H NMR (400 MHz, DMSO-d₆) δ ppm 7.30-7.39 (m, 5H) 5.09 (s, 2H) 4.14-4.34 (m, 2H) 3.98-4.11 (m, 1H) 2.86 (t, J=6.00 Hz, 2H) 2.44-2.52 (m, 2H) 1.95-2.10 (m, 1H) 1.74-1.92 (m, 1H) 1.28-1.46 (m, 9H).

### Example 4K: (S)-4-((tert-butoxycarbonyl)amino)-5-(2-cyanoethoxy)-5-oxopentanoic acid

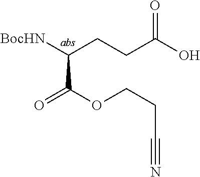 [SMILES: CC(C)(C)OC(=O)N[C@@H](CCC(=O)O)C(=O)OCCC#N]

A solution of 5-benzyl 1-(2-cyanoethyl) (tert-butoxycarbonyl)-L-glutamate (50 g, 80% Wt, 1 Eq, 0.10 mol) in THF (500 mL) was degassed with Argon, and Pd/C (dry basis) (25 g, 10% Wt, 0.23 Eq, 23 mmol) was added. The reaction mixture was evacuated and backfilled three times with hydrogen. The mixture was stirred at 25° C. for 2 hour under an atmosphere of hydrogen 15 psi. Upon completion, the reaction mixture was filtered through a pad of celite and the filtrate was concentrated under reduced pressure to afford the crude product. Pd/C is recycled into an activated metal recycling bucket. The crude product was purified by flash silica gel chromatography (Biotage®; Agela®Flash Column Silica-CS (220 g), Eluent of 0-50% EtOAc/hexanes gradient @100 mL/min) to give (S)-4-((tert-butoxycarbonyl)amino)-5-(2-cyanoethoxy)-5-oxopentanoic acid (25 g, 75 mmol, 73%, 90% Purity) as colorless oil. ¹H NMR (400 MHz, METHANOL-d₄) δ ppm 4.26-4.38 (m, 2H) 4.19 (dd, J=9.20, 5.20 Hz, 1H) 2.85 (t, J=6.00 Hz, 2H) 2.42 (t, J=7.60 Hz, 2H) 2.15 (m, 1H) 1.80-1.98 (m, 1H) 1.44 (s, 9H).

### Example 4L: 1-(tert-butyl) 20-(2-cyanoethyl) icosanedioate

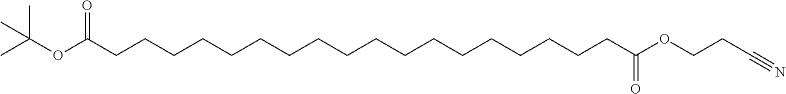 [SMILES: CC(C)(C)OC(=O)CCCCCCCCCCCCCCCCCCC(=O)OCCC#N]

To a mixture of 20-(tert-butoxy)-20-oxoicosanoic acid (25.0 g, 1 Eq, 62.7 mmol) in DCM (300 mL) were added Ethylene cyanohydrin (8.92 g, 8.56 mL, 2 Eq, 125 mmol), DCC (25.9 g, 2 Eq, 125 mmol) and DMAP (1.53 g, 0.2 Eq, 12.5 mmol) at 0° C., then the reaction mixture was stirred at 27° C. for 16 hour under N2. The reaction mixture was filtered and the filter cake was washed with DCM (100 mL*3). The filtrate was concentrated under reduced pressure to give the crude product. The crude product was purified by flash silica gel chromatography (Biotage®; Agela®Flash Column Silica-CS (220 g), Eluent of 0-10% EtOAc/hexanes gradient @100 mL/min) to give 1-(tert-butyl) 20-(2-cyanoethyl) icosanedioate (20 g, 40 mmol, 64%, 90% Purity) as a white solid. ¹H NMR (400 MHz, CHLOROFORM-d) δ ppm 4.27 (t, J=6.00 Hz, 2H) 2.70 (t, J=6.40 Hz, 2H) 2.34 (t, J=7.20 Hz, 2H) 2.18 (t, J=7.60 Hz, 2H) 1.60-1.67 (m, 2H) 1.52-1.59 (m, 2H) 1.43 (s, 9H) 1.22-1.31 (m, 28H).

### Example 4M: 20-(2-cyanoethoxy)-20-oxoicosanoic acid

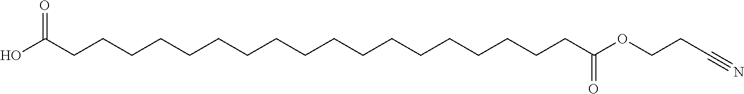 [SMILES: N#CCCOC(=O)CCCCCCCCCCCCCCCCCCC(=O)O]

To a solution of 1-(tert-butyl) 20-(2-cyanoethyl) icosanedioate (17 g, 90% Wt, 1 Eq, 34 mmol) in DCM (150 mL) was added TFA (3.9 g, 90 mL, 1 Eq, 34 mmol). The reaction mixture was stirred at 26° C. for 60 min. The reaction mixture was concentrated under reduced pressure to give 20-(2-cyanoethoxy)-20-oxoicosanoic acid (15 g, 34 mmol, 100%, 90% Purity) as a pink solid. ¹H NMR (400 MHz, DMSO-d₆) δ ppm 4.14-4.21 (m, 2H) 2.86 (t, J=6.00 Hz, 1H) 2.32 (t, J=7.20 Hz, 2H) 2.17 (br t, J=7.20 Hz, 2H) 1.50 (td, J=13.60, 7.20 Hz, 4H) 1.23 (s, 28H).

### Example 4N: 11-benzyl 23-(2-cyanoethyl) (S)-22-((tert-butoxycarbonyl)amino)-10,19-dioxo-3,6,12,15-tetraoxa-9,18-diazatricosanedioate

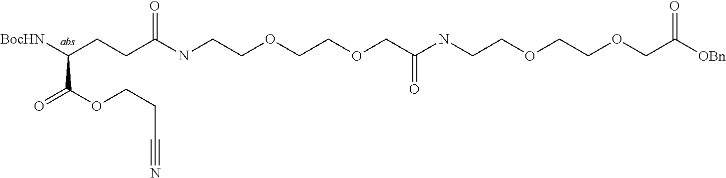 [SMILES: CC(C)(C)OC(=O)N[C@@H](CCC(=O)NCCOCCOCC(=O)NCCOCCOCC(=O)OCc1ccccc1)C(=O)OCCC#N]

To a mixture of benzyl 17-amino-10-oxo-3,6,12,15-tetraoxa-9-azaheptadecanoate hydrochloride (45 g, 80% Wt, 1 Eq, 83 mmol) in DCM (500 mL) were added DIEA (32 g, 43 mL, 3 Eq, 0.25 mol), (S)-4-((tert-butoxycarbonyl)amino)-5-(2-cyanoethoxy)-5-oxopentanoic acid (25 g, 90% Wt, 0.91 Eq, 75 mmol),1-(3-Dimethylaminopropyl)-3-ethylcarbodiimide hydrochloride (EDCI) (24 g, 1.5 Eq, 0.12 mol) and 1-hydroxy-1H-benzotriazole (17 g, 1.5 Eq, 0.12 mol) at 0° C., then the reaction mixture was stirred at 26° C. for 16 hour. The reaction mixture was concentrated under reduced pressure to give the residue. The residue was added to H2O (500 mL) and extracted with EtOAc (400 mL*2). The combined organic layers were washed with brine (400 mL*2), dried over anhydrous Na2SO4, filtered and concentrated under reduced pressure to give a crude product. The crude product was purified by flash silica gel chromatography (Biotage®; Agela®Flash Column Silica-CS (330 g), Eluent of 0-100% EtOAc/hexanes gradient @100 mL/min) to give 1-benzyl 23-(2-cyanoethyl) (S)-22-((tert-butoxycarbonyl)amino)-10,19-dioxo-3,6,12,15-tetraoxa-9,18-diazatricosanedioate (40 g, 52 mmol, 62%, 88% Purity) as a yellow oil. LCMS m/z=681.3 (M+1). ¹H NMR (400 MHz, DMSO-d₆) δ ppm 7.85 (br t, J=5.20 Hz, 1H) 7.63 (br t, J=5.60 Hz, 1H) 7.25-7.41 (m, 5H) 5.15 (s, 2H) 4.15-4.30 (m, 4H) 3.91-4.01 (m, 1H) 3.87 (s, 2H) 3.50-3.63 (m, 8H) 3.42 (dt, J=11.60, 6.00 Hz, 4H) 3.32 (s, 2H) 3.26 (q, J=6.00 Hz, 2H) 3.20 (q, J=5.60 Hz, 2H) 2.87 (t, J=5.60 Hz, 2H) 2.18 (br t, J=7.60 Hz, 2H) 1.87-1.98 (m, 1H) 1.76 (m, 1H) 1.38 (s, 9H).

### Example 40: 1-benzyl 23-(2-cyanoethyl) (S)-22-amino-10,19-dioxo-3,6,12,15-tetraoxa-9,18-diazatricosanedioate hydrochloride

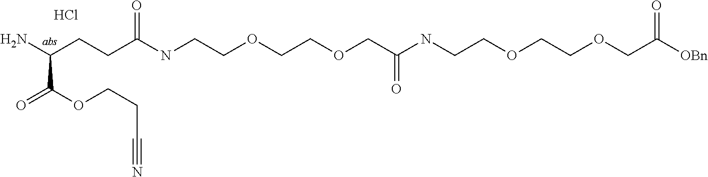 [SMILES: N#CCCOC(=O)[C@@H](N)CCC(=O)NCCOCCOCC(=O)NCCOCCOCC(=O)OCc1ccccc1 ; Cl]

A mixture of 1-benzyl 23-(2-cyanoethyl) (S)-22-((tert-butoxycarbonyl)amino)-10,19-dioxo-3,6,12,15-tetraoxa-9,18-diazatricosanedioate (38 g, 88% Wt, 1 Eq, 49 mmol) in 2.0 M HCl in EtOAc (29.2 g, 400 mL, 2 molar, 16 Eq, 800 mmol) was stirred at 27° C. for 60 min. The reaction mixture was concentrated under reduced pressure to give 1-benzyl 23-(2-cyanoethyl) (S)-22-amino-10,19-dioxo-3,6,12,15-tetraoxa-9,18-diazatricosanedioate hydrochloride (34 g, 45 mmol, 92%, 82% Purity) as a red oil. LCMS m/z=581.4 (M+1). ¹H NMR (400 MHz, DMSO-d₆) δ ppm 8.59-8.85 (m, 3H) 8.11 (br t, J=5.60 Hz, 1H) 7.69 (br t, J=5.60 Hz, 1H) 7.17-7.46 (m, 5H) 5.14 (s, 2H) 4.25-4.40 (m, 2H) 4.19 (s, 2H) 3.87 (s, 2H) 3.49-3.64 (m, 9H) 3.39-3.45 (m, 4H) 3.17-3.29 (m, 4H) 2.96 (t, J=6.00 Hz, 2H) 2.23-2.41 (m, 2H) 1.99-2.06 (m, 2H).

### Example 4P: 1-benzyl 21,41-bis(2-cyanoethyl) (S)-9,18,23-trioxo-2,5,11,14-tetraoxa-8,17,22-triazahentetracontane-1,21,41-tricarboxylate

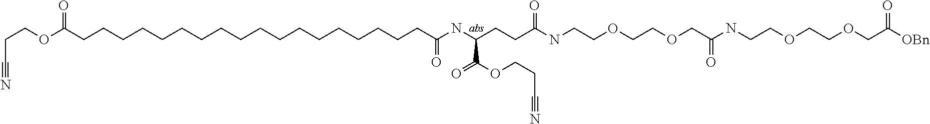 [SMILES: N#CCCOC(=O)CCCCCCCCCCCCCCCCCCC(=O)N[C@@H](CCC(=O)NCCOCCOCC(=O)NCCOCCOCC(=O)OCc1ccccc1)C(=O)OCCC#N]

To a mixture of 1-benzyl 23-(2-cyanoethyl) (S)-22-amino-10,19-dioxo-3,6,12,15-tetraoxa-9,18-diazatricosanedioate hydrochloride (20.5 g, 82% Wt, 1 Eq, 27.2 mmol) in DCM (300 mL) were added N-ethyl-N-isopropylpropan-2-amine (14.1 g, 4 Eq, 109 mmol),20-(2-cyanoethoxy)-20-oxoicosanoic acid (12.0 g, 90% Wt, 1 Eq, 27.2 mmol),1H-benzo[d][1,2,3]triazol-1-ol (5.52 g, 1.5 Eq, 40.9 mmol) and 3-(((ethylimino)methylene)amino)-N,N-dimethylpropan-1-amine hydrochloride (7.83 g, 1.5 Eq, 40.9 mmol) at 26° C., then the reaction mixture was stirred at 26° C. for 16 hour. The reaction mixture was concentrated under reduced pressure to give the residue. The residue was added to H2O (500 mL) and extracted with EtOAc (400 mL*2). The combined organic layers were washed with brine (400 mL*2), dried over anhydrous Na2SO4, filtered and concentrated under reduced pressure to give a crude product. The crude product was purified by flash silica gel chromatography (Biotage®; Agela®Flash Column Silica-CS (220 g), Eluent of 0-10% MeOH/DCM gradient @100 mL/min) to give 1-benzyl 21,41-bis(2-cyanoethyl) (S)-9,18,23-trioxo-2,5,11,14-tetraoxa-8,17,22-triazahentetracontane-1,21,41-tricarboxylate (24 g, 18 mmol, 66%, 72% Purity) as a white solid. LCMS m/z=958.5 (M+1). ¹H NMR (400 MHz, DMSO-d₆) δ ppm 8.22 (d, J=7.20 Hz, 1H) 7.86 (br t, J=5.20 Hz, 1H) 7.64 (br t, J=5.60 Hz, 1H) 7.22-7.44 (m, 5H) 5.14 (s, 2H) 4.17-4.21 (m, 5H) 3.86 (s, 2H) 3.48-3.64 (m, 9H) 3.40-3.47 (m, 4H) 3.13-3.30 (m, 5H) 2.86 (td, J=6.00, 2.80 Hz, 4H) 2.32 (t, J=7.60 Hz, 2H) 2.14-2.20 (m, 2H) 2.10 (t, J=7.60 Hz, 2H) 1.90-1.99 (m, 1H) 1.74-1.85 (m, 1H) 1.43-1.56 (m, 4H) 1.22 (s, 28H).

### Example 4Q: (S)-46-cyano-22-((2-cyanoethoxy)carbonyl)-10,19,24,43-tetraoxo-3,6,12,15,44-pentaoxa-9,18,23-triazahexatetracontanoic acid

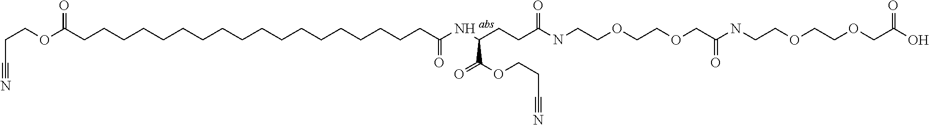 [SMILES: N#CCCOC(=O)CCCCCCCCCCCCCCCCCCC(=O)N[C@@H](CCC(=O)NCCOCCOCC(=O)NCCOCCOCC(=O)O)C(=O)OCCC#N]

A solution of 1-benzyl 21,41-bis(2-cyanoethyl) (S)-9,18,23-trioxo-2,5,11,14-tetraoxa-8,17,22-triazahentetracontane-1,21,41-tricarboxylate (12.0 g, 72% Wt, 1 Eq, 9.02 mmol) in THF (120 mL) was degassed with Argon and Pd/C (wet basis) (3.0 g, 10% Wt, 0.31 Eq, 2.8 mmol) was added. The reaction mixture was evacuated and backfilled three times with hydrogen. The mixture was stirred at 26° C. for 30 min under an atmosphere of hydrogen 15 psi. Upon completion, the reaction mixture was filtered through a pad of celite and the filtrate was concentrated under reduced pressure to afford the crude product. Pd/C is recycled into an activated metal recycling bucket. The crude product was purified by flash silica gel chromatography (Biotage®; Agela®Flash Column Silica-CS (220 g), Eluent of 0-10% MeOH/DCM gradient @60 mL/min) to give (S)-46-cyano-22-((2-cyanoethoxy)carbonyl)-10,19,24,43-tetraoxo-3,6,12,15,44-pentaoxa-9,18,23-triazahexatetracontanoic acid (11694.41 mg, 13.3 mmol, 147%, 98.4% Purity) as a white solid. LCMS m/z=869.0 (M+1). ¹H NMR (400 MHz, METHANOL-d₄) δ ppm 4.24-4.42 (m, 5H) 4.12 (s, 2H) 4.01 (s, 2H) 3.63-3.72 (m, 8H) 3.58 (dt, J=10.40, 4.80 Hz, 4H) 3.35-3.48 (m, 4H) 2.80-2.88 (m, 3H) 2.22-2.42 (m, 6H) 1.92-2.21 (m, 2H) 1.55-1.68 (m, 4H) 1.18-1.41 (m, 29H).

### Example 4R: 2-cyanoethyl (S)-29-((2-cyanoethoxy)carbonyl)-1-hydroxy-8,17,26,31-tetraoxo-10,13,19,22-tetraoxa-7,16,25,30-tetraazapentacontan-50-oate

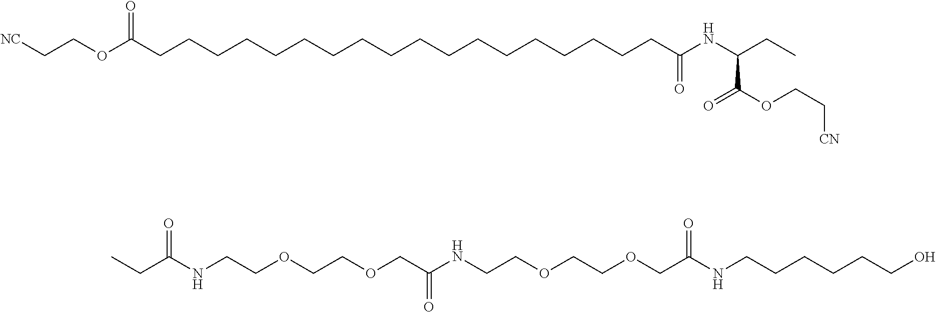 [SMILES: CC[C@H](NC(=O)CCCCCCCCCCCCCCCCCCC(=O)OCCC#N)C(=O)OCCC#N ; CCC(=O)NCCOCCOCC(=O)NCCOCCOCC(=O)NCCCCCCO]

A solution of (S)-46-cyano-22-((2-cyanoethoxy)carbonyl)-10,19,24,43-tetraoxo-3,6,12,15,44-pentaoxa-9,18,23-triazahexatetracontanoic acid (3.95 g, 4.55 mmol), EDC (1.05 g, 5.46 mmol), 6-amino-1-hexanol (0.59 g, 5.01 mmol), and DCM (23 mL) was stirred at ambient temperature for 18 h. The crude reaction was concentrated, the residue was dissolved in 3:1 CHCl₃/i-PrOH (150 mL) and washed with brine (acidified to pH 3 with 1 N HCl). The organic layer was isolated, dried (MgSO₄), and concentrated to a light yellow, waxy solid (3.20 g, 73%), 2-cyanoethyl (29S)-1-(((2-cyanoethoxy)(diisopropylamino)phosphaneyl)oxy)-29-((2-cyanoethoxy)carbonyl)-8,17,26,31-tetraoxo-10,13,19,22-tetraoxa-7,16,25,30-tetraazapentacontan-50-oate. This was used in the next preparation without further purification or characterization.

### Example 4S: C20-Diacid-CE Phosphoramidite

### 2-cyanoethyl (29S)-1-(((2-cyanoethoxy)(diisopropylamino)phosphaneyl)oxy)-29-((2-cyanoethoxy)carbonyl)-8,17,26,31-tetraoxo-10,13,19,22-tetraoxa-7,16,25,30-tetraazapentacontan-50-oate

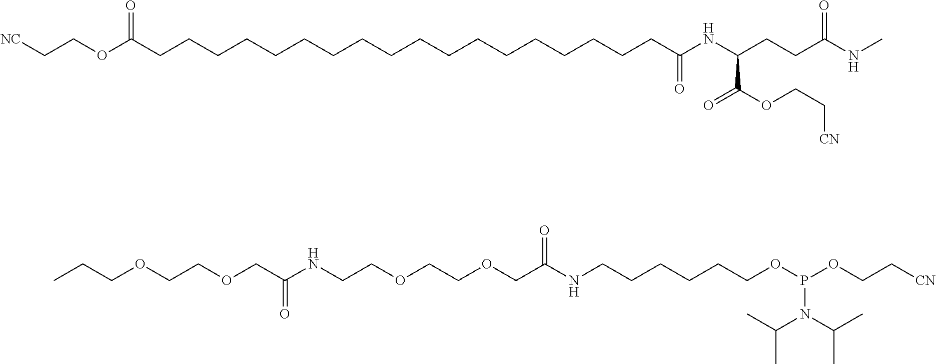

A solution of 2-cyanoethyl (S)-29-((2-cyanoethoxy)carbonyl)-1-hydroxy-8,17,26,31-tetraoxo-10,13,19,22-tetraoxa-7,16,25,30-tetraazapentacontan-50-oate (3.20 g, 3.31 mmol), 5-(ethylthio)-1H-tetrazole (0.25 M in MeCN; 6.6 mL, 1.65 mmol), 3-((bis(diisopropylamino)phosphaneyl)oxy)propanenitrile (1.26 mL, 3.97 mmol), and DCM (20 mL) was stirred at ambient temperature. After 1.5 hours, the crude reaction was poured into a slurry of silica gel (15 g) in DCM (30 mL), concentrated in vacuo to a dry powder, and purified via silica gel flash chromatography eluting with 5-30% MeOH/EtOAc to give 2-cyanoethyl (29S)-1-(((2-cyanoethoxy)(diisopropylamino)phosphaneyl)oxy)-29-((2-cyanoethoxy)carbonyl)-8,17,26,31-tetraoxo-10,13,19,22-tetraoxa-7,16,25,30-tetraazapentacontan-50-oate as a sticky, white foam (1.40 g, 36%). ¹H NMR (d6-DMSO) d 8.22 (d, 1H), 7.88 (t, 1H), 7.70-7.60 (m, 2H), 4.29-4.12 (m, 5H), 3.88 (s, 2H), 3.85 (s, 2H), 3.82-3.49 (m, 14H), 3.49-3.37 (m, 4H), 3.32-3.25 m, 2H), 3.24-3.16 (m, 2H), 3.13-3.04 (m, 2H), 2.91-2.84 (m, 4H), 2.76 (t, 2H), 2.33 (t, 2H), 2.18 (t, 2H), 2.11 (t, 2H), 1.99-1.90 (m, 1H), 1.86-1.74 (m, 1H), 1.59-1.04 (m, 52H). ³¹P NMR (d6-DMSO) d 146.3.

### Example 4T: 1-benzyl 23-methyl (S)-22-((tert-butoxycarbonyl)amino)-10,19-dioxo-3,6,12,15-tetraoxa-9,18-diazatricosanedioate

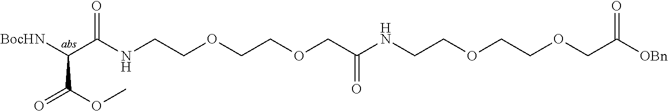 [SMILES: COC(=O)[C@@H](NC(=O)OC(C)(C)C)C(=O)NCCOCCOCC(=O)NCCOCCOCC(=O)OCc1ccccc1]

To a mixture of benzyl 17-amino-10-oxo-3,6,12,15-tetraoxa-9-azaheptadecanoate hydrochloride (35 g, 73% Wt, 1 Eq, 59 mmol) in DCM (350 mL) was added (S)-4-((tert-butoxycarbonyl)amino)-5-methoxy-5-oxopentanoic acid (16 g, 99% Wt, 1 Eq, 59 mmol, CAS number 45214-91-3), 1-(3-Dimethylaminopropyl)-3-ethylcarbodiimideHydrochloride (EDCI) (17 g, 99% Wt, 1.5 Eq, 88 mmol), 1-Hydroxy-1H-benzotriazole (12 g, 99% Wt, 1.5 Eq, 88 mmol) and Diisopropylethylamine (31 g, 41 mL, 99% Wt, 4 Eq, 0.23 mol) at 0° C., then the reaction mixture was stirred at 26° C. for 16 hour. The reaction mixture was concentrated under reduced pressure to give the residue. To the residue was added H2O (500 mL) and extracted with EtOAc (300 mL*2). The combined organic layers were washed with brine (500 mL*2), dried over anhydrous Na2SO4, filtered and concentrated under reduced pressure to give a crude product. The crude product was purified by flash silica gel chromatography (Biotage®; Agela®Flash Column Silica-CS (220 g), Eluent of 0-100% EtOAc/petroleum ether gradient @100 mL/min) to give 1-benzyl 23-methyl (S)-22-((tert-butoxycarbonyl)amino)-10,19-dioxo-3,6,12,15-tetraoxa-9,18-diazatricosanedioate (25.5 g, 38 mmol, 65%, 96% Purity) as colorless gel. LCMS m/z=642.3 (M+1)¹H NMR (400 MHz, METHANOL-d₄) δ ppm 7.24-7.44 (m, 5H) 5.19 (s, 2H) 4.21 (s, 2H) 3.98 (s, 2H) 3.69-3.73 (m, 5H) 3.60-3.68 (m, 7H) 3.55 (dt, J=10.80, 5.20 Hz, 4H) 3.40-3.46 (m, 2H) 3.34-3.39 (m, 2H) 2.30 (br t, J=7.20 Hz, 2H) 2.05-2.18 (m, 1H) 1.79-1.94 (m, 1H) 1.43 (s, 9H).

### Example 4U: 1-benzyl 23-methyl (S)-22-amino-10,19-dioxo-3,6,12,15-tetraoxa-9,18-diazatricosanedioate hydrochloride

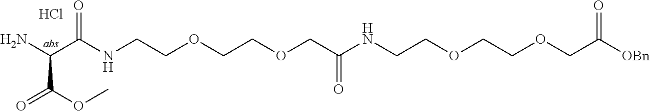 [SMILES: COC(=O)[C@@H](N)C(=O)NCCOCCOCC(=O)NCCOCCOCC(=O)OCc1ccccc1 ; Cl]

To a mixture of 1-benzyl 23-methyl (S)-22-((tert-butoxycarbonyl)amino)-10,19-dioxo-3,6,12,15-tetraoxa-9,18-diazatricosanedioate (25.5 g, 96% Wt, 1 Eq, 38.1 mmol) in EtOAc (50 mL) was added to 2M Hydrogen chloride in ethyl acetate (13.9 g, 191 mL, 2 molar, 10 Eq, 381 mmol) at 0° C. Then the reaction mixture was stirred at 26° C. for 60 min. The reaction mixture was concentrated under reduced pressure to give 1-benzyl 23-methyl (S)-22-amino-10,19-dioxo-3,6,12,15-tetraoxa-9,18-diazatricosanedioate hydrochloride (25 g, 38 mmol, 100%, 89% Purity) as a pink gel. LCMS m/z=542.3 (M+1). ¹H NMR (400 MHz, METHANOL-d₄) δ ppm 7.30-7.39 (m, 5H) 4.60 (s, 2H) 4.17 (s, 2H) 4.05 (s, 2H) 3.84 (s, 3H) 3.69-3.71 (m, 4H) 3.64-3.67 (m, 4H) 3.56-3.61 (m, 6H) 3.44-3.48 (m, 2H) 3.40 (t, J=5.60 Hz, 2H) 2.49-2.54 (m, 2H) 2.15-2.26 (m, 2H).

### Example 4V: 1-benzyl 21,41-dimethyl (S)-9,18,23-trioxo-2,5,11,14-tetraoxa-8,17,22-triazahentetracontane-1,21,41-tricarboxylate

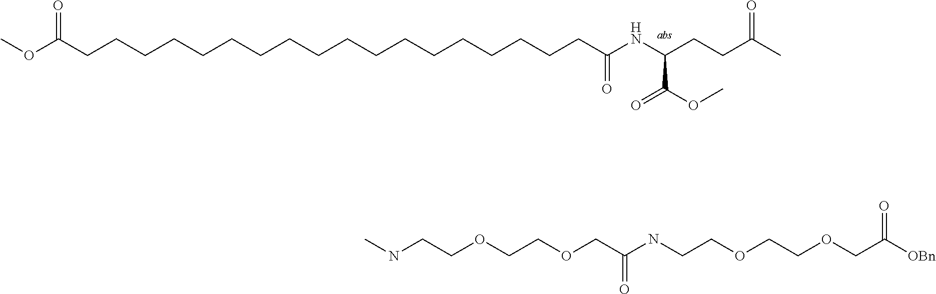

To a mixture of 1-benzyl 23-methyl (S)-22-amino-10,19-dioxo-3,6,12,15-tetraoxa-9,18-diazatricosanedioate hydrochloride (25.0 g, 89% Wt, 1 Eq, 38.5 mmol) in DCM (250 mL) were added 20-methoxy-20-oxoicosanoic acid (13.9 g, 99% Wt, 1 Eq, 38.5 mmol, CAS 1767-98-2),1-(3-Dimethylaminopropyl)-3-ethylcarbodiimideHydrochloride (EDCI) (11.2 g, 99% Wt, 1.5 Eq, 57.7 mmol),1-Hydroxy-1H-benzotriazole (7.88 g, 99% Wt, 1.5 Eq, 57.7 mmol) and Diisopropylethylamine (25.1 g, 33.5 mL, 99% Wt, 5 Eq, 192 mmol) at 0° C., then the reaction mixture was stirred at 26° C. for 16 hour. The reaction mixture was concentrated under reduced pressure to give the residue. To the residue was added H2O (300 mL) and extracted with EtOAc (300 mL*2). The combined organic layers were washed with brine (300 mL*2), dried over anhydrous Na2SO4, filtered and concentrated under reduced pressure to give a crude product. The crude product was purified by flash silica gel chromatography (Biotage®; Agela®Flash Column Silica-CS (120 g), Eluent of 0-7% MeOH/DCM gradient @80 mL/min) to give 1-benzyl 21,41-dimethyl (S)-9,18,23-trioxo-2,5,11,14-tetraoxa-8,17,22-triazahentetracontane-1,21,41-tricarboxylate (35 g, 39 mmol, 100%, 97% Purity) as a white solid. LCMS m/z=880.6 (M+1). ¹H NMR (400 MHz, DMSO-d₆) δ ppm 8.17 (d, J=7.20 Hz, 1H) 7.88 (br t, J=5.20 Hz, 1H) 7.64 (br t, J=5.60 Hz, 1H) 7.22-7.49 (m, 5H) 5.14 (s, 2H) 4.14-4.22 (m, 3H) 3.86 (s, 2H) 3.50-3.59 (m, 19H) 3.16-3.29 (m, 5H) 2.27 (t, J=7.60 Hz, 2H) 2.11 (m, 4H) 1.43-1.54 (m, 4H) 1.22 (s, 28H).

### Example 4W: (S)-24-(methoxycarbonyl)-3,22,27,36-tetraoxo-2,31,34,40,43-pentaoxa-23,28,37-triazapentatetracontan-45-oic acid

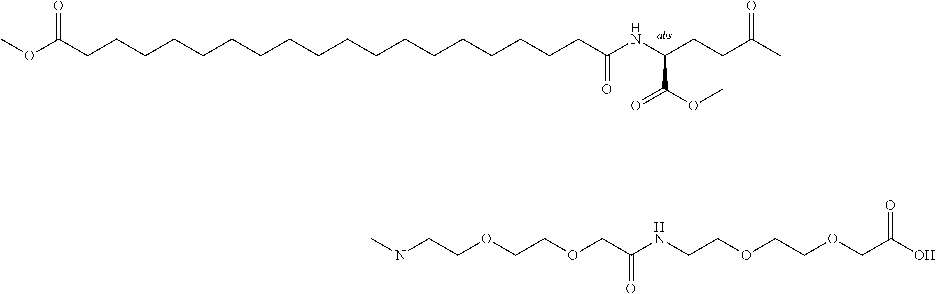

A solution of 1-benzyl 21,41-dimethyl (S)-9,18,23-trioxo-2,5,11,14-tetraoxa-8,17,22-triazahentetracontane-1,21,41-tricarboxylate (30.0 g, 97% Wt, 1 Eq, 33.1 mmol) in THF (300 mL) was degassed with Argon, and Pd/C, wet basis (14.1 g, 10% Wt, 0.4 Eq, 13.2 mmol) was added. The reaction mixture was evacuated and backfilled three times with hydrogen. The mixture was stirred at 25° C. for 120 min under an atmosphere of hydrogen 15 psi. The reaction mixture was filtered through a pad of celite and the filtrate was concentrated under reduced pressure to afford the crude product. Pd/C is recycled into a special recycling bucket. The crude product was purified by flash silica gel chromatography (Biotage®; Agela®Flash Column Silica-CS (120 g), Eluent of 0-15% MeOH/DCM gradient @80 mL/min) to give (S)-24-(methoxycarbonyl)-3,22,27,36-tetraoxo-2,31,34,40,43-pentaoxa-23,28,37-triazapentatetracontan-45-oic acid (17493.85 mg, 21.4 mmol, 64.8%, 96.8% Purity) as a white solid. LCMS m/z=791.0 (M+1). ¹H NMR (400 MHz, METHANOL-d₄) δ ppm 4.39 (dd, J=8.80, 5.20 Hz, 1H) 4.11 (s, 2H) 4.01 (s, 2H) 3.62-3.74 (m, 14H) 3.57 (dt, J=10.80, 5.20 Hz, 4H) 3.42-3.48 (m, 2H) 3.35-3.40 (m, 2H) 2.31 (td, J=7.20, 2.80 Hz, 4H) 2.24 (br t, J=7.60 Hz, 2H) 2.13 (m, 1H) 1.90-2.01 (m, 1H) 1.54-1.68 (m, 4H) 1.29 (br s, 28H).

### Example 4X: C20-Diacid-ME NHS Ester

### 1-(2,5-dioxopyrrolidin-1-yl) 21,41-dimethyl (S)-9,18,23-trioxo-2,5,11,14-tetraoxa-8,17,22-triazahentetracontane-1,21,41-tricarboxylate

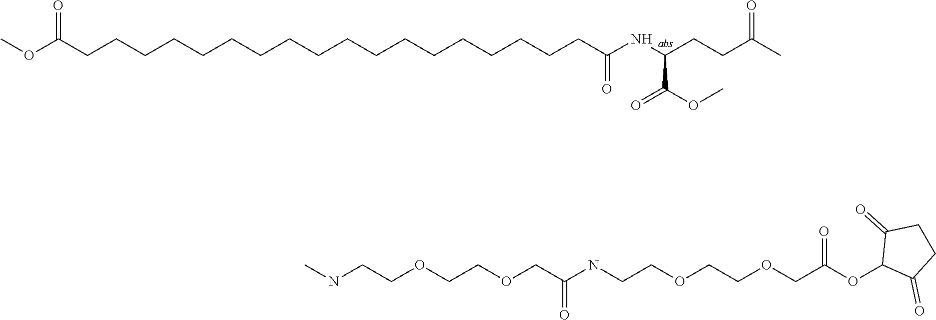

A solution of (S)-24-(methoxycarbonyl)-3,22,27,36-tetraoxo-2,31,34,40,43-pentaoxa-23,28,37-triazapentatetracontan-45-oic acid (500 mg, 1 Eq, 633 mol) 10 mL of DCM, 1-hydroxypyrrolidine-2,5-dione (109 mg, 1.5 Eq, 949 mol) and 3-(((ethylimino)methylene)amino)-N,N-dimethylpropan-1-amine hydrochloride (121 mg, 1 Eq, 633 mol), was stirred at room temperature for 18 hours. The crude reaction was concentrated, and loaded onto 12 g silica cartridge, purified via silica gel flash chromatography eluting with 0-30% MeOH/EtOAc to give 1-(2,5-dioxopyrrolidin-1-yl) 21,41-dimethyl (S)-9,18,23-trioxo-2,5,11,14-tetraoxa-8,17,22-triazahentetracontane-1,21,41-tricarboxylate as a sticky, white foam (320 mg, 54%). ¹H NMR (d6-DMSO) δ 8.15 (d, 1H), 7.86 (t, 1H), 7.65 (t, 1H), 4.61 (s, 2H), 4.16-4.22 (m, 2H), 4.01 (m, 1H), 3.61 (s, 3H), 3.58 (s, 3H), 3.52-3.59 (m, 12H), 3.38-3.48 (m, 4H), 2.29 (t, 2H), 2.07-2.18 (m, 6H), 1.43-1.56 (m, 4H), 1.24 (s, 28H).

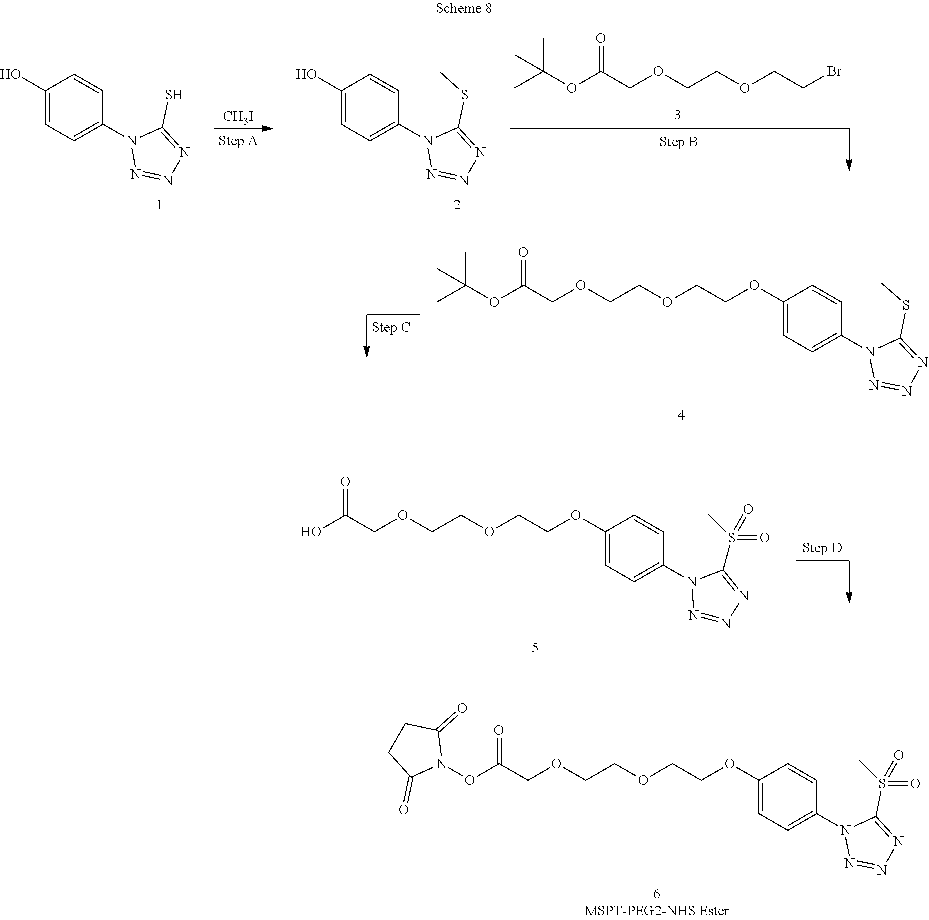

The synthesis of MSPT linker is shown in Scheme 8. The synthesis began from commercially available intermediate 4-(5-mercapto-1H-tetrazol-1-yl)phenol (CAS #52431-78-4). Alkylation of the free sulfide with methyliodide took place under the influence of DIPEA in the solvent THF to yield the methyl sulfide intermediate (2), Step A. As captured in Step B, Williamson ether reaction effected the coupling between the phenol and a bromo-PEG2 reactant that produced ether (4). Step C shows oxidation of the methyl sulfide to the methyl sulfone which used aqueous hydrogen peroxide and catalytic Molybdenum, and the crude sulfone intermediate was treated with trifluoroacetic acid in DCM to yield acid (5). The terminal carboxylic acid was transformed into the NHS-ester with EDCI and NHS—OH in Step D, which provided the activated ester (6) that is reactive to primary amines.

### Example 4Y: 4-(5-(methylthio)-1H-tetrazol-1-yl)phenol

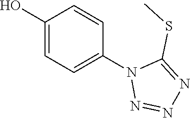

4-(5-mercapto-1H-tetrazol-1-yl)phenol (4.00 g, 20.6 mmol) was dissolved in tetrahydrofuran (50 mL), and the mixture cooled in an ice-bath, prior to the addition of N,N-diisopropylethylamine (4.31 g, 33.3 mmol). A suspension formed after stirring for 10 minutes. Iodomethane (1.54 mL, 24.7 mmol) was added dropwise via syringe over 1 min. The reaction mixture was stirred for 20 minutes with cooling in an ice-bath, then at room temperature for 12 hours. The mixture was diluted with EtOAc (100 mL), and washed with saturated aqueous NH₄Cl (2×50 mL). The organic layer was separated, then dried over sodium sulfate, filtered and concentrated in vacuo to provide 4-(5-(methylthio)-1H-tetrazol-1-yl)phenol (4.2 g, 93% yield) that can be used directly in the next step without further purification. LCMS-mz=209 (M+1).

### Example 4Z: tert-butyl 2-(2-(2-(4-(5-(methylthio)-1H-tetrazol-1-yl)phenoxy)ethoxy)ethoxy)acetate

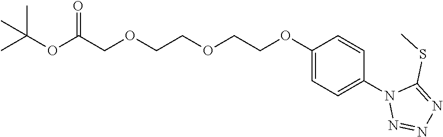 [SMILES: CSc1nnnn1-c1ccc(OCCOCCOCC(=O)OC(C)(C)C)cc1]

A 200 mL pressure vessel was charged with 4-(5-(methylthio)-1H-tetrazol-1-yl)phenol (2.50 g, 11.4 mmol), tert-butyl 2-(2-(2-bromoethoxy)ethoxy)acetate (4.33 g, 14.8 mmol) and acetone (60 mL). Potassium carbonate was added (3.15 g, 22.8 mmol), and the vessel sealed and heated at 80° C. for 8 hours with vigorous stirring. The reaction mixture was cooled to room temperature and filtered to remove potassium carbonate, then washed with acetone/DCM/EtOAc (30 mL each). The filtrate was concentrated in vacuo to provide crude material, which was purified by flash chromatography (80 g, 100% DCM for 5 minutes, then gradient to 100% EtOAc over 20 minutes). The product tert-butyl 2-(2-(2-(4-(5-(methylthio)-1H-tetrazol-1-yl)phenoxy)ethoxy)ethoxy)acetate (3.98 g, 85% yield) was isolated as a white powder. LCMS-mz=411 (M+1).

### Example 4AA: 2-(2-(2-(4-(5-(methylsulfonyl)-1H-tetrazol-1-yl)phenoxy)ethoxy)ethoxy)acetic acid

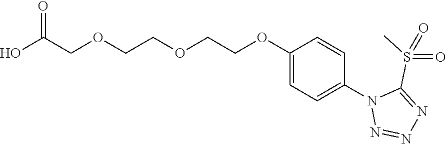 [SMILES: CS(=O)(=O)c1nnnn1-c1ccc(OCCOCCOCC(=O)O)cc1]

Tert-butyl 2-(2-(2-(4-(5-(methylthio)-1H-tetrazol-1-yl)phenoxy)ethoxy)ethoxy)acetate (3.98 g, 9.21 mmol) was dissolved in ethanol (100 mL) and cooled to 5-10° C. in an ice-water bath, prior to the addition of 30% aqueous hydrogen peroxide (19.0 mL, 184 mmol), followed by ammonium molybdate (VI) tetrahydrate (1.14 g, 0.921 mmol). The reaction mixture was stirred at room temperature for 4 hours in an ice bath, then at room temperature for 12 hours. The mixture was diluted with DCM (150 mL) and then washed with brine. The organic phase was separated, dried over sodium sulfate and concentrated in vacuo to dryness. Purification by flash column chromatography (80 g silica, 100% DCM for 5 minutes, then gradient to 100% EtOAc over 20 minutes) provided 2-(2-(2-(4-(5-(methylsulfonyl)-1H-tetrazol-1-yl)phenoxy)ethoxy)ethoxy)acetic acid (3.00 g, 80% yield) as a thick oil. LCMS-mz=385 (M−1).

### Example 4BB: MSPT-PEG2-NHS Ester

### 2,5-dioxopyrrolidin-1-yl 2-(2-(2-(4-(5-(methylsulfonyl)-1H-tetrazol-1-yl)phenoxy)ethoxy)ethoxy)acetate

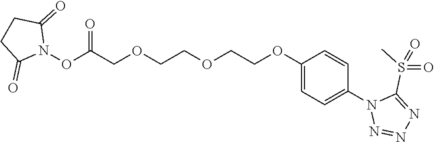 [SMILES: CS(=O)(=O)c1nnnn1-c1ccc(OCCOCCOCC(=O)ON2C(=O)CCC2=O)cc1]

1-Hydroxypyrrolidine-2,5-dione (1.33 g, 1.6 Eq, 11.6 mmol) was added to a solution of 2-(2-(2-(4-(5-(methylsulfonyl)-1H-tetrazol-1-yl)phenoxy)ethoxy)ethoxy)acetic acid (2.80 g, 7.25 mmol) in DCM (50 mL) and THF (70 mL). EDCI (1.60 g, 10.3 mmol) was added in one portion, upon which the solution became a cloudy mixture. Additional DCM (20 mL) was added to bring the mixture into solution again, followed by stirring at room temperature for 12 hours. The solvent was removed in vacuo to provide crude material as a white foam. Purification by flash column chromatography (80 g, 100% DCM for 5 minutes, then gradient to 100% EtOAc over 20 minutes) afforded 2,5-dioxopyrrolidin-1-yl 2-(2-(2-(4-(5-(methylsulfonyl)-1H-tetrazol-1-yl)phenoxy)ethoxy)ethoxy)acetate (2.61 g, 65% yield) as low melting solid. LCMS-mz=484 (M+1).

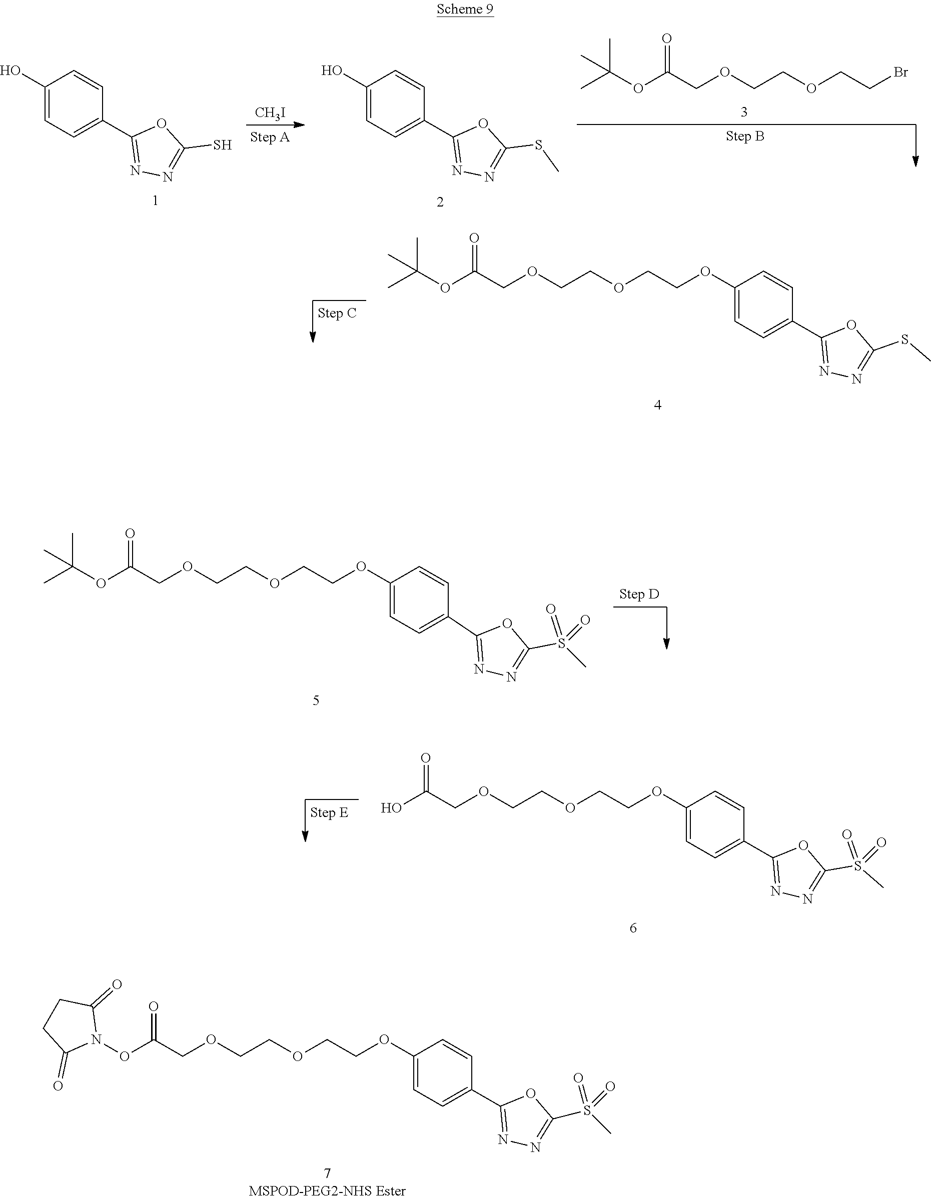

The synthesis of MSPOD linker is shown in Scheme 9. The synthesis began from commercially available intermediate 4-(5-mercapto-1,3,4-oxadiazol-2-yl)phenol (CAS #69829-90-9), and followed steps A and B as described is Scheme 8 for MPST. Oxidation with catalytic Molybdenum and aqueous hydrogen peroxide furnished the sulfone (5), Step C. Step D shows the acidic deprotection of (5) with the strong acid TFA in DCM. The terminal carboxylic acid was transformed into the NHS-ester with EDCI and NHS—OH in Step E, which provided an activated ester (7) that is reactive to primary amines.

### Example 4CC: 4-(5-(methylthio)-1,3,4-oxadiazol-2-yl)phenol

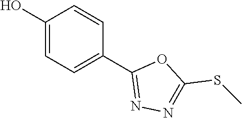

4-(5-Mercapto-1,3,4-oxadiazol-2-yl)phenol (3.00 g, 15.4 mmol) was dissolved in THF (50 mL), and cooled to 0° C. in an ice water bath. N,N-Diisopropylethylamine (3.46 mL, 2.60 g, 20.1 mmol) was added, resulting in a cloudy solution. The mixture was stirred for 5 minutes in the ice bath, then iodomethane (2.85 g, 20.1 mmol) was added drop-wise via syringe over a period of 1 minute. Upon addition, the mixture turned clear after 5 minutes. The cooling bath was removed and the mixture stirred at room temperature for 2 hours, after which it was diluted with dichloromethane (100 mL) and washed with saturated aqueous NH4Cl (pH was adjusted to ~5 by adding citric acid solution, 2×50 mL). The organic layer was separated, dried over sodium sulfate, and concentrated in vacuo to dryness to afford 4-(5-methylsulfanyl-1,3,4-oxadiazol-2-yl)phenol (520 mg, 97% yield) as a pale-yellow solid that was used in the next step without further purification. LC-MS-mz=209 (M+1). Example 4DD: Synthesis of tert-butyl 2-(2-(2-(4-(5-(methylthio)-1,3,4-oxadiazol-2-yl)phenoxy)ethoxy)ethoxy)acetate

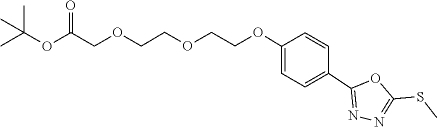 [SMILES: CSc1nnc(-c2ccc(OCCOCCOCC(=O)OC(C)(C)C)cc2)o1]

4-(5-(Methylthio)-1,3,4-oxadiazol-2-yl)phenol (3.3 g, 1 Eq, 15 mmol) and acetone (60 mL) were added to a 200 mL pressure vessel. To this solution was added tert-butyl 2-(2-(2-bromoethoxy)ethoxy)acetate (5.5 g, 20 mmol) and potassium carbonate (4.2 g, 30 mmol). The pressure vessel was sealed and heated at 70° C. for 5 hours with vigorous stirring. After cooling to room temperature, the mixture was filtered to remove solid potassium carbonate, washing with EtOAc/DCM. The filtrate was concentrated in vacuo to dryness and purified by normal phase flash column chromatography (80 g silica gold, 100% DCM for 5 minutes, then gradient to 100% EtOAc over 20 minutes). Product-containing fractions were concentrated in vacuo to afford tert-butyl 2-(2-(2-(4-(5-(methylthio)-1,3,4-oxadiazol-2-yl)phenoxy)ethoxy)ethoxy)acetate (4.8 g, 74% yield) as a white solid. LC-MS-mz=411 (M+1).

### Example 4EE: tert-butyl 2-(2-(2-(4-(5-(methylsulfonyl)-1,3,4-oxadiazol-2-yl)phenoxy)ethoxy)ethoxy)acetate

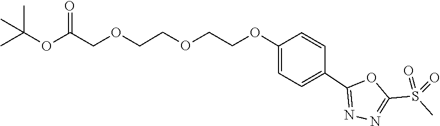 [SMILES: CC(C)(C)OC(=O)COCCOCCOc1ccc(-c2nnc(S(C)(=O)=O)o2)cc1]

Tert-butyl 2-(2-(2-(4-(5-(methylthio)-1,3,4-oxadiazol-2-yl)phenoxy)ethoxy)ethoxy)acetate (5.20 g, 12.7 mmol) was dissolved in 100 mL of ethanol, and cooled to 5-10° C. in an ice-water bath, prior to the addition of 30% hydrogen peroxide (10 mL, 97 mmol), followed by ammonium molybdate (VI) tetrahydrate (501 mg, 0.405 mmol). After two hours of vigorous stirring, an additional 15 mL of 30% hydrogen peroxide and 1 g of Ammonium molybdate (VI) tetrahydrate were added. The reaction mixture was stirred for another 6 hours, then diluted with 150 mL of DCM, and washed with brine. The organic phase was separated, dried over sodium sulfate and concentrated in vacuo to dryness. The residue was triturated with methanol to provide an initial portion of the product. The solvent was then removed from the mother liquid under reduced pressure. Purification by flash column chromatography (40 g, 100% DCM for 3 minutes, then gradient to 100% EtOAc over 20 minutes) provided additional product as a white solid. Combination of both product portions yielded tert-butyl 2-(2-(2-(4-(5-(methylsulfonyl)-1,3,4-oxadiazol-2-yl)phenoxy)ethoxy)ethoxy)acetate (5.3 g, 90% yield) as a white solid. LC-MS-mz=387 (M−tBu).

### Example 4FF: 2-(2-(2-(4-(5-(methylsulfonyl)-1,3,4-oxadiazol-2-yl)phenoxy)ethoxy)ethoxy)acetic acid

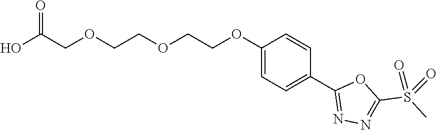 [SMILES: CS(=O)(=O)c1nnc(-c2ccc(OCCOCCOCC(=O)O)cc2)o1]

To a solution of tert-butyl 2-(2-(2-(4-(5-(methylsulfonyl)-1,3,4-oxadiazol-2-yl)phenoxy)ethoxy)ethoxy)acetate (5.60 g, 12.0 mmol) in DCM (60 mL) was added 2,2,2-trifluoroacetic acid (20 mL, 12.0 mmol). The reaction mixture was stirred at room temperature for 2 hours then concentrated under vacuo to afford a thick residue, which was purified by normal phase flash column chromatography (80 g silica gold column, 100% DCM for 3 minutes, then gradient to 100% EtOAc over 20 minutes). Combination of product-containing fractions and concentration in vacuo yielded 2-(2-(2-(4-(5-(methylsulfonyl)-1,3,4-oxadiazol-2-yl)phenoxy)ethoxy)ethoxy)acetic acid (4.12 g, 82% yield). LCMS-mz=387 (M+1).

### Example 4GG: 2,5-dioxopyrrolidin-1-yl-2-(2-(2-(4-(5-(methylsulfonyl)-1,3,4-oxadiazol-2-yl)phenoxy)ethoxy)ethoxy)acetate

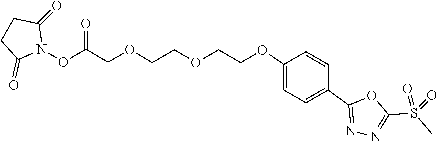 [SMILES: CS(=O)(=O)c1nnc(-c2ccc(OCCOCCOCC(=O)ON3C(=O)CCC3=O)cc2)o1]

2-(2-(2-(4-(5-(Methylsulfonyl)-1,3,4-oxadiazol-2-yl)phenoxy)ethoxy)ethoxy)acetic acid (3.00 g, 7.38 mmol) and 1-hydroxypyrrolidine-2,5-dione (1.19 g, 10.3 mmol) were dissolved in DCM (50 mL) and THF (70 mL). To this solution was added 3-(((ethylimino)methylene)amino)-N,N-dimethylpropan-1-amine (EDCI, 1.60 g, 10.3 mmol). Upon addition, the solution turned cloudy and additional DCM (20 mL) was added to bring the mixture into solution again, followed by stirring at room temperature for 12 hours. The reaction mixture was concentrated under reduced pressure, and the resulting residue dissolved in DCM purified by normal phase flash column chromatography (80 g silica gold 100% DCM for 5 minutes, then gradient to 100% EtOAc over 20 minutes). Concentration of product-containing fractions afforded 2,5-dioxopyrrolidin-1-yl-2-(2-(2-(4-(5-(methylsulfonyl)-1,3,4-oxadiazol-2-yl)phenoxy)ethoxy)ethoxy)acetate (2.61 g, 65% yield). LCMS-mz=484 (M+1).

### Example 4HH: methyl 20-((2-hydroxyethyl)amino)-20-oxoicosanoate

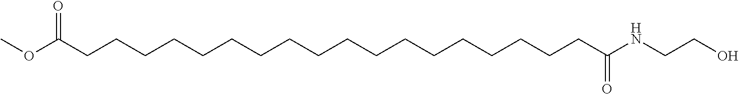 [SMILES: COC(=O)CCCCCCCCCCCCCCCCCCC(=O)NCCO]

20-methoxy-20-oxoicosanoic acid (3.00 g, 8.41 mmol), EDC (1.77 g, 9.23 mmol), HOBT (1.42 g, 9.23 mmol), DIPEA (1.62 mL, 9.23 mmol), and DCM (42 mL) were stirred at ambient temperature until all solid material dissolved (~5 min). Ethanolamine (0.56 mL. 9.23 mmol) was added at which time the reaction turned to a milky white suspension. Stirring at ambient temperature continued for 18 h. The crude reaction was concentrated, the residue was suspended in water (100 mL), acidified with 5 N HCl (2 mL), and then cooled to 0° C. for 15 min. The white solid was isolated via suction filtration, washed with water, and further dried under vacuum (3.27 g, 97%). ¹H NMR (CDCl₃) δ 6.45 (br s, 1H), 3.76 (t, 2H), 3.69 (s, 3H), 3.50-3.42 (m, 2H), 2.91 (br s, 1H), 2.37-2.24 (m, 4H), 1.72-1.58 (m, 4H), 1.39-1.20 (m, 28H).

### Example 411: methyl 20-((2-(((2-cyanoethoxy)(diisopropylamino)phosphaneyl)oxy)ethyl)amino)-20-oxoicosanoate (C20-Acid-Ethanolamide phosphoramidite)

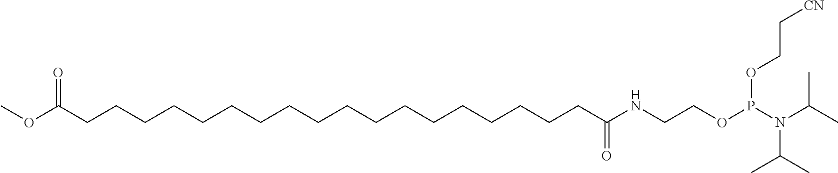 [SMILES: COC(=O)CCCCCCCCCCCCCCCCCCC(=O)NCCOP(OCCC#N)N(C(C)C)C(C)C]

A solution of methyl 20-((2-hydroxyethyl)amino)-20-oxoicosanoate (3.25 g, 8.13 mmol), 5-(ethylthio)-1H-tetrazole (0.25 M in MeCN; 16.3 mL, 4.07 mmol), 3-((bis(diisopropylamino)phosphaneyl)oxy)propanenitrile (3.36 mL, 10.6 mmol), and DCM (40 mL) was stirred at ambient temperature. After 3 hours, to crude reaction was added Celite (25 g) and the suspension was concentrated to a dry solid then purified via basic alumina flash chromatography eluting with 20-60% EtOAc/hexane to give the title compound as a white solid (4.15 g, 85%). ¹H NMR (d6-DMSO) δ 7.83 (t, 1H), 3.81-3.67 (m, 2H), 3.64-3.45 (m, 7H), 3.27-3.17 (m, 2H), 2.76 (t, 2H), 2.29 (t, 2H), 2.05 (t, 2H), 1.57-1.42 (m, 4H), 1.31-1.18 (m, 28H), 1.14 (t, 12H). ³¹P NMR (d6-DMSO) δ 146.8.

### Example 4JJ: 2-cyanoethyl (2-octyldodecyl) diisopropylphosphoramidite (C20-Octyldodecyl phosphoramidite)

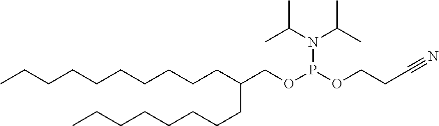 [SMILES: CCCCCCCCCCC(CCCCCCCC)COP(OCCC#N)N(C(C)C)C(C)C]

A solution of octyldodecanol (1.00 g, 3.35 mmol), dichloromethane (50 mL), 5-(ethylthio) tetrazole solution (7 mL, 0.25 M in MeCN, 2 mmol), and 3-((bis(diisopropylamino)phosphanyl)oxy)propanenitrile (1.49 mL, 4.69 mmol) was stirred at ambient temperature for 2 hours. Added Celite (12 g) and the suspension was concentrated to a dry solid then purified via 160 g neutral alumina flash chromatography eluting with 0-10% EtOAc/hexane to give the title compound as a thin oil (1.25 g, 75%). 1H NMR (CDCl3) δ 3.84 (m, 2H) 3.61 (m, 4H) 3.49 (m, 1H) 2.66 (t, 2H) 1.28 (m, 32H) 1.14 (t, 12H) 0.90 (t, 6H). 31P NMR (CDCl₃) δ 147.6.

### Example 4KK: (Z)-2-cyanoethyl octadec-9-en-1-yl diisopropylphosphoramidite (Oleyl phosphoramidite)

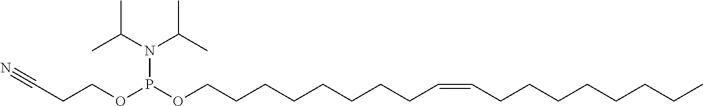 [SMILES: CCCCCCCC/C=C\CCCCCCCCOP(OCCC#N)N(C(C)C)C(C)C]

A solution of oleyl alcohol (1.00 g, 3.72 mmol), dichloromethane (50 mL), 5-(ethylthio) tetrazole solution (8 mL, 0.25 M in MeCN, 2 mmol), and 3-((bis(diisopropylamino)phosphanyl)oxy)propanenitrile (1.68 g, 5.59 mmol) was stirred at ambient temperature for 2 hours. Added Celite (12 g) and the suspension was concentrated to a dry solid then purified via 160 g neutral alumina flash chromatography eluting with 0-7% EtOAc/hexane to give the title compound as a thin oil (1.4 g, 80%). 1H NMR (d6-DMSO) δ 5.33 (t, 2H) 3.71 (m, 2H) 3.57 (m, 4H) 2.76 (t, 2H) 1.98 (m, 4H) 1.53 (q, 2H) 1.25 (m, 22H) 1.14 (t, 12H) 0.86 (t, 3H). 31P NMR (d6-DMSO) δ 146.49

### Example 4LL: 2-cyanoethyl hexadecyl diisopropylphosphoramidite (Cetyl phosphoramidite)

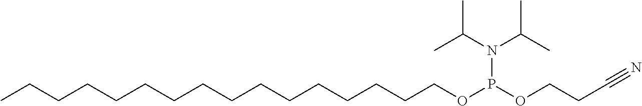 [SMILES: CCCCCCCCCCCCCCCCOP(OCCC#N)N(C(C)C)C(C)C]

A solution of cetyl alcohol (2 g, 8 mmol), dichloromethane (35 mL), 2H-tetrazole (0.45 M in MeCN, 18 mL, 7 mmol), and 3-((bis(diisopropylamino)phosphanyl)oxy)propanenitrile (3 g, 10 mmol) was stirred at ambient temperature for 3 hours. Evaporated the solvents and redissolved in hexanes. Purified via 40 g basic alumina flash chromatography eluting with 0-7% EtOAc/hexane to give the title compound as a thin oil (2.7 g, 70%). 1H NMR (CDCl3) δ 3.84 (m, 2H) 3.61 (m, 4H) 2.66 (t, 2H) 1.62 (m, 2H) 1.28 (m, 24H) 1.2 (t, 12H) 0.90 (t, 3H). 31P NMR (CDCl3) δ 147.3.

### Example 4MM: 2-cyanoethyl docosyl diisopropylphosphoramidite (C22-Docosyl phosphoramidite)

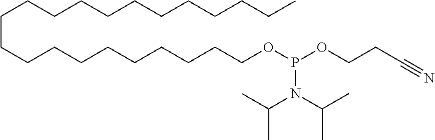 [SMILES: CCCCCCCCCCCCCCCCCCCCCCOP(OCCC#N)N(C(C)C)C(C)C]

A solution of n-docosanol (0.615 g, 1.88 mmol), dichloromethane (53 mL), 5-(ethylthio) tetrazole solution (0.25 M in MeCN, 4 mL, 1 mmol), and 3-((bis(diisopropylamino)phosphanyl)oxy)propanenitrile (851 mg, 2.82 mmol) was stirred at ambient temperature for 2 hours. Added Celite (12 g) and the suspension was concentrated to a dry solid then purified via 48 g neutral alumina flash chromatography eluting with 0-8% EtOAc/hexane to give the title compound as a white waxy solid (0.540 g, 54%). 1H NMR (CDCl3) δ 3.84 (m, 2H) 3.61 (m, 4H) 2.66 (t, 2H) 1.62 (m, 2H) 1.28 (m, 38H) 1.2 (t, 12H) 0.90 (t, 3H). 31P NMR (CDCl3) δ 147.1.

### Example 4NN: 2-((3r,5r,7r)-adamantan-1-yl)ethyl (2-cyanoethyl) diisopropylphosphoramidite (Adamantaneethanol phosphoramidite)

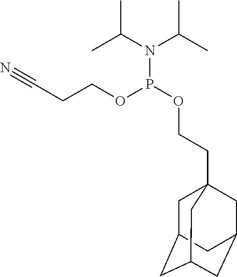 [SMILES: CC(C)N(C(C)C)P(OCCC#N)OCCC12CC3CC(CC(C3)C1)C2]

A solution of 1-admantaneethanol (2.00 g, 11.1 mmol), dichloromethane (50 mL), 5-(ethylthio) tetrazole solution (0.25 M in MeCN, 22 mL, 5.55 mmol), and 3-((bis(diisopropylamino)phosphanyl)oxy)propanenitrile (5.02 g, 16.6 mmol) was stirred at ambient temperature for 4 hours. Added Celite (12 g) and the suspension was concentrated to a dry solid then purified via 160 g neutral alumina flash chromatography eluting with 0-8% EtOAc/hexane to give the title compound as a clear thin oil (0.540 g, 54%). 1H NMR (CDCl3) δ 3.71 (m, 2H) 3.57 (m, 4H) 2.76 (t, 2H) 1.9 (s, 6H) 1.63 (q, 6H) 1.5 (m, 6H) 1.35 (t, 2H) 1.14 (t, 12H) 31P NMR (d6-DMSO) δ 146.0

### Example 4OO: methyl 20-(((2-cyanoethoxy)(diisopropylamino)phosphaneyl)oxy)icosanoate (0-C20-Acid phosphoramidite)

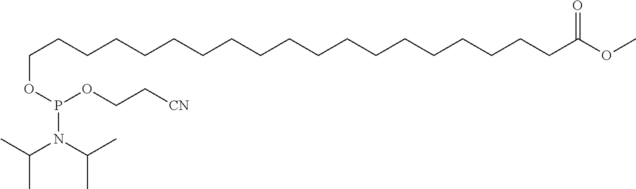 [SMILES: COC(=O)CCCCCCCCCCCCCCCCCCCOP(OCCC#N)N(C(C)C)C(C)C]

A solution of methyl 20-hydroxyicosanoate (3.10 g, 9.05 mmol), dichloromethane (45 mL), 5-(ethylthio) tetrazole solution (0.25 M in MeCN, 18 mL, 4.5 mmol), and 3-((bis(diisopropylamino)phosphanyl)oxy)propanenitrile (3.55 g, 11.8 mmol) was stirred at ambient temperature for 1 hours. Added Celite (25 g) and the suspension was concentrated to a dry solid then purified via 160 g basic alumina flash chromatography eluting with 0-10% EtOAc/hexane to give the title compound as a thick oil (4.1 g, 84%). 1H NMR (d6-DMSO) δ 3.79-3.50 (m, 9H), 2.76 (t, 2H), 2.28 (t, 2H), 1.58-1.46 (m, 4H), 1.36-1.19 (m, 30H), 1.17-1.10 (m, 12H). 31P NMR (d6-DMSO) δ 146.3.

### Example 4PP: methyl 18-(((2-cyanoethoxy)(diisopropylamino)phosphaneyl)oxy)octadecenoate (O-C18-Acid phosphoramidite)

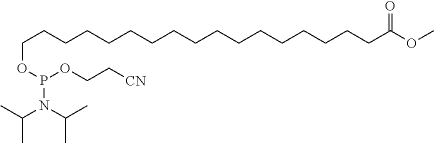 [SMILES: COC(=O)CCCCCCCCCCCCCCCCCOP(OCCC#N)N(C(C)C)C(C)C]

A solution of methyl 18-hydroxyoctadecanoate (3.15 g, 10.0 mmol), dichloromethane (50 mL), 5-(ethylthio) tetrazole solution (0.25 M in MeCN, 20 mL, 5 mmol), and 3-((bis(diisopropylamino)phosphanyl)oxy)propanenitrile (3.93 g, 13.0 mmol) was stirred at ambient temperature for 1 hours. Added Celite (25 g) and the suspension was concentrated to a dry solid then purified via 160 g basic alumina flash chromatography eluting with 0-10% EtOAc/hexane to give the title compound as a thick oil (4.0 g, 78%). 1H NMR (d6-DMSO) δ 3.79-3.50 (m, 9H), 2.76 (t, 2H), 2.28 (t, 2H), 1.58-1.46 (m, 4H), 1.36-1.19 (m, 26H), 1.17-1.10 (m, 12H). 31P NMR (d6-DMSO) δ 146.3.

### Example 4QQ: methyl 16-(((2-cyanoethoxy)(diisopropylamino)phosphaneyl)oxy)hexadecanoate (O-C16-Acid phosphoramidite)

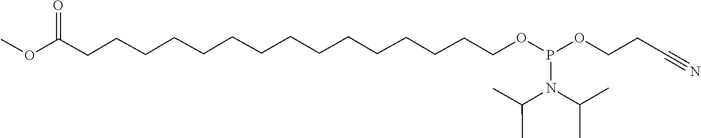 [SMILES: COC(=O)CCCCCCCCCCCCCCCOP(OCCC#N)N(C(C)C)C(C)C]

A solution of methyl 16-hydroxyhexadecanoate (3.15 g, 11.0 mmol), 5-(ethylthio)-1H-tetrazole (0.25 M in MeCN; 22.0 mL, 5.50 mmol), 3-((bis(diisopropylamino)phosphaneyl)oxy)propanenitrile (4.54 mL, 14.3 mmol), and DCM (55.0 mL) was stirred at ambient temperature. After 2 hours, to crude reaction was added Celite (25 g) and the suspension was concentrated to a dry solid then purified via basic alumina flash chromatography eluting with 100% hexane to give the title compound as a clear oil (5.03 g, 93% yield). 1H NMR (Acetonitrile-d3) δ 3.82-3.69 (m, 2H), 3.66-3.54 (m, 4H), 3.59 (s, 3H), 2.62 (t, 2H), 2.27 (t, 2H), 1.60-1.52 (m, 4H), 1.36-1.23 (m, 22H), 1.20-1.11 (m, 12H). 31P NMR (Acetonitrile-d3) δ 146.8.

### Example 4RR: methyl 16-((2-(((2-cyanoethoxy)(diisopropylamino)phosphaneyl)oxy)ethyl)amino)-16-oxohexadecanoatev (C16-Acid Ethanolamide phosphoramidite)

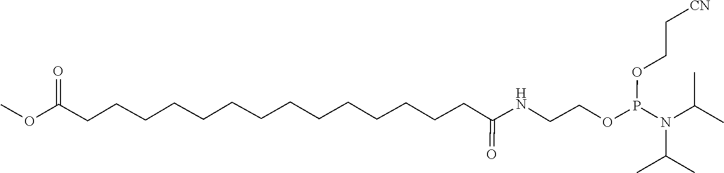 [SMILES: COC(=O)CCCCCCCCCCCCCCC(=O)NCCOP(OCCC#N)N(C(C)C)C(C)C]

A solution of 16-methoxy-16-oxohexadecanoic acid (3.05 g, 10.2 mmol), DCM (50 mL), EDC (2.14 g, 11.2 mmol), HOBt (1.71 g, 11.2 mmol), and DIPEA (1.44 g, 11.2 mmol) was stirred at ambient temperature. 2-aminoethan-1-ol (682 mg, 11.2 mmol) was added resulting in a milky white suspension and stirring continued. After 18 hours, the reaction was concentrated to remove DCM, the white solid was suspended in water (100 mL) and acidified with 5 N HCl (2 mL). The resultant aqueous suspension was cooled to 0° C. for 15 min (stirred rapidly for 5 min). White solid isolated via suction filtration, washed with 0.2 N HCl, water, then dried under vacuum to yield methyl 16-((2-hydroxyethyl)amino)-16-oxohexadecanoate (3.37 g, 97% yield).

A suspension of methyl 16-((2-hydroxyethyl)amino)-16-oxohexadecanoate (3.37 g, 9.81 mmol), 3-((bis(diisopropylamino)phosphaneyl)oxy)propanenitrile (3.84 g, 12.8 mmol), and DCM (50 mL) was stirred at ambient temperature and 5-(ethylthio)-1H-tetrazole (0.25 M in MeCN) (19.6 mL, 4.91 mmol) was added in one portion and stirring continued. The reaction turned to a clear, colorless solution after 1 hour. After 2 h, to the reaction was added Celite (~25 g), concentrated to a dry powder, and then purified via 160 g basic alumina flash chromatography eluting with 10-60% EtOAc/hexane to give the title compound as a white solid (5.0 g, 94%). 1H NMR (d6-DMSO) δ 7.83 (t, 1H), 3.81-3.67 (m, 2H), 3.81-3.67 (m, 2H), 2.76 (t, 2H), 2.28 (t, 2H), 1.58-1.46 (m, 4H), 1.36-1.19 (m, 30H), 1.17-1.10 (m, 12H). 31P NMR (d6-DMSO) δ 146.8.

### Example 4SS: methyl 16-((2-(((2-cyanoethoxy)(diisopropylamino)phosphaneyl)oxy)ethyl)amino)-16-oxohexadecanoate (γ-Glu-C16-Acid Ethanolamide phosphoramidite)

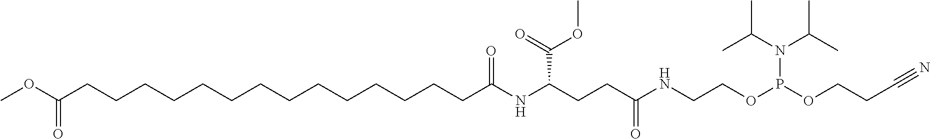 [SMILES: COC(=O)CCCCCCCCCCCCCCC(=O)N[C@@H](CCC(=O)NCCOP(OCCC#N)N(C(C)C)C(C)C)C(=O)OC]

Step 1: To a round bottomed flask was added: 16-methoxy-16-oxohexadecanoic acid (2.00 g, 1 Eq, 6.66 mmol, CAS 18451-85-9, CombiBlocks), DCM (33.3 mL), EDC (1.40 g, 1.1 Eq, 7.32 mmol), HOBt (1.12 g, 1.1 Eq, 7.32 mmol), and DIPEA (946 mg, 1.28 mL, 1.1 Eq, 7.32 mmol). The reaction mixture was stirred at room temperature for 30 minutes over the course of which solids slowly dissolved. Then to the reaction mixture was added 5-(tert-butyl) 1-methyl L-glutamate hydrochloride (1.86 g, 1.1 Eq, 7.32 mmol, CAS 6234-01-1, Alfa Aesar) and stirring at room temperature was continued. The reaction mixture was observed to be cloudy white after this addition. The reaction was allowed to proceed overnight. The next morning, reaction was still cloudy with a small amount of oily, white solid present. The reaction was diluted with DCM (50 mL) then washed with water (50 mL), again with water (50 mL) acidified with 0.5 mL 5 N HCl, and then brine. The organic layer was dried (MgSO4) and concentrated to a white solid. LCMS confirmed mass of the desired product: 5-(tert-butyl) 1-methyl (16-methoxy-16-oxohexadecanoyl)-L-glutamate. LCMS m/z=500.2 (M+1)

The product was used in subsequent reaction step without further purification.

Step 2: A round bottomed flask was charged with 5-(tert-butyl) 1-methyl (16-methoxy-16-oxohexadecanoyl)-L-glutamate (3.32 g, 1 Eq, 6.64 mmol), DCM (16.6 mL), and HCl (2.42 g, 16.6 mL, 4 molar, 10 Eq, 66.4 mmol). Stirred at RT. The reaction mixture turned light orange (from colorless) after addition of HCl. The mixture was allowed to stir for 4 hours then placed in fridge (4° C.) overnight. In the morning, the reaction mixture was removed from fridge and warmed to room temperature. The mixture was concentrated down under vacuum and then loaded onto a silica column and purified using silica gel chromatography on a gradient of 0-100% ethyl acetate in hexane. Product was observed to elute at ~100% ethyl acetate. The fractions were concentrated down, rinsed 2× with DCM and dried to afford a white solid. LCMS confirmed mass of the desired product: (S)-5-methoxy-4-(16-methoxy-16-oxohexadecanamido)-5-oxopentanoic acid. LCMS m/z=444.2 (M+1)

Step 3: A round bottomed flask was charged with (S)-5-methoxy-4-(16-methoxy-16-oxohexadecanamido)-5-oxopentanoic acid (1.3 g, 1 Eq, 2.9 mmol), DCM (15 mL), EDC (0.73 g, 1.3 Eq, 3.8 mmol), and DIPEA (0.45 g, 0.61 mL, 1.2 Eq, 3.5 mmol). The reaction mixture was stirred at room temperature for ~5 minutes as all solid material dissolved. To the reaction mixture was then added ethanolamine (0.21 g, 0.21 mL, 1.2 Eq, 3.5 mmol) in one portion and reaction immediately turned milky white. Stirring was continued at room temperature for 24 hours. The next day, the reaction mixture was concentrated en vacuo. The white solid residue was partitioned between 3:1 CHCl3/IPA (75 mL) and water (acidified with 5 N HCl). The organic layer was then washed with brine, dried (MgSO4), and concentrated and rinsed 2× with DCM and dried to afford a white solid.

LCMS confirmed mass of the desired product: methyl (S)-16-((5-((2-hydroxyethyl)amino)-1-methoxy-1,5-dioxopentan-2-yl)amino)-16-oxohexadecanoate. LCMS m/z=487.5 (M+1)

The product was used in subsequent reaction step without further purification.

Step 4: A round bottomed flask was charged with methyl (S)-16-((5-((2-hydroxyethyl)amino)-1-methoxy-1,5-dioxopentan-2-yl)amino)-16-oxohexadecanoate (1.3 g, 1 Eq, 2.7 mmol), Chloroform (13 mL), 3-((bis(diisopropylamino)phosphaneyl)oxy)propanenitrile (1.6 g, 1.7 mL, 2.0 Eq, 5.3 mmol), and 5-(ethylthio)-1H-tetrazole (0.17 g, 5.3 mL, 0.25 molar, 0.5 Eq, 1.3 mmol). The reaction mixture was stirred at room temperature. Thin layer chromatography after 1 hr indicated full consumption of starting material. The reaction was concentrated down and loaded via liquid phase for flash chromatography using a Basic Alumina column (160 g column) with 0-100% ethyl acetate/hexane gradient. A strong signal began to elute at ~90%. Fractions concentrated to a white solid.

LCMS confirmed the mass of the title product: methyl 16-(((2S)-5-((2-(((2-cyanoethoxy)(diisopropylamino)phosphaneyl)oxy)ethyl)amino)-1-methoxy-1,5-dioxopentan-2-yl)amino)-16-oxohexadecanoate. LCMS m/z=586.4 (M-Diisopropylamine fragmentation)

### Example 4TT: methyl 16-((2-(((2-cyanoethoxy)(diisopropylamino)phosphaneyl)oxy)ethyl)amino)-16-oxohexadecanoate

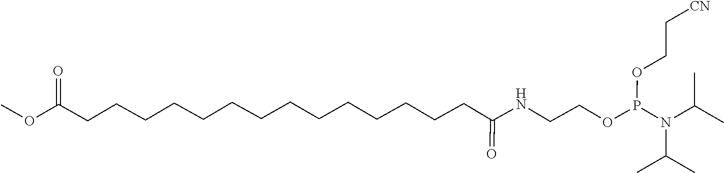 [SMILES: COC(=O)CCCCCCCCCCCCCCC(=O)NCCOP(OCCC#N)N(C(C)C)C(C)C]

A solution of 16-methoxy-16-oxohexadecanoic acid (3.05 g, 10.2 mmol), DCM (50 mL), EDC (2.14 g, 11.2 mmol), HOBt (1.71 g, 11.2 mmol), and DIPEA (1.44 g, 11.2 mmol) was stirred at ambient temperature. 2-aminoethan-1-ol (682 mg, 11.2 mmol) was added resulting in a milky white suspension and stirring continued. After 18 hours, the reaction was concentrated to remove DCM, the white solid was suspended in water (100 mL) and acidified with 5 N HCl (2 mL). The resultant aqueous suspension was cooled to 0° C. for 15 min (stirred rapidly for 5 min). White solid isolated via suction filtration, washed with 0.2 N HCl, water, then dried under vacuum to yield methyl 16-((2-hydroxyethyl)amino)-16-oxohexadecanoate (3.37 g, 97% yield).

A suspension of methyl 16-((2-hydroxyethyl)amino)-16-oxohexadecanoate (3.37 g, 9.81 mmol), 3-((bis(diisopropylamino)phosphaneyl)oxy)propanenitrile (3.84 g, 12.8 mmol), and DCM (50 mL) was stirred at ambient temperature and 5-(ethylthio)-1H-tetrazole (0.25 M in MeCN) (19.6 mL, 4.91 mmol) was added in one portion and stirring continued. The reaction turned to a clear, colorless solution after 1 hour. After 2 h, to the reaction was added Celite (~25 g), concentrated to a dry powder, and flashed (160 g basic alumina column) with 10-60% EA/H to give the title compound as a white solid (5.0 g, 94%). ¹H NMR (d6-DMSO) 7.83 (t, 1H), 3.81-3.67 (m, 2H), 3.63-3.45 (m, 7H), 3.27-3.17 (m, 2H), 2.76 (t, 2H), 2.29 (t, 2H), 2.05 (t, 2H), 1.57-1.42 (m, 4H), 1.31-1.19 (m, 20H), 1.14 (t, 12H). ³¹P NMR (d6-DMSO) d 146.8.

### Example 4UU: N-(3-(((2R,3R,4R,5R)-5-((bis(4-methoxyphenyl)(phenyl)methoxy)methyl)-2-(2,4-dioxo-3,4-dihydropyrimidin-1(2H)-yl)-4-hydroxytetrahydrofuran-3-yl)oxy)propyl)icosanamide

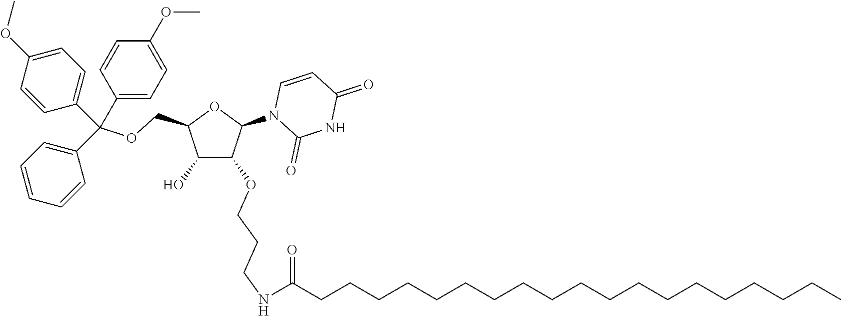 [SMILES: CCCCCCCCCCCCCCCCCCCC(=O)NCCCO[C@@H]1[C@H](O)[C@@H](COC(c2ccccc2)(c2ccc(OC)cc2)c2ccc(OC)cc2)O[C@H]1n1ccc(=O)[nH]c1=O]

A solution of 1-((2R,3R,4R,5R)-3-(3-aminopropoxy)-5-((bis(4-methoxyphenyl)(phenyl)methoxy)methyl)-4-hydroxytetrahydrofuran-2-yl)pyrimidine-2,4(1H,3H)-dione (1.00 g, 1.66 mmol), DCM (10 mL), EDC (0.35 g, 1.83 mmol), HOBt (0.28 g, 1.83 mmol), and DIPEA (0.32 mL, 1.83 mmol) was stirred at ambient temperature. icosanoic acid (0.52 g, 1.66 mmol) was added and stirring continued. After 18 hours, the reaction was diluted with DCM (30 mL) and washed with water (30 mL) acidified to pH 4 with acetic acid (95 uL). The organic layer was then washed with saturated NaHCO₃, dried (MgSO4), filtered, and concentrated to a white foam to yield methyl the title compound (1.42 g, 95% yield). ¹H NMR (d6-DMSO) 11.4 (br s, 1H), 7.76-7.70 (m, 2H), 7.42-7.21 (m, 9H), 6.91 (d, 4H), 5.79 (d, 1H), 5.28 (dd, 1H), 5.21 (d, 1H), 4.23-4.16 (m, 1H), 4.00-3.94 (m, 1H), 3.93-3.88 (m, 1H), 3.75 (s, 6H), 3.64-3.54 (m, 2H), 3.34-3.21 (m, 2H), 3.20-3.03 (m, 2H), 2.03 (t, 2H), 1.69-1.60 (m, 2H), 1.52-1.41 (m, 2H), 1.32-1.16 (m, 32H), 0.85 (t, 3H).

### Example 4VV: (2R,3R,4R,5R)-2-((bis(4-methoxyphenyl)(phenyl)methoxy)methyl)-5-(2,4-dioxo-3,4-dihydropyrimidin-1(2H)-yl)-4-(3-icosanamidopropoxy)tetrahydrofuran-3-yl (2-cyanoethyl) diisopropylphosphoramidite (2′-O-icosanamidopropyl Uridine CED phosphoramidite)

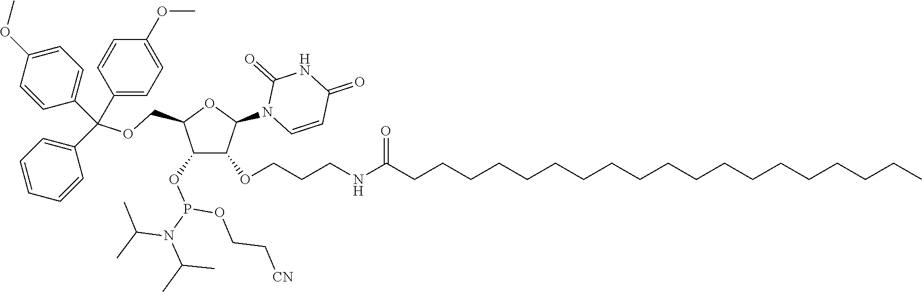 [SMILES: CCCCCCCCCCCCCCCCCCCC(=O)NCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1ccc(=O)[nH]c1=O]

A solution of N-(3-(((2R,3R,4R,5R)-5-((bis(4-methoxyphenyl)(phenyl)methoxy)methyl)-2-(2,4-dioxo-3,4-dihydropyrimidin-1(2H)-yl)-4-hydroxytetrahydrofuran-3-yl)oxy)propyl)icosanamide (1.42 g, 1.58 mmol), 3-((bis(diisopropylamino)phosphaneyl)oxy)propanenitrile (0.62 g, 2.06 mmol), and DCM (10 mL) was stirred at ambient temperature and 5-(ethylthio)-1H-tetrazole (0.25 M in MeCN) (3.15 mL, 0.79 mmol) was added in one portion and stirring continued. After 2 h, to the reaction was added Celite (~10 g), concentrated to a dry solid, and flashed (48 g basic alumina column) with 20-80% EA/H to give the title compound as a thick, colorless oil (1.58 g, 91%). ¹H NMR (d6-DMSO) 11.4 (br s, 1H), 7.84-7.76 (m, 1H), 7.71-7.65 (m, 1H), 7.42-7.21 (m, 9H), 6.94-6.86 (m, 4H), 5.83-5.78 (m, 1H), 5.30-5.21 (m, 1H), 4.46-4.31 (m, 1H), 4.15-3.96 (m, 4 H), 3.84-3.25 (m, 12H), 3.16-3.02 (m, 2H), 2.89 (t, 2H), 2.05-1.98 (m, 2H), 1.70-1.58 (m, 2H), 1.50-1.41 (m, 2H), 1.32-1.06 (m, 44H), 0.85 (t, 3H). ³¹P NMR (d6-DMSO) d 149.1, 148.6.

### Example 4WW: Preparation of 2-propyl nucleotides (PrON)

The compounds of the present disclosure may include PrON, which may be prepared by following the schemes and preparations detailed below. These schemes and preparations are not limiting in their scope.

Certain abbreviations are defined as follows: “AS” refers to antisense strand; “CT” refers to cycle threshold; “DCM” refers to dichloromethane; “DMF” refers to dimethylformamide; “DIEA” refers to N,N-diisopropylethylamine; “PE” refers to petroleum ether; “TFA” refers to trifluoroacetic acid; “DMT” refers to dimethoxytrityl; “DMTCl” refers to dimethoxytrityl chloride; “DMSO” refers to dimethyl sulfoxide; “dsRNA” refers to double stranded ribonucleic acid; “EtOH” refers to ethanol; “EtOAc” refers to ethyl acetate; “hiPSC” refers to human induced pluripotent stem cell; “MeCN” refers to acetonitrile; “MeOH” refers to methanol and methyl alcohol; “PBS” phosphate-buffered saline; “PCR” refers to polymerase chain reaction; “RT-PCR” refers to reverse transcription polymerase chain reaction; “siRNA” refers to small interfering RNA; “SS” refers to sense strand; “TEA” refers to triethylamine; “IBX” refers to 2-iodoxybenzoic acid “9-BBN” refers to 9-borabicyclo[3.3.1]nonane; “2, 4, 6 TMP” refers to 2, 4, 6 trimethylpyridine; “TBAF” refers to tetra-butylammonium fluoride; “TBDMSCl” refers to tert-butyldimethylsilyl chloride and “TBDMS” refers to tert-butyldimethylsilyl.

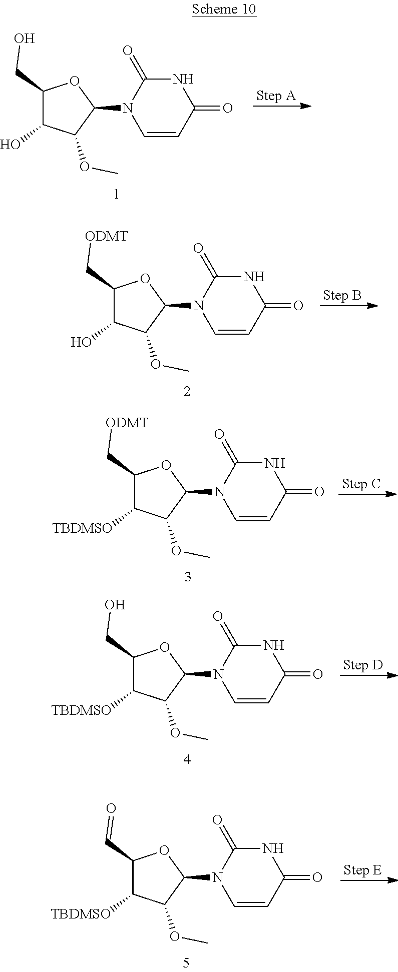

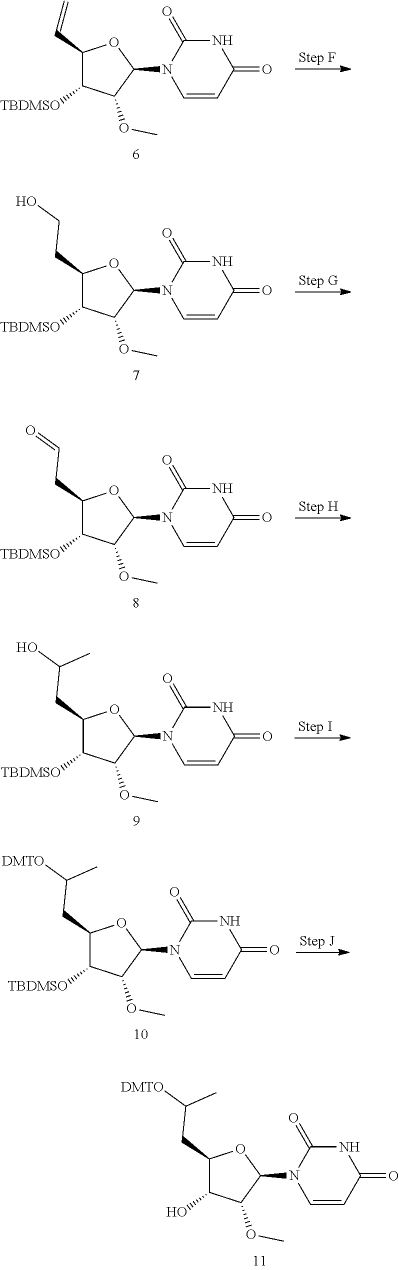

Scheme 10, step A depicts the synthesis of compound (2) by DMT protection of compound (1), the conditions of which will be known by one skilled in the art. Step B shows the conversion of compound (2) to compound (3) by protection of the 3′ alcohol using TBDMSCl in an appropriate solvent such as DMF. For step C, the DMT group was removed from compound (3) to give compound (4), the conditions of which will be known by one skilled in the art. Step D depicts the oxidation of compound (4), using IBX in an appropriate solvent such as EtOAc, to provide compound (5). Conversion of compound (5) to compound (6) via a Wittig reaction, in step E, was accomplished using methyltriphenylphosphonium bromide in an appropriate solvent such as THF. Hydroboration of compound (6) in step F, using 9-BBN in an appropriate solvent such as THF, gave compound (7) which was then oxidized in step G, using IBX and an appropriate solvent such as ACN, to provide compound (8). A Grignard reaction, depicted in step H, was used to convert compound (8) to compound (9) using methylmagnesium bromide in an appropriate solvent such as THF. DMT protection of compound (9), depicted in Step I, provided compound (10) using DMTCl, 2, 4, 6-TMP, and AgNO₃ in an appropriate solvent such as DCM. The TBDMS group was removed from compound (10) to provide compound (11), the conditions of which will be known by one skilled in the art.

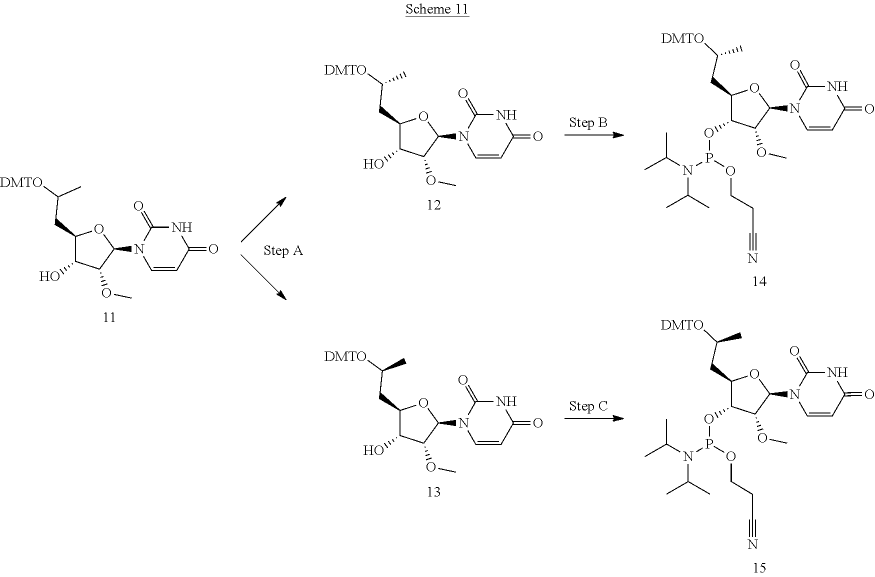

Scheme 11, step A depicts the SFC purification of compound (11) to give compounds (12) and (13) as diastereomers. In step B, the 3′ alcohol of compound (12) was phosphitylated with 3-((chloro(diisopropylamino)phosphaneyl)oxy)propanenitrile in an appropriate solvent such as dichloromethane to provide compound (14). Step C, using the same conditions as step B, provided compound (15) from compound (13).

### Preparation 1

### 1-((2R,3R,4R,5R)-5-((bis(4-methoxyphenyl)(phenyl)methoxy)methyl)-4-hydroxy-3-methoxytetrahydrofuran-2-yl)pyrimidine-2,4(1H,3H)-dione

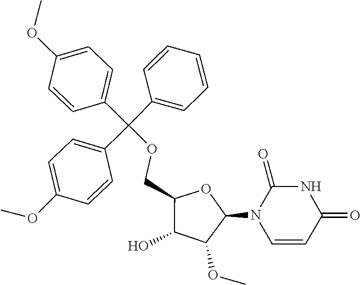 [SMILES: COc1ccc(C(OC[C@H]2O[C@@H](n3ccc(=O)[nH]c3=O)[C@H](OC)[C@@H]2O)(c2ccccc2)c2ccc(OC)cc2)cc1]

DIEA (50.1 g, 67.5 mL, 387 mmol) and 1-[Chloro-(4-methoxyphenyl)-phenyl-methyl]-4-methoxy-benzene (72.2 g, 213 mmol) were added to a solution of 1-((2R,3R,4R,5R)-4-hydroxy-5-(hydroxymethyl)-3-methoxytetrahydrofuran-2-yl)pyrimidine-2,4(1H,3H)-dione (50.0 g, 194 mmol) dissolved in DCM (500 mL). The mixture was stirred at ambient temperature for 15 hours. The reaction mixture was quenched with water (500 ml) and the organic layer was removed. The aqueous layer was extracted three times with DCM (200 ml). The organic layers were combined, dried over sodium sulfate, filtered, and concentrated under reduced pressure to give a residue. The residue was purified by silica gel flash chromatography, eluting with 0% to 100% EtOAc/PE, to give the title compound (100 g, 82%) as a yellow solid. ES/MS (m/z): 583.3 (M+23).

### Preparation 2

### 1-((2R,3R,4R,5R)-5-((bis(4-methoxyphenyl)(phenyl)methoxy)methyl)-4-((tert-butyldimethylsilyl)oxy)-3-methoxytetrahydrofuran-2-yl)pyrimidine-2,4(1H,3H)-dione

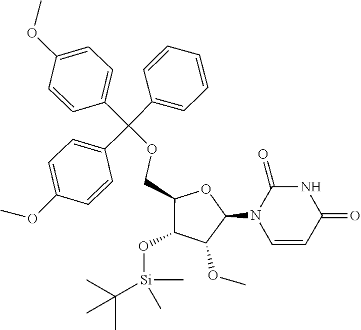 [SMILES: COc1ccc(C(OC[C@H]2O[C@@H](n3ccc(=O)[nH]c3=O)[C@H](OC)[C@@H]2O[Si](C)(C)C(C)(C)C)(c2ccccc2)c2ccc(OC)cc2)cc1]

Imidazole (216 g, 3.18 mol) and tert-butyldimethylchlorosilane (47.96 g, 52.9 mL, 318 mmol) were added to a solution of 1-((2R,3R,4R,5R)-5-((bis(4-methoxyphenyl)(phenyl)methoxy)methyl)-4-hydroxy-3-methoxytetrahydrofuran-2-yl)pyrimidine-2,4(1H,3H)-dione (100.0 g, 159 mmol) in DMF (500 mL). The mixture was stirred under nitrogen at 50° C. for 15 hours. After cooling to ambient temperature, the mixture was quenched with water (1 L). The aqueous layer was extracted three times with EtOAc (800 ml). The organic layers were combined, dried over sodium sulfate, filtered, and concentrated under reduced pressure to give a residue. The residue was purified by silica gel flash chromatography, eluting with 0% to 100% EtOAc/PE, to give the title compound (99 g, 86%) as a yellow solid. ES/MS (m/z): 697.4 (M+23).

### Preparation 3

### 1-((2R,3R,4R,5R)-4-((tert-butyldimethylsilyl)oxy)-5-(hydroxymethyl)-3-methoxytetrahydrofuran-2-yl)pyrimidine-2,4(1H,3H)-dione

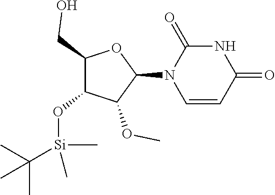 [SMILES: CO[C@@H]1[C@H](O[Si](C)(C)C(C)(C)C)[C@@H](CO)O[C@H]1n1ccc(=O)[nH]c1=O]

TFA (59.0 g, 40 ml, 520 mmol) was added to a solution of 1-((2R,3R,4R,5R)-5-((bis(4-methoxyphenyl)(phenyl)methoxy)methyl)-4-((tert-butyldimethylsilyl)oxy)-3-methoxytetrahydrofuran-2-yl)pyrimidine-2,4(1H,3H)-dione (99.0 g, 136 mmol) dissolved in DCM (1000 mL). The mixture was stirred under nitrogen at ambient temperature for 2 hours, cooled to 0° C. in an ice bath, and then quenched by the addition of aqueous sodium bicarbonate (300 mL). The aqueous was then extracted 3 times with DCM (200 mL) and the organic layers were combined, dried over sodium sulfate, filtered, and concentrated under reduced pressure to give a residue. The residue was purified by silica gel flash chromatography, eluting with 0% to 80% EtOAc/PE, to give the title compound (30 g, 57%) as a yellow solid. ES/MS (m/z): 373.1 (M+1).

### Preparation 4

(2S,3S,4R,5R)-3-((tert-butyldimethylsilyl)oxy)-5-(2,4-dioxo-3,4-dihydropyrimidin-1(2H)-yl)-4-methoxytetrahydrofuran-2-carbaldehyde

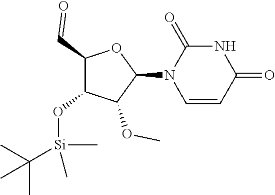 [SMILES: CO[C@@H]1[C@H](O[Si](C)(C)C(C)(C)C)[C@@H](C=O)O[C@H]1n1ccc(=O)[nH]c1=O]

IBX (43.8 g, 156 mmol) was added to a solution of 1-((2R,3R,4R,5R)-4-((tert-butyldimethylsilyl)oxy)-5-(hydroxymethyl)-3-methoxytetrahydrofuran-2-yl)pyrimidine-2,4(1H,3H)-dione (30.0 g, 78.1 mmol) in EtOAc (400 mL). The mixture was stirred under nitrogen at 80° C. for 5 hours. The reaction mixture was then cooled to ambient temperature, filtered, and concentrated under reduced pressure to give to give the title compound (33 g, 86%) as a pink solid. ES/MS (m/z): 371.1 (M+1).

### Preparation 5

### 1-((2R,3R,4R,5R)-4-((tert-butyldimethylsilyl)oxy)-3-methoxy-5-vinyltetrahydrofuran-2-yl)pyrimidine-2,4(1H,3H)-dione

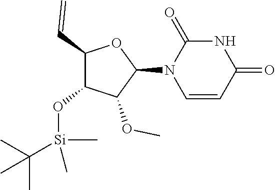 [SMILES: C=C[C@H]1O[C@@H](n2ccc(=O)[nH]c2=O)[C@H](OC)[C@@H]1O[Si](C)(C)C(C)(C)C]

N-Butyllithium (14.6 g, 91.1 mL, 228 mmol, 2.5M in hexane) was added to a solution of methyltriphenylphosphonium bromide (81.4 g, 228 mmol) in THF (600 mL) under nitrogen at −70° C. The solution was warmed to 0° C., stirred for 30 minutes, and (2S,3S,4R,5R)-3-((tert-butyldimethylsilyl)oxy)-5-(2,4-dioxo-3,4-dihydropyrimidin-1(2H)-yl)-4-methoxytetrahydrofuran-2-carbaldehyde (37.5 g, 75.9 mmol) dissolved in THF (300 mL) was then added. The mixture was then warmed to ambient temperature and stirred under nitrogen for 16 hours. After quenching with saturated ammonium chloride (1200 ml) the mixture was further diluted with water (500 ml). The aqueous was extracted 3 times with EtOAc (1000 ml) and the organic layers were combined, washed with brine (800 ml), dried over sodium sulfate, and then reduced to residue. The residue was purified by silica gel flash chromatography, eluting with 0% to 50% EtOAc/PE, to give the title compound (16 g, 56%) as a white solid. ES/MS (m/z): 369.6 (M+1).

### Preparation 6

### 1-((2R,3R,4R,5R)-4-((tert-butyldimethylsilyl)oxy)-5-(2-hydroxyethyl)-3-methoxytetrahydrofuran-2-yl)pyrimidine-2,4(1H,3H)-dione

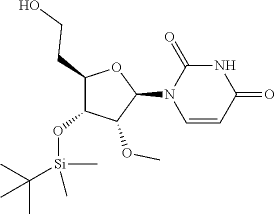 [SMILES: CO[C@@H]1[C@H](O[Si](C)(C)C(C)(C)C)[C@@H](CCO)O[C@H]1n1ccc(=O)[nH]c1=O]

1-((2R,3R,4R,5R)-4-((tert-butyldimethylsilyl)oxy)-3-methoxy-5-vinyltetrahydrofuran-2-yl)pyrimidine-2,4(1H,3H)-dione (11.1 g, 28.6 mmol) was dissolved in THF (200 mL) under nitrogen and the solution was cooled to 0° C. 9-BBN in THF (45.2 g, 143 mmol, 0.5M in THF) was added to the solution and it was stirred at 0° C. for 30 minutes. The mixture was then warmed to ambient temperature. After stirring for 16 hours, the solution was cooled to 0° C. and MeOH (12 mL) was added dropwise. When gas evolution had ceased, water (6.0 mL) was added followed by a mixture of sodium hydroxide (2.29 g, 28.6 mL, 57.2 mmol) and hydrogen peroxide (22.7 g, 20.5 ml, 200 mmol). The ice bath was then removed, and the mixture was stirred vigorously at ambient temperature for 2 hours. Aqueous sodium sulfite (400 mL) was then added, and the mixture was extracted 3 times with EtOAc (500 mL). The organic layers were then combined, washed 2 times with brine (200 ml), dried over sodium sulfate, and then reduced to residue. The residue was purified by silica gel flash chromatography, eluting with 0% to 50% EtOAc/PE, to give the title compound (8.6 g, 78%) as a colorless oil. ES/MS (m/z): 387.2 (M+1).

### Preparation 7

### 2-((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)acetaldehyde

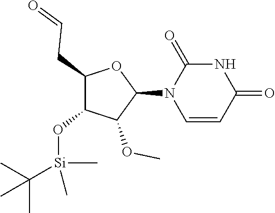 [SMILES: CO[C@@H]1[C@H](O[Si](C)(C)C(C)(C)C)[C@@H](CC=O)O[C@H]1n1ccc(=O)[nH]c1=O]

1-((2R,3R,4R,5R)-4-((tert-butyldimethylsilyl)oxy)-5-(2-hydroxyethyl)-3-methoxytetrahydrofuran-2-yl)pyrimidine-2,4(1H,3H)-dione (12.0 g, 22.4 mmol) was dissolved in MeCN (150 mL) and IBX (12.5 g, 44.7 mmol) was added. After stirring at 80° C. for 1 hour, the mixture was filtered through a Celite pad and concentrated to residue. The residue was purified by silica gel flash chromatography, eluting with 0% to 60% EtOAc/PE, to give the title compound (8.4 g, 86%) as a white solid. ES/MS (m/z): 385.1 (M+1).

### Preparation 8

### 1-((2R,3R,4R,5R)-4-((tert-butyldimethylsilyl)oxy)-5-(2-hydroxypropyl)-3-methoxytetrahydrofuran-2-yl)pyrimidine-2,4(1H,3H)-dione

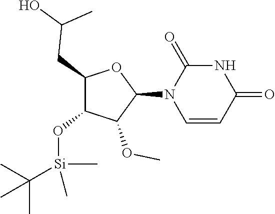 [SMILES: CO[C@@H]1[C@H](O[Si](C)(C)C(C)(C)C)[C@@H](CC(C)O)O[C@H]1n1ccc(=O)[nH]c1=O]

Methylmagnesium bromide (3.49 g, 9.77 mL, 29.3 mmol, 3M in THF) was added to a solution of 2-((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)acetaldehyde (3.20 g, 7.32 mmol) in THF (100 mL) at 0° C. under nitrogen. The solution was stirred at 0° C. for 1 hour. The reaction mixture was quenched by the addition of aqueous saturated ammonium chloride (150 mL) and then diluted with water (100 mL). The aqueous was extracted with EtOAc (200 mL). The organic layer was washed with brine (100 mL), dried over sodium sulfate, filtered, and then reduced to residue. The residue was purified by silica gel flash chromatography, eluting with 20% to 60% EtOAc/PE to give the title compound (1.8 g, 61%) as a colorless oil. ES/MS (m/z): 401.1 (M+1).

### Preparation 9

### 1-((2R,3R,4R,5R)-5-(2-(bis(4-methoxyphenyl)(phenyl)methoxy)propyl)-4-((tert-butyldimethylsilyl)oxy)-3-methoxytetrahydrofuran-2-yl)pyrimidine-2,4(1H,3H)-dione

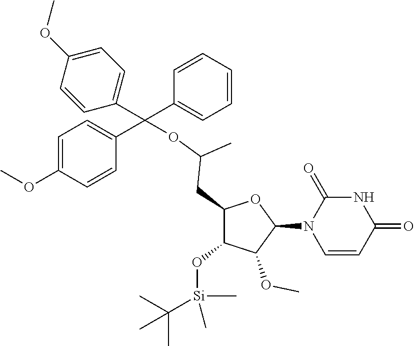 [SMILES: COc1ccc(C(OC(C)C[C@H]2O[C@@H](n3ccc(=O)[nH]c3=O)[C@H](OC)[C@@H]2O[Si](C)(C)C(C)(C)C)(c2ccccc2)c2ccc(OC)cc2)cc1]

Silver nitrate (15.2 g, 89.2 mmol), 2,4,6-trimethylpyridine (14.6 g, 15.9 mL, 119 mmol), and 4,4′-(chloro(phenyl)methylene)bis(methoxybenzene) (20.2 g, 59.7 mmol) were added to a solution of 1-((2R,3R,4R,5R)-4-((tert-butyldimethylsilyl)oxy)-5-((RS)-2-hydroxypropyl)-3-methoxytetrahydrofuran-2-yl)pyrimidine-2,4(1H,3H)-dione (5.90 g, 11.9 mmol) in DCM (120 mL) at 0° C. The ice bath was removed, and the mixture was stirred at ambient temperature for 16 hours under nitrogen. The reaction mixture was quenched by the addition of water (200 mL) and the aqueous was then extracted three times with EtOAc (200 mL). The organic layers were then combined, washed 2 times with brine (200 ml), dried over sodium sulfate, and then reduced to residue. The residue was purified by silica gel flash chromatography, eluting with 0% to 70% EtOAc/PE, to give the title compound (11 g, 74%) as an orange solid. ES/MS (m/z): 725.3 (M+23).

### Preparation 10

### 1-((2R,3R,4R,5R)-5-(2-(bis(4-methoxyphenyl)(phenyl)methoxy)propyl)-4-hydroxy-3-methoxytetrahydrofuran-2-yl)pyrimidine-2,4(1H,3H)-dione

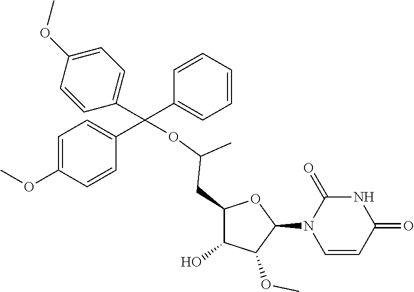 [SMILES: COc1ccc(C(OC(C)C[C@H]2O[C@@H](n3ccc(=O)[nH]c3=O)[C@H](OC)[C@@H]2O)(c2ccccc2)c2ccc(OC)cc2)cc1]

Tetrabutylammonium fluoride (2.50 g, 9.70 mL 9.70 mmol, 1M in THF) was added to a solution of 1-((2R,3R,4R,5R)-5-(2-(bis(4-methoxyphenyl)(phenyl)methoxy)propyl)-4-((tert-butyldimethylsilyl)oxy)-3-methoxytetrahydrofuran-2-yl)pyrimidine-2,4(1H,3H)-dione (10.0 g, 9.70 mmol) in THF (100 mL) and the mixture was stirred at ambient temperature under nitrogen. After 30 minutes of stirring, the reaction mixture was concentrated under reduced pressure to give a residue. The residue was purified by silica gel flash chromatography, eluting with 0% to 100% EtOAc/PE, to give the title compound (5.0 g, 80%) as a white solid. ES/MS (m/z): 587.2 (M−1).

### Preparation 11

### 1-((2R,3R,4R,5R)-5-((S)-2-(bis(4-methoxyphenyl)(phenyl)methoxy)propyl)-4-hydroxy-3-methoxytetrahydrofuran-2-yl)pyrimidine-2,4(1H,3H)-dione and 1-((2R,3R,4R,5R)-5-((R)-2-(bis(4-methoxyphenyl)(phenyl)methoxy)propyl)-4-hydroxy-3-methoxytetrahydrofuran-2-yl)pyrimidine-2,4(1H,3H)-dione

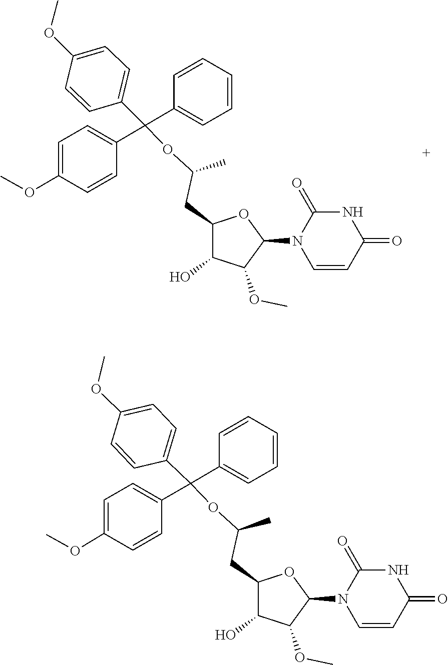 [SMILES: COc1ccc(C(O[C@H](C)C[C@H]2O[C@@H](n3ccc(=O)[nH]c3=O)[C@H](OC)[C@@H]2O)(c2ccccc2)c2ccc(OC)cc2)cc1 ; COc1ccc(C(O[C@@H](C)C[C@H]2O[C@@H](n3ccc(=O)[nH]c3=O)[C@H](OC)[C@@H]2O)(c2ccccc2)c2ccc(OC)cc2)cc1]

1-((2R,3R,4R,5R)-5-(2-(bis(4-methoxyphenyl)(phenyl)methoxy)propyl)-4-hydroxy-3-methoxytetrahydrofuran-2-yl)pyrimidine-2,4(1H,3H)-dione (5.00 g, 8.15 mmol) was purified by SFC (Condition: CO₂-EtOH; Column: Daicel Chiralpak IBN 250 mm×50 mm×10 um; Begin B: 50%; End B: 50%; Gradient Time (min): 150 min; Flowrate: 200 mL/min.) to give 1-((2R,3R,4R,5R)-5-((S)-2-(bis(4-methoxyphenyl)(phenyl)methoxy)propyl)-4-hydroxy-3-methoxytetrahydrofuran-2-yl)pyrimidine-2,4(1H,3H)-dione (first eluting isomer, 1.85 g, 38%) and 1-((2R,3R,4R,5R)-5-((R)-2-(bis(4-methoxyphenyl)(phenyl)methoxy)propyl)-4-hydroxy-3-methoxytetrahydrofuran-2-yl)pyrimidine-2,4(1H,3H)-dione (second eluting isomer, 3.01 g, 61%) as white solids. ES/MS (m/z): 587.2 (M−1).

### Preparation 12

### (2R,3R,4R,5R)-2-((S)-2-(bis(4-methoxyphenyl)(phenyl)methoxy)propyl)-5-(2,4-dioxo-3,4-dihydropyrimidin-1(2H)-yl)-4-methoxytetrahydrofuran-3-yl (2-cyanoethyl) diisopropylphosphoramidite

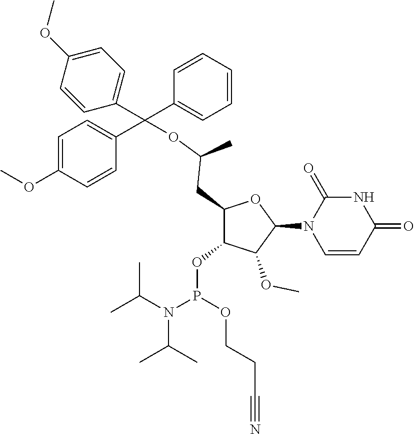 [SMILES: COc1ccc(C(O[C@@H](C)C[C@H]2O[C@@H](n3ccc(=O)[nH]c3=O)[C@H](OC)[C@@H]2OP(OCCC#N)N(C(C)C)C(C)C)(c2ccccc2)c2ccc(OC)cc2)cc1]

1-((2R,3R,4R,5R)-5-((S)-2-(bis(4-methoxyphenyl)(phenyl)methoxy)propyl)-4-hydroxy-3-methoxytetrahydrofuran-2-yl)pyrimidine-2,4(1H,3H)-dione (1.85 g, 3.14 mmol) was dissolved in a mixture of DIEA (1.22 g, 1.64 mL, 9.43 mmol) in DCM (25 mL) and the solution was chilled to 0° C. in an ice bath. 3-((chloro(diisopropylamino)phosphaneyl)oxy)propanenitrile (1.64 g, 1.54 mL, 6.91 mmol) was then added dropwise and the ice bath was removed. The mixture was stirred at ambient temperature for 1.5 hours and the solvent was then removed under vacuum at 25° C. to leave a residue. The residue was purified by silica gel flash chromatography, eluting with 0% to 100% EtOAc/hexanes (eluents contain 1% TEA) to give the title compound (1.54 g, 62%) as an off-white foam. ³¹P NMR (DMSO) δ 148.90, 148.80; ES/MS (m/z): 789.4 (M+1).

### Preparation 13

### (2R,3R,4R,5R)-2-((R)-2-(bis(4-methoxyphenyl)(phenyl)methoxy)propyl)-5-(2,4-dioxo-3,4-dihydropyrimidin-1(2H)-yl)-4-methoxytetrahydrofuran-3-yl (2-cyanoethyl) diisopropylphosphoramidite

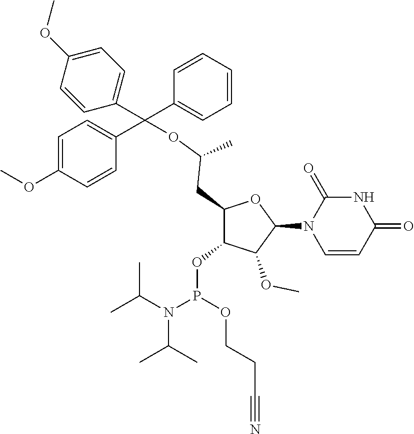 [SMILES: COc1ccc(C(O[C@H](C)C[C@H]2O[C@@H](n3ccc(=O)[nH]c3=O)[C@H](OC)[C@@H]2OP(OCCC#N)N(C(C)C)C(C)C)(c2ccccc2)c2ccc(OC)cc2)cc1]

1-((2R,3R,4R,5R)-5-((R)-2-(bis(4-methoxyphenyl)(phenyl)methoxy)propyl)-4-hydroxy-3-methoxytetrahydrofuran-2-yl)pyrimidine-2,4(1H,3H)-dione (3.01 g, 5.11 mmol) was dissolved in a mixture of DIEA (1.98 g, 2.67 mL, 15.3 mmol) in DCM (40 mL) and the solution was chilled to 0° C. in an ice bath. 3-((chloro(diisopropylamino)phosphaneyl)oxy)propanenitrile (2.66 g, 2.51 mL, 11.2 mmol) was then added dropwise and the ice bath was removed. The mixture was stirred at ambient temperature for 1.5 hours and the solvent was then removed under vacuum at 25° C. to leave a residue. The residue was purified by silica gel flash chromatography, eluting with 0% to 100% EtOAc/hexanes (eluents contain 1% TEA) to give the title compound (3.8 g, 95%) as an off-white foam. ³¹P NMR (DMSO) δ 149.06, 148.99; ES/MS (m/z): 789.4 (M+1).

### Preparation 14

### N-(1-((2R,3R,4R,5R)-5-(2-(bis(4-methoxyphenyl)(phenyl)methoxy)propyl)-4-((tert-butyldimethylsilyl)oxy)-3-methoxytetrahydrofuran-2-yl)-2-oxo-1,2-dihydropyrimidin-4-yl)acetamide

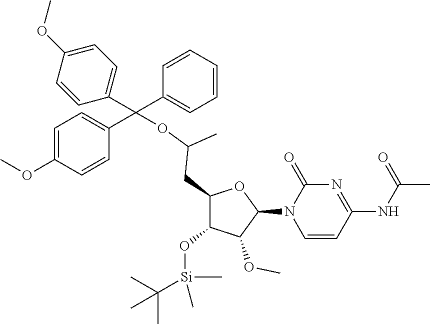 [SMILES: COc1ccc(C(OC(C)C[C@H]2O[C@@H](n3ccc(NC(C)=O)nc3=O)[C@H](OC)[C@@H]2O[Si](C)(C)C(C)(C)C)(c2ccccc2)c2ccc(OC)cc2)cc1]

2, 4, 6-triisopropylbenzenesulfonyl chloride (99.1 g, 327 mmol) was added to a solution of 1-((2R,3R,4R,5R)-5-(2-(bis(4-methoxyphenyl)(phenyl)methoxy)propyl)-4-((tert-butyldimethylsilyl)oxy)-3-methoxytetrahydrofuran-2-yl)pyrimidine-2,4(1H,3H)-dione (115 g, 163 mmol), triethylamine (33.1 g, 327 mmol), and DMAP (40.0 g, 327 mmol) dissolved in ACN (800 ml) at 0° C. The ice bath was removed, and the reaction mixture was stirred at ambient temperature for 2 hours. Ammonium hydroxide (101 g, 834 mmol, 29%) was then added, and the mixture was allowed to stir at ambient temperature for an additional two hours. After stirring was complete, the solution was diluted with water (500 ml), extracted twice with ethyl acetate (500 ml), and the combined organic was washed with brine (500 ml), dried over sodium sulfate and concentrated under reduced pressure to give a brown oil. The oil was dissolved in pyridine (1.22 L), acetic anhydride (76.4 g, 249 mmol) was added, and the solution was stirred at ambient temperature for 2 hours. The solution was then diluted with water (500 ml), extracted twice with ethyl acetate (500 ml), and the combined organic was washed with brine (500 ml), dried over sodium sulfate, and concentrated under reduced pressure to give a residue. The residue was purified by silica gel flash chromatography using 20% to 50% EtOAc/PE to give the title compound (100 g, 27%). ES/MS (m/z): 744.4 (M+1).

Conversion of uracil nucleobase to cytosine may also be conducted generally as described in WO2019/217459.

### Preparation 15

### N-(1-((2R,3R,4R,5R)-5-(2-(bis(4-methoxyphenyl)(phenyl)methoxy)propyl)-4-hydroxy-3-methoxytetrahydrofuran-2-yl)-2-oxo-1,2-dihydropyrimidin-4-yl)acetamide

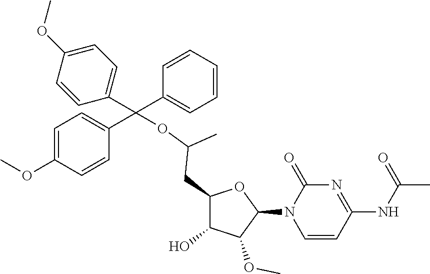 [SMILES: COc1ccc(C(OC(C)C[C@H]2O[C@@H](n3ccc(NC(C)=O)nc3=O)[C@H](OC)[C@@H]2O)(c2ccccc2)c2ccc(OC)cc2)cc1]

TBAF (700 g, 269 mmol, 1 M) was added to a solution of N-(1-((2R,3R,4R,5R)-5-(2-(bis(4-methoxyphenyl)(phenyl)methoxy)propyl)-4-((tert-butyldimethylsilyl)oxy)-3-methoxytetrahydrofuran-2-yl)-2-oxo-1,2-dihydropyrimidin-4-yl)acetamide (100 g, 134 mmol) dissolved in THF (700 ml) and the mixture was stirred at ambient temperature for 2 hours. The mixture was then diluted with water (500 ml), extracted twice ethyl acetate (500 ml), and the combined organic was washed with brine (500 ml), dried over sodium sulfate, and concentrated under reduced pressure to give a residue. The residue was purified by silica gel flash chromatography using 20% to 100% EtOAc/PE to give the title compound (67 g, 79%) as an off-white solid. ES/MS (m/z): 630.2 (M+1).

### Preparation 16

### N-(1-((2R,3R,4R,5R)-5-((S)-2-(bis(4-methoxyphenyl)(phenyl)methoxy)propyl)-4-hydroxy-3-methoxytetrahydrofuran-2-yl)-2-oxo-1,2-dihydropyrimidin-4-yl)acetamide and N-(1-((2R,3R,4R,5R)-5-((R)-2-(bis(4-methoxyphenyl)(phenyl)methoxy)propyl)-4-hydroxy-3-methoxytetrahydrofuran-2-yl)-2-oxo-1,2-dihydropyrimidin-4-yl)acetamide

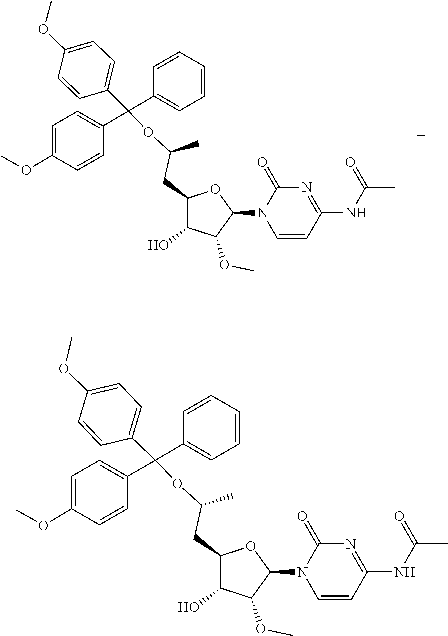

N-(1-((2R,3R,4R,5R)-5-(2-(bis(4-methoxyphenyl)(phenyl)methoxy)propyl)-4-hydroxy-3-methoxytetrahydrofuran-2-yl)-2-oxo-1,2-dihydropyrimidin-4-yl)acetamide (67 g, 106 mmol) was purified by SFC (Condition: CO₂-IPA:ACN; Column: Daicel Chiralpak IM 250 mm×25 mm×10 um; Begin B: 50%; End B: 50%) to give N-(1-((2R,3R,4R,5R)-5-((S)-2-(bis(4-methoxyphenyl)(phenyl)methoxy)propyl)-4-hydroxy-3-methoxytetrahydrofuran-2-yl)-2-oxo-1,2-dihydropyrimidin-4-yl)acetamide (17.3 g, 26%) and N-(1-((2R,3R,4R,5R)-5-((R)-2-(bis(4-methoxyphenyl)(phenyl)methoxy)propyl)-4-hydroxy-3-methoxytetrahydrofuran-2-yl)-2-oxo-1,2-dihydropyrimidin-4-yl)acetamide (32.0 g, 48%) as off-white solids. ES/MS (m/z): 630.3 (M+1).

2-propyl nucleotides (PrON) were incorporated into oligonucleotides using standard phosphoramidite chemistry and standard synthetic methods.

### Example 5: General Procedure for Cpa/Cys Conjugation of FA1 to RNA Sense Strand

In a suitable reaction vessel, 5′ StBu-Cys protected sense strand siRNA material was dissolved in 1 mL of 1×PBS and to it was added a 6-molar excess of Ac-Cpa-Lys(AEEA2-yGlu-C20-OH)—NH2 dissolved in 50/50 acetonitrile/water (~200-300 uL). The pH of the solution was adjusted to ~7.5 with 1 M Tris HCl, pH 8 (~100 uL). About 10 eq of 1,4-Dithiothreitol (DTT, Sigma Aldrich) were added to the solution and the mixture was incubated at 40′C for −18 hrs. The reaction was monitored by analytical HPLC to observe the disappearance of StBu-Cy-siRNA and the appearance of the derivatized product sense strand. After the reaction was complete, the solution was diluted to 15 ml with water and purified by RP-HPLC. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific).

### Example 6: General Procedure for Acylation of RNA Sense Strand 3′ or 5′ Reactive Amino Terminus or Interior Reactive Amino Prior to Peptide Conjugation, with: DBCO-NHS, SPDP-NHS, MSPT-PEG2-NHS, MSPOD-PEG2-NHS

In a suitable reaction vessel, the required reactive amino siRNA strand (typically 30-40 mg) was dissolved in 200-400 uL of PBS Buffer [pH 7] and 600-800 uL of DMSO along with 50-80 uL of DIPEA. The desired active ester (pre-dissolved in a minimum amount of DMSO) was then added in small increments, and the reaction was monitored accordingly by analytical HPLC. After the reaction was complete, the solution was diluted to 15 ml with water and purified by RP-HPLC. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific).

Alternate Procedure for Acylation of RNA Sense Strand 3′ or 5′ Reactive Amino Terminus or Interior Nucleotide Reactive Amino Prior to Peptide Conjugation, with: SMCC-NHS, Chloroacetoxy NHS or C20-Diacid-ME NHS Ester

A stock solution of NHS Ester was prepared in either a minimal concentration of acetonitrile or DMSO depending on solubility. A stock solution of sodium bicarbonate was dissolved in water at 20 mg/mL. In a suitable reaction vessel, the required reactive amino oligonucleotide strand (typically 30-40 mg) was solubilized with 15-30 molar equivalents of the stock solution of sodium bicarbonate. Then, 10-15 equivalents of reactive NHS ester solution was introduced to the reaction vessel with shaking at room temperature. Optionally, extra organic component of either DMSO or acetonitrile may be added to improve NHS ester solubility, typically between a 1:2 to 2:1 ratio of aqueous to organic. The reaction was monitored by LTQ-MS until the starting oligonucleotide was fully consumed. For reaction with chloroacetoxy NHS Ester, the reaction directly was diluted with water to >10% organics and subjected to 3KMWCO spin filtration with multiple washes to remove excess reagent and side products and no further purification was employed.

For reaction with SMCC-NHS Ester, the reaction was quenched to pH 5 with 1N HCl and diluted to >10% organics. The reaction mixture was filtered through a 0.2 micron filter to remove insoluble SMCC-NHS ester byproducts, then subjected to 3KMWCO spin filtration with multiple washes to remove excess reagent and side products and no further purification was employed. The molecular weight of the product was confirmed by LC-MS analysis using a Linear Ion Trap Mass Spectrometer (LTQ XL, Thermo Fisher), and purity was typically confirmed by IP-RP UPLC. The product was quickly frozen at −20° C. or below to prevent maleimide hydrolysis and lyophilized.

For reaction with C20-Diacid-ME NHS Ester, the methyl esters were hydrolyzed with the addition of 100 equivalents of LiOH (2M LiOH solution) and shaken at room temperature for 30 minutes prior to dilution to >5% organics and spin filtration (3KMWCO). The crude functionalized oligonucleotide product was loaded onto an ES Industry Source™ 15RPC with MPA: 10 mM NaOAc 2% Acetonitrile and MPB: 80% Acetonitrile in water. Fractions were analyzed by IP-RP LCMS and those which contained a mass purity greater than 85% without impurities >5% were combined. The molecular weight of the product was confirmed by LC-MS analysis using a Linear Ion Trap Mass Spectrometer (LTQ XL, Thermo Fisher), and purity was confirmed by IP-RP UPLC analysis.

### Example 7: General Procedure for Conjugation of Functionalized RNA Sense Strands to Functionalized Peptides

For reactive dithiol-containing siRNA strands such as those derived from SPDP-NHS or C6S-SC6 moieties, any remaining dithio bonds were reduced with a reagent such as excess (tris(2-carboxyethyl)phosphine)-HCl (TCEP) prior to conjugation. The siRNA then purified from excess reducing agent using ultrafiltration (3KMWCO filter) or optionally chromatographic purification using similar methods as in Examples 4 and 10.

The conjugation of the reactive peptides and sense strands were typically done with 20-25 mg of the sense strand siRNA and a 1.5 to 2× excess of the peptide. The siRNA was dissolved in ~1.5 mL of Ultra-pure DNAse/RNAse free water, the peptide was then added as a solid. The pH of the solution was raised to ~7.5 with 1 M Tris HCl, pH 8 (~20-30 uL) The solution was incubated at 30° C. for ~1.5 hrs, additional peptide was added as needed. Reaction was monitored by analytical HPLC to observe the disappearance of siRNA and the appearance of the conjugate. After the reaction was complete, the solution was diluted to 15 ml with water and purified by RP-HPLC. The molecular weight of the product was confirmed by LC-MS analysis using a Linear Ion Trap Mass Spectrometer (LTQ XL, Thermo Fisher).

### Example 8: General Procedure for Maleimide Ring-Opening of SMCC- and MalDap-Linked Peptide-RNA Sense Strand Conjugates

For SMCC-linked conjugates: Following completion of the SMCC-thiol conjugation reaction, the pH of the reaction mixture was raised to ~9 with 1M Sodium Bicarbonate buffer and incubated at RT. The ring opening reaction was monitored by LC-MS to observe the addition of 18 Daltons. Incubation times vary from a few hours to overnight, to reach complete ring opening.

For MalDap-linked conjugates: Ring-opening can occur immediately in solution following coupling with no additional synthetic manipulation. To confirm that the ring opening is complete prior to purification, the pH of the solution was raised to ~8 with 1M Tris HCl and incubated at RT for 10-20 minutes.

### Example 9: General Procedure for HPLC Analysis of Peptide-RNA Sense Strand Conjugates

Analytical HPLC was used to monitor the conjugation reaction of the peptide and the sense strand siRNA and to check the final purity of the purified Peptide-siRNA conjugates. Analysis was done on an analytical HPLC system (Agilent 1100) using an XBridge Protein BEH C4 analytical RP-HPLC column (Waters; 3.5 μm, 300 Å; 4.6×100 mm). The running buffers used were A: 8.6 mM TEA, with 100 mM HFIP pH 8.3 and B: MeOH/Acetonitrile (50/50 v/v, Fisher Chemicals). The gradient used was a linear 0-85% B gradient over 15 min, at a flow of 1.5 mL/min, with column heating set at 60° C. UV monitoring was done at 254 nm. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific).

### Example 10: General Procedure for Purification of Peptide-RNA Sense Strand Conjugates by RP-HPLC

Solutions containing the peptide-siRNA conjugates were purified using a preparative HPLC system (Shimadzu LC-8A Binary Preparative HPLC Systems) using an XBridge Protein BEH C4 semi-preparative RP-HPLC column (Waters; 5 μm, 300 Å; 10×250 mm). The running buffers used were A: 8.6 mM TEA, with 100 mM HFIP pH 8.3 and B: MeOH/Acetonitrile (50/50 v/v, Fisher Chemicals). The gradient used was a linear 0-90% B gradient over 25 min, at a flow of 25 mL/min, with column heating set at 60° C. UV monitoring was done at 254 and 220 nm. Fractions containing the desired product (HPLC analysis by Agilent HPLC) were pooled, frozen, and lyophilized to give a white amorphous solid product. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific).

### Example 11: General Procedure for Annealing Peptide-RNA Sense Strand Conjugates to Form Duplexed dsRNA-Peptide Conjugates

In a suitable vessel, the lyophilized ssRNA-Peptide was dissolved in 2-3 mL of RNase free water and mixed thoroughly by vortexing. The concentration of the ssRNA peptide conjugate was then determined by using a NanoPhotometer NP80 (Implen) measuring optical density at 260 nm (OD260). Example calculation shown below:

$$\frac{( OD 260 )}{ε ( {mM}^{- 1} {cm}^{- 1} )} = mM$$

Where ε=extinction coefficient of the ssRNA (Peptide contribution is negligible)
mM=millimolar concentration (mmol/L)

$$( \frac{( Conc . ) mmol}{L} ) * Vol mL = m moles$$

The concentration of a corresponding solution of anti-sense (asRNA), was also measured by OD260 using the same method. To anneal the strands, a molar excess of 1.5% asRNA was used with the annealing calculations shown below:

mmoles ssRNA-Peptide*1.015×Equivalence of asRNA=mmoles asRNA required

The volume of the asRNA solution required was then calculated. The required volume of the asRNA solution was combined with the ssRNA-Peptide solution. To anneal the strands, the mixture was incubated at 65° C. for 10 min. using a thermomixer (Eppendorf thermomixer C), and then slowly cooled to 22° C. over 40 min.

The duplexed product was then transferred to a 100,000MWL centrifugation filter tube (Millipore) and spun at 7400 rpm for 15 min. to remove any potential endotoxins. To desalt the PRC, the filtrate was then transferred into an AmiconUltra 3,000MWL centrifugation filter tube (Millipore) at 7400 rpm for 7.5 min., washed twice with 2 mL water each, and then twice with 2 mL of 1×PBS. Following endotoxin removal, water washes, and buffer exchange; the concentrated duplex solution was then transferred to a cryogenic storage vial (Corning) of the appropriate volume. The final concentration was then determined using the NanoPhotometer with OD260 measurements. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific). The results of this characterization are listed in Table 18.

TABLE 18
Characterization of synthesized NPR-C-binding peptide-dsRNA
conjugates with 5′-VP antisense by mass spectrometry

| | | (Calc'd) | Measured | (Calc'd) | Measured |
|---|---|---|---|---|---|
| | (Calc'd) | Sense | Sense | Antisense | Antisense |
| Conjugate | Conjugate | Strand | Strand | Strand | Strand |
| NO | MW/Da | MW/Da | MW/Da | MW/Da | MW/Da |
| C1 | 17728.15 | 9896.05 | 9896.7 | 7832.09 | 7831.4 |
| C2 | 17727.16 | 10009.1 | 10009.7 | 7718.0 | 7717.3 |
| C3 | 17743.16 | 9929.03 | 9928.9 | 7814.13 | 7814.3 |
| C4 | 16877.2 | 9163.0 | 9163.29 | 7714.0 | 7713.54 |
| C5 | 16878.2 | 9140.0 | 9140.25 | 7738.0 | 7737.56 |
| C6 | 16902.2 | 9164.0 | 9164.26 | 7738.0 | 7737.56 |
| C7 | 16970.3 | 9184.0 | 9184.35 | 7786.0 | 7785.57 |
| C8 | 16862.2 | 9439.5 | 9439.55 | 7422.7 | 7422.25 |
| C9 | 16970.3 | 9138.21 | 9137.28 | 7832.1 | 7831.65 |
| C10 | 16985.3 | 9171.0 | 9171.27 | 7814.0 | 7813.68 |
| C11 | 17450.9 | 10012 | 10011.5 | 7439 | 7438.4 |
| C12 | 16987.3 | 9549 | 9548.2 | 7439 | 7438.7 |
| C13 | 19360.7 | 11922 | 11921.6 | 7439 | 7438.5 |
| C14 | 18897.1 | 11458.3 | 11459.4 | 7439 | 7439.3 |
| C15 | 18935.1 | 11285.1 | 11285.0 | 7650.0 | 7650.4 |
| C16 | 18991.2 | 11159 | 11159.8 | 7832 | 7832.6 |
| C17 | 18937.1 | 11183.0 | 11183 | 7754.08 | 7754.09 |
| C18 | 18897.1 | 11153.9 | 11153.9 | 7743.2 | 7743.16 |
| C19 | 18920.1 | 11177 | 11176.7 | 7743.2 | 7743.2 |
| C20 | 18897.1 | 11246.1 | 11246.1 | 7651 | 7651.5 |
| C21 | 18897.1 | 11286.1 | 11286.4 | 7611 | 7611.3 |

dsRNAs with a conjugated 3′-GalNAc ligand on the sense strand (phosephate linkage) and 5′ phosphate or 5′-vinylphophonate on the antisense strand were annealed and characterized in a substantially similar manner. The results of this characterization are listed in Table 19A and 19B.

TABLE 19A
Characterization of synthesized PDE3B-Targeting dsRNA Gal-2
conjugates with (5′P) antisense modification

| | | | MW | MW |
|---|---|---|---|---|
| dsRNA | | SEQ | Cal. | Obs. |
| NO | Strand | ID NO | (g/mol) | (g/mol) |
| 46 | s | 170 | 8655.43 | 8655.06 |
| | as | 224 | 7533.86 | 7533.66 |
| 47 | s | 171 | 8637.47 | 8637.09 |
| | as | 225 | 7544.79 | 7544.6 |
| 48 | s | 172 | 8518.38 | 8517.99 |
| | as | 226 | 7687.9 | 7687.72 |
| 49 | s | 173 | 8441.23 | 8440.86 |
| | as | 227 | 7818.12 | 7817.92 |
| 50 | s | 174 | 8433.26 | 8432.88 |
| | as | 228 | 7717.99 | 7717.79 |
| 51 | s | 175 | 8589.36 | 8589 |
| | as | 229 | 7697.01 | 7696.83 |
| 52 | s | 176 | 8521.28 | 8520.9 |
| | as | 230 | 7722.07 | 7721.87 |
| 53 | s | 177 | 8408.25 | 8407.87 |
| | as | 231 | 7836.09 | 7835.9 |
| 54 | s | 178 | 8454.33 | 8453.95 |
| | as | 232 | 7790.01 | 7789.82 |
| 55 | s | 179 | 8385.21 | 8384.83 |
| | as | 233 | 7795.08 | 7794.88 |
| 57 | s | 181 | 8441.23 | 8440.86 |
| | as | 235 | 7779.08 | 7778.88 |
| 58 | s | 182 | 8685.51 | 8685.13 |
| | as | 236 | 7465.73 | 7465.53 |
| 59 | s | 183 | 8490.27 | 8489.89 |
| | as | 237 | 7701 | 7700.8 |
| 60 | s | 184 | 8495.34 | 8494.95 |
| | as | 238 | 7710.94 | 7710.75 |
| 61 | s | 185 | 8408.25 | 8407.87 |
| | as | 239 | 7836.09 | 7835.89 |
| 62 | s | 186 | 8442.22 | 8441.85 |
| | as | 240 | 7779.08 | 7778.88 |
| 63 | s | 187 | 8537.33 | 8536.95 |
| | as | 241 | 7598.9 | 7598.7 |
| 64 | s | 188 | 8389.15 | 8388.77 |
| | as | 242 | 7731.08 | 7730.87 |
| 65 | s | 189 | 8428.19 | 8427.81 |
| | as | 243 | 7731.08 | 7730.87 |
| 66 | s | 190 | 8405.15 | 8404.77 |
| | as | 244 | 7731.08 | 7730.87 |
| 67 | s | 191 | 8515.28 | 8514.9 |
| | as | 245 | 7685 | 7684.8 |
| 68 | s | 192 | 8428.19 | 8427.82 |
| | as | 246 | 7731.08 | 7730.87 |
| 69 | s | 193 | 8528.32 | 8527.94 |
| | as | 247 | 7700.02 | 7699.82 |
| 70 | s | 194 | 8497.31 | 8496.92 |
| | as | 248 | 7638.92 | 7638.73 |
| 71 | s | 195 | 8405.15 | 8404.78 |
| | as | 249 | 7731.08 | 7730.87 |
| 72 | s | 196 | 8442.22 | 8441.85 |
| | as | 250 | 7762.1 | 7761.89 |
| 73 | s | 197 | 8410.22 | 8409.84 |
| | as | 251 | 7742.01 | 7741.81 |
| 74 | s | 198 | 8445.17 | 8444.8 |
| | as | 252 | 7731.08 | 7730.87 |
| 75 | s | 199 | 8520.35 | 8519.96 |
| | as | 253 | 7678.95 | 7678.75 |
| 76 | s | 200 | 8500.26 | 8499.88 |
| | as | 254 | 7589.94 | 7589.73 |
| 77 | s | 201 | 8434.24 | 8433.86 |
| | as | 255 | 7742.01 | 7741.81 |
| 78 | s | 202 | 8536.35 | 8535.96 |
| | as | 256 | 7637.94 | 7637.74 |
| 79 | s | 203 | 8615.41 | 8615.03 |
| | as | 257 | 7550.85 | 7550.65 |
| 80 | s | 204 | 8655.43 | 8655.06 |
| | as | 258 | 7550.85 | 7550.65 |
| 81 | s | 205 | 8606.45 | 8606.06 |
| | as | 259 | 7529.78 | 7529.58 |
| 82 | s | 206 | 8442.22 | 8441.84 |
| | as | 260 | 7779.08 | 7778.88 |
| 83 | s | 207 | 8458.22 | 8457.85 |
| | as | 261 | 7762.1 | 7761.89 |
| 84 | s | 208 | 8382.11 | 8381.74 |
| | as | 262 | 7755.11 | 7754.9 |
| 85 | s | 209 | 8434.24 | 8433.86 |
| | as | 263 | 7742.01 | 7741.81 |
| 86 | s | 210 | 8381.12 | 8380.75 |
| | as | 264 | 7769.14 | 7768.93 |
| 87 | s | 211 | 8421.15 | 8420.78 |
| | as | 265 | 7769.14 | 7768.93 |
| 88 | s | 212 | 8491.25 | 8490.88 |
| | as | 266 | 7708.04 | 7707.84 |
| 89 | s | 213 | 8709.54 | 8709.15 |
| | as | 267 | 7426.69 | 7426.49 |
| 90 | s | 214 | 8466.24 | 8465.88 |
| | as | 268 | 7701 | 7700.8 |
| 179 | s | 383 | 8529.3 | 8529.7 |
| | as | 485 | 7660.9 | 7660.6 |
| 180 | s | 384 | 8530.2 | 8530.7 |
| | as | 486 | 7660.9 | 7660. |
| 181 | s | 385 | 8529.3 | 8529.7 |
| | as | 487 | 7700.0 | 7699.6 |
| 182 | s | 386 | 8568.3 | 8568.7 |
| | as | 488 | 7636.9 | 7636.5 |
| 183 | s | 387 | 8568.3 | 8568.7 |
| | as | 489 | 7635.9 | 7635.6 |
| 184 | s | 388 | 8489.2 | 8489.7 |
| | as | 490 | 7677.9 | 7677.6 |
| 185 | s | 389 | 8466.2 | 8466.6 |
| | as | 491 | 7740. | 7739.6 |
| 186 | s | 390 | 8473.2 | 8473.7 |
| | as | 492 | 7701.0 | 7700.6 |
| 187 | s | 391 | 8457.2 | 8457.7 |
| | as | 493 | 7677.9 | 7677.6 |
| 188 | s | 392 | 8678.4 | 8678.9 |
| | as | 494 | 7527.8 | 7527.4 |
| 189 | s | 393 | 8632.3 | 8632.8 |
| | as | 495 | 7534.8 | 7534.4 |
| 190 | s | 394 | 8466.2 | 8466.6 |
| | as | 496 | 7739.0 | 7738.6 |
| 191 | s | 395 | 8397.1 | 8397.5 |
| | as | 497 | 7793.1 | 7792.7 |
| 192 | s | 396 | 8537.3 | 8537.7 |
| | as | 498 | 7597.9 | 7597.5 |
| 193 | s | 397 | 8496.3 | 8496.7 |
| | as | 499 | 7701.9 | 7701.6 |
| 194 | s | 398 | 8495.3 | 8495.7 |
| | as | 500 | 7717.9 | 7717.6 |
| 195 | s | 399 | 8496.3 | 8496.7 |
| | as | 501 | 7717.9 | 7717.6 |
| 196 | s | 400 | 8553.3 | 8553.7 |
| | as | 502 | 7597.9 | 7597.5 |
| 197 | s | 401 | 8553.3 | 8553.7 |
| | as | 503 | 7597.9 | 7597.5 |
| 198 | s | 402 | 8421.1 | 8421.6 |
| | as | 504 | 7731.0 | 7730.7 |
| 199 | s | 403 | 8317.0 | 8317.5 |
| | as | 505 | 7912.2 | 7911.8 |
| 200 | s | 404 | 8529.3 | 8529.7 |
| | as | 506 | 7675.9 | 7675.6 |
| 201 | s | 405 | 8506.2 | 8506.7 |
| | as | 507 | 7700.0 | 7699.6 |
| 202 | s | 406 | 8506.2 | 8506.7 |
| | as | 508 | 7659.9 | 7659.6 |
| 203 | s | 407 | 8410.2 | 8410.6 |
| | as | 509 | 7742.0 | 7741.6 |
| 204 | s | 408 | 8655.4 | 8655.8 |
| | as | 510 | 7533.8 | 7533.5 |
| 205 | s | 409 | 8631.4 | 8631.8 |
| | as | 511 | 7550.8 | 7550.4 |
| 206 | s | 410 | 8543.3 | 8543.8 |
| | as | 512 | 7592.8 | 7592.4 |
| 207 | s | 411 | 8725.5 | 8725.9 |
| | as | 513 | 7425.7 | 7425.3 |

TABLE 19B
Characterization of synthesized PDE3B-Targeting dsRNA Gal-2
conjugates with (5′VP) antisense modification

| | | | MW | MW |
|---|---|---|---|---|
| dsRNA | | SEQ | Cal. | Obs. |
| NO | Strand | ID NO | (g/mol) | (g/mol) |
| 50 | s | 174 | 8433.0 | 8432.7 |
| | as | 228 | 7714.0 | 7713.5 |
| 54 | s | 178 | 8454.0 | 8454.4 |
| | as | 232 | 7786.0 | 7786.2 |
| 63 | s | 187 | 8537.0 | 8537.0 |
| | as | 241 | 7595.0 | 7594.5 |
| 64 | s | 188 | 8389.0 | 8389.0 |
| | as | 242 | 7727.0 | 7726.7 |
| 65 | s | 189 | 8428.0 | 8427.8 |
| | as | 243 | 7727.0 | 7726.7 |
| 66 | s | 190 | 8405.0 | 8404.8 |
| | as | 244 | 7727.0 | 7727.0 |
| 67 | s | 191 | 8515.0 | 8515.0 |
| | as | 245 | 7681.0 | 7680.6 |
| 68 | s | 192 | 8428.0 | 8427.9 |
| | as | 246 | 7727.0 | 7727.0 |
| 69 | s | 193 | 8528.0 | 8528.0 |
| | as | 247 | 7696.0 | 7695.8 |
| 70 | s | 194 | 8497.0 | 8497.0 |
| | as | 248 | 7635.0 | 7634.6 |
| 71 | s | 195 | 8405.0 | 8405.1 |
| | as | 249 | 7727.0 | 7726.8 |
| 72 | s | 196 | 8442.0 | 8441.8 |
| | as | 250 | 7758.0 | 7757.6 |
| 73 | s | 197 | 8410.0 | 8409.9 |
| | as | 251 | 7738.0 | 7737.4 |
| 74 | s | 198 | 8445.0 | 8444.5 |
| | as | 252 | 7727.0 | 7726.7 |
| 75 | s | 199 | 8520.0 | 8520.0 |
| | as | 253 | 7675.0 | 7674.5 |
| 76 | s | 200 | 8500.0 | 8499.8 |
| | as | 254 | 7585.0 | 7585.6 |
| 77 | s | 201 | 8434.0 | 8433.7 |
| | as | 255 | 7738.0 | 7737.4 |
| 78 | s | 202 | 8536.0 | 8535.9 |
| | as | 256 | 7634.0 | 7633.5 |
| 79 | s | 203 | 8615.0 | 8615.0 |
| | as | 257 | 7547.0 | 7546.8 |
| 80 | s | 204 | 8655.0 | 8655.4 |
| | as | 258 | 7546.0 | 7547.0 |
| 81 | s | 205 | 8606.0 | 8606.4 |
| | as | 259 | 7525.0 | 7525.9 |
| 82 | s | 206 | 8442.0 | 8442.4 |
| | as | 260 | 7775.0 | 7774.7 |
| 83 | s | 207 | 8458.0 | 8458.3 |
| | as | 261 | 7758.0 | 7757.9 |
| 84 | s | 208 | 8382.0 | 8382.1 |
| | as | 262 | 7751.0 | 7751.4 |
| 85 | s | 209 | 8434.0 | 8434.3 |
| | as | 263 | 7738.0 | 7737.7 |
| 86 | s | 210 | 8381.0 | 8381.6 |
| | as | 264 | 7765.0 | 7765.0 |
| 87 | s | 211 | 8421.0 | 8421.3 |
| | as | 265 | 7765.0 | 7765.1 |
| 88 | s | 212 | 8491.0 | 8491.5 |
| | as | 266 | 7704.0 | 7704.0 |
| 89 | s | 213 | 8709.0 | 8709.5 |
| | as | 267 | 7422.0 | 7422.5 |
| 90 | s | 214 | 8466.0 | 8466.8 |
| | as | 268 | 7697.0 | 7697.0 |
| 159 | s | 187 | 8537.0 | 8537.4 |
| | as | 465 | 7673.0 | 7674.1 |
| 160 | s | 188 | 8389.0 | 8389.4 |
| | as | 466 | 7807.0 | 7807.1 |
| 161 | s | 194 | 8497.0 | 8497.4 |
| | as | 467 | 7714.0 | 7714.2 |
| 162 | s | 197 | 8410.0 | 8410.3 |
| | as | 468 | 7816.0 | 7815.8 |
| 163 | s | 205 | 8606.0 | 8606.4 |
| | as | 469 | 7604.0 | 7604.9 |
| 164 | s | 213 | 8709.0 | 8709.5 |
| | as | 470 | 7501.0 | 7502.0 |
| 165 | s | 370 | 8717.0 | 7611.0 |
| | as | 471 | 8717.0 | 7610.6 |
| 166 | s | 37 | 8708 | 7712 |
| | as | 472 | 8707.7 | 7711.5 |
| 167 | s | 372 | 8700.0 | 7691.0 |
| | as | 473 | 8699.75 | 7691.46 |
| 168 | s | 373 | 8677.0 | 7651.0 |
| | as | 474 | 8676.68 | 7651.41 |
| 169 | s | 374 | 8608.0 | 7743.0 |
| | as | 475 | 8607.60 | 7742.59 |
| 170 | s | 375 | 8585.0 | 7743.0 |
| | as | 476 | 8584.54 | 7742.55 |
| 171 | s | 376 | 8614.0 | 7754.0 |
| | as | 477 | 8613.66 | 7753.52 |
| 172 | s | 377 | 8716.0 | 7650.0 |
| | as | 478 | 8715.76 | 7650.45 |
| 173 | s | 378 | 8890.0 | 7439.0 |
| | as | 479 | 8889.94 | 7439.21 |
| 174 | s | 379 | 8590.0 | 7832.0 |
| | as | 480 | 8589.61 | 7831.56 |
| 175 | s | 379 | 8590.0 | 7754.0 |
| | as | 48 | 8589.7 | 7753.8 |
| 176 | s | 380 | 8695.0 | 7697.0 |
| | as | 482 | 8694.69 | 7696.51 |
| 177 | s | 38 | 8622.0 | 7774.0 |
| | as | 483 | 8621.9 | 7773.6 |
| 178 | s | 382 | 8588 | 7848 |
| | as | 484 | 8587.7 | 7847.6 |
| 208 | s | 412 | 8709.0 | 8709.6 |
| | as | 514 | 7673.0 | 7673.1 |
| 209 | s | 413 | 8710.0 | 8709.9 |
| | as | 515 | 7673.0 | 7672.7 |
| 210 | s | 414 | 8709.0 | 8709.1 |
| | as | 516 | 7712.0 | 7711.7 |
| 211 | s | 415 | 8748.0 | 8747.7 |
| | as | 517 | 7649.0 | 7648.5 |
| 212 | s | 416 | 8748.0 | 8747.7 |
| | as | 518 | 7648.0 | 7647.6 |
| 213 | s | 417 | 8669.0 | 8669.0 |
| | as | 519 | 7690.0 | 7689.8 |
| 214 | s | 418 | 8646.0 | 8646.0 |
| | as | 520 | 7752.0 | 7751.7 |
| 215 | s | 419 | 8653.0 | 8652.7 |
| | as | 52 | 7713.0 | 7713.1 |
| 216 | s | 420 | 8637.0 | 8637.1 |
| | as | 522 | 7690.0 | 7689.8 |
| 217 | s | 421 | 8859.0 | 8858.0 |
| | as | 523 | 7540.0 | 7539.7 |
| 218 | s | 422 | 8812.0 | 8812.2 |
| | as | 524 | 7547.0 | 7546.9 |
| 219 | s | 423 | 8646.0 | 8645.9 |
| | as | 525 | 7751.0 | 7751.0 |
| 220 | s | 424 | 8577.0 | 8576.5 |
| | as | 526 | 7805.0 | 04.654 |
| 221 | s | 425 | 8717.0 | 8717.3 |
| | as | 527 | 7610.0 | 7609.8 |
| 222 | s | 426 | 8676.0 | 8676.2 |
| | as | 528 | 7714.0 | 7713.4 |
| 223 | s | 427 | 8675.0 | 8675.4 |
| | as | 529 | 7730.0 | 7730.1 |
| 224 | s | 428 | 8676.0 | 8676.4 |
| | as | 530 | 7730.0 | 7729.8 |
| 225 | s | 429 | 8733.0 | 8733.1 |
| | as | 531 | 7610.0 | 7610.0 |
| 226 | s | 430 | 8733.0 | 8733.5 |
| | as | 532 | 7610.0 | 7610.4 |
| 227 | s | 431 | 8601.0 | 8601.2 |
| | as | 533 | 7743.0 | 7742.5 |
| 228 | s | 432 | 8497.0 | 8497.8 |
| | as | 534 | 7924.0 | 7924.3 |
| 229 | s | 433 | 8709.0 | 8709.0 |
| | as | 535 | 7688.0 | 7687.8 |
| 230 | s | 434 | 8686.0 | 8686.5 |
| | as | 536 | 7712.0 | 7711.8 |
| 231 | s | 435 | 8686.0 | 8685.7 |
| | as | 537 | 7672.0 | 7671.7 |
| 232 | s | 436 | 8590.0 | 8590.6 |
| | as | 538 | 7754.0 | 7754.3 |
| 233 | s | 437 | 8836.0 | 8835.1 |
| | as | 539 | 7546.0 | 7545.5 |
| 234 | s | 438 | 8812.0 | 8811.7 |
| | as | 540 | 7563.0 | 7562.9 |
| 235 | s | 439 | 8723.0 | 8723.0 |
| | as | 541 | 7605.0 | 7604.3 |
| 236 | s | 440 | 8906.0 | 8905.7 |
| | as | 542 | 7438.0 | 7438.0 |
| 237 | s | 370 | 8717.0 | 8717.5 |
| | as | 543 | 7668.0 | 7668.1 |
| 238 | s | 441 | 9432.0 | 9432.0 |
| | as | 544 | 7610.0 | 7610.4 |
| 239 | s | 370 | 8717.0 | 8716.7 |
| | as | 545 | 7690.0 | 7690.4 |
| 240 | s | 370 | 8717.0 | 8717.5 |
| | as | 546 | 7028.0 | 7027.4 |
| 241 | s | 442 | 8693.0 | 8692.9 |
| | as | 547 | 7650.0 | 7650.1 |
| 242 | s | 443 | 8374.0 | 8374.1 |
| | as | 548 | 7347.0 | 7347.7 |
| 243 | s | 444 | 8747.5 | 8748.8 |
| | as | 471 | 7611.0 | 7611.5 |
| 244 | s | 445 | 8725.4 | 8725.6 |
| | as | 471 | 7611.0 | 7611.7 |
| 245 | s | 446 | 8785.5 | 8785.9 |
| | as | 471 | 7611.0 | 7611.7 |
| 246 | s | 447 | 8622.0 | 8621.5 |
| | as | 549 | 7742.0 | 7742.5 |
| 247 | s | 447 | 8622.0 | 8621.5 |
| | as | 550 | 7742.0 | 7742.5 |
| 248 | s | 447 | 8622.3 | 8622.5 |
| | as | 477 | 7754.1 | 7754.6 |
| 249 | s | 448 | 9329.0 | 9329.0 |
| | as | 55 | 7713.0 | 7713.5 |
| 250 | s | 376 | 8614.0 | 8614.4 |
| | as | 552 | 7131.0 | 7130.4 |
| 251 | s | 376 | 8614.0 | 8614.7 |
| | as | 553 | 7793.0 | 7793.2 |
| 252 | s | 376 | 8614.0 | 8614.3 |
| | as | 554 | 7782.0 | 7782.1 |
| 253 | s | 449 | 8590.0 | 8590.4 |
| | as | 555 | 7793.0 | 7792.8 |
| 254 | s | 450 | 8702.4 | 8702.7 |
| | as | 477 | 7754.1 | 7754.5 |
| 255 | s | 376 | 8614.0 | 8614.3 |
| | as | 556 | 7451.0 | 7450.7 |
| 256 | s | 45 | 8271.0 | 8271.4 |
| | as | 557 | 7451.0 | 7451.3 |
| 257 | s | 452 | 8630.0 | 8629.6 |
| | as | 477 | 7754.0 | 7754.5 |
| 258 | s | 376 | 8614.0 | 8614.9 |
| | as | 558 | 7718.0 | 7718.4 |
| 259 | s | 447 | 8622.0 | 8622.7 |
| | as | 558 | 7718.0 | 7718.5 |
| 260 | s | 376 | 8614.0 | 8614.8 |
| | as | 559 | 7754.0 | 7755.0 |
| 261 | s | 379 | 8590.0 | 8590.4 |
| | as | 560 | 7810.0 | 7809.9 |
| 262 | s | 453 | 8566.0 | 8566.1 |
| | as | 561 | 7752.0 | 7752.1 |
| 263 | s | 379 | 8590.0 | 8590.3 |
| | as | 563 | 7170.0 | 7169.7 |
| 264 | s | 454 | 9305.0 | 9304.8 |
| | as | 564 | 7731.0 | 7731.1 |
| 265 | s | 379 | 8590.3 | 8590.7 |
| | as | 565 | 7754.1 | 7754.6 |
| 266 | s | 455 | 8590.3 | 8590.6 |
| | as | 481 | 7754.1 | 7754.5 |
| 267 | s | 379 | 8590.3 | 8591.1 |
| | as | 566 | 7754.1 | 7754.6 |
| 268 | s | 379 | 8590.3 | 8590.8 |
| | as | 567 | 7754.1 | 7754.4 |
| 269 | s | 456 | 8613.0 | 8612.6 |
| | as | 481 | 7754.0 | 7754.4 |
| 270 | s | 457 | 9199.0 | 9198.8 |
| | as | 481 | 7754.0 | 7753.9 |
| 271 | s | 458 | 8606.0 | 8606.6 |
| | as | 568 | 7793.0 | 7793.3 |
| 272 | s | 459 | 8883.5 | 8883.8 |
| | as | 479 | 7438.7 | 7439.2 |
| 273 | s | 460 | 8865.6 | 8865.9 |
| | as | 569 | 7477.8 | 7478.2 |
| 274 | s | 461 | 8977.7 | 8978.0 |
| | as | 479 | 7438.7 | 7439.2 |
| 275 | s | 462 | 8546.0 | 8546.3 |
| | as | 570 | 7176.0 | 7175.4 |
| 276 | s | 463 | 8905.6 | 8905.9 |
| | as | 479 | 7438.8 | 7439.2 |
| 277 | s | 464 | 8905.6 | 8905.9 |
| | as | 479 | 7438.8 | 7439.2 |

dsRNAs with a conjugated 2′-XHD on the sense strand and a 5′-vinylphophonate on the antisense strand were annealed and characterized in a substantially similar manner. The results of this characterization are listed in Table 20.

TABLE 20
Exemplary LC/MS data for PDE3B siRNAs with 2′-XHD
modifications and 5′-vinylphophonate (VP) antisense modification

| SEQ | | | | MW |
|---|---|---|---|---|
| ID | dsRNA | | MW Cal. | Obs. |
| NO | NO. | Strand | (g/mol) | (g/mol) |
| 215 | 91 | s | 7151.056 | 7151.1 |
| 240 | | as | 7775.091 | 7774.9 |
| 216 | 92 | s | 7394.354 | 7394.7 |
| 236 | | as | 7461.741 | 7461.9 |
| 217 | 93 | s | 7364.273 | 7364.6 |
| 224 | | as | 7529.875 | 7530.1 |
| 218 | 94 | s | 7231.105 | 7231.3 |
| 234 | | as | 7718.083 | 7718.2 |
| 219 | 95 | s | 7094.048 | 7094.1 |
| 233 | | as | 7791.09 | 7791.2 |
| 220 | 96 | s | 7150.072 | 7150.1 |
| 227 | | as | 7814.13 | 7814.3 |
| 221 | 97 | s | 7117.088 | 7117.4 |
| 231 | | as | 7832.099 | 7832.0 |
| 222 | 98 | s | 7117.088 | 7117.7 |
| 231 | | as | 7832.099 | 7831.9 |
| 223 | 99 | s | 7230.1 | 7230.2 |
| 230 | | as | 7718.083 | 7718.1 |

### Example 12: In Vitro Knockdown of Human PDE3B in Hep3B Cells with Gal-1 Conjugated PDE3B siRNA by Transfection (CRC)

For Hep3B (ATCC, Part #: HB-8064) cells, transfection was carried out by adding 24.7 μL of Opti-MEM (Gibco, Part #: 31985062) plus 0.3 μL of Lipofectamine RNAiMAX (Life Technologies, Part #: 13778-150) per well to 25 μL of each siRNA duplex to an individual well in a 96-well plate (Costar, Part #3596). The mixture was then incubated at room temperature for 20 minutes. Fifty microliters of complete growth media without antibiotic containing Hep3B at 400,000 cells/mL were then added to the siRNA mixture. 7 points CRC was done at siRNA duplex final concentration starts at 10 nM, then 1:5 serial dilution. Cells were incubated for 24 hours prior to qPCR.

Treated cells were washed with cold 1×PBS and lysed directly into the 96 well cell plate using TaqMan Fast Advanced Cell-to-Ct kit (Life Technologies, Part #: AM1729). cDNA was synthesized using the following steps in a thermocycler: 37° C. for 30 minutes, 95° C. for 5 minutes, and 4° C. hold. Polymerase Chain Reaction (PCR) using the following cycles temperatures and times: 50° C. for 2 minutes, 95° C. for 20 seconds, 40 cycles of 95° C. for 1 seconds and 60° C. for 20 seconds.

GraphPad Prism v10.1.2 was used to determine IC₅₀. The human PDE3B levels were normalized to human RPLP0 (Life Technologies) and represented the relative knockdown of human PDE3B mRNA expression as compared to vehicle-treated control cells.

TABLE 21
Summary of % KD for RNAi agents conjugated to Gal-1 CRC
in Hep3B cells, 24 hour treatment, RNAimax transfection

| | KD @ | |
|---|---|---|
| dsRNA | 10 nM | |
| NO: | (%) | IC₅₀ (nM) |
| 46 | 81.29 | 0.129 |
| 47 | 82.60 | 0.048 |
| 48 | 81.13 | 0.024 |
| 49 | 79.23 | 0.065 |
| 50 | 79.68 | 0.015 |
| 51 | 14.21 | — |
| 52 | 85.11 | 0.050 |
| 53 | 86.54 | 0.111 |
| 54 | 81.93 | 0.019 |
| 55 | 83.60 | 0.114 |
| 56 | 81.99 | 0.029 |
| 57 | 86.49 | 0.078 |
| 58 | 81.72 | 0.034 |
| 59 | 79.36 | — |
| 60 | 81.31 | 0.035 |
| 61 | 81.17 | 0.080 |
| 62 | 84.32 | 0.044 |

### Example 13A: In Vitro Knockdown of Human PDE3B in Hep3B Cells with Gal-2 Conjugated PDE3B siRNA by Transfection

For Hep3B (ATCC, Part #: HB-8064) cells, transfection was carried out by adding 24.7 μL of Opti-MEM (Gibco, Part #: 31985062) plus 0.3 μL of Lipofectamine RNAiMAX (Life Technologies, Part #: 13778-150) per well to 25 μL of each siRNA duplex to an individual well in a 96-well plate (Costar, Part #3596). The mixture was then incubated at room temperature for 20 minutes. 50 μL of complete growth media without antibiotic containing Hep3B at 400,000 cells/mL were then added to the siRNA mixture. Single point was done at 1 nM and 0.01 nM final siRNA duplex concentration. Cells were incubated for 24 hours prior to qPCR.

Treated cells were washed with cold 1×PBS and lysed directly into the 96 well cell plate using TaqMan Fast Advanced Cell-to-Ct kit (Life Technologies, Part #: AM1729). cDNA was synthesized using the following steps in a thermocycler: 37° C. for 30 minutes, 95° C. for 5 minutes, and 4° C. hold. Polymerase Chain Reaction (PCR) using the following cycles temperatures and times: 50° C. for 2 minutes, 95° C. for 20 seconds, 40 cycles of 95° C. for 1 seconds and 60° C. for 20 seconds.

The human PDE3B levels were normalized to human RPLP0 (Life Technologies) and represented the relative knockdown of human PDE3B mRNA expression as compared to vehicle-treated control cells.

TABLE 22A
Summary of % KD for RNAi agents conjugated to Gal-2 tested at two
concentrations in Hep3B cells, 24 hour treatment, RNAimax transfection

| dsRNA | % KD at 1 | % KD at 0.01 |
|---|---|---|
| NO: | nM | nM |
| 63 | 86.69 | 66.50 |
| 64 | 86.46 | 68.18 |
| 65 | 85.67 | 69.49 |
| 66 | 93.58 | 59.72 |
| 67 | 85.24 | 64.91 |
| 68 | 83.06 | 65.90 |
| 69 | 89.63 | 63.68 |
| 70 | 85.50 | 61.30 |
| 71 | 83.16 | 69.06 |
| 72 | 82.98 | 68.67 |
| 73 | 86.27 | 77.32 |
| 74 | 82.02 | 64.40 |
| 75 | 85.40 | 60.94 |
| 76 | 87.92 | 59.12 |
| 77 | 82.72 | 71.10 |
| 78 | 82.80 | 61.76 |
| 79 | 81.72 | 63.62 |
| 80 | 80.09 | 69.80 |
| 81 | 80.21 | 64.44 |
| 82 | 87.40 | 59.89 |
| 83 | 82.12 | 65.02 |
| 84 | 81.16 | 65.23 |
| 85 | 84.57 | 60.74 |
| 86 | 78.99 | 67.11 |
| 87 | 81.05 | 62.24 |
| 88 | 81.08 | 62.80 |
| 89 | 78.50 | 63.82 |
| 90 | 86.26 | 56.95 |
| 46 | 87.30 | 45.25 |
| 47 | 84.76 | 53.49 |
| 48 | 80.45 | 57.95 |
| 49 | 86.24 | 50.01 |
| 50 | 83.13 | 71.40 |
| 51 | 13.74 | 0.14 |
| 52 | 86.03 | 42.80 |
| 53 | 86.11 | 29.22 |
| 54 | 80.92 | 66.72 |
| 55 | 85.78 | 47.13 |
| 57 | 89.02 | 50.40 |
| 58 | 82.59 | 58.52 |
| 59 | 74.94 | 33.36 |
| 60 | 80.48 | 59.64 |
| 61 | 74.00 | 9.22 |
| 62 | 88.64 | 49.29 |
| 179 | 83.25 | 57.39 |
| 180 | 84.79 | 65.6 |
| 181 | 89.11 | 71.98 |
| 182 | 87.74 | 60.65 |
| 183 | 88.57 | 68.92 |
| 184 | 87.58 | 65.15 |
| 185 | 87.14 | 67.39 |
| 186 | 88.59 | 65.75 |
| 187 | 89.11 | 57.9 |
| 188 | 90.13 | 71.64 |
| 189 | 89.8 | 64.75 |
| 190 | 87.16 | 70.25 |
| 191 | 85.52 | 68.49 |
| 192 | 91.67 | 71.91 |
| 193 | 88.29 | 76.61 |
| 194 | 85.89 | 65.49 |
| 195 | 88.66 | 66.07 |
| 196 | 86.57 | 54.33 |
| 197 | 83.12 | 60.69 |
| 198 | 83.19 | 53.52 |
| 199 | 83.13 | 59.11 |
| 200 | 83.72 | 58.14 |
| 201 | 85.1 | 66.89 |
| 202 | 83.89 | 61.29 |
| 203 | 83.7 | 62.05 |
| 204 | 84.55 | 70.26 |
| 205 | 81.45 | 60 |
| 206 | 83.77 | 61.67 |
| 207 | 82.52 | 58.94 |

### Example 13B: In Vitro Knockdown of Human PDE3B in Hep3B Cells with Gal-2 Conjugated PDE3B siRNA by Transfection Vitro Knockdown of Human PDE3B in Hep3B Cells with LYGal-Conjugated PDE3B siRNA by Transfection

For Hep3B (ATCC, Part #: HB-8064) cells, transfection was carried out by adding 24.7 μL of Opti-MEM (Gibco, Part #: 31985062) plus 0.3 μL of Lipofectamine RNAiMAX (Life Technologies, Part #: 13778-150) per well to 25 μL of each siRNA duplex to an individual well in a 96-well plate (Costar, Part #3596). The mixture was then incubated at room temperature for 20 minutes. 50 μL of complete growth media without antibiotic containing Hep3B at 400,000 cells/mL were then added to the siRNA mixture. Single point was done at 1 nM and 0.01 nM final siRNA duplex concentration. Cells were incubated for 24 hours prior to qPCR.

Treated cells were washed with cold 1×PBS and lysed directly into the 96 well cell plate using TaqMan Cell-to-Ct Express kit (Life Technologies, Part #: A57988). cDNA was synthesized using the following steps in a thermocycler: 25° C. for 10 minutes, 50° C. for 10 minutes, 85° C. for 5 minutes and 4° C. hold. Polymerase Chain Reaction (PCR) using the following cycles temperatures and times: 50° C. for 2 minutes, 95° C. for 20 seconds, 40 cycles of 95° C. for 1 seconds and 60° C. for 20 seconds.

The human PDE3B levels were normalized to human RPLP0 (Life Technologies) and represented the relative knockdown of human PDE3B mRNA expression as compared to vehicle-treated control cells (Table 22B).

TABLE 22B
Hep3B, 24 h, RNAimax, % remaining mRNA

| dsRNA | | |
|---|---|---|
| NO: | 0.1 nM | 0.01 nM |
| 165 | 16.28 | 65.04 |
| 237 | 22 | 61.56 |
| 238 | 36.34 | 81.41 |
| 239 | 20.09 | 53.06 |
| 240 | 23.26 | 55.48 |
| 241 | 29.33 | 67.84 |
| 242 | 18.45 | 44.06 |
| 243 | 28.96 | 62.02 |
| 244 | 23.1 | 115.47 |
| 245 | 21.26 | 73.1 |
| 171 | 16.15 | 32.17 |
| 246 | 20.45 | 47.13 |
| 247 | 45.49 | 74.02 |
| 248 | 20.87 | 47.68 |
| 249 | 57.88 | 93.1 |
| 250 | 28.91 | 53 |
| 251 | 19.79 | 60.32 |
| 252 | 22.37 | 47.43 |
| 253 | 19.77 | 39.44 |
| 254 | 19.94 | 91.15 |
| 255 | 25.61 | 59.04 |
| 256 | 20.27 | 29.22 |
| 257 | 18.6 | 30.02 |
| 258 | 30.05 | 70.17 |
| 259 | 20.43 | 53.12 |
| 260 | 26.58 | 84.75 |
| 175 | 16.75 | 31.85 |
| 261 | 18.25 | 40.93 |
| 262 | 17.76 | 38.17 |
| 263 | 16.77 | 31.33 |
| 264 | 30.37 | 107.43 |
| 265 | 20.09 | 63.66 |
| 266 | 20.65 | 50.13 |
| 267 | 17.82 | 27.85 |
| 268 | 22.62 | 47.14 |
| 269 | 17.8 | 38.12 |
| 270 | 17.52 | 29.09 |
| 271 | 17.22 | 38.88 |
| 173 | 18.64 | 55.53 |
| 272 | 26.52 | 65.42 |
| 273 | 24.32 | 51.35 |
| 274 | 25.49 | 38.16 |
| 275 | 24.77 | 58.99 |
| 276 | 18.95 | 42.07 |
| 277 | 18.91 | 46.61 |

### Example 14A: In Vivo Efficacy of PDE3B siRNA Sequences

To assess the in vivo efficacy of the human active PDE3B siRNA sequences for PDE3B knockdown, the PDE3B siRNA molecules were evaluated in an AAV human PDE3B mouse model. Female C57BL/6 mice greater than 10-weeks of age were intravenously injected with 1×10¹¹ genome copies per animal of adeno-associated virus (AAV) designed to express the human PDE3B gene. At least 3-weeks after AAV-injection, mice (n=5 per group) received a single subcutaneous dose of vehicle (PBS) or the PDE3B siRNA molecule conjugated to Gal-2 at a dose of 3 mg/kg. Liver was collected 2-weeks post siRNA administration. Hepatic mRNA expression of human PDE3B was determined using qPCR, with RPLP0 used as the reference gene. Average PDE3B mRNA expression for each compound was normalized relative to the vehicle control group (PBS, Table 23A).

TABLE 23A
In Vivo Knockdown of PDE3B mRNA Expression
in Liver Samples Collected from PDE3B AAV Mice Dosed
with PDE3B siRNA with 3′ Gal-2 conjugates

| | 5′ Antisense | Dose | | |
|---|---|---|---|---|
| dsRNA NO | Modification | (mg/kg) | Mean | SEM |
| Control 1 | | 0 | 1 | 0.22 |
| 208 | VP | 3 | 0.24 | 0.03 |
| 212 | VP | 3 | 0.60 | 0.05 |
| 216 | VP | 3 | 0.45 | 0.08 |
| 220 | VP | 3 | 0.56 | 0.10 |
| 224 | VP | 3 | 0.31 | 0.03 |
| 228 | VP | 3 | 0.43 | 0.01 |
| 232 | VP | 3 | 0.52 | 0.09 |
| 236 | VP | 3 | 0.34 | 0.06 |
| Control 2 | VP | 3 | 1 | 0.17 |
| 209 | VP | 3 | 0.59 | 0.07 |
| 213 | VP | 3 | 0.42 | 0.06 |
| 217 | VP | 3 | 0.48 | 0.05 |
| 221 | VP | 3 | 0.41 | 0.06 |
| 225 | VP | 3 | 0.57 | 0.09 |
| 229 | VP | 3 | 0.51 | 0.03 |
| 233 | VP | 3 | 0.47 | 0.03 |
| 178 | VP | 3 | 0.47 | 0.07 |
| Control 3 | VP | 3 | 1 | 0.13 |
| 210 | VP | 3 | 0.53 | 0.08 |
| 214 | VP | 3 | 0.41 | 0.05 |
| 218 | VP | 3 | 0.50 | 0.09 |
| 222 | VP | 3 | 0.33 | 0.02 |
| 226 | VP | 3 | 0.51 | 0.04 |
| 230 | VP | 3 | 0.35 | 0.06 |
| 234 | VP | 3 | 0.46 | 0.16 |
| 181 | VP | 3 | 0.28 | 0.03 |
| Control 4 | VP | 3 | 1 | 0.05 |
| 211 | VP | 3 | 0.44 | 0.03 |
| 215 | VP | 3 | 0.37 | 0.05 |
| 219 | VP | 3 | 0.31 | 0.05 |
| 223 | VP | 3 | 0.96 | 0.12 |
| 227 | VP | 3 | 0.35 | 0.06 |
| 231 | VP | 3 | 0.39 | 0.05 |
| 235 | VP | 3 | 0.38 | 0.05 |
| 186 | VP | 3 | 0.39 | 0.01 |
| Control 5 | VP | — | 1 | 0.07 |
| 168 | VP | 3 | 0.60 | 0.02 |
| 170 | VP | 3 | 0.67 | 0.08 |
| 174 | VP | 3 | 0.61 | 0.03 |
| Control 6 | VP | — | 1 | 0.12 |
| 53 | VP | 3 | 0.50 | 0.07 |
| 73 | VP | 3 | 0.16 | 0.04 |
| 162 | VP | 3 | 0.25 | 0.02 |
| 65 | VP | 3 | 0.22 | 0.04 |
| 63 | VP | 3 | 0.26 | 0.01 |
| 159 | VP | 3 | 0.40 | 0.09 |
| 64 | VP | 3 | 0.51 | 0.09 |
| 160 | VP | 3 | 0.35 | 0.05 |
| 67 | VP | 3 | 0.22 | 0.01 |
| 69 | VP | 3 | 0.28 | 0.03 |
| Control 7 | VP | — | 1 | 0.14 |
| 70 | VP | 3 | 0.38 | 0.04 |
| 161 | VP | 3 | 0.37 | 0.04 |
| 75 | VP | 3 | 0.34 | 0.03 |
| 66 | VP | 3 | 0.35 | 0.06 |
| 50 | VP | 3 | 0.44 | 0.06 |
| 68 | VP | 3 | 0.33 | 0.02 |
| 71 | VP | 3 | 0.27 | 0.06 |
| 72 | VP | 3 | 0.26 | 0.03 |
| 74 | VP | 3 | 0.49 | 0.08 |
| Control 8 | VP | — | 1 | 0.15 |
| 77 | VP | 3 | 0.48 | 0.04 |
| 78 | VP | 3 | 0.34 | 0.06 |
| 76 | VP | 3 | 0.32 | 0.04 |
| 79 | VP | 3 | 0.51 | 0.07 |
| 80 | VP | 3 | 0.56 | 0.05 |
| 81 | VP | 3 | 0.47 | 0.07 |
| 163 | VP | 3 | 0.54 | 0.06 |
| 82 | VP | 3 | 0.32 | 0.03 |
| 83 | VP | 3 | 0.37 | 0.03 |
| Control 9 | VP | — | 1 | 0.15 |
| 84 | VP | 3 | 0.88 | 0.13 |
| 85 | VP | 3 | 0.77 | 0.04 |
| 86 | VP | 3 | 0.45 | 0.04 |
| 87 | VP | 3 | 0.42 | 0.03 |
| 54 | VP | 3 | 0.48 | 0.02 |
| 88 | VP | 3 | 0.48 | 0.06 |
| 89 | VP | 3 | 0.41 | 0.03 |
| 164 | VP | 3 | 0.40 | 0.08 |
| 90 | VP | 3 | 0.44 | 0.01 |

### Example 14B: In Vivo Knockdown of PDE3B with XHD-PDE3B siRNA Conjugates

To assess the efficacy of the PDE3B siRNA sequences, PDE3B siRNA molecules modified with a 5′-vinylphophonate on the antisense strand were evaluated for in vivo efficacy in female C57BL/6 mice. Animals (n=5 per group) received a single subcutaneous injection of vehicle (PBS) or the PDE3B siRNA conjugated to a lipid (XHD=2′-hexadecyl X, X=A, U, C, G) conjugated on 2′-nucleotide 6 position of sense strand counting from 5′-end) at a dose of 100 nmol/kg. Adipose tissue samples were collected 2 weeks following administration of the siRNA administration. Expression of mouse PDE3B mRNA was determined using qPCR, with RPLP0 used as the reference gene. PDE3B mRNA expression for each compound was normalized relative to the vehicle control group (PBS, Table 23B). The results show a general trend of knockdown of PDE3B by PDE3B siRNA-XHD conjugates in various types of adipose tissue.

TABLE 23B
In Vivo Knockdown (KD) of PDE3B mRNA Expression in Adipose Tissue
Collected from Lean Mice 2 Weeks After Receiving a Single Subcutaneous Dose of PDE3B
siRNAs with 2′-XHD conjugates (at nucleotide 6 counting from 5′-end)

| | | | eWAT | | iWAT | | iBAT | |
|---|---|---|---|---|---|---|---|---|
| | | | Mean | | Mean | | Mean | |
| | | | KD | | KD | | KD | |
| | 5′ Antisense | Dose | relative | | relative | | relative | |
| dsRNA NO | Modification | (nmol/kg) | to control | SEM | to control | SEM | to control | SEM |
| Control 1 | — | 0 | 1 | 0.08 | 1 | 0.06 | 1 | 0.09 |
| 91 | VP | 100 | 0.59 | 0.07 | 0.58 | 0.05 | 0.22 | 0.02 |
| 92 | VP | 100 | 0.54 | 0.09 | 0.55 | 0.09 | 0.25 | 0.03 |
| 93 | VP | 100 | 1.09 | 0.12 | 1.19 | 0.17 | 0.37 | 0.09 |
| 94 | VP | 100 | 0.64 | 0.11 | 0.54 | 0.08 | 0.27 | 0.03 |
| 95 | VP | 100 | 0.80 | 0.13 | 0.71 | 0.12 | 0.25 | 0.02 |
| Control 2 | — | 0 | 1 | 0.08 | 1 | 0.15 | 1 | 0.02 |
| 96 | VP | 100 | 0.45 | 0.04 | 0.50 | 0.09 | 0.33 | 0.04 |
| 97 | VP | 100 | 0.58 | 0.09 | 0.67 | 0.14 | 0.49 | 0.03 |
| 98 | VP | 100 | 0.68 | 0.09 | 0.40 | 0.08 | 0.31 | 0.06 |
| 99 | VP | 100 | 0.51 | 0.07 | 0.40 | 0.03 | 0.42 | 0.04 |
| | VP | 100 | | | | | | |

n = 5 per group.
Epidydimal WAT (eWAT), inguinal WAT (iWAT) and interscapular BAT (iBAT)

### Example 14C: In Vivo Knockdown of PDE3B in Obese Mice

To evaluate the in vivo knockdown of PDE3B siRNA for PDE3B in adipose tissue, five ANP-peptide PDE3B siRNA conjugates were evaluated study in mice. Male obese C57BL/6 mice (n=6 per group) received a single subcutaneous dose of vehicle (PBS) or the PDE3B siRNA ANP-peptide conjugates. Adipose tissue samples were collected 2-weeks post dose. Adipose tissue mRNA expression of murine PDE3B was examined via qPCR, with RPLP0 used as the reference gene. Average PDE3B mRNA expression was normalized relative to the vehicle control group (Table 23C).

TABLE 23C
In Vivo Knockdown (KD) of PDE3B mRNA Expression in Adipose Tissue
Collected from Obese Mice 2 Weeks After Receiving a Single Subcutaneous Dose of PDE3B
siRNAs with ANP-Peptide Conjugates)

| | | | eWAT | | iWAT | | iBAT | |
|---|---|---|---|---|---|---|---|---|
| | | | Mean | | Mean | | Mean | |
| | | | KD | | KD | | KD | |
| | | | relative | | relative | | relative | |
| | 5' Antisense | Dose | to | | to | | to | |
| dsRNA NO | Modification | (nmol/kg) | control | SEM | control | SEM | control | SEM |
| Control | — | 0 | 1 | 0.09 | 1 | 0.03 | 1 | 0.05 |
| 50 | VP | 250 | 0.32 | 0.01 | 0.17 | 0.02 | 0.28 | 0.04 |
| 73 | VP | 250 | 0.27 | 0.01 | 0.11 | 0.08 | 0.17 | 0.01 |
| 77 | VP | 250 | 0.33 | 0.01 | 0.17 | 0.01 | 0.14 | 0.01 |
| 54 | VP | 250 | 0.29 | 0.02 | 0.12 | 0.01 | 0.20 | 0.01 |
| 89 | VP | 250 | 0.37 | 0.02 | 0.20 | 0.02 | 0.33 | 0.03 |

n = 5 per group.
Epidydimal WAT (eWAT), inguinal WAT (iWAT) and interscapular BAT (iBAT)

### Example 14D: Durability of Knockdown by PDE3B RNAi Agents

To evaluate the durability of PDE3B mRNA knockdown in adipose tissue, four ANP-peptide PDE3B siRNA conjugates were evaluated in a time course study in mice. Male obese C57BL/6 mice (n=6 per group) received a single subcutaneous dose of vehicle (PBS) or the PDE3B siRNA ANP-peptide conjugates. Adipose tissue samples were collected 2-, 4-, 8-weeks post siRNA administration. Adipose tissue mRNA expression of murine PDE3B was examined via qPCR, with RPLP0 used as the reference gene. Average PDE3B mRNA expression was normalized relative to the vehicle control group (PBS, Table 23D).

TABLE 23D
In Vivo Knockdown of PDE3B mRNA Expression in
Subcutanouse White Adipose Tissue Collected From Obese
Mice 2-, 4, and 8 Weeks Post Administration of ANP-peptide
PDE3B siRNA conjugates

| | In Vivo Knockdown (%) of PDE3B mRNA | | | | | |
|---|---|---|---|---|---|---|
| | in Subcutaneous Collected from Obese Mice | | | | | |
| Conjugate | 2 weeks | | 4 weeks | | 8 weeks | |
| NO | Mean | SEM | Mean | SEM | Mean | SEM |
| Control | 1 | 0.16 | 1 | 0.14 | 1 | 0.11 |
| C11 | 0.20 | 0.01 | 0.24 | 0.01 | 0.28 | 0.03 |
| C12 | 0.38 | 0.03 | 0.53 | 0.03 | 0.42 | 0.05 |
| C13 | 0.20 | 0.01 | 0.25 | 0.01 | 0.21 | 0.02 |
| C14 | 0.29 | 0.01 | 0.37 | 0.02 | 0.32 | 0.03 |

n = 6 per group

### Example 14E: Dose Response of PDE3B RNAi Agents

To further evaluate the in vivo efficacy of the PDE3B siRNA conjugates, 5 compounds were evaluated in a dose-response in the AAV human PDE3B mouse model. Female C57BL/6 mice greater than 10-weeks of age were intravenously injected with 1×10¹¹ genome copies per animal of adeno-associated virus (AAV) designed to express the human PDE3B gene. At least 3-weeks after AAV-injection, animals (n=5 per group) received a single subcutaneous dose of vehicle (PBS) or the PDE3B siRNA molecule conjugated to Gal-2 at a dose (0.3 mg/kg, 1 mg/kg or 3 mg/kg). Liver was collected 2-weeks post siRNA administration. Hepatic mRNA expression of human PDE3B was determined using qPCR, with RPLP0 used as the reference gene. Average PDE3B mRNA expression for each compound was normalized relative to the vehicle control group (PBS, Table 23E).

TABLE 23E
Dose Response In Vivo Knockdown of PDE3B mRNA Expression in Liver
Samples Collected from PDE3B AAV Mice Dosed with PDE3B siRNA
with 3′ Gal-2 conjugates

| dsRNA | | | 0.3 mg/kg | | 1 mg/kg | | 3 mg/kg | |
|---|---|---|---|---|---|---|---|---|
| No | Mean | SEM | Mean | SEM | Mean | SEM | Mean | SEM |
| Control | 1 | 0.06 | — | — | — | — | — | — |
| 178 | — | — | 0.39 | 0.07 | 0.40 | 0.04 | 0.38 | 0.02 |
| 165 | — | — | 0.59 | 0.05 | 0.41 | 0.05 | 0.39 | 0.07 |
| 175 | — | — | 0.48 | 0.07 | 0.45 | 0.06 | 0.25 | 0.03 |
| 177 | — | — | 0.50 | 0.01 | 0.19 | 0.05 | 0.31 | 0.01 |
| 176 | — | — | 0.77 | 0.05 | 0.50 | 0.04 | 0.39 | 0.03 |
| Control | 1 | 0.20 | — | — | — | — | — | — |
| 167 | — | — | 0.34 | 0.02 | 0.29 | 0.05 | 0.32 | 0.01 |
| 169 | 1 | — | 0.39 | 0.05 | 0.25 | 0.02 | 0.23 | 0.02 |
| 172 | — | — | 0.43 | 0.06 | 0.35 | 0.07 | 0.21 | 0.03 |
| Control | 1 | 0.07 | — | — | — | — | — | — |
| 171 | — | — | 0.56 | 0.04 | 0.04 | 0.41 | 0.21 | 0.02 |
| 173 | — | — | 0.35 | 0.03 | 0.24 | 0.02 | 0.31 | 0.03 |
| 166 | — | — | 0.52 | 0.07 | 0.30 | 0.02 | 0.23 | 0.01 |

n = 5 per group

### Example 15: Efficacy of PDE3B siRNA with 3′-ANP Peptide Conjugate in Obese Mice

Two (n=2) PDE3B-siRNA sequences with homology to the mouse PDE3B gene transcript were conjugated to a peptide (SEQ ID NO: 20) with affinity for the Natriuretic Peptide Clearance Receptor C (NPRC) to evaluate the effect of PDE3B knockdown on weight loss efficacy in C57BL/6 diet-induced obese (DIO) male mice fed a 60% high-fat diet housed at thermoneutrality (27° C.). Mice were approximately 20-weeks of age at the start of the study. Mice received two subcutaneous doses (5 mL/kg) of either PBS (n=6), or the ANP-conjugated PDE3B siRNA (250 nmol/kg, n=6 per group) prior to treatment with the incretin semaglutide (SEQ ID NO: 269), a GLP-1R agonist therapeutic. Following 28 days of treatment, animals received a final dose of the PDE3B siRNA (250 nmol/kg), and daily dosing with the GLP-1RA (3 nmol/kg) was initiated (n=6 per group). Body weight was monitored daily, body composition was analyzed prior to incretin dosing and at the end of study. The ANP-PDE3B siRNA conjugates augmented weight loss induced by the semaglutide. Additionally, the ANP-PDE3B siRNA conjugates stimulated greater fat mass when dosed in combination with the GLP-1RA (Table 24).

On day 15 post start of GLP-1RA dosing, animals were euthanized. Adipose tissue samples were collected, and mouse PDE3B mRNA expression was determined using qPCR, with RPLP0 used as the reference gene. PDE3B mRNA expression for each compound was normalized relative to the vehicle control group (PBS, Table 25).

TABLE 24
In Vivo Efficacy in Obese Mice Treated with PDE3B siRNA with
3′-ANP peptide conjugate

| | Body Weight | | Fat Mass | | Fat-Free Mass | |
|---|---|---|---|---|---|---|
| | Change compared | | Change from | | Change from | |
| Conjugate | to Vehicle (%) | | Baseline (%) | | Baseline (%) | |
| NO | Mean | SEM | Mean | SEM | Mean | SEM |
| Vehicle | 0.00 | 0.195 | 6.78 | 0.63 | 1.45 | 0.46 |
| C1 | −4.88 | 0.74 | −10.58 | 2.5 | 1.86 | 0.50 |
| C2 | −0.95 | 0.92 | 4.05 | 2.01 | 1.19 | 0.48 |
| sema | −13.28 | 1.23 | −11.64 | 4.02 | −9.06 | 0.47 |
| C1 + sema | −22.78 | 1.45 | −41.76 | 2.76 | −8.06 | 0.53 |
| C2 + sema | −21.36 | 2.94 | −31.71 | 6.45 | −9.93 | 0.55 |

n = 6 per group

TABLE 25
In Vivo Knockdown (KD) of PDE3B mRNA Expression in Adipose Tissue of
DIO mice after five doses of 3′-ANP siRNA conjugate

| | | | e WAT | | iWAT | | iBAT | |
|---|---|---|---|---|---|---|---|---|
| | | | Mean | | Mean | | Mean | |
| | | | KD | | KD | | KD | |
| | | Dose | relative | | relative | | relative | |
| Conjugate | 5′ Antisense | (nmol/ | to | | to | | to | |
| NO | Modification | kg) | control | SEM | control | SEM | control | SEM |
| Control | — | — | 1 | 0.129 | 1 | 0.05 | 1 | 0.08 |
| C1 | VP | 250 | 0.26 | 0.02 | 0.15 | 0.01 | 0.12 | 0.01 |
| C2 | VP | 250 | 0.32 | 0.02 | 0.12 | 0.01 | 0.16 | 0.01 |
| sema | VP | 3 | 0.88 | 0.07 | 1.09 | 0.03 | 1.002 | 0.13 |
| C1 + | VP | — | 0.34 | 0.02 | 0.17 | 0.01 | 0.17 | 0.01 |
| sema | | | | | | | | |
| C2 + | VP | — | 0.15 | 0.03 | 0.13 | 0.07 | 0.15 | 0.01 |
| sema | | | | | | | | |

n = 6 per group.
Epidydimal WAT (eWAT), inguinal WAT (iWAT) and interscapular BAT (iBAT)

SEQUENCE LISTING

| SEQ | |
|---|---|
| ID | |
| NO | Sequence |
| 1 | GX11IDX14I where X11 is R, r, P, p Hyp or D-Hyp; X14 is R, |
| | r or Arg(Me) |
| 2 | GRIDRI |
| 3 | SX7X8X9GX11IDX14I where X7 is G, g, A, a, P, p, Hyp, D-Hyp S, |
| | s, C, or c; X8 is F, f or Cha, X9 is G, g, A, a or S, s; |
| | X11 is R, r, P, p Hyp or D-Hyp; X14 is R, r or Arg(Me) |
| 4 | SCFGGRIDRI |
| 5 | FGGRIDRIGA |
| 6 | RSSX7FGGRIDRI where X7 is S, s, C or c |
| 7 | RSSX7FGGRIDRIGA where X7 is S, s, C or c |
| 8 | X7-Cha-X9GX11IDX14I where X7 is P, p, Hyp, D-Hyp, G, g, C, c, |
| | A, or a; X9 is A, a, S, s, G or g; X11 is P, p, Hyp R or r; |
| | and X14 is R, r or Arg(Me) |
| 9 | fSp-Cha-aGPIDRI |
| 10 | SLRRSS[CFGGRIDRIGAQSGLGC]NSFRY-NH2 |
| 11 | RSS[CFGGRIDRIGAQSGLGC]-NH2 |
| 12 | RSSCFGGRIDRIGAQSGLGC-NH2 |
| 13 | RSSSFGGRIDRIGAQSGLGS-NH2 |
| 14 | RSS[CFGGRIDRIGAQSGLGC]-OH |
| 15 | RSSCFGGRIDRIGAQSGLGC-OH |
| 16 | RSSSFGGRIDRIGAQSGLGS-OH |
| 17 | RSSSFGGRIDRIGAQSGLGS-NH2 |
| 18 | RSS[CFGGRIDRIGAC]-NH2 |
| 19 | RSS[CFGGRIDRIGAQSGLGC]^{CH2}-OH |
| 20 | RSS[CFGGRIDRIGAQSGLGC]^{CH2}-NH2 |
| 21 | fSp-Cha-aGPIDRI-NH2 |
| 22 | fS-(D-Hyp)-Cha-sG-Hyp-ID-Arg(Me)-I-NHCH3 |
| 23 | fSp-Cha-aGPIDRIGSPSSGAPPPS-NH2 |
| 24 | fSp-Cha-aGPIDRI-OH |
| 25 | Orn-fSp-Cha-aGPIDSI-NH2 |
| 26 | fSp-Cha-aGPIDSI-Dap-NH2 |
| 27 | fS-(D-Pip)-Cha-aGPIDSI-NH2 |
| 28 | FSG-Cha-GGRIDRI-NH2 |
| 29 | FSG-Cha-Aib-GRIDRI-NH2 |
| 30 | fSp-Cha-aGPIERI-NH2 |
| 31 | fSp-Cha-aGPID-(NMe-Arg)-I-NH2 |
| 32 | RSFSGFGGRIDRI-NH2 |
| 33 | RSFSGFGGRIDRIGAQSGLGS-NH2 |
| 34 | RSFSGFGGRIDRIG-NH2 |
| 35 | C-Cha-GGRIDRIG-NH2 |
| 36 | Ac-C-Cha-GGRIDRIG-NH2 |
| 37 | S-Cha-GGRIDRIG-NH2 |
| 38 | A-Cha-GGRIDRIG-NH2 |
| 39 | Cha-GGRIDRIG-NH2 |
| 40 | Ac-Cha-GGRIDRIG-NH2 |
| 41 | G-Cha-GGRIDRIG-NH2 |
| 42 | SG-Cha-GGRIDRIG-NH2 |
| 43 | FSG-Cha-GGRIDRIG-NH2 |
| 44 | SFSG-Cha-GGRIDRIG-NH2 |
| 45 | RSFSG-Cha-GGRIDRIG-NH2 |
| 46 | RSSG-Cha-GGRIDRIG-NH2 |
| 47 | fSG-Cha-GGRIDRIG-NH2 |
| 48 | Ac-fSG-Cha-GGRIDRIG-NH2 |
| 49 | fSp-Cha-GGRIDRIG-NH2 |
| 50 | fSG-Cha-aGRIDRIG-NH2 |
| 51 | fSG-Cha-GGPIDRIG-NH2 |
| 52 | Ac-fSp-Cha-aGPIDRIG-NH2 |
| 53 | Ac-fSp-Cha-aGPIDRI-NH2 |
| 54 | Ac-fSp-Cha-aGPIDRI-NHCH3 |
| 55 | Ac-fSp-Cha-aGPID-Arg(Me)-I-NHCH3 |
| 56 | Ac-fS-(D-Hyp)-Cha-aG-Hyp-ID-Arg(Me)-I-NHCH3 |
| 57 | (HOCH2CO)-fS-(D-Hyp)-Cha-sG-Hyp-ID-Arg(Me-)I-NHCH3 |
| 58 | AEEA3-fSp-Cha-aGPIDRIGGGGSGGGGS |
| 59 | GGGGSGGGGSfSp-Cha-aGPIDRIGGGGSGGGGS |
| 60 | GGGGSGGGGSGGGGSGGGGS |
| 61 | AEEA3-GGGGSGGGGSGGGGSGGGGS |
| 62 | GGGGHGGGGHGGGGHGGGGH |
| 63 | GGGG-4Pal-GGGG-4Pal-GGGG-4Pal-GGGG-4Pal |
| 64 | GGGGSHHHGGGGSHHHGGGGS |
| 65 | AEEA3-GGGGSGGGGS |
| 66 | GGGGSGGGGSGGGGSGGGGSGGGGSGGGGS |
| 67 | GGGGSGGGGSGGGGSGGGGSP |
| 68 | GGGG-4Pal-GGGG-4Pal-GGGG-4Pal-GGGG-4Pal-P |
| 69 | GGGG-4Pal-GGGG-4Pal-GGGG-4Pal-GGGG-4Pal-GGGG-4Pal-GGGG-4Pal |
| 70 | GGGGEGGGGEGGGGSGGGGSGGGGSGGGGS |
| 71 | GGGGEGGGGSGGGGEGGGGSGGGGEGGGGS |
| 72 | GGGGEGGGGEGGGGEKEKEKGGGGSGGGGS |
| 73 | GSPSSGAPPPS |
| 74 | MPSLLVLTFSPCVLLGWALLAGGTGGGGVGGGGGGAGIGGGRQEREALPPQKIEVLV |
| | LLPQDDSYLFSLTRVRPAIEYALRSVEGNGTGRRLLPPGTRFQVAYEDSDCGNRALFSL |
| | VDRVAAARGAKPDLILGPVCEYAAAPVARLASHWDLPMLSAGALAAGFQHKDSEYS |
| | HLTRVAPAYAKMGEMMLALFRHHHWSRAALVYSDDKLERNCYFTLEGVHEVFQEE |
| | GLHTSIYSFDETKDLDLEDIVRNIQASERVVIMCASSDTIRSIMLVAHRHGMTSGDYAF |
| | FNIELFNSSSYGDGSWKRGDKHDFEAKQAYSSLQTVTLLRTVKPEFEKFSMEVKSSVE |
| | KQGLNMEDYVNMFVEGFHDAILLYVLALHEVLRAGYSKKDGGKIIQQTWNRTFEGIA |
| | GQVSIDANGDRYGDFSVIAMTDVEAGTQEVIGDYFGKEGRFEMRPNVKYPWGPLKLR |
| | IDENRIVEHTNSSPCKSSGGLEESAVTGIVVGALLGAGLLMAFYFFRKKYRITIERRTQQ |
| | EESNLGKHELREDSIRSHFSVA |
| 75 | MPSLLVLTFSPCVLLGWALLAGGTGGGGVGGGGGGAGIGGGRQEREALPPQKIEVLV |
| | LLPQDDSYLFSLTRVRPAIEYALRSVEGNGTGRRLLPPGTRFQVAYEDSDCGNRALFSL |
| | VDRVAAARGAKPDLILGPVCEYAAAPVARLASHWDLPMLSAGALAAGFQHKDSEYS |
| | HLTRVAPAYAKMGEMMLALFRHHHWSRAALVYSDDKLERNCYFTLEGVHEVFQEE |
| | GLHTSIYSFDETKDLDLEDIVRNIQASERVVIMCASSDTIRSIMLVAHRHGMTSGDYAF |
| | FNIELFNSSSYGDGSWKRGDKHDFEAKQAYSSLQTVTLLRTVKPEFEKFSMEVKSSVE |
| | KQGLNMEDYVNMFVEGFHDAILLYVLALHEVLRAGYSKKDGGKIIQQTWNRTFEGIA |
| | GQVSIDANGDRYGDFSVIAMTDVEAGTQEVIGDYFGKEGRFEMRPNVKYPWGPLKLR |
| | IDENRIVEHTNSSPCKSCGLEESAVTGIVVGALLGAGLLMAFYFFRKKYRITIERRTQQE |
| | ESNLGKHRELREDSIRSHFSVA |
| 76 | agtcccgagaggtgcccgagggaaaaggaggcggcagctaaactggtcctggagagaagc |
| | cccttccgcccctctcctcagccagcatgtcccggactccgccgctcctcagtccgcgcg |
| | gtggggaccccgggccgtggcggccggcgcagccctgacgggttgcgaaccagggggcgc |
| | cccgaacgcgggggttggggtctgggagcgcgagcggccgctacggtacgagcggggtgt |
| | gctgagtcccgtggccacccccggccccagccatgaggagggacgagcgagacgccaaag |
| | ccatgcggtccctgcagccgccggatggggccggctcgccccccgagagtctgaggaacg |
| | gctacgtgaagagctgcgtgagccccttgcggcaggaccctccgcgcggcttcttcttcc |
| | acctctgccgcttctgcaacgtggagctgcggccgccgccggcctctccccagcagccgc |
| | ggcgctgctcccccttctgccgggcgcgcctctcgctgggcgccctggctgcctttgtcc |
| | tcgccctgctgctgggcgcggaacccgagagctgggctgccggggccgcctggctgcgga |
| | cgctgctgagcgtgtgttcgcacagcttgagccccctcttcagcatcgcctgtgccttct |
| | tcttcctcacctgcttcctcacccggaccaagcggggacccggcccgggccggagctgcg |
| | gctcctggtggctgctggcgctgcccgcctgctgttacctgggggacttcttggtgtggc |
| | agtggtggtcttggccttggggggatggcgacgcagggtccgcggccccgcacacgcccc |
| | cggaggcggcagcgggcaggttgctgctggtgctgagctgcgtagggctgctgctgacgc |
| | tcgcgcacccgctgcggctccggcactgcgttctggtgctgctcctggccagcttcgtct |
| | ggtgggtctccttcaccagcctcgggtcgctgccctccgccctcaggccgctgctctccg |
| | gcctggtggggggcgctggctgcctgctggccctggggttggatcacttctttcaaatca |
| | gggaagcgcctcttcatcctcgactgtccagtgccgccgaagaaaaagtgcctgtgatcc |
| | gaccccggaggaggtccagctgcgtgtcgttaggagaaactgcagccagttactatggca |
| | gttgcaaaatattcaggagaccgtcgttgccttgtatttccagagaacagatgattcttt |
| | gggattgggacttaaaacaatggtataagcctcattatcaaaattctggaggtggaaatg |
| | gagttgatctttcagtgctaaatgaggctcgcaatatggtgtcagatcttctgactgatc |
| | caagccttccaccacaagtcatttcctctctacggagtattagtagcttaatgggtgctt |
| | tctcaggttcctgtaggccaaagattaatcctctcacaccatttcctggattttacccct |
| | gttctgaaatagaggacccagctgagaaaggggatagaaaacttaacaagggactaaata |
| | ggaatagtttgccaactccacagctgaggagaagctcaggaacttcaggattgctacctg |
| | ttgaacagtcttcaaggtgggatcgtaataatggcaaaagacctcaccaagaatttggca |
| | tttcaagtcaaggatgctatctaaatgggccttttaattcaaatctactgactatcccga |
| | agcaaaggtcatcttctgtatcactgactcaccatgtaggtctcagaagagctggtgttt |
| | tgtccagtctgagtcctgtgaattcttccaaccatggaccagtgtctactggctctctaa |
| | ctaatcgatcacccatagaatttcctgatactgctgattttcttaataagccaagcgtta |
| | tcttgcagagatctctgggcaatgcacctaatactccagatttttatcagcaacttagaa |
| | attctgatagcaatctgtgtaacagctgtggacatcaaatgctgaaatatgtttcaacat |
| | ctgaatcagatggtacagattgctgcagtggaaaatcaggtgaagaagaaaacattttct |
| | cgaaagaatcattcaaacttatggaaactcaacaagaagaggaaacagagaagaaagaca |
| | gcagaaaattatttcaggaaggtgataagtggctaacagaagaggcacagagtgaacagc |
| | aaacaaatattgaacaggaagtatcactggacctgattttagtagaagagtatgactcat |
| | taatagaaaagatgagcaactggaattttccaatttttgaacttgtagaaaagatgggag |
| | agaaatcaggaaggattctcagtcaggttatgtataccttatttcaagacactggtttat |
| | tggaaatatttaaaattcccactcaacaatttatgaactattttcgtgcattagaaaatg |
| | gctatcgagacattccttatcacaatcgtatacatgccacagatgtgctacatgcagttt |
| | ggtatctgacaacacggccagttcctggcttacagcagatccacaatggttgtggaacag |
| | gaaatgaaacagattctgatggtagaattaaccatgggcgaattgcttatatttcttcga |
| | agagctgctctaatcctgatgagagttatggctgcctgtcttcaaacattcctgcattag |
| | aattgatggctctatacgtggcagctgccatgcatgattatgatcacccagggaggacaa |
| | atgcatttctagtggctacaaatgcccctcaggcagttttatacaatgacagatctgttc |
| | tggaaaatcatcatgctgcgtcagcttggaatctatatctttctcgcccagaatacaact |
| | tccttcttcatcttgatcatgtggaattcaagcgctttcgttttttagtcattgaagcaa |
| | tccttgctacggatcttaaaaagcattttgattttctcgcagaattcaatgccaaggcaa |
| | atgatgtaaatagtaatggcatagaatggagtaatgaaaatgatcgcctcttggtatgcc |
| | aggtgtgcatcaaactggcagatataaatggcccagcaaaagttcgagacttgcatttga |
| | aatggacagaaggcattgtcaatgaattttatgagcagggagatgaagaagcaaatcttg |
| | gtctgcccatcagtccattcatggatcgttcttctcctcaactagcaaaactccaagaat |
| | cttttatcacccacatagtgggtcccctgtgtaactcctatgatgctgctggtttgctac |
| | caggtcagtggttagaagcagaagaggataatgatactgaaagtggtgatgatgaagacg |
| | gtgaagaattagatacagaagatgaagaaatggaaaacaatctaaatccaaaaccaccaa |
| | gaaggaaaagcagacggcgaatattttgtcagctaatgcaccacctcactgaaaaccaca |
| | agatatggaaggaaatcgtagaggaagaagaaaaatgtaaagctgatgggaataaactgc |
| | aggtggagaattcctccttacctcaagcagatgagattcaggtaattgaagaggcagatg |
| | aagaggaatagcgacagtttgagtaaaagaaaagtcatattgaagaagcccagagggttg |
| | tgcccaggggcagaaatcattgcctagtgttcaccggctgactctcaactgaccattccc |
| | atgtggacaggccttaatactgtgagaggatccttgctctgctggcagtttcccactcct |
| | atgcactttcacaggaactagaaaactattcttaaaccaaaaataccatccgtgttgacc |
| | catgttgcagagcccttacttaaatccttcactggtgtatgaatactttgtcataatgct |
| | gctttgctgggtagtgagctcttatttttcactgggggtcagctataactaaaaactcaa |
| | gtgacatatttcagttaccaaagtggccaggaactttttgcttttatgaaaatagattca |
| | tattgtatttcccagtgtgtcttttatgtctttgaatgttttggagaaaagtctatgcct |
| | gtctaaaaatgaatccagtgttgcctttctgagggatttctgctcaatgcaatacactgt |
| | tcagtgctattctcccagctaggtttatccatgaaggactgagtgacctttgttgtattt |
| | aacaaaatccaggtgcatcaatttctgatgctttttactattgtgtattatctactatgt |
| | gtgttttatttctgctgagagtattcaggtttgccatggacatcagaagtttgaattcca |
| | gtcttatcttatgttccatggctgaattttaaagctgtttaggtttaacaatgaagggat |
| | ttattctttagtcaaaattgttgtttttactctagctcaggattcgtatttttaaagatt |
| | tagttaatatgaacacagcacagatttgttagaagaaaaaaaatttgctgtaataccaaa |
| | actaacctcatcaaagatacagaaaaaaagaaatatagtgagccctaaaggacacataca |
| | ttgaataaataattggaacatgtggttatctttagatccacatcttagctgtcatttgtt |
| | cactctaaaactgatgttcatctttctgttaatttccctctgcctaaagactacatgaca |
| | gaaatgacctatcactacttattatttctgaagcctaactgcaagactgatttctgagaa |
| | caagtaaagaactggaatacttatttttcatataaaaatctaaatgtgttaataaatcat |
| | ttcatacaaaagtacattattaaataaccacattattaaaataattgcaagaaaatggac |
| | catatttacaatgttttgtaaacttgctagtgtgtggatatgtaccctacttgtgaaata |
| | catttgaagatataaagagcagccaaaatgatggcaaaatggtaggctaatattttctat |
| | tattattggagaacatatcatattttggaatcatgcaattttgcacacagtgaaaccatt |
| | aattttccaaggtaattcctttagaatatggtattggcatgcagtttcttacttatctag |
| | aatatttggcttatctgaaagatatcaatttaagatctctggaagtgttagaatttttga |
| | tccttcacagtgtcaatatttaatgaatcactaagctttatttattagacgtgttgagtg |
| | agtgctgagttccttgctgccacttttgttaccattgtcacacactatgtgtaaaccagt |
| | cccaccacttattactaataaaattttgactgataatttatatttgcacttacaatatat |
| | atatcctgtccttatatttctctagagtacattttccatcatgtttaagtgtatttctgc |
| | tattatttcctctcctgcagaatacatacaagtgtatgtgtataaagtcatacatgtaca |
| | agcatgcatattgagattgaatcacatttccatactgtctgttattttattgggttttat |
| | attgggtttctttagtttatgttgttttctcaaaagcagcattttaaattacgaatactg |
| | gacttattggatttaattataaatccaattactactggaaactcatttttacataatata |
| | gtccttaaattatttaacccttgctaagtaattgacatatgtaacaataactagcctaaa |
| | gaaacccaaaaaagtatctctcccgagctgaaacttaaaaattcgtaagtgtaagaaaga |
| | atgtgagaatatattaaatgcacactgtaccattagatgaaatcttacttgagaaattgc |
| | cataagccatattacagatcttactttgttactgaatcagattaatttcttgttataata |
| | attttcatcataaattttctatttttaaagccgctggtactagaaatattcttttaatgc |
| | tatatctatgtacctactgacacatttttctccataaaagtacttttaaaaatta |
| 77 | MRRDERDAKAMRSLQPPDGAGSPPESLRNGYVKSCVSPLRQDPP |
| | RGFFFHLCRFCNVELRPPPASPQQPRRCSPFCRARLSLGALAAFVLALLLGAEPESWA |
| | AGAAWLRTLLSVCSHSLSPLFSIACAFFFLTCFLTRTKRGPGPGRSCGSWWLLALPAC |
| | CYLGDFLVWQWWSWPWGDGDAGSAAPHTPPEAAAGRLLLVLSCVGLLLTLAHPLRLRH |
| | CVLVLLLASFVWWVSFTSLGSLPSALRPLLSGLVGGAGCLLALGLDHFFQIREAPLHP |
| | RLSSAAEEKVPVIRPRRRSSCVSLGETAASYYGSCKIFRRPSLPCISREQMILWDWDL |
| | KQWYKPHYQNSGGGNGVDLSVLNEARNMVSDLLTDPSLPPQVISSLRSISSLMGAFSG |
| | SCRPKINPLTPFPGFYPCSEIEDPAEKGDRKLNKGLNRNSLPTPQLRRSSGTSGLLPV |
| | EQSSRWDRNNGKRPHQEFGISSQGCYLNGPFNSNLLTIPKQRSSSVSLTHHVGLRRAG |
| | VLSSLSPVNSSNHGPVSTGSLTNRSPIEFPDTADFLNKPSVILQRSLGNAPNTPDFYQ |
| | QLRNSDSNLCNSCGHQMLKYVSTSESDGTDCCSGKSGEEENIFSKESFKLMETQQEEE |
| | TEKKDSRKLFQEGDKWLTEEAQSEQQTNIEQEVSLDLILVEEYDSLIEKMSNWNFPIF |
| | ELVEKMGEKSGRILSQVMYTLFQDTGLLEIFKIPTQQFMNYFRALENGYRDIPYHNRI |
| | HATDVLHAVWYLTTRPVPGLQQIHNGCGTGNETDSDGRINHGRIAYISSKSCSNPDES |
| | YGCLSSNIPALELMALYVAAAMHDYDHPGRTNAFLVATNAPQAVLYNDRSVLENHHAA |
| | SAWNLYLSRPEYNFLLHLDHVEFKRFRFLVIEAILATDLKKHFDFLAEFNAKANDVNS |
| | NGIEWSNENDRLLVCQVCIKLADINGPAKVRDLHLKWTEGIVNEFYEQGDEEANLGLP |
| | ISPFMDRSSPQLAKLQESFITHIVGPLCNSYDAAGLLPGQWLEAEEDNDTESGDDEDG |
| | EELDTEDEEMENNLNPKPPRRKSRRRIFCQLMHHLTENHKIWKEIVEEEEKCKADGNK |
| | LQVENSSLPQADEIQVIEEADEEE |
| 78 | aactgttgcagcgcgcgggagctgggaggcgacgccgccgtctcccgtcccgagccgcgc |
| | ttgagggaaaagggaagaggcgtccgcggcggtcccgggacaccaagaagcccttctcgc |
| | cgctctcagtggccggcatgaggcggacgccgccgcccctcagcccgcgcgccggggatc |
| | ccggccggtggcggcggcgggcgcagccgtgaccgctctgcgaagctcggggcgcccaga |
| | acgcggagggcggcgcggggtcggcaagcgccagcggccccccggcggcccggggccatg |
| | aggaaagacgagcgcgagcgggacgcgccagccatgaggtccccgccgccgccgccagcc |
| | tcggccgcctcgccccccgagagcctgcgcaacggctacgtgaagagctgcgtgagcccg |
| | ctgcggcaggaccctccgcgcagcttcttcttccacctctgccgcttctgcaacgtggag |
| | ccgccggcggcctcgctccgcgccggggcacgcctctcgctcggcgtcctggccgccttt |
| | gtcctggccgcgctgctgggcgcgaggcccgagcgctgggcggccgcggcagccgggctc |
| | cggacgctgctgagcgcctgctcgctcagcctcagcccgctcttcagcatcgcctgtgcc |
| | ttcttcttcctcacctgtttcctgacgcgcgcgcagcgcggcccgggccgcggcgccggc |
| | tcctggtggctgctggcgctgcccgcctgctgctacctgggcgacttcgcggcgtggcag |
| | tggtggtcgtggctgcgtggggagccggcggcggcgggccggctctgcctggtgctgagc |
| | tgcgtggggctgctgacgctcgcgccccgcgtgaggctgcggcacggcgtcctggtgctg |
| | ctcttcgccggcctggtgtggtgggtgtccttctccggcctcggggctctgccgcccgcg |
| | ctcaggcctctgctgtcgtgcctggtcgggggcgcgggatgcctgctagccctgggcttg |
| | gaccacttctttcacgtccggggagcctcccctccgccgcgctcggcgagtaccgcggag |
| | gaaaaagtgcctgtgatcagaccccggaggaggtccagctgcgtgtcgctgggagaaagc |
| | gcagccggttactatggcagtggcaagatgttcaggagaccgtcgttgccttgtatttcc |
| | cgagaacagatgatcctctgggactgggacttgaagcagtggtgtaaacctcattaccag |
| | aattctggaggtggaaatggagttgatctttcagtgctaaatgaagctcgcaatatggtg |
| | tcagacctgctgattgacccaagccttcccccacaagtcatttcttctctgcggagtatc |
| | agtagcttgatgggtgctttctcaggttcctgtaggccaaagattaattcttttacacca |
| | tttcctggattttatccctgctctgaagtagaagatccagttgagaaaggagatcgaaaa |
| | cttcacaagggattgagtggcagaaccagtttcccaactccacagctgaggaggagctct |
| | ggagcctcatcgttgctgactaatgagcactgttcaaggtgggatcgcagcagtggtaag |
| | aggtcttaccaagaactcagtgtttcaagtcatggatgccacctaaatgggccttttagt |
| | tcaaatctttttacaattccaaagcagaggtcatcgtctgtgtcactgacgcaccatgca |
| | ggtctgagaagagctggtgctttgcccagccacagtcttctgaattcttcaagccatgta |
| | ccagtgtctgctggctctctaactaatcgatcacccataggatttcctgataccactgat |
| | tttcttactaagccaaatattattttacatagatcactgggcagtgtatcaagtgcagca |
| | gatttccatcagtaccttaggaactctgacagcaatctgtgtagcagctgtggacaccaa |
| | atactcaaatatgtttcaacatgtgaacccgatggtacagaccaccccagtgaaaaatca |
| | ggtgaagaagacagcagtgttttctcaaaagaaccattgaacattgtggaaacccaagaa |
| | gaagagaccatgaagaaagcctgcagggagttatttttggaaggtgatagtcacctgatg |
| | gaagaggcacagcaaccaaatatcgatcaggaagtgtcactggatccaatgttagtagaa |
| | gattatgattcattaatagaaaagatgaacaactggaattttcagatttttgagcttgta |
| | gaaaagatgggagaaaaatcaggaaggattctcagtcaggttatgtatactttatttcaa |
| | gatactggtttattggaaacatttaaaattcccactcaagaatttatgaattattttcgt |
| | gccttagaaaatggctaccgggacattccatatcacaatcgtgtgcatgccacagatgtc |
| | ctacatgctgtttggtatttgacaacccgaccaattcctggcttacctcagatccataat |
| | aaccatgaaacggaaaccaaagcagattcagatggtagacttggttctggacagattgct |
| | tacatttcttcgaagagttgctgtattccagatatgagttatggctgcctatcttcaaac |
| | atccctgcattagaattgatggctttgtatgtggcagctgccatgcacgattacgatcac |
| | ccaggaagaacaaatgcgtttctagtggctacaaatgcacctcaggcagttttatacaat |
| | gacagatctgttctagagaatcatcatgctgcatcagcttggaatctgtatctttctcgc |
| | ccagagtacaacttcctccttaaccttgatcacatggaattcaagcggtttcgattttta |
| | gttatagaagcaatccttgctacagatctcaaaaaacattttgatttccttgcagaattc |
| | aatgccaaggccaatgatgtaaatagtaacggtatagaatggagcagtgaaaacgatcgc |
| | ctcttggtctgccaggtgtgcatcaaattagcagatatcaatggcccagcaaaagatcgg |
| | gacctacatttgagatggacagaaggcattgtgaatgaattttatgagcagggagatgaa |
| | gaagcaaccctgggtctacctattagtcccttcatggatcgttcttctcctcaactagca |
| | aaactccaagaatcttttatcacccacattgtgggccccctgtgcaactcctatgatgct |
| | gctggcttgctgccgggtcagtggatagaaacagaagagggtgatgatacagaaagtgat |
| | gatgatgatgatgatgatgatggtggtggtgaagaattagattcagatgatgaagaaaca |
| | gaagacaatctaaatcctaaaccacaaagaaggaaaggcaggcggcgaatattttgccaa |
| | ctaatgcaccacctcactgaaaaccacaagatatggaaggaaatcatagaagaagaagaa |
| | gaaaaatgtaaagctgaggggaacaaactgcaggtggataatgcctccttacctcaggca |
| | gatgagattcaggttattgaagaagcagatgaagaggaagaacaaatgtttgaatgagaa |
| | gagaactcatgctgaaggagcctggggtgctgttcccagggtcattacctaatgctcact |
| | gtactgattctcaactgaccattcccatgtgggcacgccttaatactgtgagaggatcct |
| | ggctaccctggcagttctcactcctaagcactttcattacaaacaagaaactgattctta |
| | gagcttgctgcagttgactcatgctgcaaagccttcctcaaagccttcactggtgtgtaa |
| | atagtgtgtcataatgctgctttgctgggtagtgagctcttatttctcatgatgagggag |
| | ggaaggctaaaactgaaaagtcaagtgacttcggtttttgaagtaaccatatttctttta |
| | tgaaaacaggattcatcttgtatttccaaatatatcttttatgtctttgtttgggggtgg |
| | ggtggtgcaagaaacagacctctatgcttaatacaaaaatgaattcatggtggcctttct |
| | gagtgatttctgctcgtggcagcacactgttcagtgtggttctcctggcttcatcgagga |
| | aggactgaggacctttgttatacttagcaaaacccagatgcatcaatttctgatgctttt |
| | tattgttgtgtataatctatttacttgttttatttctgcctaaagtattcaggtttgctg |
| | tggacatcagaggtctgaattctgttcttactgattttgttccatggttgaatttaaaag |
| | tatataataatgaaggagctttattctttattcaaatctgttcttattcttattctttct |
| | caggataagtaattttaaacaattattataaatattgcatagatttgccagagaaaaaaa |
| | cattgtcctgatattgatgttatcttttatgaaatgagcaccattttattttaattagag |
| | tcaagaaaaaggagctggccatcttttgccttaatggaaaatcaatgaaaccaatcccat |
| | cgaagatattgcctaaatatttagttatctttagatatatgtcttaggtgtctagtaaaa |
| | ttgacattttttattgttaatatctttctgcttcaagagtacatggcagagatgacacat |
| | cactatttagtaatattacagccccacagacagaattttgaagtaaagtacataatggga |
| | atgcatattaatttttaataaagtgctataaaataaatatgctatgaatagttcaataaa |
| | taattccataaatgcttataaataaataatttactaagaaaacaggacacatttatgttt |
| | atataatctcattagagtgtggctatgtgccccagttgtgaaaaattattgaagttatac |
| | aaagcaaccaaaataataccaaaatggtaaactggtttcttcttatcctttatacattat |
| | tttgaagccattgaactttgtgcacagttaactgtcagtttccacagtatttcctatata |
| | gtgtgatgctggaatttcttacctacctaagatatttgggttgtctacaaggccacagtt |
| | aaagattgattggagtactggtatttttgatcctgtaggtaccatatttaatgaatcact |
| | acactttatttattaaacatgttgagtgctgagttccttcctgtcacttttgctaccaca |
| | actacatcatttgtaaactggtcccaccatatattactaagtaataaaattttaactgat |
| | aatctaaaaaaaaaaaaaaaaaaaaaaaaaaaaa |
| 79 | MRKDERERDAPAMRSPPPPPASAASPPESLRNGYVKSCVSPLRQ |
| | DPPRSFFFHLCRFCNVEPPAASLRAGARLSLGVLAAFVLAALLGARPERWAAAAAGLR |
| | TLLSACSLSLSPLFSIACAFFFLTCFLTRAQRGPGRGAGSWWLLALPACCYLGDFAAW |
| | QWWSWLRGEPAAAGRLCLVLSCVGLLTLAPRVRLRHGVLVLLFAGLVWWVSFSGLGAL |
| | PPALRPLLSCLVGGAGCLLALGLDHFFHVRGASPPPRSASTAEEKVPVIRPRRRSSCV |
| | SLGESAAGYYGSGKMFRRPSLPCISREQMILWDWDLKQWCKPHYQNSGGGNGVDLSVL |
| | NEARNMVSDLLIDPSLPPQVISSLRSISSLMGAFSGSCRPKINSFTPFPGFYPCSEVE |
| | DPVEKGDRKLHKGLSGRTSFPTPQLRRSSGASSLLTNEHCSRWDRSSGKRSYQELSVS |
| | SHGCHLNGPFSSNLFTIPKQRSSSVSLTHHAGLRRAGALPSHSLLNSSSHVPVSAGSL |
| | TNRSPIGFPDTTDFLTKPNIILHRSLGSVSSAADFHQYLRNSDSNLCSSCGHQILKYV |
| | STCEPDGTDHPSEKSGEEDSSVFSKEPLNIVETQEEETMKKACRELFLEGDSHLMEEA |
| | QQPNIDQEVSLDPMLVEDYDSLIEKMNNWNFQIFELVEKMGEKSGRILSQVMYTLFQD |
| | TGLLETFKIPTQEFMNYFRALENGYRDIPYHNRVHATDVLHAVWYLTTRPIPGLPQIH |
| | NNHETETKADSDGRLGSGQIAYISSKSCCIPDMSYGCLSSNIPALELMALYVAAAMHD |
| | YDHPGRTNAFLVATNAPQAVLYNDRSVLENHHAASAWNLYLSRPEYNFLLNLDHME |
| | FKRFRFLVIEAILATDLKKHFDFLAEFNAKANDVNSNGIEWSSENDRLLVCQVCIKLADI |
| | NGPAKDRDLHLRWTEGIVNEFYEQGDEEATLGLPISPFMDRSSPQLAKLQESFITHIV |
| | GPLCNSYDAAGLLPGQWIETEEGDDTESDDDDDDDDGGGEELDSDDEETEDNLNPKP |
| | QRRKGRRRIFCQLMHHLTENHKIWKEIIEEEEEKCKAEGNKLQVDNASLPQADEIQVIE |
| | EADEEEEQMFE |
| 80 | AACUUGUAGAAAAGAUGGGAA |
| 81 | ACCACAAGAUAUGGAAGGAAA |
| 82 | ACUAGCAAAACUCCAAGAAUA |
| 83 | ACUGGCUCUCUAACUAAUCGA |
| 84 | CAAAACUCCAAGAAUCUUUUA |
| 85 | CCCCGGAGGAGGUCCAGCUGA |
| 86 | CCUGCAUUAGAAUUGAUGGCA |
| 87 | CUAACUAAUCGAUCACCCAUA |
| 88 | CUCAACUAGCAAAACUCCAAA |
| 89 | CUCUCUAACUAAUCGAUCACA |
| 90 | CUGCAUUAGAAUUGAUGGCUA |
| 91 | CUGGCUCUCUAACUAAUCGAA |
| 92 | GAAGAAGAAAAAUGUAAAGCA |
| 93 | GUUUUAUACAAUGACAGAUCA |
| 94 | UAGCAAAACUCCAAGAAUCUA |
| 95 | UCUAACUAAUCGAUCACCCAA |
| 96 | UGGCUCUCUAACUAAUCGAUA |
| 97 | AAGAGUAUGACUCAUUAAUAA |
| 98 | AAUUUUCCAAUUUUUGAACUA |
| 99 | GAAUUUUCCAAUUUUUGAACA |
| 100 | GUUAUGUAUACCUUAUUUCAA |
| 101 | AAUUUUUGAACUUGUAGAAAA |
| 102 | UAUGUAUACCUUAUUUCAAGA |
| 103 | CAAGUCAAGGAUGCUAUCUAA |
| 104 | AAGAAUCAUUCAAACUUAUGA |
| 105 | AUUGCUUAUAUUUCUUCGAAA |
| 106 | GCUAUCGAGACAUUCCUUAUA |
| 107 | AACUCCAAGAAUCUUUUAUCA |
| 108 | UGGAAUUUUCCAAUUUUUGAA |
| 109 | GAAAGAAUCAUUCAAACUUAA |
| 110 | UUUAUUGGAAAUAUUUAAAAA |
| 111 | AAACUCCAAGAAUCUUUUAUA |
| 112 | GAAGAAGAAAACAUUUUCUCA |
| 113 | ACGGUGAAGAAUUAGAUACAA |
| 114 | AGGGACUAAAUAGGAAUAGUA |
| 115 | AGAAAUGGAAAACAAUCUAAA |
| 116 | AAGGUCAUCUUCUGUAUCACA |
| 117 | CAGUCCAUUCAUGGAUCGUUA |
| 118 | UUUUCCAAUUUUUGAACUUGA |
| 119 | CUCAACAAUUUAUGAACUAUA |
| 120 | CGAAUUGCUUAUAUUUCUUCA |
| 121 | GAAUUGCUUAUAUUUCUUCGA |
| 122 | CAAUUUUUGAACUUGUAGAAA |
| 123 | AGGAAGAAGAAAAAUGUAAAA |
| 124 | UGGUAUAAGCCUCAUUAUCAA |
| 125 | UUCCCAUCUUUUCUACAAGUUCA |
| 126 | UUUCCUUCCAUAUCUUGUGGUUU |
| 127 | UAUUCUUGGAGUUUUGCUAGUUG |
| 128 | UCGAUUAGUUAGAGAGCCAGUAG |
| 129 | UAAAAGAUUCUUGGAGUUUUGCU |
| 130 | UCAGCUGGACCUCCUCCGGGGUC |
| 131 | UGCCAUCAAUUCUAAUGCAGGAA |
| 132 | UAUGGGUGAUCGAUUAGUUAGAG |
| 133 | UUUGGAGUUUUGCUAGUUGAGGA |
| 134 | UGUGAUCGAUUAGUUAGAGAGCC |
| 135 | UAGCCAUCAAUUCUAAUGCAGGA |
| 136 | UUCGAUUAGUUAGAGAGCCAGUA |
| 137 | UGCUUUACAUUUUUCUUCUUCCU |
| 138 | UGAUCUGUCAUUGUAUAAAACUG |
| 139 | UAGAUUCUUGGAGUUUUGCUAGU |
| 140 | UUGGGUGAUCGAUUAGUUAGAGA |
| 141 | UAUCGAUUAGUUAGAGAGCCAGU |
| 142 | UUAUUAAUGAGUCAUACUCUUCU |
| 143 | UAGUUCAAAAAUUGGAAAAUUCC |
| 144 | UGUUCAAAAAUUGGAAAAUUCCA |
| 145 | UUGAAAUAAGGUAUACAUAACCU |
| 146 | UUUUCUACAAGUUCAAAAAUUGG |
| 147 | UCUUGAAAUAAGGUAUACAUAAC |
| 148 | UUAGAUAGCAUCCUUGACUUGAA |
| 149 | UCAUAAGUUUGAAUGAUUCUUUC |
| 150 | UUUCGAAGAAAUAUAAGCAAUUC |
| 151 | UAUAAGGAAUGUCUCGAUAGCCA |
| 152 | UGAUAAAAGAUUCUUGGAGUUUU |
| 153 | UUCAAAAAUUGGAAAAUUCCAGU |
| 154 | UUAAGUUUGAAUGAUUCUUUCGA |
| 155 | UUUUUAAAUAUUUCCAAUAAACC |
| 156 | UAUAAAAGAUUCUUGGAGUUUUG |
| 157 | UGAGAAAAUGUUUUCUUCUUCAC |
| 158 | UUGUAUCUAAUUCUUCACCGUCU |
| 159 | UACUAUUCCUAUUUAGUCCCUUG |
| 160 | UUUAGAUUGUUUUCCAUUUCUUC |
| 161 | UGUGAUACAGAAGAUGACCUUUG |
| 162 | UAACGAUCCAUGAAUGGACUGAU |
| 163 | UCAAGUUCAAAAAUUGGAAAAUU |
| 164 | UAUAGUUCAUAAAUUGUUGAGUG |
| 165 | UGAAGAAAUAUAAGCAAUUCGCC |
| 166 | UCGAAGAAAUAUAAGCAAUUCGC |
| 167 | UUUCUACAAGUUCAAAAAUUGGA |
| 168 | UUUUACAUUUUUCUUCUUCCUCU |
| 169 | UUGAUAAUGAGGCUUAUACCAUU |
| 170 | mA*mA*mCmUmUmGmUmAfGfAfAmAmAmGmAmUmGmGmG*mA*mA |
| 171 | mA*mC*mCmAmCmAmAmGfAfUfAmUmGmGmAmAmGmGmA*mA*mA |
| 172 | mA*mC*mUmAmGmCmAmAfAfAfCmUmCmCmAmAmGmAmA*mU*mA |
| 173 | mA*mC*mUmGmGmCmUmCfUfCfUmAmAmCmUmAmAmUmC*mG*mA |
| 174 | mC*mA*mAmAmAmCmUmCfCfAfAmGmAmAmUmCmUmUmU*mU*mA |
| 175 | mC*mC*mCmCmGmGmAmGfGfAfGmGmUmCmCmAmGmCmU*mG*mA |
| 176 | mC*mC*mUmGmCmAmUmUfAfGfAmAmUmUmGmAmUmGmG*mC*mA |
| 177 | mC*mU*mAmAmCmUmAmAfUfCfGmAmUmCmAmCmCmCmA*mU*mA |
| 178 | mC*mU*mCmAmAmCmUmAfGfCfAmAmAmAmCmUmCmCmA*mA*mA |
| 179 | mC*mU*mCmUmCmUmAmAfCfUfAmAmUmCmGmAmUmCmA*mC*mA |
| 180 | mC*mU*mGmCmAmUmUmAfGfAfAmUmUmGmAmUmGmGmC*mU*mA |
| 181 | mC*mU*mGmGmCmUmCmUfCfUfAmAmCmUmAmAmUmCmG*mA*mA |
| 182 | mG*mA*mAmGmAmAmGmAfAfAfAmAmUmGmUmAmAmAmG*mC*mA |
| 183 | mG*mU*mUmUmUmAmUmAfCfAfAmUmGmAmCmAmGmAmU*mC*mA |
| 184 | mU*mA*mGmCmAmAmAmAfCfUfCmCmAmAmGmAmAmUmC*mU*mA |
| 185 | mU*mC*mUmAmAmCmUmAfAfUfCmGmAmUmCmAmCmCmC*mA*mA |
| 186 | mU*mG*mGmCmUmCmUmCfUfAfAmCmUmAmAmUmCmGmA*mU*mA |
| 187 | mA*mA*mGmAmGmUmAmUfGfAfCmUmCmAmUmUmAmAmU*mA*mA |
| 188 | mA*mA*mUmUmUmUmCmCfAfAfUmUmUmUmUmGmAmAmC*mU*mA |
| 189 | mG*mA*mAmUmUmUmUmCfCfAfAmUmUmUmUmUmGmAmA*mC*mA |
| 190 | mG*mU*mUmAmUmGmUmAfUfAfCmCmUmUmAmUmUmUmC*mA*mA |
| 191 | mA*mA*mUmUmUmUmUmGfAfAfCmUmUmGmUmAmGmAmA*mA*mA |
| 192 | mU*mA*mUmGmUmAmUmAfCfCfUmUmAmUmUmUmCmAmA*mG*mA |
| 193 | mC*mA*mAmGmUmCmAmAfGfGfAmUmGmCmUmAmUmCmU*mA*mA |
| 194 | mA*mA*mGmAmAmUmCmAfUfUfCmAmAmAmCmUmUmAmU*mG*mA |
| 195 | mA*mUmUmGmCmUmUmAfUfAfUmUmUmCmUmUmCmGmA*mA*mA |
| 196 | mG*mC*mUmAmUmCmGmAfGfAfCmAmUmUmCmCmUmUmA*mU*mA |
| 197 | mA*mA*mCmUmCmCmAmAfGfAfAmUmCmUmUmUmUmAmU*mC*mA |
| 198 | mU*mG*mGmAmAmUmUmUfUfCfCmAmAmUmUmUmUmUmG*mA*mA |
| 199 | mG*mA*mAmAmGmAmAmUfCfAfUmUmCmAmAmAmCmUmU*mA*mA |
| 200 | mU*mU*mUmAmUmUmGmGfAfAfAmUmAmUmUmUmAmAmA*mA*mA |
| 201 | mA*mA*mAmCmUmCmCmAfAfGfAmAmUmCmUmUmUmUmA*mU*mA |
| 202 | mG*mA*mAmGmAmAmGmAfAfAfAmCmAmUmUmUmUmCmU*mC*mA |
| 203 | mA*mC*mGmGmUmGmAmAfGfAfAmUmUmAmGmAmUmAmC*mA*mA |
| 204 | mA*mG*mGmGmAmCmUmAfAfAfUmAmGmGmAmAmUmAmG*mU*mA |
| 205 | mA*mG*mAmAmAmUmGmGfAfAfAmAmCmAmAmUmCmUmA*mA*mA |
| 206 | mA*mA*mGmGmUmCmAmUfCfUfUmCmUmGmUmAmUmCmA*mC*mA |
| 207 | mC*mA*mGmUmCmCmAmUfUfCfAmUmGmGmAmUmCmGmU*mU*mA |
| 208 | mU*mU*mUmUmCmCmAmAfUfUfUmUmUmGmAmAmCmUmU*mG*mA |
| 209 | mC*mU*mCmAmAmCmAmAfUfUfUmAmUmGmAmAmCmUmA*mU*mA |
| 210 | mC*mG*mAmAmUmUmGmCfUfUfAmUmAmUmUmUmCmUmU*mC*mA |
| 211 | mG*mA*mAmUmUmGmCmUfUfAfUmAmUmUmUmCmUmUmC*mG*mA |
| 212 | mC*mA*mAmUmUmUmUmUfGfAfAmCmUmUmGmUmAmGmA*mA*mA |
| 213 | mA*mG*mGmAmAmGmAmAfGfAfAmAmAmAmUmGmUmAmA*mA*mA |
| 214 | mU*mG*mGmUmAmUmAmAfGfCfCmUmCmAmUmUmAmUmC*mA*mA |
| 215 | mU*mG*mGmCmU[Chd]mUmCfUfAfAmCmUmAmAmUmCmGmA*mU*mA |
| 216 | mG*mA*mAmGmA[Ahd]mGmAfAfAfAmAmUmGmUmAmAmAmG*mC*mA |
| 217 | mA*mA*mCmUmU[Ghd]mUmAfGfAfAmAmAmGmAmUmGmGmG*mA*mA |
| 218 | mC*mU*mGmCmA[Uhd]mUmAfGfAfAmUmUmGmAmUmGmGmC*mU*mA |
| 219 | mC*mU*mCmUmC[Uhd]mAmAfCfUfAmAmUmCmGmAmUmCmA*mC*mA |
| 220 | mA*mC*mUmGmG[Chd]mUmCfUfCfUmAmAmCmUmAmAmUmC*mG*mA |
| 221 | mU*mC*mUmAmA[Chd]mUmAfAfUfCmGmAmUmCmAmCmCmC*mA*mA |
| 222 | mC*mU*mAmAmC[Uhd]mAmAfUfCfGmAmUmCmAmCmCmCmA*mU*mA |
| 223 | mC*mC*mUmGmC[Ahd]mUmUfAfGfAmAmUmUmGmAmUmGmG*mC*mA |
| 224 | mU*fU*mCmCfCmAfUmCmUmUmUmUmCfUmAfCmAmAmGmUmU*mC*mA |
| 225 | mU*fU*mUmCfCmUfUmCmCmAmUmAmUfCmUfUmGmUmGmGmU*mU*mU |
| 226 | mU*fA*mUmUfCmUfUmGmGmAmGmUmUfUmUfGmCmUmAmGmU*mU*mG |
| 227 | mU*fC*mGmAfUmUfAmGmUmUmAmGmAfGmAfGmCmCmAmGmU*mA*mG |
| 228 | mU*fA*mAmAfAmGfAmUmUmCmUmUmGfGmAfGmUmUmUmUmG*mC*mU |
| 229 | mU*fC*mAmGfCmUfGmGmAmCmCmUmCfCmUfCmCmGmGmGmG*mU*mC |
| 230 | mU*fG*mCmCfAmUfCmAmAmUmUmCmUfAmAfUmGmCmAmGmG*mA*mA |
| 231 | mU*fA*mUmGfGmGfUmGmAmUmCmGmAfUmUfAmGmUmUmAmG*mA*mG |
| 232 | mU*fU*mUmGfGmAfGmUmUmUmUmGmCfUmAfGmUmUmGmAmG*mG*mA |
| 233 | mU*fG*mUmGfAmUfCmGmAmUmUmAmGfUmUfAmGmAmGmAmG*mC*mC |
| 234 | mU*fA*mGmCfCmAfUmCmAmAmUmUmCfUmAfAmUmGmCmAmG*mG*mA |
| 235 | mU*fU*mCmGfAmUfUmAmGmUmUmAmGfAmGfAmGmCmCmAmG*mU*mA |
| 236 | mU*fG*mCmUfUmUfAmCmAmUmUmUmUfUmCfUmUmCmUmUmC*mC*mU |
| 237 | mU*fG*mAmUfCmUfGmUmCmAmUmUmGfUmAfUmAmAmAmAmC*mU*mG |
| 238 | mU*fA*mGmAfUmUfCmUmUmGmGmAmGfUmUfUmUmGmCmUmA*mG*mU |
| 239 | mU*fU*mGmGfGmUfGmAmUmCmGmAmUfUmAfGmUmUmAmGmA*mG*mA |
| 240 | mU*fA*mUmCfGmAfUmUmAmGmUmUmAfGmAfGmAmGmCmCmA*mG*mU |
| 241 | mU*fU*mAmUfUmAfAmUmGmAmGmUmCfAmUfAmCmUmCmUmU*mC*mU |
| 242 | mU*fA*mGmUfUmCfAmAmAmAmAmUmUfGmGfAmAmAmAmUmU*mC*mC |
| 243 | mU*fG*mUmUfCmAfAmAmAmAmUmUmGfGmAfAmAmAmUmUmC*mC*mA |
| 244 | mU*fU*mGmAfAmAfUmAmAmGmGmUmAfUmAfCmAmUmAmAmC*mC*mU |
| 245 | mU*fU*mUmUfCmUfAmCmAmAmGmUmUfCmAfAmAmAmAmUmU*mG*mG |
| 246 | mU*fC*mUmUfGmAfAmAmUmAmAmGmGfUmAfUmAmCmAmUmA*mA*mC |
| 247 | mU*fU*mAmGfAmUfAmGmCmAmUmCmCfUmUfGmAmCmUmUmG*mA*mA |
| 248 | mU*fC*mAmUfAmAfGmUmUmUmGmAmAfUmGfAmUmUmCmUmU*mU*mC |
| 249 | mU*fU*mUmCfGmAfAmGmAmAmAmUmAfUmAfAmGmCmAmAmU*mU*mC |
| 250 | mU*fA*mUmAfAmGfGmAmAmUmGmUmCfUmCfGmAmUmAmGmC*mC*mA |
| 251 | mU*fG*mAmUfAmAfAmAmGmAmUmUmCfUmUfGmGmAmGmUmU*mU*mU |
| 252 | mU*fU*mCmAfAmAfAmAmUmUmGmGmAfAmAfAmUmUmCmCmA*mG*mU |
| 253 | mU*fU*mAmAfGmUfUmUmGmAmAmUmGfAmUfUmCmUmUmUmC*mG*mA |
| 254 | mU*fU*mUmUfUmAfAmAmUmAmUmUmUfCmCfAmAmUmAmAmA*mC*mC |
| 255 | mU*fA*mUmAfAmAfAmGmAmUmUmCmUfUmGfGmAmGmUmUmU*mU*mG |
| 256 | mU*fG*mAmGfAmAfAmAmUmGmUmUmUfUmCfUmUmCmUmUmC*mA*mC |
| 257 | mU*fU*mGmUfAmUfCmUmAmAmUmUmCfUmUfCmAmCmCmGmU*mC*mU |
| 258 | mU*fA*mCmUfAmUfUmCmCmUmAmUmUfUmAfGmUmCmCmCmU*mU*mG |
| 259 | mU*fU*mUmAfGmAfUmUmGmUmUmUmUfCmCfAmUmUmUmCmU*mU*mC |
| 260 | mU*fG*mUmGfAmUfAmCmAmGmAmAmGfAmUfGmAmCmCmUmU*mU*mG |
| 261 | mU*fA*mAmCfGmAfUmCmCmAmUmGmAfAmUfGmGmAmCmUmG*mA*mU |
| 262 | mU*fC*mAmAfGmUfUmCmAmAmAmAmAfUmUfGmGmAmAmAmA*mU*mU |
| 263 | mU*fA*mUmAfGmUfUmCmAmUmAmAmAfUmUfGmUmUmGmAmG*mU*mG |
| 264 | mU*fG*mAmAfGmAfAmAmUmAmUmAmAfGmCfAmAmUmUmCmG*mC*mC |
| 265 | mU*fC*mGmAfAmGfAmAmAmUmAmUmAfAmGfCmAmAmUmUmC*mG*mC |
| 266 | mU*fU*mUmCfUmAfCmAmAmGmUmUmCfAmAfAmAmAmUmUmG*mG*mA |
| 267 | mU*fU*mUmUfAmCfAmUmUmUmUmUmCfUmUfCmUmUmCmCmU*mC*mU |
| 268 | mU*fU*mGmAfUmAfAmUmGmAmGmGmCfUmUfAmUmAmCmCmA*mU*mU |
| 269 | H-Aib-EGTFTSDVSSYLEGQAAK((2-[2-(2-Amino-ethoxy)-ethoxy]- |
| | acetyl)₂-(γGlu)-CO—(CH₂)₁₆—CO₂H)EFIAWLVRGRG |
| 270 | GGGGEGGGGEGGGGEKEKEKGGGGSGGGGS-fSp-Cha-aGPIDRI-NH₂ |
| 271 | CUAUGGCAGUUGCAAAAUAUA |
| 272 | UAUGGCAGUUGCAAAAUAUUA |
| 273 | AUGGCAGUUGCAAAAUAUUCA |
| 274 | GGCAGUUGCAAAAUAUUCAGA |
| 275 | GCAGUUGCAAAAUAUUCAGGA |
| 276 | CAAUGGUAUAAGCCUCAUUAA |
| 277 | AUGGUAUAAGCCUCAUUAUCA |
| 278 | GUAUAAGCCUCAUUAUCAAAA |
| 279 | UAUAAGCCUCAUUAUCAAAAA |
| 280 | AAGGGACUAAAUAGGAAUAGA |
| 281 | GGGACUAAAUAGGAAUAGUUA |
| 282 | AAAGGUCAUCUUCUGUAUCAA |
| 283 | UUCCUGAUACUGCUGAUUUUA |
| 284 | UGAAGAAGAAAACAUUUUCUA |
| 285 | UCUCGAAAGAAUCAUUCAAAA |
| 286 | CUCGAAAGAAUCAUUCAAACA |
| 287 | UCGAAAGAAUCAUUCAAACUA |
| 288 | UAGAAGAGUAUGACUCAUUAA |
| 289 | AGAGUAUGACUCAUUAAUAGA |
| 290 | UUGCUUAUAUUUCUUCGAAGA |
| 291 | UCCUUCUUCAUCUUGAUCAUA |
| 292 | AAAAGUUCGAGACUUGCAUUA |
| 293 | AAAGUUCGAGACUUGCAUUUA |
| 294 | UUCGAGACUUGCAUUUGAAAA |
| 295 | ACUCCAAGAAUCUUUUAUCAA |
| 296 | AAGACGGUGAAGAAUUAGAUA |
| 297 | GACGGUGAAGAAUUAGAUACA |
| 298 | AAUGGAAAACAAUCUAAAUCA |
| 299 | GGAAGAAGAAAAAUGUAAAGA |
| 300 | GGAAGAGUAUGACUCAUUAAUAA |
| 301 | CAGAGUAUGACUCAUUAAUAA |
| 302 | AGAGUAUGACUCAUUAAUAA |
| 303 | AAACUCCAAGAATCUUUUAUA |
| 304 | GGAAACUCCAAGAAUCUUUUAUA |
| 305 | CAACUCCAAGAAUCUUUUAUA |
| 306 | AACUCCAAGAAUCUUUUAUA |
| 307 | AAACUCCAAGAAUCUUUUAUG |
| 308 | CACUCCAAGAAUCUUUUAUCA |
| 309 | GGAACUCCAAGAAUCUUUUAUCA |
| 310 | AACUCCAAGAAUCUAUUAUCA |
| 311 | AACUCCAAGAAUCUUUUAUCATT |
| 312 | AACUCCAAGAAUCUUUUAUCG |
| 313 | CGGAAGAAGAAAAAUGUAAAA |
| 314 | GGAAGAAGAAAAAUGUAAAA |
| 315 | AGGAAGAAGAAAAAUGUAAAG |
| 316 | AGGAAGAAGGAAAAUGUAAAA |
| 317 | UUGAUAAUGAGGCUUAUACCAUU |
| 318 | UUAUUAAUGAGUCAUACUCUUGG |
| 319 | UAGUUCAAAAAUUGGAAAAUUGG |
| 320 | UCAUAAGUUUGAAUGAUUCUUGG |
| 321 | UGAUAAAAGAUUCUUGGAGUUGG |
| 322 | UUUAGAUUGUUUUCCAUUUCUGG |
| 323 | UUUUACAUUUUUCUUCUUCCUGG |
| 324 | UAUAUUUUGCAACUGCCAUAGUA |
| 325 | UAAUAUUUUGCAACUGCCAUAGU |
| 326 | UGAAUAUUUUGCAACUGCCAUAG |
| 327 | UCUGAAUAUUUUGCAACUGCCAU |
| 328 | UCCUGAAUAUUUUGCAACUGCCA |
| 329 | UUAAUGAGGCUUAUACCAUUGUU |
| 330 | UGAUAAUGAGGCUUAUACCAUUG |
| 331 | UUUUGAUAAUGAGGCUUAUACCA |
| 332 | UUUUUGAUAAUGAGGCUUAUACC |
| 333 | UCUAUUCCUAUUUAGUCCCUUGU |
| 334 | UAACUAUUCCUAUUUAGUCCCUU |
| 335 | UUGAUACAGAAGAUGACCUUUGC |
| 336 | UAAAAUCAGCAGUAUCAGGAAAU |
| 337 | UAGAAAAUGUUUUCUUCUUCACC |
| 338 | UUUUGAAUGAUUCUUUCGAGAAA |
| 339 | UGUUUGAAUGAUUCUUUCGAGAA |
| 340 | UAGUUUGAAUGAUUCUUUCGAGA |
| 341 | UUAAUGAGUCAUACUCUUCUACU |
| 342 | UCUAUUAAUGAGUCAUACUCUUC |
| 343 | UCUUCGAAGAAAUAUAAGCAAUU |
| 344 | UAUGAUCAAGAUGAAGAAGGAAG |
| 345 | UAAUGCAAGUCUCGAACUUUUGC |
| 346 | UAAAUGCAAGUCUCGAACUUUUG |
| 347 | UUUUCAAAUGCAAGUCUCGAACU |
| 348 | UUGAUAAAAGAUUCUUGGAGUUU |
| 349 | UAUCUAAUUCUUCACCGUCUUCA |
| 350 | UGUAUCUAAUUCUUCACCGUCUU |
| 351 | UGAUUUAGAUUGUUUUCCAUUUC |
| 352 | UCUUUACAUUUUUCUUCUUCCUC |
| 353 | UUAUUAAUGAGUCAUACUCUUUU |
| 354 | UUAUUAAUGAGUCAUACUCUUCC |
| 355 | UUAUUAAUGAGUCAUACUCUU |
| 356 | UUAUUAAUGAGUCAUACUCUGCU |
| 357 | UUAUUAAUGAGUCAUACUCUUU |
| 358 | UAUAAAAGAUUCUUGGAGUUUCC |
| 359 | UAUAAAAGAUUCUUGGAGUUU |
| 360 | UAUAAAAGAUUCUUGGAGUUUGG |
| 361 | UAUAAAAGAUUCUUGGAGUUGUG |
| 362 | UAUAAAAGAUUCUUGGAGUUUU |
| 363 | UGAUAAAAGAUUCUUGGAGUUCC |
| 364 | UGAUAAAAGAUUCUUGGAGUU |
| 365 | UGAUAAUAGAUUCUUGGAGUUUU |
| 366 | UGAUAAAAGAUUCUUGGAGUGUU |
| 367 | UUUUACAUUUUUCUUCUUCCGCU |
| 368 | UUUUACAUUUUUCUUCUUCCUU |
| 369 | iAb*mA*mAmGmAmGmUmAmUfGfAfCmUmCmAmUmUmAmAmU*mA*mA |
| 370 | iAb*mC*mAmAmGmUmCmAmAfGfGfAmUmGmCmUmAmUmCmU*mA*mA |
| 371 | iAb*mG*mAmAmAmGmAmAmUfCfAfUmUmCmAmAmAmCmUmU*mA*mA |
| 372 | iAb*mA*mAmGmAmAmUmCmAfUfUfCmAmAmAmCmUmUmAmU*mG*mA |
| 373 | iAb*mU*mAmUmGmUmAmUmAfCfCfUmUmAmUmUmUmCmAmA*mG*mA |
| 374 | iAb*mA*mUmUmGmCmUmUmAfUfAfUmUmUmCmUmUmCmGmA*mA*mA |
| 375 | iAb*mA*mAmAmCmUmCmCmAfAfGfAmAmUmCmUmUmUmUmA*mU*mA |
| 376 | iAb*mG*mAmAmGmAmAmGmAfAfAfAmCmAmUmUmUmUmCmU*mC*mA |
| 377 | iAb*mA*mGmGmAmAmGmAmAfGfAfAmAmAmAmUmGmUmAmA*mA*mA |
| 378 | iAb*mA*mAmCmUmCmCmAmAfGfAfAmUmCmUmUmUmUmAmU*mC*mA |
| 379 | iAb*mA*mAmUmUmUmUmUmGfAfAfCmUmUmGmUmAmGmAmA*mA*mA |
| 380 | iAb*mG*mCmUmAmUmCmGmAfGfAfCmAmUmUmCmCmUmUmA*mU*mA |
| 381 | iAb*mC*mUmAmAmCmUmAmAfUfCfGmAmUmCmAmCmCmCmA*mU*mA |
| 382 | mC*mU*mAmUmGmGmCmAfGfUfUmGmCmAmAmAmAmUmA*mU*mA |
| 383 | mU*mA*mUmGmGmCmAmGfUfUfGmCmAmAmAmAmUmAmU*mU*mA |
| 384 | mA*mU*mGmGmCmAmGmUfUfGfCmAmAmAmAmUmAmUmU*mC*mA |
| 385 | mG*mG*mCmAmGmUmUmGfCfAfAmAmAmUmAmUmUmCmA*mG*mA |
| 386 | mG*mC*mAmGmUmUmGmCfAfAfAmAmUmAmUmUmCmAmG*mG*mA |
| 387 | mC*mA*mAmUmGmGmUmAfUfAfAmGmCmCmUmCmAmUmU*mA*mA |
| 388 | mA*mU*mGmGmUmAmUmAfAfGfCmCmUmCmAmUmUmAmU*mC*mA |
| 389 | mG*mU*mAmUmAmAmGmCfCfUfCmAmUmUmAmUmCmAmA*mA*mA |
| 390 | mU*mA*mUmAmAmGmCmCfUfCfAmUmUmAmUmCmAmAmA*mA*mA |
| 391 | mA*mA*mGmGmGmAmCmUfAfAfAmUmAmGmGmAmAmUmA*mG*mA |
| 392 | mG*mG*mGmAmCmUmAmAfAfUfAmGmGmAmAmUmAmGmU*mU*mA |
| 393 | mA*mA*mAmGmGmUmCmAfUfCfUmUmCmUmGmUmAmUmC*mA*mA |
| 394 | mU*mU*mCmCmUmGmAmUfAfCfUmGmCmUmGmAmUmUmU*mU*mA |
| 395 | mU*mG*mAmAmGmAmAmGfAfAfAmAmCmAmUmUmUmUmC*mU*mA |
| 396 | mU*mC*mUmCmGmAmAmAfGfAfAmUmCmAmUmUmCmAmA*mA*mA |
| 397 | mC*mU*mCmGmAmAmAmGfAfAfUmCmAmUmUmCmAmAmA*mC*mA |
| 398 | mUmC*mGmAmAmAmGmAfAfUfCmAmUmUmCmAmAmAmC*mU*mA |
| 399 | mU*mA*mGmAmAmGmAmGfUfAfUmGmAmCmUmCmAmUmU*mA*mA |
| 400 | mA*mG*mAmGmUmAmUmGfAfCfUmCmAmUmUmAmAmUmA*mG*mA |
| 401 | mU*mU*mGmCmUmUmAmUfAfUfUmUmCmUmUmCmGmAmA*mG*mA |
| 402 | mU*mC*mCmUmUmCmUmUfCfAfUmCmUmUmGmAmUmCmA*mU*mA |
| 403 | mA*mA*mAmAmGmUmUmCfGfAfGmAmCmUmUmGmCmAmU*mU*mA |
| 404 | mA*mA*mAmGmUmUmCmGfAfGfAmCmUmUmGmCmAmUmU*mU*mA |
| 405 | mU*mU*mCmGmAmGmAmCfUfUfGmCmAmUmUmUmGmAmA*mA*mA |
| 406 | mA*mC*mUmCmCmAmAmGfAfAfUmCmUmUmUmUmAmUmC*mA*mA |
| 407 | mA*mA*mGmAmCmGmGmUfGfAfAmGmAmAmUmUmAmGmA*mU*mA |
| 408 | mG*mA*mCmGmGmUmGmAfAfGfAmAmUmUmAmGmAmUmA*mC*mA |
| 409 | mA*mA*mUmGmGmAmAmAfAfCfAmAmUmCmUmAmAmAmU*mC*mA |
| 410 | mG*mG*mAmAmGmAmAmGfAfAfAmAmAmUmGmUmAmAmA*mG*mA |
| 411 | iAb*mC*mUmAmUmGmGmCmAfGfUfUmGmCmAmAmAmAmUmA*mU*mA |
| 412 | iAb*mU*mAmUmGmGmCmAmGfUfUfGmCmAmAmAmAmUmAmU*mU*mA |
| 413 | iAb*mA*mUmGmGmCmAmGmUfUfGfCmAmAmAmAmUmAmUmU*mC*mA |
| 414 | iAb*mG*mGmCmAmGmUmUmGfCfAfAmAmAmUmAmUmUmCmA*mG*mA |
| 415 | iAb*mG*mCmAmGmUmUmGmCfAfAfAmAmUmAmUmUmCmAmG*mG*mA |
| 416 | iAb*mC*mAmAmUmGmGmUmAfUfAfAmGmCmCmUmCmAmUmU*mA*mA |
| 417 | iAb*mA*mUmGmGmUmAmUmAfAfGfCmCmUmCmAmUmUmAmU*mC*mA |
| 418 | iAb*mG*mUmAmUmAmAmGmCfCfUfCmAmUmUmAmUmCmAmA*mA*mA |
| 419 | iAb*mU*mAmUmAmAmGmCmCfUfCfAmUmUmAmUmCmAmAmA*mA*mA |
| 420 | iAb*mA*mAmGmGmGmAmCmUfAfAfAmUmAmGmGmAmAmUmA*mG*mA |
| 421 | iAb*mG*mGmGmAmCmUmAmAfAfUfAmGmGmAmAmUmAmGmU*mU*mA |
| 422 | iAb*mA*mAmAmGmGmUmCmAfUfCfUmUmCmUmGmUmAmUmC*mA*mA |
| 423 | iAb*mU*mUmCmCmUmGmAmUfAfCfUmGmCmUmGmAmUmUmU*mU*mA |
| 424 | iAb*mU*mGmAmAmGmAmAmGfAfAfAmAmCmAmUmUmUmUmC*mU*mA |
| 425 | iAb*mU*mCmUmCmGmAmAmAfGfAfAmUmCmAmUmUmCmAmA*mA*mA |
| 426 | iAb*mC*mUmCmGmAmAmAmGfAfAfUmCmAmUmUmCmAmAmA*mC*mA |
| 427 | iAb*mU*mCmGmAmAmAmGmAfAfUfCmAmUmUmCmAmAmAmC*mU*mA |
| 428 | iAb*mU*mAmGmAmAmGmAmGfUfAfUmGmAmCmUmCmAmUmU*mA*mA |
| 429 | iAb*mA*mGmAmGmUmAmUmGfAfCfUmCmAmUmUmAmAmUmA*mG*mA |
| 430 | iAb*mU*mUmGmCmUmUmAmUfAfUfUmUmCmUmUmCmGmAmA*mG*mA |
| 431 | iAb*mU*mCmCmUmUmCmUmUfCfAfUmCmUmUmGmAmUmCmA*mU*mA |
| 432 | iAb*mA*mAmAmAmGmUmUmCfGfAfGmAmCmUmUmGmCmAmU*mU*mA |
| 433 | iAb*mA*mAmAmGmUmUmCmGfAfGfAmCmUmUmGmCmAmUmU*mU*mA |
| 434 | iAb*mU*mUmCmGmAmGmAmCfUfUfGmCmAmUmUmUmGmAmA*mA*mA |
| 435 | iAb*mA*mCmUmCmCmAmAmGfAfAfUmCmUmUmUmUmAmUmC*mA*mA |
| 436 | iAb*mA*mAmGmAmCmGmGmUfGfAfAmGmAmAmUmUmAmGmA*mU*mA |
| 437 | iAb*mG*mAmCmGmGmUmGmAfAfGfAmAmUmUmAmGmAmUmA*mC*mA |
| 438 | iAb*mA*mAmUmGmGmAmAmAfAfCfAmAmUmCmUmAmAmAmU*mC*mA |
| 439 | iAb*mG*mGmAmAmGmAmAmGfAfAfAmAmAmUmGmUmAmAmA*mG*mA |
| 440 | iAb*[GL]*[GL]mAmAmGmAmGmUmAmUfGfAfCmUmCmAmUmUmAmAmU*mA*mA |
| 441 | iAb*mC*mAmGmAmGmUmAmUfGfAfCmUmCmAmUmUmAmAmU*mA*mA |
| 442 | iAb*mA*mGmAmGmUmAmUfGfAfCmUmCmAmUmUmAmAmU*mA*mA |
| 443 | iAb*mA*[DAP]mGmAmGmUmAmUfGfAfCmUmCmAmUmUmA[DAP]mU*mA*mA |
| 444 | iAb*[AL]*[AL]mGmAmGmUmAmUfGfAfCmU[CL]mAmUmUmAmAmU*mA*mA |
| 445 | iAb*[DAP]*[DAP]mGmAmGmUmAmUfGfAfCmU[C5P-C]mAmUmUmAmAmU*mA*mA |
| 446 | iAb*[AL]*[AL]mAmCmUmCmCmAfAfGfAmA[TL]mCmUmUmUmUmA*mU*mA |
| 447 | iAb*[GL]*[GL]mAmAmAmCmUmCmCmAfAfGfAmAmUmCmUmUmUmUmA*mU*mA |
| 448 | iAb*mC*mAmAmCmUmCmCmAfAfGfAmAmUmCmUmUmUmUmA*mU*mA |
| 449 | iAb*eA*eAmAmCmUmCmCmAfAfGfAmAmUmCmUmUmUmUmA*mU*mA |
| 450 | iAb*mA*mAmCmUmCmCmAfAfGfAmAmUmCmUmUmUmUmA*mU*mA |
| 451 | iAb*mA*mAmAmCmUmCmCmAfAfGfAmAmUmCmUmUmUmUmA*mU*mG |
| 452 | iAb*[GL]*[GL]mAmAmCmUmCmCmAmAfGfAfAmUmCmUmUmUmUmAmU*mC*mA |
| 453 | iAb*mA*mAmCmUmCmCmAmAfGfAfAmUmCmUmAmUmUmAmU*mC*mA |
| 454 | iAb*mA*mAmCΨmCmCmAmAfGfAfAΨmCmUmUmUmUmAmU*mC*mA |
| 455 | iAb*mA*mAmCmUmCmCmAmAfGfAfAmUmCmUmUmUmUmAmU*mC*mG |
| 456 | iAb*mA*mAmCmUmCmCmAmAfGfAfAmUmCmUmUmUmUmAmU*mC*mAdTdT |
| 457 | iAb*mC*mAmCmUmCmCmAmAfGfAfAmUmCmUmUmUmUmAmU*mC*mA |
| 458 | iAb*[AL]*[GL]mGmAmAmGmAmAfGfAfAmA[AL]mAmUmGmUmAmA*mA*mA |
| 459 | iAb*mC*mGmGmAmAmGmAmAfGfAfAmAmAmAmUmGmUmAmA*mA*mA |
| 460 | iAb*eA*eGmGmAmAmGmAmAfGfAfAmAmAmAmUmGmUmAmA*mA*mA |
| 461 | iAb*mG*mGmAmAmGmAmAfGfAfAmAmAmAmUmGmUmAmA*mA*mA |
| 462 | iAb*mA*mGmGmAmAmGmAmAfGfAfAmAmAmAmUmGmUmAmA*mA*mG |
| 463 | iAb*mA*mGmGmAmAmGmAmAfGfGfAmAmAmAmUmGmUmAmA*mA*mA |
| 464 | mU*fU*mAmUfUmAfAmUmGmAmGmUmCfAmUfAmCmUmCmUmU*mG*mG |
| 465 | mU*fA*mGmUfUmCfAmAmAmAmAmUmUfGmGfAmAmAmAmUmU*mG*mG |
| 466 | mU*fC*mAmUfAmAfGmUmUmUmGmAmAfUmGfAmUmUmCmUmU*mG*mG |
| 467 | mU*fG*mAmUfAmAfAmAmGmAmUmUmCfUmUfGmGmAmGmUmU*mG*mG |
| 468 | mU*fU*mUmAfGmAfUmUmGmUmUmUmUfCmCfAmUmUmUmCmU*mG*mG |
| 469 | mU*fU*mUmUfAmCfAmUmUmUmUmUmCfUmUfCmUmUmCmCmU*mG*mG |
| 470 | mU*fU*mAmUfUmAfAmUmGmAmGmUmCfAmUfAmCmUmCmU*mU*mC*mU |
| 471 | mU*fU*mAmGfAmUfAmGmCmAmUmCmCfUmUfGmAmCmUmU*mG*mA*mA |
| 472 | mU*fU*mAmAfGmUfUmUmGmAmAmUmGfAmUfUmCmUmUmU*mC*mG*mA |
| 473 | mU*fC*mAmUfAmAfGmUmUmUmGmAmAfUmGfAmUmUmCmU*mU*mU*mC |
| 474 | mU*fC*mUmUfGmAfAmAmUmAmAmGmGfUmAfUmAmCmAmU*mA*mA*mC |
| 475 | mU*fU*mUmCfGmAfAmGmAmAmAmUmAfUmAfAmGmCmAmA*mU*mU*mC |
| 476 | mU*fA*mUmAfAmAfAmGmAmUmUmCmUfUmGfGmAmGmUmU*mU*mU*mG |
| 477 | mU*fG*mAmGfAmAfAmAmUmGmUmUmUfUmCfUmUmCmUmU*mC*mA*mC |
| 478 | mU*fU*mUmUfAmCfAmUmUmUmUmUmCfUmUfCmUmUmCmC*mU*mC*mU |
| 479 | mU*fG*mAmUfAmAfAmAmGmAmUmUmCfUmUfGmGmAmGmU*mU*mG*mG |
| 480 | mU*fG*mAmUfAmAfAmAmGmAmUmUmCfUmUfGmGmAmGmU*mU*mU*mU |
| 481 | mU*fU*mUmUfCmUfAmCmAmAmGmUmUfCmAfAmAmAmAmU*mU*mG*mG |
| 482 | mU*fA*mUmAfAmGfGmAmAmUmGmUmCfUmCfGmAmUmAmG*mC*mC*mA |
| 483 | mU*fA*mUmGfGmGfUmGmAmUmCmGmAfUmUfAmGmUmUmA*mG*mA*mG |
| 484 | mU*fA*mUmAfUmUfUmUmGmCmAmAmCfUmGfCmCmAmUmAmG*mU*mA |
| 485 | mU*fA*mAmUfAmUfUmUmUmGmCmAmAfCmUfGmCmCmAmUmA*mG*mU |
| 486 | mU*fG*mAmAfUmAfUmUmUmUmGmCmAfAmCfUmGmCmCmAmU*mA*mG |
| 487 | mU*fC*mUmGfAmAfUmAmUmUmUmUmGfCmAfAmCmUmGmCmC*mA*mU |
| 488 | mU*fC*mCmUfGmAfAmUmAmUmUmUmUfGmCfAmAmCmUmGmC*mC*mA |
| 489 | mU*fU*mAmAfUmGfAmGmGmCmUmUmAfUmAfCmCmAmUmUmG*mU*mU |
| 490 | mU*fG*mAmUfAmAfUmGmAmGmGmCmUfUmAfUmAmCmCmAmU*mU*mG |
| 491 | mU*fU*mUmUfGmAfUmAmAmUmGmAmGfGmCfUmUmAmUmAmC*mC*mA |
| 492 | mU*fU*mUmUfUmGfAmUmAmAmUmGmAfGmGfCmUmUmAmUmA*mC*mC |
| 493 | mU*fC*mUmAfUmUfCmCmUmAmUmUmUfAmGfUmCmCmCmUmU*mG*mU |
| 494 | mU*fA*mAmCfUmAfUmUmCmCmUmAmUfUmUfAmGmUmCmCmC*mU*mU |
| 495 | mU*fU*mGmAfUmAfCmAmGmAmAmGmAfUmGfAmCmCmUmUmU*mG*mC |
| 496 | mU*fA*mAmAfAmUfCmAmGmCmAmGmUfAmUfCmAmGmGmAmA*mA*mU |
| 497 | mU*fA*mGmAfAmAfAmUmGmUmUmUmUfCmUfUmCmUmUmCmA*mC*mC |
| 498 | mU*fU*mUmUfGmAfAmUmGmAmUmUmCfUmUfUmCmGmAmGmA*mA*mA |
| 499 | mU*fG*mUmUfUmGfAmAmUmGmAmUmUfCmUfUmUmCmGmAmG*mA*mA |
| 500 | mU*fA*mGmUfUmUfGmAmAmUmGmAmUfUmCfUmUmUmCmGmA*mG*mA |
| 501 | mU*fU*mAmAfUmGfAmGmUmCmAmUmAfCmUfCmUmUmCmUmA*mC*mU |
| 502 | mU*fC*mUmAfUmUfAmAmUmGmAmGmUfCmAfUmAmCmUmCmU*mU*mC |
| 503 | mU*fC*mUmUfCmGfAmAmGmAmAmAmUfAmUfAmAmGmCmAmA*mU*mU |
| 504 | mU*fA*mUmGfAmUfCmAmAmGmAmUmGfAmAfGmAmAmGmGmA*mA*mG |
| 505 | mU*fA*mAmUfGmCfAmAmGmUmCmUmCfGmAfAmCmUmUmUmU*mG*mC |
| 506 | mU*fA*mAmAfUmGfCmAmAmGmUmCmUfCmGfAmAmCmUmUmU*mU*mG |
| 507 | mU*fU*mUmUfCmAfAmAmUmGmCmAmAfGmUfCmUmCmGmAmA*mC*mU |
| 508 | mU*fU*mGmAfUmAfAmAmAmGmAmUmUfCmUfUmGmGmAmGmU*mU*mU |
| 509 | mU*fA*mUmCfUmAfAmUmUmCmUmUmCfAmCfCmGmUmCmUmU*mC*mA |
| 510 | mU*fG*mUmAfUmCfUmAmAmUmUmCmUfUmCfAmCmCmGmUmC*mU*mU |
| 511 | mU*fG*mAmUfUmUfAmGmAmUmUmGmUfUmUfUmCmCmAmUmU*mU*mC |
| 512 | mU*fC*mUmUfUmAfCmAmUmUmUmUmUfCmUfUmCmUmUmCmC*mU*mC |
| 513 | mU*fA*mUmAfUmUfUmUmGmCmAmAmCfUmGfCmCmAmUmA*mG*mU*mA |
| 514 | mU*fA*mAmUfAmUfUmUmUmGmCmAmAfCmUfGmCmCmAmU*mA*mG*mU |
| 515 | mU*fG*mAmAfUmAfUmUmUmUmGmCmAfAmCfUmGmCmCmA*mU*mA*mG |
| 516 | mU*fC*mUmGfAmAfUmAmUmUmUmUmGfCmAfAmCmUmGmC*mC*mA*mU |
| 517 | mU*fC*mCmUfGmAfAmUmAmUmUmUmUfGmCfAmAmCmUmG*mC*mC*mA |
| 518 | mU*fU*mAmAfUmGfAmGmGmCmUmUmAfUmAfCmCmAmUmU*mG*mU*mU |
| 519 | mU*fG*mAmUfAmAfUmGmAmGmGmCmUfUmAfUmAmCmCmA*mU*mU*mG |
| 520 | mU*fU*mUmUfGmAfUmAmAmUmGmAmGfGmCfUmUmAmUmA*mC*mC*mA |
| 521 | mU*fU*mUmUfUmGfAmUmAmAmUmGmAfGmGfCmUmUmAmU*mA*mC*mC |
| 522 | mU*fC*mUmAfUmUfCmCmUmAmUmUmUfAmGfUmCmCmCmU*mU*mG*mU |
| 523 | mU*fA*mAmCfUmAfUmUmCmCmUmAmUfUmUfAmGmUmCmC*mC*mU*mU |
| 524 | mU*fU*mGmAfUmAfCmAmGmAmAmGmAfUmGfAmCmCmUmU*mU*mG*mC |
| 525 | mU*fA*mAmAfAmUfCmAmGmCmAmGmUfAmUfCmAmGmGmA*mA*mA*mU |
| 526 | mU*fA*mGmAfAmAfAmUmGmUmUmUmUfCmUfUmCmUmUmC*mA*mC*mC |
| 527 | mU*fU*mUmUfGmAfAmUmGmAmUmUmCfUmUfUmCmGmAmG*mA*mA*mA |
| 528 | mU*fG*mUmUfUmGfAmAmUmGmAmUmUfCmUfUmUmCmGmA*mG*mA*mA |
| 529 | mU*fA*mGmUfUmUfGmAmAmUmGmAmUfUmCfUmUmUmCmG*mA*mG*mA |
| 530 | mU*fU*mAmAfUmGfAmGmUmCmAmUmAfCmUfCmUmUmCmU*mA*mC*mU |
| 531 | mU*fC*mUmAfUmUfAmAmUmGmAmGmUfCmAfUmAmCmUmC*mU*mU*mC |
| 532 | mU*fC*mUmUfCmGfAmAmGmAmAmAmUfAmUfAmAmGmCmA*mA*mU*mU |
| 533 | mU*fA*mUmGfAmUfCmAmAmGmAmUmGfAmAfGmAmAmGmG*mA*mA*mG |
| 534 | mU*fA*mAmUfGmCfAmAmGmUmCmUmCfGmAfAmCmUmUmU*mU*mG*mC |
| 535 | mU*fA*mAmAfUmGfCmAmAmGmUmCmUfCmGfAmAmCmUmU*mU*mU*mG |
| 536 | mU*fU*mUmUfCmAfAmAmUmGmCmAmAfGmUfCmUmCmGmA*mA*mC*mU |
| 537 | mU*fU*mGmAfUmAfAmAmAmGmAmUmUfCmUfUmGmGmAmG*mU*mU*mU |
| 538 | mU*fA*mUmCfUmAfAmUmUmCmUmUmCfAmCfCmGmUmCmU*mU*mC*mA |
| 539 | mU*fG*mUmAfUmCfUmAmAmUmUmCmUfUmCfAmCmCmGmU*mC*mU*mU |
| 540 | mU*fG*mAmUfUmUfAmGmAmUmUmGmUfUmUfUmCmCmAmU*mU*mU*mC |
| 541 | mU*fC*mUmUfUmAfCmAmUmUmUmUmUfCmUfUmCmUmUmC*mC*mU*mC |
| 542 | mU*fU*mAmUfUmAfAmUmGmAmGmUmCfAmUfAmCmUmCmU*mU*PrON*PrON |
| 543 | mU*fU*mAmUfUmAfAmUmGmAmGmUmCfAmUfAmCmUmCmU*mU*mC*mC |
| 544 | mU*fU*mAmUfUmAfAmUmGmAmGmUmCfAmUfAmCmUmCmU*mU*mG*mG |
| 545 | mU*fU*mAmUfUmAfAmUmGmAmGmUmCfAmUfAmCmU*mC*PrON*PrON |
| 546 | mU*fU*mAmUfUmAfAmUmGmAmGmUmCfAmUfAmCmUmCmU*mG*mC*mU |
| 547 | mU*fU*mAmUfUmAfAmUmGmAmGmUmCfAmUfAmCmUmC*mU*PrON*PrON |
| 548 | mU*fA*mUmAfAmAfAmGmAmUmUmCmUfUmGfGmA[UNA-G]mUmU*mU*mU*mG |
| 549 | mU*fA*mUmAfAmAfAmGmAmUmU[UNA-C]mUfUmGfGmAmGmUmU*mU*mU*mG |
| 550 | mU*fA*mUmAfAmAfAmGmAmUmUmCmUfUmGfGmAmGmUmU*mU*mC*mC |
| 551 | mU*fA*mUmAfAmAfAmGmAmUmUmCmUfUmGfGmAmG*mU*PrON*PrON |
| 552 | mU*fA*mUmAfAmAfAmGmAmUmUmCmUfUmGfGmAmGmUmU*mU*mG*mG |
| 553 | mU*fA*mUmAfAmAfAmGmAmUmUmCmUfUmGfGmAmGmUmU*mU*PRON*mG |
| 554 | mU*fA*mUmAfAmAfAmGmAmUmUmCmUfUmGfGmAmGmUmU*mG*mU*mG |
| 555 | mU*fA*mUmAfAmAfAmGmAmUmUmCmUfUmGfGmAmGmU*mU*PrON*PrON |
| 556 | mU*fA*mUmAfAmAfAmGmAmUmUmCmUfUmGfGmAmGmU*mU*PrON*PrON |
| 557 | mU*fA*mUdAmAmAdAmGmAmUmUmCmUfUmGfGmAmGmUmU*mU*mU*mG |
| 558 | mU*fA*mUmAfAmAmAfGmAmUmUmCmUfUmGfGmAmGmUmU*mU*mU*mG |
| 559 | mU*fG*mAmUfAmAfAmAmGmAmUmUmCfUmUfGmGmAmGmU*mU*PrON*PrON |
| 560 | mU*fG*mAmUfAmAfAmAmGmAmUmUmCfUmUfGmGmAmGmU*mU*mC*mC |
| 561 | mU*fG*mAmUfAmAfAmAmGmAmUmUmCfUmUfGmGmA*mG*PrON*PrON |
| 562 | mU*fG*mAmUfAmAfUmAmGmAmUmUmCfUmUfGmGmAmGmU*mU*mU*mU |
| 563 | mU*fG*mAmUfAmAfAmAmGmAmUmUmCfUΨfGmGmAmGΨ*Ψ*mU*mU |
| 564 | mU*fG*mAΨfAmAfAmAmGmAΨΨmCfUmUfGmGmAmGmU*mU*mU*mU |
| 565 | mU*fG*mAmUfAmAfAmAmGmAΨΨmCfUmUfGmGmAmGmU*mU*mU*mU |
| 566 | mU*fG*mAmUfAmAfAmAmGmAmUmUmCfUmUfGmGmAmGmU*mG*mU*mU |
| 567 | mU*fU*mUmUfAmCfAmUmUmUmUmUmCfUmUfCmUmUmCmC*mG*mC*mU |
| 568 | mU*fU*mUmUfAmCfAmUmUmUmUmUmCfUmUfCmUmUmC*mC*PrON*PrON |
| 569 | VPmU*fG*mAmGfAmAfAmAmUmGmUmUmUfUmCfUmUmCmUmU*mC*mA*mC |
| 570 | VPmU*fU*mUmUfAmCfAmUmUmUmUmUmCfUmUfCmUmUmCmC*mU*mC*mU |
| 571 | VPmU*fG*mAmUfAmAfAmAmGmAmUmUmCfUmUfGmGmAmGmU*mU*mG*mG |
| 572 | VPmU*fA*mUmAfAmAfAmGmAmUmUmCmUfUmGfGmAmGmUmU*mU*mU*mG |
| 573 | VPmU*fU*mUmCfGmAfAmGmAmAmAmUmAfUmAfAmGmCmAmA*mU*mU*mC |
| 574 | VPmU*fC*mUmUfGmAfAmAmUmAmAmGmGfUmAfUmAmCmAmU*mA*mA*mC |
| 575 | VPmU*fC*mAmUfAmAfGmUmUmUmGmAmAfUmGfAmUmUmCmU*mU*mU*mC |
| 576 | VPmU*fU*mAmUfUmAfAmUmGmAmGmUmCfAmUfAmCmUmCmU*mU*mC*mU |
| 577 | iAb*mA*mGmGmAmAmGmAmAfGfAfAmAmAmAmU[Ghd]mUmAmA*mA*mA |
| 578 | iAb*mG*mAmAmGmAmAmGmAfAfAfAmCmAmUmU[Uhd]mUmCmU*mC*mA |
| 579 | iAb*mA*mAmCmUmCmCmAmAfGfAfAmUmCmUmU[Uhd]mUmAmU*mC*mA |
| 580 | iAb*mA*mAmAmCmUmCmCmAfAfGfAmAmUmCmU[Uhd]mUmUmA*mU*mA |
| 581 | iAb*mA*mUmUmGmCmUmUmAfUfAfUmUmUmCmU[Uhd]mCmGmA*mA*mA |
| 582 | iAb*mU*mAmUmGmUmAmUmAfCfCfUmUmAmUmU[Uhd]mCmAmA*mG*mA |
| 583 | iAb*mA*mAmGmAmAmUmCmAfUfUfCmAmAmAmC[Uhd]mUmAmU*mG*mA |
| 584 | iAb*mA*mAmGmAmGmUmAmUfGfAfCmUmCmAmU[Uhd]mAmAmU*mA*mA |

1. A PDE3B RNAi agent comprising Formula: 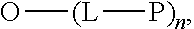
wherein O is a double stranded RNA (dsRNA) comprising a sense stand and an antisense strand, wherein the antisense strand is complementary to PDE3B mRNA;
wherein L is a linker or a bond;
wherein P is an NPR-C-binding peptide comprising SEQ ID NO: 1 (GX₁₁IDX₁₄I), wherein X₁₁ is arginine, proline, or hydroxyproline, and X₁₄ is arginine or N-methylarginine; and
wherein n is 1, 2, 3, or 4.

2. (canceled)

3. (canceled)

4. The PDE3B RNAi agent of claim 1, wherein P comprises one of SEQ ID NO: 2-7.

5.-13. (canceled)

14. The PDE3B RNAi agent of claim 1, wherein P comprises SEQ ID NO: 8 (X₇-Cha-X₉GX₁₁IDX₁₄I), wherein:
X₇ is proline, hydroxyproline, glycine, cysteine, or alanine;
X₉ is alanine, serine, or glycine;
X₁₁ is proline, hydroxyproline, or arginine; and
X₁₄ is arginine or N-methylarginine.

15. The PDE3B RNAi agent of claim 14, wherein P comprises SEQ ID NO: 9 (fSp-Cha-aGPIDRI).

16. The PDE3B RNAi agent of claim 1, wherein P comprises a sequence selected from any one of SEQ ID NOs: 11-57.

17. The PDE3B RNAi agent of claim 1, wherein P is a linear peptide.

18. The PDE3B RNAi agent of claim 1, wherein P is a cyclic peptide.

19. (canceled)

20. (canceled)

21. (canceled)

22. The PDE3B RNAi agent of claim 1, wherein P comprises a C-terminal hydroxyl.

23. The PDE3B RNAi agent of claim 1, wherein P comprises a C-terminal amide.

24. The PDE3B RNAi agent of claim 1, wherein L comprises a linker core and one or more spacers.

25. The PDE3B RNAi agent of claim 24, wherein L comprises Spacer1-Linker Core-Spacer2.

26. The PDE3B RNAi agent of claim 25, wherein the Linker Core is selected from Table 7.

27. The PDE3B RNAi agent of claim 26, wherein the Spacer1 and Spacer 2 are selected from Table 8.

28. The PDE3B RNAi agent of claim 1, wherein L is selected from Table 9.

29. The PDE3B RNAi agent of claim 1, wherein n is 1 or 2.

30. The PDE3B RNAi agent of claim 1, wherein L is attached to the 5′ end of the sense strand and to the N-terminal end of the peptide.

31. The PDE3B RNAi agent of claim 1, wherein L is attached to the 5′ end of the sense strand and to the C-terminal end of the peptide.

32. The PDE3B RNAi agent of claim 1, wherein L is attached to the 3′ end of the sense strand and to the N-terminal end of the peptide.

33. The PDE3B RNAi agent of claim 1, wherein L is attached to the 3′ end of the sense strand and to the C-terminal end of the peptide.

34. The PDE3B RNAi agent of claim 1, wherein L comprises the formula:

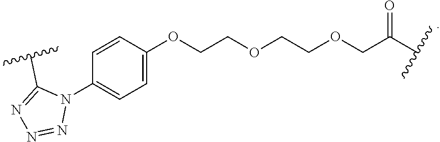 [SMILES: *C(=O)COCCOCCOc1ccc(-n2nnnc2*)cc1]

35. The PDE3B RNAi agent of claim 1, wherein L comprises the formula: 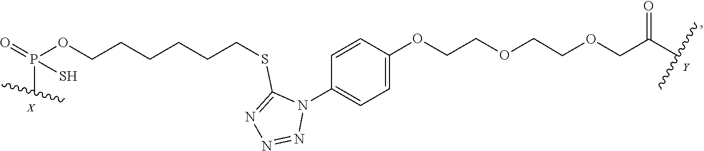
wherein X represents a position to which the 3′ end of the sense strand of O is conjugated, and
wherein Y represents a position to which an N-terminal end of P is conjugated.

36. The PDE3B RNAi agent of claim 1, wherein the conjugate further comprises a fatty acid (FA), wherein FA is attached to O or to P.

37. (canceled)

38. (canceled)

39. The PDE3B RNAi agent of claim 36, wherein the conjugate comprises (FA)m-O-L-P or O-L-P-(FA)ₘ, wherein m is an integer of 1 to 4.

40. The PDE3B RNAi agent of claim 36, wherein the conjugate further comprises a Spacer3, and wherein the PDE3B RNAi agent is of a formula selected from (FA-Spacer3)ₘ-O-L-P, O-L-P-(Spacer3-FA)ₘ, O-L-(FA-Spacer3)ₘ-P, and O-(Spacer3-FA)ₘ-L-P, wherein m is an integer of 1 to 4.

41. (canceled)

42. (canceled)

43. (canceled)

44. The PDE3B RNAi agent of claim 36, wherein the fatty acid is selected from the group consisting of FA1-FA27 of Table 10.

45. The PDE3B RNAi agent of claim 1, wherein the sense strand and the antisense strand form a duplex, and wherein the sense strand and antisense strand are selected from the group consisting of:
the sense strand comprises a first nucleic acid sequence of SEQ ID NO: 80, and the antisense strand comprises a second nucleic acid sequence of SEQ ID NO: 125;
the sense strand comprises a first nucleic acid sequence of SEQ ID NO: 81, and the antisense strand comprises a second nucleic acid sequence of SEQ ID NO: 126;
the sense strand comprises a first nucleic acid sequence of SEQ ID NO: 82, and the antisense strand comprises a second nucleic acid sequence of SEQ ID NO: 127;
the sense strand comprises a first nucleic acid sequence of SEQ ID NO: 83, and the antisense strand comprises a second nucleic acid sequence of SEQ ID NO: 128;
the sense strand comprises a first nucleic acid sequence of SEQ ID NO: 84, and the antisense strand comprises a second nucleic acid sequence of SEQ ID NO: 129;
the sense strand comprises a first nucleic acid sequence of SEQ ID NO: 85, and the antisense strand comprises a second nucleic acid sequence of SEQ ID NO: 130;
the sense strand comprises a first nucleic acid sequence of SEQ ID NO: 86, and the antisense strand comprises a second nucleic acid sequence of SEQ ID NO: 131; and
the sense strand comprises a first nucleic acid sequence of SEQ ID NO: 87, and the antisense strand comprises a second nucleic acid sequence of SEQ ID NO: 132;
the sense strand comprises a first nucleic acid sequence of SEQ ID NO: 88, and the antisense strand comprises a second nucleic acid sequence of SEQ ID NO: 133;
the sense strand comprises a first nucleic acid sequence of SEQ ID NO: 89, and the antisense strand comprises a second nucleic acid sequence of SEQ ID NO: 134;
the sense strand comprises a first nucleic acid sequence of SEQ ID NO: 90, and the antisense strand comprises a second nucleic acid sequence of SEQ ID NO: 135;
the sense strand comprises a first nucleic acid sequence of SEQ ID NO: 91, and the antisense strand comprises a second nucleic acid sequence of SEQ ID NO: 136;
the sense strand comprises a first nucleic acid sequence of SEQ ID NO: 92, and the antisense strand comprises a second nucleic acid sequence of SEQ ID NO: 137;
the sense strand comprises a first nucleic acid sequence of SEQ ID NO: 93, and the antisense strand comprises a second nucleic acid sequence of SEQ ID NO: 138;
the sense strand comprises a first nucleic acid sequence of SEQ ID NO: 94, and the antisense strand comprises a second nucleic acid sequence of SEQ ID NO: 139;
the sense strand comprises a first nucleic acid sequence of SEQ ID NO: 95, and the antisense strand comprises a second nucleic acid sequence of SEQ ID NO: 140;
the sense strand comprises a first nucleic acid sequence of SEQ ID NO: 96, and the antisense strand comprises a second nucleic acid sequence of SEQ ID NO: 141; and
the sense strand comprises a first nucleic acid sequence of SEQ ID NO: 97, and the antisense strand comprises a second nucleic acid sequence of SEQ ID NO: 142;
the sense strand comprises a first nucleic acid sequence of SEQ ID NO: 98, and the antisense strand comprises a second nucleic acid sequence of SEQ ID NO: 143;
the sense strand comprises a first nucleic acid sequence of SEQ ID NO: 99, and the antisense strand comprises a second nucleic acid sequence of SEQ ID NO: 144;
the sense strand comprises a first nucleic acid sequence of SEQ ID NO: 100, and the antisense strand comprises a second nucleic acid sequence of SEQ ID NO: 145;
the sense strand comprises a first nucleic acid sequence of SEQ ID NO: 101, and the antisense strand comprises a second nucleic acid sequence of SEQ ID NO: 146;
the sense strand comprises a first nucleic acid sequence of SEQ ID NO: 102, and the antisense strand comprises a second nucleic acid sequence of SEQ ID NO: 147;
the sense strand comprises a first nucleic acid sequence of SEQ ID NO: 103, and the antisense strand comprises a second nucleic acid sequence of SEQ ID NO: 148;
the sense strand comprises a first nucleic acid sequence of SEQ ID NO: 104, and the antisense strand comprises a second nucleic acid sequence of SEQ ID NO: 149;
the sense strand comprises a first nucleic acid sequence of SEQ ID NO: 105, and the antisense strand comprises a second nucleic acid sequence of SEQ ID NO: 150;
the sense strand comprises a first nucleic acid sequence of SEQ ID NO: 106, and the antisense strand comprises a second nucleic acid sequence of SEQ ID NO: 151; and
the sense strand comprises a first nucleic acid sequence of SEQ ID NO: 107, and the antisense strand comprises a second nucleic acid sequence of SEQ ID NO: 152;
the sense strand comprises a first nucleic acid sequence of SEQ ID NO: 108, and the antisense strand comprises a second nucleic acid sequence of SEQ ID NO: 153;
the sense strand comprises a first nucleic acid sequence of SEQ ID NO: 109, and the antisense strand comprises a second nucleic acid sequence of SEQ ID NO: 154;
the sense strand comprises a first nucleic acid sequence of SEQ ID NO: 110, and the antisense strand comprises a second nucleic acid sequence of SEQ ID NO: 155;
the sense strand comprises a first nucleic acid sequence of SEQ ID NO: 111, and the antisense strand comprises a second nucleic acid sequence of SEQ ID NO: 156;
the sense strand comprises a first nucleic acid sequence of SEQ ID NO: 112, and the antisense strand comprises a second nucleic acid sequence of SEQ ID NO: 157;
the sense strand comprises a first nucleic acid sequence of SEQ ID NO: 113, and the antisense strand comprises a second nucleic acid sequence of SEQ ID NO: 158;
the sense strand comprises a first nucleic acid sequence of SEQ ID NO: 114, and the antisense strand comprises a second nucleic acid sequence of SEQ ID NO: 159;
the sense strand comprises a first nucleic acid sequence of SEQ ID NO: 115, and the antisense strand comprises a second nucleic acid sequence of SEQ ID NO: 160;
the sense strand comprises a first nucleic acid sequence of SEQ ID NO: 116, and the antisense strand comprises a second nucleic acid sequence of SEQ ID NO: 161; and
the sense strand comprises a first nucleic acid sequence of SEQ ID NO: 117, and the antisense strand comprises a second nucleic acid sequence of SEQ ID NO: 162;
the sense strand comprises a first nucleic acid sequence of SEQ ID NO: 118, and the antisense strand comprises a second nucleic acid sequence of SEQ ID NO: 163;
the sense strand comprises a first nucleic acid sequence of SEQ ID NO: 119, and the antisense strand comprises a second nucleic acid sequence of SEQ ID NO: 164;
the sense strand comprises a first nucleic acid sequence of SEQ ID NO: 120, and the antisense strand comprises a second nucleic acid sequence of SEQ ID NO: 165;
the sense strand comprises a first nucleic acid sequence of SEQ ID NO: 121, and the antisense strand comprises a second nucleic acid sequence of SEQ ID NO: 166;
the sense strand comprises a first nucleic acid sequence of SEQ ID NO: 122, and the antisense strand comprises a second nucleic acid sequence of SEQ ID NO: 167;
the sense strand comprises a first nucleic acid sequence of SEQ ID NO: 123, and the antisense strand comprises a second nucleic acid sequence of SEQ ID NO: 168;
the sense strand comprises a first nucleic acid sequence of SEQ ID NO: 124, and the antisense strand comprises a second nucleic acid sequence of SEQ ID NO: 169,
the sense strand comprises a first nucleic acid sequence of SEQ ID NO: 124, and the antisense strand comprises a second nucleic acid sequence of SEQ ID NO: 317;
the sense strand comprises a first nucleic acid sequence of SEQ ID NO: 97, and the antisense strand comprises a second nucleic acid sequence of SEQ ID NO: 318;
the sense strand comprises a first nucleic acid sequence of SEQ ID NO: 98, and the antisense strand comprises a second nucleic acid sequence of SEQ ID NO: 319;
the sense strand comprises a first nucleic acid sequence of SEQ ID NO: 104, and the antisense strand comprises a second nucleic acid sequence of SEQ ID NO: 320;
the sense strand comprises a first nucleic acid sequence of SEQ ID NO: 107, and the antisense strand comprises a second nucleic acid sequence of SEQ ID NO: 321;
the sense strand comprises a first nucleic acid sequence of SEQ ID NO: 115, and the antisense strand comprises a second nucleic acid sequence of SEQ ID NO: 322;
the sense strand comprises a first nucleic acid sequence of SEQ ID NO: 123, and the antisense strand comprises a second nucleic acid sequence of SEQ ID NO: 323;
the sense strand comprises a first nucleic acid sequence of SEQ ID NO: 271, and the antisense strand comprises a second nucleic acid sequence of SEQ ID NO: 324;
the sense strand comprises a first nucleic acid sequence of SEQ ID NO: 272, and the antisense strand comprises a second nucleic acid sequence of SEQ ID NO: 325;
the sense strand comprises a first nucleic acid sequence of SEQ ID NO: 273, and the antisense strand comprises a second nucleic acid sequence of SEQ ID NO: 326;
the sense strand comprises a first nucleic acid sequence of SEQ ID NO: 274, and the antisense strand comprises a second nucleic acid sequence of SEQ ID NO: 327;
the sense strand comprises a first nucleic acid sequence of SEQ ID NO: 275, and the antisense strand comprises a second nucleic acid sequence of SEQ ID NO: 328;
the sense strand comprises a first nucleic acid sequence of SEQ ID NO: 276, and the antisense strand comprises a second nucleic acid sequence of SEQ ID NO: 329;
the sense strand comprises a first nucleic acid sequence of SEQ ID NO: 277, and the antisense strand comprises a second nucleic acid sequence of SEQ ID NO: 330;
the sense strand comprises a first nucleic acid sequence of SEQ ID NO: 278, and the antisense strand comprises a second nucleic acid sequence of SEQ ID NO: 331;
the sense strand comprises a first nucleic acid sequence of SEQ ID NO: 279, and the antisense strand comprises a second nucleic acid sequence of SEQ ID NO: 332;
the sense strand comprises a first nucleic acid sequence of SEQ ID NO: 280, and the antisense strand comprises a second nucleic acid sequence of SEQ ID NO: 333;
the sense strand comprises a first nucleic acid sequence of SEQ ID NO: 281, and the antisense strand comprises a second nucleic acid sequence of SEQ ID NO: 334;
the sense strand comprises a first nucleic acid sequence of SEQ ID NO: 282, and the antisense strand comprises a second nucleic acid sequence of SEQ ID NO: 335;
the sense strand comprises a first nucleic acid sequence of SEQ ID NO: 283, and the antisense strand comprises a second nucleic acid sequence of SEQ ID NO: 336;
the sense strand comprises a first nucleic acid sequence of SEQ ID NO: 284, and the antisense strand comprises a second nucleic acid sequence of SEQ ID NO: 337;
the sense strand comprises a first nucleic acid sequence of SEQ ID NO: 285, and the antisense strand comprises a second nucleic acid sequence of SEQ ID NO: 338;
the sense strand comprises a first nucleic acid sequence of SEQ ID NO: 286, and the antisense strand comprises a second nucleic acid sequence of SEQ ID NO: 339;
the sense strand comprises a first nucleic acid sequence of SEQ ID NO: 287, and the antisense strand comprises a second nucleic acid sequence of SEQ ID NO: 340;
the sense strand comprises a first nucleic acid sequence of SEQ ID NO: 288, and the antisense strand comprises a second nucleic acid sequence of SEQ ID NO: 341;
the sense strand comprises a first nucleic acid sequence of SEQ ID NO: 289, and the antisense strand comprises a second nucleic acid sequence of SEQ ID NO: 342;
the sense strand comprises a first nucleic acid sequence of SEQ ID NO: 290, and the antisense strand comprises a second nucleic acid sequence of SEQ ID NO: 343;
the sense strand comprises a first nucleic acid sequence of SEQ ID NO: 291, and the antisense strand comprises a second nucleic acid sequence of SEQ ID NO: 344;
the sense strand comprises a first nucleic acid sequence of SEQ ID NO: 292, and the antisense strand comprises a second nucleic acid sequence of SEQ ID NO: 345;
the sense strand comprises a first nucleic acid sequence of SEQ ID NO: 293, and the antisense strand comprises a second nucleic acid sequence of SEQ ID NO: 346;
the sense strand comprises a first nucleic acid sequence of SEQ ID NO: 294, and the antisense strand comprises a second nucleic acid sequence of SEQ ID NO: 347;
the sense strand comprises a first nucleic acid sequence of SEQ ID NO: 295, and the antisense strand comprises a second nucleic acid sequence of SEQ ID NO: 348;
the sense strand comprises a first nucleic acid sequence of SEQ ID NO: 296, and the antisense strand comprises a second nucleic acid sequence of SEQ ID NO: 349;
the sense strand comprises a first nucleic acid sequence of SEQ ID NO: 297, and the antisense strand comprises a second nucleic acid sequence of SEQ ID NO: 350;
the sense strand comprises a first nucleic acid sequence of SEQ ID NO: 298, and the antisense strand comprises a second nucleic acid sequence of SEQ ID NO: 351;
the sense strand comprises a first nucleic acid sequence of SEQ ID NO: 299, and the antisense strand comprises a second nucleic acid sequence of SEQ ID NO: 352;
the sense strand comprises a first nucleic acid sequence of SEQ ID NO: 97, and the antisense strand comprises a second nucleic acid sequence of SEQ ID NO: 353;
the sense strand comprises a first nucleic acid sequence of SEQ ID NO: 300, and the antisense strand comprises a second nucleic acid sequence of SEQ ID NO: 354;
the sense strand comprises a first nucleic acid sequence of SEQ ID NO: 97, and the antisense strand comprises a second nucleic acid sequence of SEQ ID NO: 355;
the sense strand comprises a first nucleic acid sequence of SEQ ID NO: 301, and the antisense strand comprises a second nucleic acid sequence of SEQ ID NO: 356;
the sense strand comprises a first nucleic acid sequence of SEQ ID NO: 302, and the antisense strand comprises a second nucleic acid sequence of SEQ ID NO: 357;
the sense strand comprises a first nucleic acid sequence of SEQ ID NO: 303, and the antisense strand comprises a second nucleic acid sequence of SEQ ID NO: 156;
the sense strand comprises a first nucleic acid sequence of SEQ ID NO: 304, and the antisense strand comprises a second nucleic acid sequence of SEQ ID NO: 358;
the sense strand comprises a first nucleic acid sequence of SEQ ID NO: 111, and the antisense strand comprises a second nucleic acid sequence of SEQ ID NO: 359;
the sense strand comprises a first nucleic acid sequence of SEQ ID NO: 111, and the antisense strand comprises a second nucleic acid sequence of SEQ ID NO: 360;
the sense strand comprises a first nucleic acid sequence of SEQ ID NO: 305, and the antisense strand comprises a second nucleic acid sequence of SEQ ID NO: 361;
the sense strand comprises a first nucleic acid sequence of SEQ ID NO: 111, and the antisense strand comprises a second nucleic acid sequence of SEQ ID NO: 362;
the sense strand comprises a first nucleic acid sequence of SEQ ID NO: 306, and the antisense strand comprises a second nucleic acid sequence of SEQ ID NO: 362;
the sense strand comprises a first nucleic acid sequence of SEQ ID NO: 307, and the antisense strand comprises a second nucleic acid sequence of SEQ ID NO: 156;
the sense strand comprises a first nucleic acid sequence of SEQ ID NO: 308, and the antisense strand comprises a second nucleic acid sequence of SEQ ID NO: 363;
the sense strand comprises a first nucleic acid sequence of SEQ ID NO: 107, and the antisense strand comprises a second nucleic acid sequence of SEQ ID NO: 364;
the sense strand comprises a first nucleic acid sequence of SEQ ID NO: 309, and the antisense strand comprises a second nucleic acid sequence of SEQ ID NO: 365;
the sense strand comprises a first nucleic acid sequence of SEQ ID NO: 310, and the antisense strand comprises a second nucleic acid sequence of SEQ ID NO: 152;
the sense strand comprises a first nucleic acid sequence of SEQ ID NO: 311, and the antisense strand comprises a second nucleic acid sequence of SEQ ID NO: 152;
the sense strand comprises a first nucleic acid sequence of SEQ ID NO: 312, and the antisense strand comprises a second nucleic acid sequence of SEQ ID NO: 366;
the sense strand comprises a first nucleic acid sequence of SEQ ID NO: 313, and the antisense strand comprises a second nucleic acid sequence of SEQ ID NO: 367;
the sense strand comprises a first nucleic acid sequence of SEQ ID NO: 314, and the antisense strand comprises a second nucleic acid sequence of SEQ ID NO: 368;
the sense strand comprises a first nucleic acid sequence of SEQ ID NO: 315, and the antisense strand comprises a second nucleic acid sequence of SEQ ID NO: 168; and
the sense strand comprises a first nucleic acid sequence of SEQ ID NO: 316, and the antisense strand comprises a second nucleic acid sequence of SEQ ID NO: 168;
wherein optionally one or more nucleotides of the sense strand and the antisense strand are independently modified nucleotides, and
wherein optionally one or more internucleotide linkages of the sense strand and the antisense strand are modified internucleotide linkages.

46. The PDE3B RNAi agent of claim 1, wherein the sense strand and the antisense strand comprise a pair of nucleic acid sequences selected from the group consisting of:
the sense strand comprises a first nucleic acid sequence of SEQ ID NO: 170, and the antisense strand comprises a second nucleic acid sequence of SEQ ID NO: 224;
the sense strand comprises a first nucleic acid sequence of SEQ ID NO: 171, and the antisense strand comprises a second nucleic acid sequence of SEQ ID NO: 225;
the sense strand comprises a first nucleic acid sequence of SEQ ID NO: 172, and the antisense strand comprises a second nucleic acid sequence of SEQ ID NO: 226;
the sense strand comprises a first nucleic acid sequence of SEQ ID NO: 173, and the antisense strand comprises a second nucleic acid sequence of SEQ ID NO: 227;
the sense strand comprises a first nucleic acid sequence of SEQ ID NO: 174, and the antisense strand comprises a second nucleic acid sequence of SEQ ID NO: 228;
the sense strand comprises a first nucleic acid sequence of SEQ ID NO: 175, and the antisense strand comprises a second nucleic acid sequence of SEQ ID NO: 229;
the sense strand comprises a first nucleic acid sequence of SEQ ID NO: 176, and the antisense strand comprises a second nucleic acid sequence of SEQ ID NO: 230;
the sense strand comprises a first nucleic acid sequence of SEQ ID NO: 177, and the antisense strand comprises a second nucleic acid sequence of SEQ ID NO: 231;
the sense strand comprises a first nucleic acid sequence of SEQ ID NO: 178, and the antisense strand comprises a second nucleic acid sequence of SEQ ID NO: 232;
the sense strand comprises a first nucleic acid sequence of SEQ ID NO: 179, and the antisense strand comprises a second nucleic acid sequence of SEQ ID NO: 233;
the sense strand comprises a first nucleic acid sequence of SEQ ID NO: 180, and the antisense strand comprises a second nucleic acid sequence of SEQ ID NO: 234;
the sense strand comprises a first nucleic acid sequence of SEQ ID NO: 181, and the antisense strand comprises a second nucleic acid sequence of SEQ ID NO: 235;
the sense strand comprises a first nucleic acid sequence of SEQ ID NO: 182, and the antisense strand comprises a second nucleic acid sequence of SEQ ID NO: 236;
the sense strand comprises a first nucleic acid sequence of SEQ ID NO: 183, and the antisense strand comprises a second nucleic acid sequence of SEQ ID NO: 237;
the sense strand comprises a first nucleic acid sequence of SEQ ID NO: 184, and the antisense strand comprises a second nucleic acid sequence of SEQ ID NO: 238;
the sense strand comprises a first nucleic acid sequence of SEQ ID NO: 185, and the antisense strand comprises a second nucleic acid sequence of SEQ ID NO: 239;
the sense strand comprises a first nucleic acid sequence of SEQ ID NO: 186, and the antisense strand comprises a second nucleic acid sequence of SEQ ID NO: 240;
the sense strand comprises a first nucleic acid sequence of SEQ ID NO: 187, and the antisense strand comprises a second nucleic acid sequence of SEQ ID NO: 241;
the sense strand comprises a first nucleic acid sequence of SEQ ID NO: 188, and the antisense strand comprises a second nucleic acid sequence of SEQ ID NO: 242;
the sense strand comprises a first nucleic acid sequence of SEQ ID NO: 189, and the antisense strand comprises a second nucleic acid sequence of SEQ ID NO: 243;
the sense strand comprises a first nucleic acid sequence of SEQ ID NO: 190, and the antisense strand comprises a second nucleic acid sequence of SEQ ID NO: 244;
the sense strand comprises a first nucleic acid sequence of SEQ ID NO: 191, and the antisense strand comprises a second nucleic acid sequence of SEQ ID NO: 245;
the sense strand comprises a first nucleic acid sequence of SEQ ID NO: 192, and the antisense strand comprises a second nucleic acid sequence of SEQ ID NO: 246;
the sense strand comprises a first nucleic acid sequence of SEQ ID NO: 193, and the antisense strand comprises a second nucleic acid sequence of SEQ ID NO: 247;
the sense strand comprises a first nucleic acid sequence of SEQ ID NO: 194, and the antisense strand comprises a second nucleic acid sequence of SEQ ID NO: 248;
the sense strand comprises a first nucleic acid sequence of SEQ ID NO: 195, and the antisense strand comprises a second nucleic acid sequence of SEQ ID NO: 249;
the sense strand comprises a first nucleic acid sequence of SEQ ID NO: 196, and the antisense strand comprises a second nucleic acid sequence of SEQ ID NO: 250;
the sense strand comprises a first nucleic acid sequence of SEQ ID NO: 197, and the antisense strand comprises a second nucleic acid sequence of SEQ ID NO: 251;
the sense strand comprises a first nucleic acid sequence of SEQ ID NO: 198, and the antisense strand comprises a second nucleic acid sequence of SEQ ID NO: 252;
the sense strand comprises a first nucleic acid sequence of SEQ ID NO: 199, and the antisense strand comprises a second nucleic acid sequence of SEQ ID NO: 253;
the sense strand comprises a first nucleic acid sequence of SEQ ID NO: 200, and the antisense strand comprises a second nucleic acid sequence of SEQ ID NO: 254;
the sense strand comprises a first nucleic acid sequence of SEQ ID NO: 201, and the antisense strand comprises a second nucleic acid sequence of SEQ ID NO: 255;
the sense strand comprises a first nucleic acid sequence of SEQ ID NO: 202, and the antisense strand comprises a second nucleic acid sequence of SEQ ID NO: 256;
the sense strand comprises a first nucleic acid sequence of SEQ ID NO: 203, and the antisense strand comprises a second nucleic acid sequence of SEQ ID NO: 257;
the sense strand comprises a first nucleic acid sequence of SEQ ID NO: 204, and the antisense strand comprises a second nucleic acid sequence of SEQ ID NO: 258;
the sense strand comprises a first nucleic acid sequence of SEQ ID NO: 205, and the antisense strand comprises a second nucleic acid sequence of SEQ ID NO: 259;
the sense strand comprises a first nucleic acid sequence of SEQ ID NO: 206, and the antisense strand comprises a second nucleic acid sequence of SEQ ID NO: 260;
the sense strand comprises a first nucleic acid sequence of SEQ ID NO: 207, and the antisense strand comprises a second nucleic acid sequence of SEQ ID NO: 261;
the sense strand comprises a first nucleic acid sequence of SEQ ID NO: 208, and the antisense strand comprises a second nucleic acid sequence of SEQ ID NO: 262;
the sense strand comprises a first nucleic acid sequence of SEQ ID NO: 209, and the antisense strand comprises a second nucleic acid sequence of SEQ ID NO: 263;
the sense strand comprises a first nucleic acid sequence of SEQ ID NO: 210, and the antisense strand comprises a second nucleic acid sequence of SEQ ID NO: 264;
the sense strand comprises a first nucleic acid sequence of SEQ ID NO: 211, and the antisense strand comprises a second nucleic acid sequence of SEQ ID NO: 265;
the sense strand comprises a first nucleic acid sequence of SEQ ID NO: 212, and the antisense strand comprises a second nucleic acid sequence of SEQ ID NO: 266;
the sense strand comprises a first nucleic acid sequence of SEQ ID NO: 213, and the antisense strand comprises a second nucleic acid sequence of SEQ ID NO: 267;
the sense strand comprises a first nucleic acid sequence of SEQ ID NO: 214, and the antisense strand comprises a second nucleic acid sequence of SEQ ID NO: 268;
the sense strand comprises a first nucleic acid sequence of SEQ ID NO: 215, and the antisense strand comprises a second nucleic acid sequence of SEQ ID NO: 240;
the sense strand comprises a first nucleic acid sequence of SEQ ID NO: 216, and the antisense strand comprises a second nucleic acid sequence of SEQ ID NO: 236;
the sense strand comprises a first nucleic acid sequence of SEQ ID NO: 217, and the antisense strand comprises a second nucleic acid sequence of SEQ ID NO: 224;
the sense strand comprises a first nucleic acid sequence of SEQ ID NO: 218, and the antisense strand comprises a second nucleic acid sequence of SEQ ID NO: 234;
the sense strand comprises a first nucleic acid sequence of SEQ ID NO: 219, and the antisense strand comprises a second nucleic acid sequence of SEQ ID NO: 233;
the sense strand comprises a first nucleic acid sequence of SEQ ID NO: 220, and the antisense strand comprises a second nucleic acid sequence of SEQ ID NO: 227;
the sense strand comprises a first nucleic acid sequence of SEQ ID NO: 221, and the antisense strand comprises a second nucleic acid sequence of SEQ ID NO: 239;
the sense strand comprises a first nucleic acid sequence of SEQ ID NO: 222, and the antisense strand comprises a second nucleic acid sequence of SEQ ID NO: 231;
the sense strand comprises a first nucleic acid sequence of SEQ ID NO: 223, and the antisense strand comprises a second nucleic acid sequence of SEQ ID NO: 230;
the sense strand comprises a first nucleic acid sequence of SEQ ID NO: 187, and the antisense strand comprises a second nucleic acid sequence of SEQ ID NO: 464;
the sense strand comprises a first nucleic acid sequence of SEQ ID NO: 188, and the antisense strand comprises a second nucleic acid sequence of SEQ ID NO: 465;
the sense strand comprises a first nucleic acid sequence of SEQ ID NO: 194, and the antisense strand comprises a second nucleic acid sequence of SEQ ID NO: 466;
the sense strand comprises a first nucleic acid sequence of SEQ ID NO: 197, and the antisense strand comprises a second nucleic acid sequence of SEQ ID NO: 467;
the sense strand comprises a first nucleic acid sequence of SEQ ID NO: 205, and the antisense strand comprises a second nucleic acid sequence of SEQ ID NO: 468;
the sense strand comprises a first nucleic acid sequence of SEQ ID NO: 213, and the antisense strand comprises a second nucleic acid sequence of SEQ ID NO: 469;
the sense strand comprises a first nucleic acid sequence of SEQ ID NO: 369, and the antisense strand comprises a second nucleic acid sequence of SEQ ID NO: 470;
the sense strand comprises a first nucleic acid sequence of SEQ ID NO: 370, and the antisense strand comprises a second nucleic acid sequence of SEQ ID NO: 471;
the sense strand comprises a first nucleic acid sequence of SEQ ID NO: 371, and the antisense strand comprises a second nucleic acid sequence of SEQ ID NO: 472;
the sense strand comprises a first nucleic acid sequence of SEQ ID NO: 372, and the antisense strand comprises a second nucleic acid sequence of SEQ ID NO: 473;
the sense strand comprises a first nucleic acid sequence of SEQ ID NO: 373, and the antisense strand comprises a second nucleic acid sequence of SEQ ID NO: 474;
the sense strand comprises a first nucleic acid sequence of SEQ ID NO: 374, and the antisense strand comprises a second nucleic acid sequence of SEQ ID NO: 475;
the sense strand comprises a first nucleic acid sequence of SEQ ID NO: 375, and the antisense strand comprises a second nucleic acid sequence of SEQ ID NO: 476;
the sense strand comprises a first nucleic acid sequence of SEQ ID NO: 376, and the antisense strand comprises a second nucleic acid sequence of SEQ ID NO: 477;
the sense strand comprises a first nucleic acid sequence of SEQ ID NO: 377, and the antisense strand comprises a second nucleic acid sequence of SEQ ID NO: 478;
the sense strand comprises a first nucleic acid sequence of SEQ ID NO: 378, and the antisense strand comprises a second nucleic acid sequence of SEQ ID NO: 479;
the sense strand comprises a first nucleic acid sequence of SEQ ID NO: 378, and the antisense strand comprises a second nucleic acid sequence of SEQ ID NO: 480;
the sense strand comprises a first nucleic acid sequence of SEQ ID NO: 379, and the antisense strand comprises a second nucleic acid sequence of SEQ ID NO: 481;
the sense strand comprises a first nucleic acid sequence of SEQ ID NO: 380, and the antisense strand comprises a second nucleic acid sequence of SEQ ID NO: 482;
the sense strand comprises a first nucleic acid sequence of SEQ ID NO: 381, and the antisense strand comprises a second nucleic acid sequence of SEQ ID NO: 483;
the sense strand comprises a first nucleic acid sequence of SEQ ID NO: 382, and the antisense strand comprises a second nucleic acid sequence of SEQ ID NO: 484;
the sense strand comprises a first nucleic acid sequence of SEQ ID NO: 383, and the antisense strand comprises a second nucleic acid sequence of SEQ ID NO: 485;
the sense strand comprises a first nucleic acid sequence of SEQ ID NO: 384, and the antisense strand comprises a second nucleic acid sequence of SEQ ID NO: 486;
the sense strand comprises a first nucleic acid sequence of SEQ ID NO: 385, and the antisense strand comprises a second nucleic acid sequence of SEQ ID NO: 487;
the sense strand comprises a first nucleic acid sequence of SEQ ID NO: 386, and the antisense strand comprises a second nucleic acid sequence of SEQ ID NO: 488;
the sense strand comprises a first nucleic acid sequence of SEQ ID NO: 387, and the antisense strand comprises a second nucleic acid sequence of SEQ ID NO: 489;
the sense strand comprises a first nucleic acid sequence of SEQ ID NO: 388, and the antisense strand comprises a second nucleic acid sequence of SEQ ID NO: 490;
the sense strand comprises a first nucleic acid sequence of SEQ ID NO: 389, and the antisense strand comprises a second nucleic acid sequence of SEQ ID NO: 491;
the sense strand comprises a first nucleic acid sequence of SEQ ID NO: 390, and the antisense strand comprises a second nucleic acid sequence of SEQ ID NO: 492;
the sense strand comprises a first nucleic acid sequence of SEQ ID NO: 391, and the antisense strand comprises a second nucleic acid sequence of SEQ ID NO: 493;
the sense strand comprises a first nucleic acid sequence of SEQ ID NO: 392, and the antisense strand comprises a second nucleic acid sequence of SEQ ID NO: 494;
the sense strand comprises a first nucleic acid sequence of SEQ ID NO: 393, and the antisense strand comprises a second nucleic acid sequence of SEQ ID NO: 495;
the sense strand comprises a first nucleic acid sequence of SEQ ID NO: 394, and the antisense strand comprises a second nucleic acid sequence of SEQ ID NO: 496;
the sense strand comprises a first nucleic acid sequence of SEQ ID NO: 395, and the antisense strand comprises a second nucleic acid sequence of SEQ ID NO: 497;
the sense strand comprises a first nucleic acid sequence of SEQ ID NO: 396, and the antisense strand comprises a second nucleic acid sequence of SEQ ID NO: 498;
the sense strand comprises a first nucleic acid sequence of SEQ ID NO: 397, and the antisense strand comprises a second nucleic acid sequence of SEQ ID NO: 499;
the sense strand comprises a first nucleic acid sequence of SEQ ID NO: 398, and the antisense strand comprises a second nucleic acid sequence of SEQ ID NO: 500;
the sense strand comprises a first nucleic acid sequence of SEQ ID NO: 399, and the antisense strand comprises a second nucleic acid sequence of SEQ ID NO: 501;
the sense strand comprises a first nucleic acid sequence of SEQ ID NO: 400, and the antisense strand comprises a second nucleic acid sequence of SEQ ID NO: 502;
the sense strand comprises a first nucleic acid sequence of SEQ ID NO: 401, and the antisense strand comprises a second nucleic acid sequence of SEQ ID NO: 503;
the sense strand comprises a first nucleic acid sequence of SEQ ID NO: 402, and the antisense strand comprises a second nucleic acid sequence of SEQ ID NO: 504;
the sense strand comprises a first nucleic acid sequence of SEQ ID NO: 403, and the antisense strand comprises a second nucleic acid sequence of SEQ ID NO: 505;
the sense strand comprises a first nucleic acid sequence of SEQ ID NO: 404, and the antisense strand comprises a second nucleic acid sequence of SEQ ID NO: 506;
the sense strand comprises a first nucleic acid sequence of SEQ ID NO: 405, and the antisense strand comprises a second nucleic acid sequence of SEQ ID NO: 507;
the sense strand comprises a first nucleic acid sequence of SEQ ID NO: 406, and the antisense strand comprises a second nucleic acid sequence of SEQ ID NO: 508;
the sense strand comprises a first nucleic acid sequence of SEQ ID NO: 407, and the antisense strand comprises a second nucleic acid sequence of SEQ ID NO: 509;
the sense strand comprises a first nucleic acid sequence of SEQ ID NO: 408, and the antisense strand comprises a second nucleic acid sequence of SEQ ID NO: 510;
the sense strand comprises a first nucleic acid sequence of SEQ ID NO: 409, and the antisense strand comprises a second nucleic acid sequence of SEQ ID NO: 511;
the sense strand comprises a first nucleic acid sequence of SEQ ID NO: 410, and the antisense strand comprises a second nucleic acid sequence of SEQ ID NO: 512;
the sense strand comprises a first nucleic acid sequence of SEQ ID NO: 411, and the antisense strand comprises a second nucleic acid sequence of SEQ ID NO: 513;
the sense strand comprises a first nucleic acid sequence of SEQ ID NO: 412, and the antisense strand comprises a second nucleic acid sequence of SEQ ID NO: 541;
the sense strand comprises a first nucleic acid sequence of SEQ ID NO: 413, and the antisense strand comprises a second nucleic acid sequence of SEQ ID NO: 515;
the sense strand comprises a first nucleic acid sequence of SEQ ID NO: 414, and the antisense strand comprises a second nucleic acid sequence of SEQ ID NO: 516;
the sense strand comprises a first nucleic acid sequence of SEQ ID NO: 415, and the antisense strand comprises a second nucleic acid sequence of SEQ ID NO: 517;
the sense strand comprises a first nucleic acid sequence of SEQ ID NO: 416, and the antisense strand comprises a second nucleic acid sequence of SEQ ID NO: 518;
the sense strand comprises a first nucleic acid sequence of SEQ ID NO: 417, and the antisense strand comprises a second nucleic acid sequence of SEQ ID NO: 519;
the sense strand comprises a first nucleic acid sequence of SEQ ID NO: 418, and the antisense strand comprises a second nucleic acid sequence of SEQ ID NO: 520;
the sense strand comprises a first nucleic acid sequence of SEQ ID NO: 419, and the antisense strand comprises a second nucleic acid sequence of SEQ ID NO: 521;
the sense strand comprises a first nucleic acid sequence of SEQ ID NO: 420, and the antisense strand comprises a second nucleic acid sequence of SEQ ID NO: 522;
the sense strand comprises a first nucleic acid sequence of SEQ ID NO: 421, and the antisense strand comprises a second nucleic acid sequence of SEQ ID NO: 523;
the sense strand comprises a first nucleic acid sequence of SEQ ID NO: 422, and the antisense strand comprises a second nucleic acid sequence of SEQ ID NO: 524;
the sense strand comprises a first nucleic acid sequence of SEQ ID NO: 423, and the antisense strand comprises a second nucleic acid sequence of SEQ ID NO: 525;
the sense strand comprises a first nucleic acid sequence of SEQ ID NO: 424, and the antisense strand comprises a second nucleic acid sequence of SEQ ID NO: 526;
the sense strand comprises a first nucleic acid sequence of SEQ ID NO: 425, and the antisense strand comprises a second nucleic acid sequence of SEQ ID NO: 527;
the sense strand comprises a first nucleic acid sequence of SEQ ID NO: 426, and the antisense strand comprises a second nucleic acid sequence of SEQ ID NO: 528;
the sense strand comprises a first nucleic acid sequence of SEQ ID NO: 427, and the antisense strand comprises a second nucleic acid sequence of SEQ ID NO: 529;
the sense strand comprises a first nucleic acid sequence of SEQ ID NO: 428, and the antisense strand comprises a second nucleic acid sequence of SEQ ID NO: 530;
the sense strand comprises a first nucleic acid sequence of SEQ ID NO: 429, and the antisense strand comprises a second nucleic acid sequence of SEQ ID NO: 531;
the sense strand comprises a first nucleic acid sequence of SEQ ID NO: 430, and the antisense strand comprises a second nucleic acid sequence of SEQ ID NO: 532;
the sense strand comprises a first nucleic acid sequence of SEQ ID NO: 431, and the antisense strand comprises a second nucleic acid sequence of SEQ ID NO: 533;
the sense strand comprises a first nucleic acid sequence of SEQ ID NO: 432, and the antisense strand comprises a second nucleic acid sequence of SEQ ID NO: 534;
the sense strand comprises a first nucleic acid sequence of SEQ ID NO: 433, and the antisense strand comprises a second nucleic acid sequence of SEQ ID NO: 535;
the sense strand comprises a first nucleic acid sequence of SEQ ID NO: 434, and the antisense strand comprises a second nucleic acid sequence of SEQ ID NO: 536;
the sense strand comprises a first nucleic acid sequence of SEQ ID NO: 435, and the antisense strand comprises a second nucleic acid sequence of SEQ ID NO: 537;
the sense strand comprises a first nucleic acid sequence of SEQ ID NO: 436, and the antisense strand comprises a second nucleic acid sequence of SEQ ID NO: 538;
the sense strand comprises a first nucleic acid sequence of SEQ ID NO: 437, and the antisense strand comprises a second nucleic acid sequence of SEQ ID NO: 539;
the sense strand comprises a first nucleic acid sequence of SEQ ID NO: 438, and the antisense strand comprises a second nucleic acid sequence of SEQ ID NO: 540;
the sense strand comprises a first nucleic acid sequence of SEQ ID NO: 439, and the antisense strand comprises a second nucleic acid sequence of SEQ ID NO: 541;
the sense strand comprises a first nucleic acid sequence of SEQ ID NO: 369, and the antisense strand comprises a second nucleic acid sequence of SEQ ID NO: 542;
the sense strand comprises a first nucleic acid sequence of SEQ ID NO: 440, and the antisense strand comprises a second nucleic acid sequence of SEQ ID NO: 543;
the sense strand comprises a first nucleic acid sequence of SEQ ID NO: 369, and the antisense strand comprises a second nucleic acid sequence of SEQ ID NO: 544;
the sense strand comprises a first nucleic acid sequence of SEQ ID NO: 369, and the antisense strand comprises a second nucleic acid sequence of SEQ ID NO: 545;
the sense strand comprises a first nucleic acid sequence of SEQ ID NO: 441, and the antisense strand comprises a second nucleic acid sequence of SEQ ID NO: 546;
the sense strand comprises a first nucleic acid sequence of SEQ ID NO: 442, and the antisense strand comprises a second nucleic acid sequence of SEQ ID NO: 547;
the sense strand comprises a first nucleic acid sequence of SEQ ID NO: 443, and the antisense strand comprises a second nucleic acid sequence of SEQ ID NO: 470;
the sense strand comprises a first nucleic acid sequence of SEQ ID NO: 444, and the antisense strand comprises a second nucleic acid sequence of SEQ ID NO: 470;
the sense strand comprises a first nucleic acid sequence of SEQ ID NO: 445, and the antisense strand comprises a second nucleic acid sequence of SEQ ID NO: 470;
the sense strand comprises a first nucleic acid sequence of SEQ ID NO: 446, and the antisense strand comprises a second nucleic acid sequence of SEQ ID NO: 548;
the sense strand comprises a first nucleic acid sequence of SEQ ID NO: 446, and the antisense strand comprises a second nucleic acid sequence of SEQ ID NO: 549;
the sense strand comprises a first nucleic acid sequence of SEQ ID NO: 446, and the antisense strand comprises a second nucleic acid sequence of SEQ ID NO: 476;
the sense strand comprises a first nucleic acid sequence of SEQ ID NO: 447, and the antisense strand comprises a second nucleic acid sequence of SEQ ID NO: 550;
the sense strand comprises a first nucleic acid sequence of SEQ ID NO: 375, and the antisense strand comprises a second nucleic acid sequence of SEQ ID NO: 551;
the sense strand comprises a first nucleic acid sequence of SEQ ID NO: 375, and the antisense strand comprises a second nucleic acid sequence of SEQ ID NO: 552;
the sense strand comprises a first nucleic acid sequence of SEQ ID NO: 375, and the antisense strand comprises a second nucleic acid sequence of SEQ ID NO: 553;
the sense strand comprises a first nucleic acid sequence of SEQ ID NO: 448, and the antisense strand comprises a second nucleic acid sequence of SEQ ID NO: 554;
the sense strand comprises a first nucleic acid sequence of SEQ ID NO: 449, and the antisense strand comprises a second nucleic acid sequence of SEQ ID NO: 476;
the sense strand comprises a first nucleic acid sequence of SEQ ID NO: 375, and the antisense strand comprises a second nucleic acid sequence of SEQ ID NO: 555;
the sense strand comprises a first nucleic acid sequence of SEQ ID NO: 450, and the antisense strand comprises a second nucleic acid sequence of SEQ ID NO: 556;
the sense strand comprises a first nucleic acid sequence of SEQ ID NO: 451, and the antisense strand comprises a second nucleic acid sequence of SEQ ID NO: 476;
the sense strand comprises a first nucleic acid sequence of SEQ ID NO: 375, and the antisense strand comprises a second nucleic acid sequence of SEQ ID NO: 557;
the sense strand comprises a first nucleic acid sequence of SEQ ID NO: 446, and the antisense strand comprises a second nucleic acid sequence of SEQ ID NO: 557;
the sense strand comprises a first nucleic acid sequence of SEQ ID NO: 375, and the antisense strand comprises a second nucleic acid sequence of SEQ ID NO: 558;
the sense strand comprises a first nucleic acid sequence of SEQ ID NO: 378, and the antisense strand comprises a second nucleic acid sequence of SEQ ID NO: 559;
the sense strand comprises a first nucleic acid sequence of SEQ ID NO: 452, and the antisense strand comprises a second nucleic acid sequence of SEQ ID NO: 560;
the sense strand comprises a first nucleic acid sequence of SEQ ID NO: 378, and the antisense strand comprises a second nucleic acid sequence of SEQ ID NO: 561;
the sense strand comprises a first nucleic acid sequence of SEQ ID NO: 453, and the antisense strand comprises a second nucleic acid sequence of SEQ ID NO: 562;
the sense strand comprises a first nucleic acid sequence of SEQ ID NO: 378, and the antisense strand comprises a second nucleic acid sequence of SEQ ID NO: 563;
the sense strand comprises a first nucleic acid sequence of SEQ ID NO: 454, and the antisense strand comprises a second nucleic acid sequence of SEQ ID NO: 480;
the sense strand comprises a first nucleic acid sequence of SEQ ID NO: 378, and the antisense strand comprises a second nucleic acid sequence of SEQ ID NO: 564;
the sense strand comprises a first nucleic acid sequence of SEQ ID NO: 378, and the antisense strand comprises a second nucleic acid sequence of SEQ ID NO: 565;
the sense strand comprises a first nucleic acid sequence of SEQ ID NO: 455, and the antisense strand comprises a second nucleic acid sequence of SEQ ID NO: 480;
the sense strand comprises a first nucleic acid sequence of SEQ ID NO: 456, and the antisense strand comprises a second nucleic acid sequence of SEQ ID NO: 480;
the sense strand comprises a first nucleic acid sequence of SEQ ID NO: 457, and the antisense strand comprises a second nucleic acid sequence of SEQ ID NO: 566;
the sense strand comprises a first nucleic acid sequence of SEQ ID NO: 458, and the antisense strand comprises a second nucleic acid sequence of SEQ ID NO: 478;
the sense strand comprises a first nucleic acid sequence of SEQ ID NO: 459, and the antisense strand comprises a second nucleic acid sequence of SEQ ID NO: 567;
the sense strand comprises a first nucleic acid sequence of SEQ ID NO: 460, and the antisense strand comprises a second nucleic acid sequence of SEQ ID NO: 478;
the sense strand comprises a first nucleic acid sequence of SEQ ID NO: 461, and the antisense strand comprises a second nucleic acid sequence of SEQ ID NO: 568;
the sense strand comprises a first nucleic acid sequence of SEQ ID NO: 462, and the antisense strand comprises a second nucleic acid sequence of SEQ ID NO: 478;
the sense strand comprises a first nucleic acid sequence of SEQ ID NO: 463, and the antisense strand comprises a second nucleic acid sequence of SEQ ID NO: 478;
the sense strand comprises a first nucleic acid sequence of SEQ ID NO: 376, and the antisense strand comprises a second nucleic acid sequence of SEQ ID NO: 569;
the sense strand comprises a first nucleic acid sequence of SEQ ID NO: 377, and the antisense strand comprises a second nucleic acid sequence of SEQ ID NO: 570;
the sense strand comprises a first nucleic acid sequence of SEQ ID NO: 378, and the antisense strand comprises a second nucleic acid sequence of SEQ ID NO: 571;
the sense strand comprises a first nucleic acid sequence of SEQ ID NO: 375, and the antisense strand comprises a second nucleic acid sequence of SEQ ID NO: 572;
the sense strand comprises a first nucleic acid sequence of SEQ ID NO: 374, and the antisense strand comprises a second nucleic acid sequence of SEQ ID NO: 573;
the sense strand comprises a first nucleic acid sequence of SEQ ID NO: 373, and the antisense strand comprises a second nucleic acid sequence of SEQ ID NO: 574;
the sense strand comprises a first nucleic acid sequence of SEQ ID NO: 372, and the antisense strand comprises a second nucleic acid sequence of SEQ ID NO: 575;
the sense strand comprises a first nucleic acid sequence of SEQ ID NO: 369, and the antisense strand comprises a second nucleic acid sequence of SEQ ID NO: 576;
the sense strand comprises a first nucleic acid sequence of SEQ ID NO: 577, and the antisense strand comprises a second nucleic acid sequence of SEQ ID NO: 478;
the sense strand comprises a first nucleic acid sequence of SEQ ID NO: 578, and the antisense strand comprises a second nucleic acid sequence of SEQ ID NO: 569;
the sense strand comprises a first nucleic acid sequence of SEQ ID NO: 579, and the antisense strand comprises a second nucleic acid sequence of SEQ ID NO: 571;
the sense strand comprises a first nucleic acid sequence of SEQ ID NO: 580, and the antisense strand comprises a second nucleic acid sequence of SEQ ID NO: 572;
the sense strand comprises a first nucleic acid sequence of SEQ ID NO: 581, and the antisense strand comprises a second nucleic acid sequence of SEQ ID NO: 573;
the sense strand comprises a first nucleic acid sequence of SEQ ID NO: 582, and the antisense strand comprises a second nucleic acid sequence of SEQ ID NO: 574;
the sense strand comprises a first nucleic acid sequence of SEQ ID NO: 583, and the antisense strand comprises a second nucleic acid sequence of SEQ ID NO: 575; and
the sense strand comprises a first nucleic acid sequence of SEQ ID NO: 584, and the antisense strand comprises a second nucleic acid sequence of SEQ ID NO: 576.

47. A PDE3B RNAi agent of Formula: 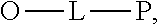
wherein O comprises a double stranded RNA (dsRNA) of claim 1, comprising a sense stand and an antisense strand;
wherein L is a linker comprising the formula: 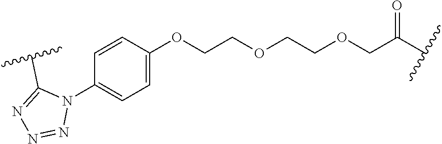
wherein P is an NPR-C-binding peptide comprising SEQ ID NO: 270.

48. (canceled)

49. The PDE3B RNAi agent of claim 47, wherein:
the sense strand comprises a first nucleic acid sequence of SEQ ID NO: 112, and the antisense strand comprises a second nucleic acid sequence of SEQ ID NO: 157;
the sense strand comprises a first nucleic acid sequence of SEQ ID NO: 123, and the antisense strand comprises a second nucleic acid sequence of SEQ ID NO: 168;
the sense strand comprises a first nucleic acid sequence of SEQ ID NO: 107, and the antisense strand comprises a second nucleic acid sequence of SEQ ID NO: 321;
the sense strand comprises a first nucleic acid sequence of SEQ ID NO: 111, and the antisense strand comprises a second nucleic acid sequence of SEQ ID NO: 156;
the sense strand comprises a first nucleic acid sequence of SEQ ID NO: 105, and the antisense strand comprises a second nucleic acid sequence of SEQ ID NO: 150;
the sense strand comprises a first nucleic acid sequence of SEQ ID NO: 102, and the antisense strand comprises a second nucleic acid sequence of SEQ ID NO: 147;
the sense strand comprises a first nucleic acid sequence of SEQ ID NO: 104, and the antisense strand comprises a second nucleic acid sequence of SEQ ID NO: 149; or
the sense strand comprises a first nucleic acid sequence of SEQ ID NO: 97, and the antisense strand comprises a second nucleic acid sequence of SEQ ID NO: 142.

50. The PDE3B RNAi agent of claim 47, wherein the antisense strand is modified with 2′-fluoro at each of positions 2, 5, 7, 14, and 16; and wherein all other positions of the antisense strand are modified with 2′-OMe.

51. (canceled)

52. The PDE3B RNAi agent of claim 47, wherein the antisense strand comprises phosphorothioate linkages between positions 1 and 2, 2 and 3, 20 and 21, 21 and 22, and 22 and 23.

53. The PDE3B RNAi agent of claim 47, wherein the antisense comprises a vinylphosphonate moiety conjugated to the 5′ end.

54. The PDE3B RNAi agent of claim 47, wherein the sense strand is modified with 2′-fluoro at each of positions 9, 10, and 11, and wherein all other positions of the sense strand are modified with 2′-OMe.

55. (canceled)

56. The PDE3B RNAi agent of claim 47, wherein the sense strand comprises phosphorothioate linkages between positions 1 and 2, 19 and 20, and 20 and 21.

57. The PDE3B RNAi agent of claim 56, wherein the sense strand comprises the inverted abasic moiety conjugated at the 5′ end, wherein the inverted abasic moiety is conjugated to the sense strand by a phosphorothioate linkage.

58. The PDE3B RNAi agent of claim 47, wherein the linker L is attached to a 3′ end of the sense strand.

59. The PDE3B RNAi agent of claim 47, wherein the linker is of formula: 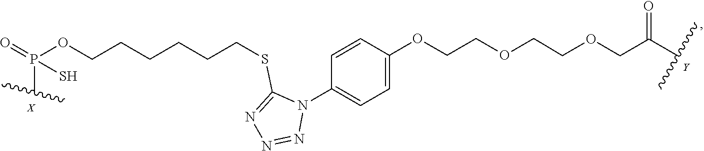
wherein X represents a position to which the 3′ end of the sense strand of O is conjugated, and
wherein Y represents a position to which an N-terminal end of P is conjugated.

60.-88. (canceled)

89. A pharmaceutical composition comprising the PDE3B RNAi agent of claim 1, and a pharmaceutically acceptable carrier.

90. A method of treating a disease or condition of adipose tissue in a patient in need thereof, comprising administering to the patient an effective amount of the PDE3B RNAi agent of claim 1.

91. The method of claim 90, wherein the disease or condition is obesity or obesity-related comorbidity.

92. The method of claim 90, wherein the conjugate or pharmaceutical composition is administered intravenously or subcutaneously.

93.-97. (canceled)

98. A method of delivering an oligonucleotide to an adipose tissue, comprising administering to a subject PDE3B RNAi agent of claim 1.
